(19) United States
(12) Patent Application Publication
(10) Pub. No.: US 2024/0165250 A1
(43) Pub. Date: May 23, 2024

(54) Bifunctional Folate Receptor Binding Compounds

(71) Applicant: Lycia Therapeutics, Inc., South San Francisco, CA (US)

(72) Inventors: Jason G. Lewis, Castro Valley, CA (US); Eric D. Turtle, Belmont, CA (US); Brett Bradley Busch, San Diego, CA (US); Justin Thomas Ernst, San Diego, CA (US); Garrick K. Packard, San Diego, CA (US); Matthew James Shurtleff, San Diego, CA (US)

(21) Appl. No.: 18/271,230

(22) PCT Filed: Jan. 10, 2022
(86) PCT No.: PCT/US2022/011857

§ 371 Date: Jul. 6, 2023

Related U.S. Application Data
(60) Provisional application No. 63/135,510, filed on Jan. 8, 2021.
(60) Provisional application No. 63/214,774, filed on Jun. 24, 2021.

(51) Int. Cl. A61K 47/68; A61K 31/517; A61K 31/519; C07D 239/88; C07D 403/06; C07D 409/12; C07D 487/04

(52) U.S. Cl. CPC A61K 47/6801; A61K 31/517; A61K 31/519; A61K 47/6849; C07D 239/88; C07D 403/06; C07D 409/12; C07D 487/04

(57) ABSTRACT

The present disclosure provides a class of compounds including a ligand moiety that specifically binds to a cell surface receptor, such as a folate receptor. The cell surface folate binding compound can trigger the receptor to internalize into the cell a bound compound. The ligand moieties of this disclosure can be linked to a variety of moieties of interest without impacting the specific binding to, and function of, the cell surface receptor, e.g., folate receptor. Also provided are compounds that are conjugates of the ligand moieties linked to a biomolecule, such as an antibody, which conjugates can harness cellular pathways to remove specific proteins of interest from the cell surface or from the extracellular milieu. Also provided herein are methods of using the conjugates to target a polypeptide of interest for sequestration and/or lysosomal degradation.

### 1. CROSS REFERENCE TO RELATED APPLICATIONS

This application claims the benefit of U.S. Provisional Application No. 63/135,510, filed Jan. 8, 2021, and U.S. Provisional Application No. 63/214,774, filed Jun. 24, 2021, which are hereby incorporated in their entirety by reference.

### 2. INTRODUCTION

Folate receptors on cells can bind ligands such as folate and reduced folic acid derivatives to mediate delivery of molecules containing such ligands into the interior of the cells. Human proteins from the family include folate receptor 1, folate receptor 2, and folate receptor gamma. Folate-based diagnostic and therapeutic agents are used intracellularly.

Many therapeutics act by binding a functionally important site on a target protein, thereby modulating the activity of that protein, or by recruiting immune effectors, as with many monoclonal antibody drugs, to act upon the target protein. However, there is an untapped reservoir of medically important human proteins that are considered to be “undruggable” because these proteins are not readily amenable to currently available therapeutic targeting approaches. Thus, there is a need for therapies that can target a wider range of proteins.

Thus, there is a need for therapies that can target a wider range of proteins. For example, there is a need for therapies that can harness a subject's own cellular pathways to remove specific proteins of interest from the cell surface or the extracellular milieu.

### 3. SUMMARY OF THE INVENTION

The present disclosure provides a class of compounds including a ligand moiety that specifically binds to a cell surface receptor such as a folate receptor. The cell surface folate binding compound can trigger the receptor to internalize into the cell a bound compound. The ligand moieties of this disclosure can be linked to a variety of moieties of interest without impacting the specific binding to, and function of, the cell surface folate receptor, and provide for internalization of the linked moieties of interest into the cell. In some embodiments, the linked moiety of interest is itself targeted for delivery or internalization in the cell. Also provided are compounds that are conjugates of the ligand moieties linked to a biomolecule, such as an antibody, which conjugates can harness cellular pathways to remove specific proteins of interest from the cell surface or from the extracellular milieu. For example, the conjugates described herein may sequester and/or degrade a target molecule of interest in a cell's lysosome. Also provided herein are compositions comprising such conjugates and methods of using the conjugates to target a polypeptide of interest for sequestration and/or lysosomal degradation, and methods of using the conjugates to treat disorders or disease.

A first aspect of this disclosure includes a cell surface folate receptor binding compound of formula (I):

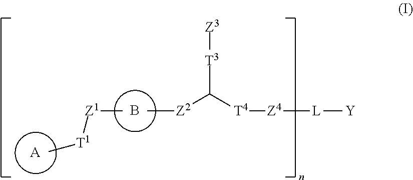

or a salt thereof,
wherein:
T¹ is an optionally substituted (C₁-C₃)alkylene;
Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;
B is a ring system selected from optionally substituted aryl, optionally substituted heteroaryl, optionally substituted heterocycle, optionally substituted cycloalkyl, and optionally substituted bridged bicycle;
Z² is absent, or a linking moiety selected from optionally substituted amide, optionally substituted urea, optionally substituted sulfonamide, optionally substituted thiourea, —NR²¹—, —O—, —S—, and optionally substituted (C₁-C₆)alkylene;
Z³ is carboxyl or carboxyl bioisostere, or a prodrug thereof;
T³ is absent, or is selected from optionally substituted (C₁-C₆)alkylene;
T⁴ is optionally substituted (C₁-C₆)alkylene (e.g., —CH₂CH₂—), or is absent;
Z⁴ is a linking moiety (e.g., a linking moiety selected from ester, amide, urea, thiourea, amine, sulfonamide, ether, optionally substituted aryl, optionally substituted heterocycle, and optionally substituted heteroaryl);
each R²¹ is independently selected from H, and optionally substituted (C₁-C₆)alkyl;
n is 1 to 100;
L is a linker;
Y is a moiety of interest; and
A is a ring system of formula (II):

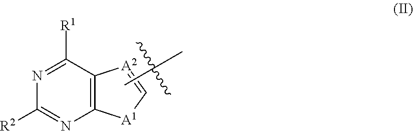

or a tautomer thereof,
wherein:
R¹ and R² are independently selected from OH, NR²¹, and optionally substituted (C₁-C₆)alkyl (e.g., —CH₃ or —CH₂OH);
A¹ is selected from —N═CR³—, —CR³═N—, —CR³═CR³—, NR²¹, S, O, and C(R⁴)₂;
A² is selected from N, and CR³;
each R³ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each R⁴ is independently selected from H, halogen (e.g., F), and optionally substituted (C₁-C₆)alkyl;
with the proviso that at least one of following applies:
1) T³ is optionally substituted (C₁-C₆)alkylene (e.g., —CH₂CH₂—);
2) L is a non-cleavable linker and Y is an extracellular target-binding moiety;
3) when A is of formula (II-A) or (II-A′), or a tautomer thereof:

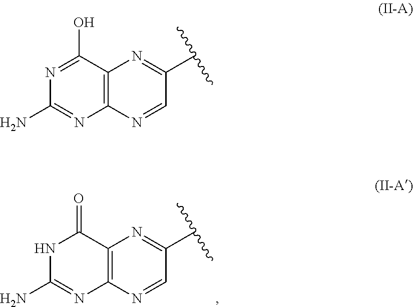 [SMILES: *c1cnc2nc(N)nc(O)c2n1 ; *c1cnc2nc(N)[nH]c(=O)c2n1]

then Z¹ is not NR²¹, and/or B is not 1,4-linked phenyl;
4) when A is of formula (II-B), or a tautomer thereof:

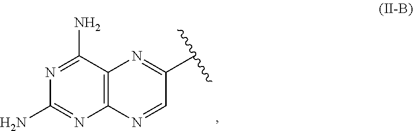 [SMILES: *c1cnc2nc(N)nc(N)c2n1]

then Z¹ is not NR²¹, and/or B is not 1,4-linked phenyl; and/or
5) when A is of formula (II-C) or (II-C′), or a tautomer thereof:

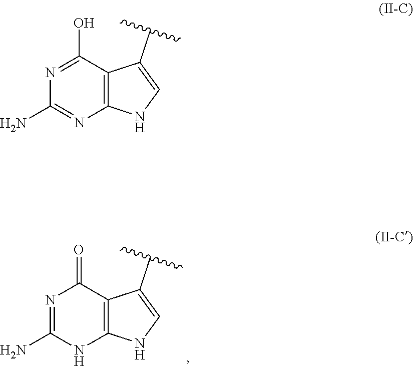 [SMILES: *c1c[nH]c2nc(N)nc(O)c12 ; *c1c[nH]c2[nH]c(N)nc(=O)c12]

then T¹-Z¹ is not —CH₂CH₂—, and/or B is not phenyl.

In some embodiments of formula (I), Y is antibody or antibody fragment that specifically binds the target protein and the compound is of formula (VIIIa):

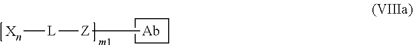

or a pharmaceutically acceptable salt thereof,
wherein:
n is 1 to 20;
m1 is an average loading of 1 to 80;
each X is a moiety that binds to a cell surface folate receptor;
each L is a linker;
each Z is a residual moiety resulting from the covalent linkage of a chemoselective ligation group to a compatible group of Ab; and
Ab is the antibody or antibody fragment that specifically binds the target protein.

A second aspect of this disclosure includes a method of internalizing a target protein in a cell comprising a cell surface receptor selected from a folate receptor, where the method includes contacting a cellular sample comprising the cell and the target protein with an effective amount of a compound (e.g., as described herein) that specifically binds the target protein and specifically binds the cell surface receptor to facilitate cellular uptake of the target protein.

A third aspect of this disclosure includes a method of reducing levels of a target protein in a biological system, where the method includes contacting the biological system with an effective amount of a compound (e.g., as described herein) that specifically binds the target protein and specifically binds a cell surface receptor of cells in the biological system to facilitate cellular uptake and degradation of the target protein.

A fourth aspect of this disclosure includes a method of treating a disease or disorder associated with a target protein, where the method includes administering to a subject in need thereof an effective amount of a compound (e.g., as described herein), wherein the compound specifically binds the target protein.

In certain embodiments the disease or disorder is an inflammatory disease, an autoimmune disease, or a cancer.

### 4. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows surface plasmon resonance (SPR) sensorgrams measuring 1:1 binding of compound (1-21) to folate receptor 2 (FOLR2). Further details are described in Example 51 of the experimental section.

FIGS. 2A-2C show SPR sensorgrams which illustrate binding kinetics of exemplary compounds to TNFα trimer. SPR binding of compound (1-16) to TNFα trimer (FIG. 2A), compound (1-21) to TNFα trimer (FIG. 2B), and compound (1-25) to TNFα trimer (FIG. 2C). Further details are described in Example 52 of the experimental section.

FIGS. 3A-3B show SPR sensorgrams which demonstrate co-engagement of exemplary compound (1-21) TNFα trimer complex to folate receptor (FIG. 3A), and exemplary compound (1-18) TNFα trimer complex to folate receptor (FIG. 3B). Further details are described in Example 53 of the experimental section.

FIG. 4 shows that exemplary compound (1-17) stimulated uptake of labelled TNFα in THP-1 cells in a dose-dependent manner as measured by median fluorescence intensity of pHrodo dye. Further details are described in Example 54 of the experimental section.

FIG. 5 shows that exemplary compound (I-17) mediated degradation and rescue of TNFα in THP-1 cells. Further details are described in Example 55 of the experimental section.

FIG. 6 shows that exemplary compound (1-17) mediated depletion of TNFα from the media of THP-1 cells. Further details are described in Example 56 of the experimental section.

FIGS. 7A-7B demonstrate folate receptor-dependent uptake of target protein IgE using an exemplary omalizumab-folate receptor ligand conjugate of this disclosure. FIG. 7A shows cell uptake of IgE-Alexa647 was enhanced across the dose range in both the wild type (WT) and folate receptor 2 (FOLR2) over expressing cells with the exemplary omalizumab-folate receptor ligand conjugate. FIG. 7B shows increased uptake was observed using the exemplary conjugate in FOLR2 overexpressing THP-1 cells compared to WT cells, and addition of folic acid decreased that uptake back to the WT level. Further details are described in Example 57 of the experimental section.

FIGS. 8A-8B illustrates the stimulation of uptake and degradation of target protein DQ-BSA by an exemplary conjugate. FIG. 8A shows the exemplary conjugate enhanced uptake of DQ-BSA and resulted in proteolysis and dequenching of BODIPY dye in the endolysosomal pathway. In the presence of protease inhibitors (PI), the intracellular fluorescent signal was diminished. FIG. 8B shows that the uptake and degradation is folate receptor mediated. In the presence of folic acid (FA) the fluorescent signal of anti-BSA control antibody without folate (anti-BSA) was the same as anti-BSA conjugate with the folate receptor ligand (anti-BSA/Compound I-4B conjugate).

### 5. DETAILED DESCRIPTION OF THE INVENTION

As summarized above, this disclosure provides classes of compounds including a ligand moiety that specifically binds to a cell surface receptor. Also provided herein are conjugates that comprise a moiety, X, that binds to such a cell surface receptor, for example, an internalizing cell surface receptor, for example, for sequestration and/or lysosomal degradation. In certain embodiments, the cell surface receptor is a folate receptor.

This disclosure includes compounds of formulae (I), (IIIA) and (IIIB) (e.g., as described in more detail herein below).

The compounds and conjugates and methods of this disclosure are described in greater detail below. A particular class of folate binding compounds is described. Also described are biomolecule conjugates that include a cell surface receptor binding moiety (X) that binds to a folate receptor. Linkers (L) and moieties of interest (Y) which find use in the folate binding compounds, and the biomolecule conjugates are also described. Methods in which the compounds and conjugates of this disclosure find use are also described.

### 5.1. Folate Receptor Binding Compounds

As summarized above, this disclosure provides a class of compounds including a ligand moiety that specifically binds to a cell surface folate receptor. The folate receptor ligand moieties of this disclosure can be linked to a variety of moieties of interest without impacting the specific binding to, and function of, the cell surface folate receptor. The inventors have demonstrated that compounds of this disclosure can utilize the functions of cell surface folate receptors in a biological system, e.g., for internalization and sequestration of a compound to the lysosome of a cell, and in some cases subsequent lysosomal degradation. The compounds of this disclosure find use in a variety of applications.

The compounds of this disclosure can specifically bind to a cell surface folate receptor, for example, an internalizing folate cell surface receptor. In particular embodiments, the surface folate receptor is a human folate receptor. In particular embodiments, the folate receptor is folate receptor 1 (FRα). In certain cases, the folate receptor is folate receptor 2 (FRβ).

The folate binding compounds of this disclosure include a moiety (X) that specifically binds to the cell surface folate receptor. The folate binding compounds can be monovalent or multivalent (e.g., bivalent or trivalent or of higher valency), where a monovalent compound includes a single folate receptor ligand moiety, and a monovalent compound includes two or more such moieties.

A compound comprising such X (e.g., as described herein), may bind to other receptors, for example, may bind with lower affinity as determined by, e.g., immunoassays or other assays known in the art. In a specific embodiment, X, or a compound as described herein comprising such X specifically binds to the cell surface folate receptor with an affinity that is at least 2 logs, 2.5 logs, 3 logs, 4 logs or greater than the affinity when X or the compound or the conjugate bind to another cell surface receptor. In a specific embodiment, X, or a compound as described herein comprising X, specifically binds to a folate receptor with an affinity (K_{d}) less than or equal to 20 mM. In particular embodiments, such binding is with an affinity (K_{d}) less than or equal to about 20 mM, about 10 mM, about 1 mM, about 100 uM, about 10 uM, about 1 uM, about 100 nM, about 10 nM, or less than or equal to about 1 nM. Unless otherwise noted, “binds,” “binds to,” “specifically binds” or “specifically binds to” in this context are used interchangeably.

In certain embodiments, the folate receptor binding moiety X is able to bind to a folate specific cell surface receptor, and direct (or target) the molecule to this receptor. In certain embodiments, the folate receptor binding moiety X is capable of binding to the folate receptor and directing (or targeting) a compound or conjugate described herein for internalization and sequestration to the lysosome, and/or subsequent lysosomal degradation.

In some embodiments, the folate binding moiety X includes a folate heterocyclic ring, or analog thereof, that is linked via a linking moiety comprising a cyclic group (e.g., aryl, heteroaryl, heterocycle, or cycloalkyl) to an amino acid derivative (e.g., a glutamic acid). The linking moiety can be of 1-10 atoms in length, such as 1-6, or 1-5 atoms in length. The linking moieties cyclic group can be any convenient group including, aryl, (e.g., phenyl), heteroaryl, (e.g., pyridine, thiophene), heterocyclic (e.g., piperidine), cycloalkyl (e.g., cyclohexyl), and bicycloalkyl groups. In some embodiments, the linking moieties cyclic group is aryl. The amino acid derivative can be any convenient amino acid group including, glutamic acid, and aspartic acid.

In some embodiments, the folate heterocyclic ring of X is linked via an optionally substituted aryl or heteroaryl group to an amino acid derivative (e.g., a glutamic acid) that together provide a moiety having a desirable binding affinity and activity at the folate receptor of interest. Multiple folate binding moieties X can be linked together to provide multivalent binding to the folate receptor. The folate binding moiety or moieties X can be further linked to any convenient moiety or molecule of interest (e.g., as described herein). In certain embodiments, the folate binding moiety X includes a glutamic acid moiety that is linked to a molecule of interest via a linker. In certain cases, the folate binding moiety X is linked to the molecule of interest via a linker covalently bonded to the gamma position of the glutamic acid moiety. In other cases, the folate binding moiety X is linked to the molecule of interest via a linker covalently bonded to the alpha position of the glutamic acid moiety.

Accordingly, provided herein are folate binding moiety X, of formula (Ia):

wherein:

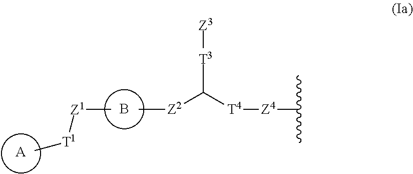

A is a ring system of formula (XII):

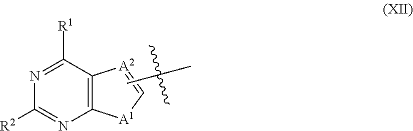

or a tautomer thereof, wherein:
R¹ and R² are independently selected from OH, NR²¹, and optionally substituted (C₁-C₆)alkyl (e.g., —CH₃ or —CH₂OH);
A¹ is selected from —N═CR³—, —CR³═N—, —CR³═CR³—, NR²¹, S, O, and C(R⁴)₂;
A² is selected from N, and CR³;
each R³ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each R⁴ is independently selected from H, halogen (e.g., F), and optionally substituted (C₁-C₆)alkyl
T¹ is an optionally substituted (C₁-C₃)alkylene;
Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;
B is a ring system selected from optionally substituted aryl, optionally substituted heteroaryl, optionally substituted heterocycle, optionally substituted cycloalkyl, and optionally substituted bridged bicycle;
Z² is absent, or a linking moiety selected from optionally substituted amide, optionally substituted sulfonamide, optionally substituted urea, optionally substituted thiourea, —NR²¹—, —O—, —S—, and optionally substituted (C₁-C₆)alkylene;
Z³ is carboxyl or carboxyl bioisostere, or a produg thereof,
T³ is absent, or is selected from optionally substituted (C₁-C₆)alkylene;
T⁴ is optionally substituted (C₁-C₆)alkylene (e.g., —CH₂CH₂—), or is absent;
Z⁴ is a linking moiety (e.g., a linking moiety selected from ester, amide, urea, thiourea, sulfonamide, amine, ether, optionally substituted aryl, optionally substituted heterocycle, and optionally substituted heteroaryl);
each R²¹ is independently selected from H, and optionally substituted (C₁-C₆)alkyl; and [symbol] represents the point of attachment to -L-Y (e.g., as described herein).

The folate binding moiety X of formula (Ia) can be incorporated into the compounds of this disclosure by attachment of a moiety of interest (Y) to the Z⁴ group via a linking moiety. It is understood that in the compounds of formula (Ia), the group or linking moiety attached to Z⁴ can, in some cases, be considered to be part of the folate binding moiety (X) and provide for desirable binding to the folate receptor. In certain other cases, the group or linking moiety attached to Z⁴ can be considered part of the linker L (e.g., of formula (IV) as described herein).

Accordingly, in one embodiment, provided herein are folate binding compounds of formula (I):

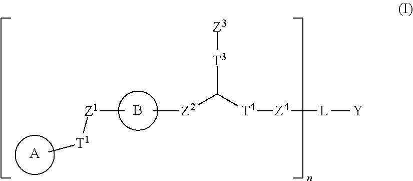

or a salt thereof,
wherein:
T¹ is an optionally substituted (C₁-C₃)alkylene;
Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;
B is a ring system selected from optionally substituted aryl, optionally substituted heteroaryl, optionally substituted heterocycle, optionally substituted cycloalkyl, and optionally substituted bridged bicycle;
Z² is absent, or a linking moiety selected from optionally substituted amide, optionally substituted sulfonamide, optionally substituted urea, optionally substituted thiourea, —NR²¹—, —O—, —S—, and optionally substituted (C₁-C₆)alkylene;
Z³ is carboxyl or carboxyl bioisostere, or a prodrug thereof,
T³ is absent, or is selected from optionally substituted (C₁-C₆)alkylene;
T⁴ is optionally substituted (C₁-C₆)alkylene (e.g., —CH₂CH₂—), or is absent;
Z⁴ is a linking moiety (e.g., a linking moiety selected from ester, amide, urea, thiourea, amine, sulfonamide, ether, optionally substituted aryl, optionally substituted heterocycle, and optionally substituted heteroaryl);
each R²¹ is independently selected from H, and optionally substituted (C₁-C₆)alkyl;
n is 1 to 100;
L is a linker;
Y is a moiety of interest; and
A is a ring system of formula (II):

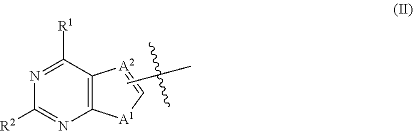

or a tautomer thereof, wherein:
R¹ and R² are independently selected from OH, NR²¹, and optionally substituted (C₁-C₆)alkyl (e.g., —CH₃ or —CH₂OH);
A¹ is selected from —N═CR³—, —CR³═N—, —CR³═CR³—, NR²¹, S, O, and C(R⁴)₂;
A² is selected from N, and CR³;
each R³ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each R⁴ is independently selected from H, halogen (e.g., F), and optionally substituted (C₁-C₆)alkyl.

In some embodiments of formula (I), at least one of following applies:
1) T³ is optionally substituted (C₁-C₆)alkylene (e.g., —CH₂CH₂—);
2) L is a non-cleavable linker and Y is an extracellular target-binding moiety;
3) when A is of formula (II-A) or (II-A′), or a tautomer thereof:

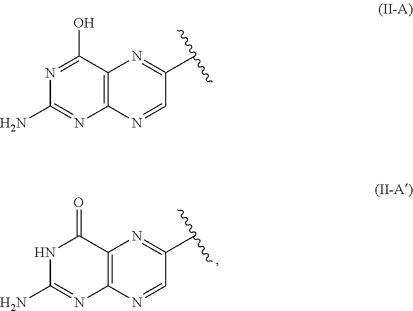 [SMILES: *c1cnc2nc(N)nc(O)c2n1 ; *c1cnc2nc(N)[nH]c(=O)c2n1]

then Z¹ is not NR²¹, and/or B is not 1,4-linked phenyl;
4) when A is of formula (II-B), or a tautomer thereof:

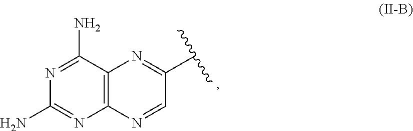 [SMILES: *c1cnc2nc(N)nc(N)c2n1]

then Z¹ is not NR²¹, and/or B is not 1,4-linked phenyl; and/or
5) when A is of formula (II-C) or (II-C′), or a tautomer thereof:

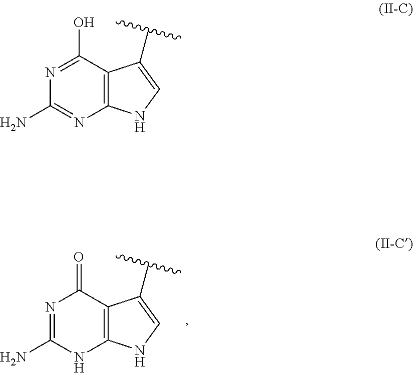 [SMILES: *c1c[nH]c2nc(N)nc(O)c12 ; *c1c[nH]c2[nH]c(N)nc(=O)c12]

then T¹-Z¹ is not —CH₂CH₂—, and/or B is not phenyl.

In some embodiments of formula (I), T³ is optionally substituted (C₁-C₆)alkylene. In certain cases, T³ is (C₁-C₆)alkylene, i.e., hexyl, pentyl, butyl, propyl, ethyl or methyl. In certain cases, T³ is (C₁-C₃)alkylene. In certain cases, T³ is-CH₂CH₂CH₂—. In certain cases, T³ is —CH₂CH₂—. In certain cases, T³ is —CH₂—.

In some embodiments of formula (I), T⁴ is absent. Accordingly, in some embodiments, the compound is of formula (IIIA):

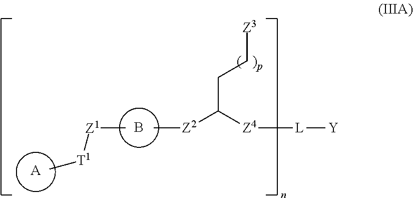

wherein p is 0 or 1.

In certain other embodiments of formula (I), T⁴ is optionally substituted (C₁-C₆)alkylene. In certain cases, each T⁴ is (C₁-C₆)alkylene, i.e., hexyl, pentyl, butyl, propyl, ethyl or methyl. In certain cases, each T⁴ is (C₁-C₃)alkylene. In certain cases, each T⁴ is-CH₂CH₂CH₂—. In certain cases, each T⁴ is —CH₂CH₂—. In certain cases, each T⁴ is —CH₂—.

In some embodiments of formula (I), T³ is absent. Accordingly, in some embodiments, the compound is of formula (IIIB):

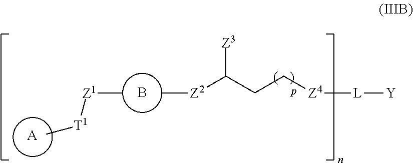

wherein p is 0 or 1.

In certain embodiments of any one of formulae (I), (IIIA) or (IIIB), Z³ is a carboxyl group, or a prodrug thereof. In certain other embodiments, Z³ is a carboxyl bioisostere, or a prodrug thereof. A carboxyl bioisostere is a group with similar physical or chemical properties to a carboxyl group. In certain cases, the carboxyl bioisostere produces broadly similar biological properties to the corresponding carboxyl group. In certain cases, the carboxyl bioisostere may modify the activity of the compound, and may alter the metabolism of the compound. The subject compounds can include both acyclic and cyclic carboxylic acid bioisosteres. Carboxyl bioisosteres that can be utilized in the subject compounds includes, but is not limited to, hydroxamic acids, phosphonic acids, sulphonic acids, sulfonamides, acylsulfonamides, sulfonylureas, tetrazoles, thiazolidinediones, oxazolidinediones, 5-oxo-1,2,4-oxadiazole, 5-oxo-1,2,4-thiadiazole, 5-thioxo-1,2,4-oxadiazole, isothiazoles, difluorophenols, tetramic acids, squaric acids, 3-hydroxyquinolin-2-ones, and 4-hydroxyquinolin-2-ones. In certain embodiments, the carboxyl bioisostere is a moiety as described in Ballatore et al. 2013, ChemMedChem., 8(3): 385-395.

In certain embodiments, a prodrug derivative of the carboxyl bioisostere group (Z³) may be incorporated into the compounds. For example, an ester prodrug group (e.g., —CO₂Et, or —CO₂CH₂CH₂—R″, where R″ is a heterocycle, e.g., N-morpholino) is included instead of a carboxylic acid group. Exemplary ester containing compounds are described herein. See e.g., compounds 1-56 and 1-57 of Table 6.

The term “pro-drug” refers to an agent which is converted into the drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The pro-drug may also have improved solubility in pharmacological compositions over the parent drag. An example, without limitation, of a pro-drug would be a compound of the present disclosure wherein it is administered as an ester (the “pro-drug”) to facilitate transmittal across a cell membrane where water solubility is not beneficial, but then it is metabolically hydrolyzed to the carboxylic acid once inside the cell where water solubility is beneficial.

In certain embodiments, the carboxyl bioisostere, or a prodrug thereof, is a moiety of one of the following structures:

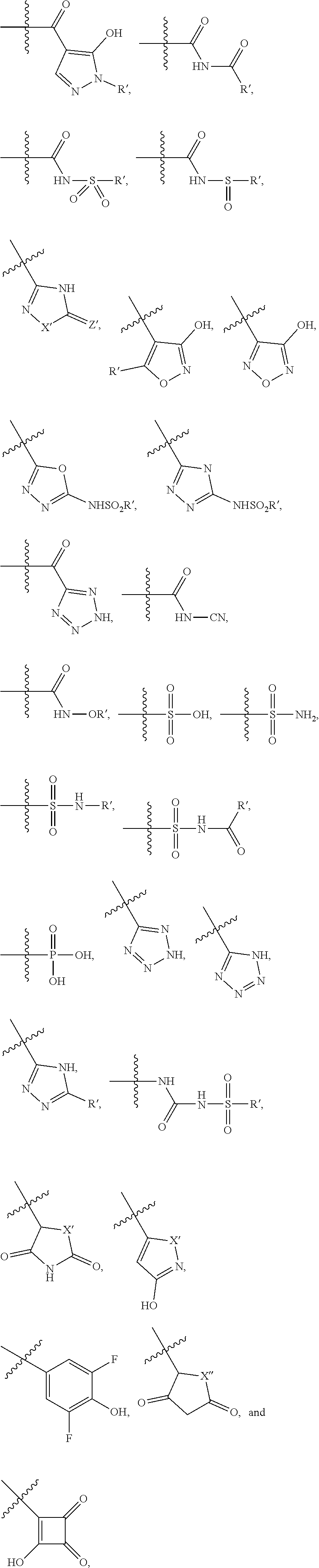

where:
each R′ is independently H or an optionally substituted moiety selected from (C₁₋₁₀)alkyl, (C₂₋₁₀)alkenyl, (C₂₋₁₀)heteroalkyl, (C₃₋₈)cyclic ring selected from cycloalkyl, aryl, heterocycle, or heteroaryl;
each X′ is independently O or S; and
X″ is NH, O, or CH₂.

In certain embodiments, Z³ is selected from —COOH, —COOR²², —CH₂OH, —CH₂OR²², —CN, and tetrazole, wherein R²² is optionally substituted (C₁-C₆)alkyl. In certain cases, Z³ is —COOH. In certain cases, Z³ is —COOR²², and R²² is optionally substituted (C₁₋₃)alkyl. In certain cases, R²² is methyl, ethyl or propyl. In certain cases, R²² is substituted methyl, ethyl, or propyl. In certain cases, Z³ is —CH₂OH, or —CH₂OR²², and R²² is optionally substituted (C₁₋₃)alkyl. In certain cases, Z³ is —CN. In certain cases, Z³ is tetrazole.

In certain embodiments, Z³ is selected from one of the following structures:

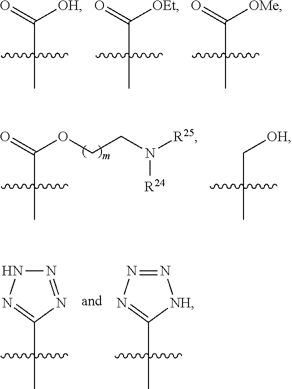

wherein:
R²⁴ and R²⁵ are independently selected from H and optionally substituted (C₁-C₆)alkyl, or R²⁴ and R²⁵ are cyclically linked to provide an optionally substituted 5 or 6-membered heterocycle; and
m is 1 to 5. In certain cases, R²⁴ and R²⁵ are H. In certain embodiments, R²⁴ and R²⁵ is optionally substituted (C₁₋₃)alkyl. In certain cases, R²⁴ and R²⁵ are cyclically linked to provide an optionally substituted 5-membered heterocycle. In certain other cases, R²⁴ and R²⁵ are cyclically linked to provide an optionally substituted 6-membered heterocycle. In certain cases, Z³ is of the following structure:

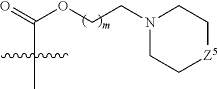

wherein
Z⁵ is O, NH or NR²¹; and
R²¹ is (C₁-C₆)alkyl.

In certain cases, Z⁵ is O and m is 1. In certain cases, Z⁵ is NH, and m is 1. In certain cases, Z⁵ is NCH₃, and m is 1.

In some embodiments of any one of formulae (I), (IIIA) or (IIIB), Z² is a linking moiety (e.g., as described herein). In certain cases, Z² is an optionally substituted amide. In certain cases, Z² is an optionally substituted sulfonamide. In certain cases, Z² is an optionally substituted urea. In certain cases, Z² is an optionally substituted thiourea. In certain embodiments, Z² is —CONR²¹—. In certain cases, Z² is —O—. In certain case, Z² is —S—. In certain cases, Z² is an optionally substituted (C₁-C₆)alkylene. In certain cases, Z² is an amide bioisostere (e.g., as described herein below).

In certain embodiments, Z² is —CONR²¹—, wherein R²¹ is selected from H, and optionally substituted (C₁-C₆)alkyl. In certain cases, R²¹ is H. In certain other cases, R²¹ is optionally substituted (C₁-C₃)alkyl. In certain cases, R²¹ is methyl. In certain cases, R²¹ is ethyl.

In certain embodiments, Z² is —CONR²¹—, —SO₂NR²¹—, —NR²¹CO—, —NR²¹C(═O)NR²¹—, or —NR²¹C(═S)NR²¹, wherein each R²¹ is independently selected from H, and optionally substituted (C₁-C₆)alkyl. In certain cases, each R²¹ is H. In certain other cases, each R²¹ is optionally substituted (C₁-C₃)alkyl. In certain cases, R²¹ is methyl. In certain cases, R²¹ is ethyl.

In certain cases of any one of formulae (I), (IIIA) or (IIIB), Z⁴ is a linking moiety selected from ester, amide, sulfonamide, urea, thiourea, amine, ether, thioether, optionally substituted aryl, optionally substituted heterocycle, and optionally substituted heteroaryl. In certain cases, Z⁴ is a linking moiety selected from amide or amide bioisostere. In certain cases, Z⁴ is an amine. In certain cases, Z⁴ is an ether. In certain cases, Z⁴ is a thioether. In certain cases, Z⁴ is an optionally substituted aryl. In certain cases, Z⁴ is a 1,4-phenyl group. In certain cases, Z⁴ is an optionally substituted heteroaryl. In certain cases, Z⁴ is a oxadiazole. In certain cases, Z⁴ is a triazole.

In certain cases, Z⁴ is an amide bioisotere. An amide bioisostere is a group with similar physical or chemical properties to an amide group. In certain cases, the amide bioisostere produces broadly similar biological properties to the corresponding amide group. In certain cases, the amide bioisostere may modify the activity of the compound, and may alter the metabolism of the compound. The subject compounds can include both acyclic and cyclic amide bioisosteres. Amide bioisosteres that can be utilized in the subject compounds includes, but is not limited to, imidazoles, triazoles, thiazoles, oxadiazoles, tetrazoles, indoles, olefins, fluoroalkenes, ureas, esters, thioamides, phosphonamidates, sulfonamides, trifluoro ethylamines, amidines, and carbamates. In some cases, the amide bioisotere is a 5-membered ring heterocycle, e.g., a triazole, an oxadiazole, an imidazole, a tetrazole, or a pyrazole. In certain cases, the amide bioisostere is a six membered heteroaryl, e.g., a pyrazine or a pyridine. In certain cases, the amide bioisostere is a retroinverted, or reverse amide, e.g., —NHC(O)— converted to —C(O)NH—. In certain cases, the amide bioisostere is a urea. In certain cases, the amide bioisostere is a carbamate. In certain cases, the amide bioisostere is an amidine. In certain cases, the amide bioisostere is a thioamide. In certain cases, the amide bioisostere is a trifluoroethylamine. In certain cases, the amide bioisotere is a sulfonamide. In certain cases, the amide bioisostere is a phosphonamidate. In certain cases, the amide bioisostere is an olefin. In certain embodiments, the amide bioisotere is a moiety as described in Kumari et al. 2020, J. Med. Chem., 63: 12290-12358. In certain embodiments, the amide bioisostere is a moiety of one of the following structures:

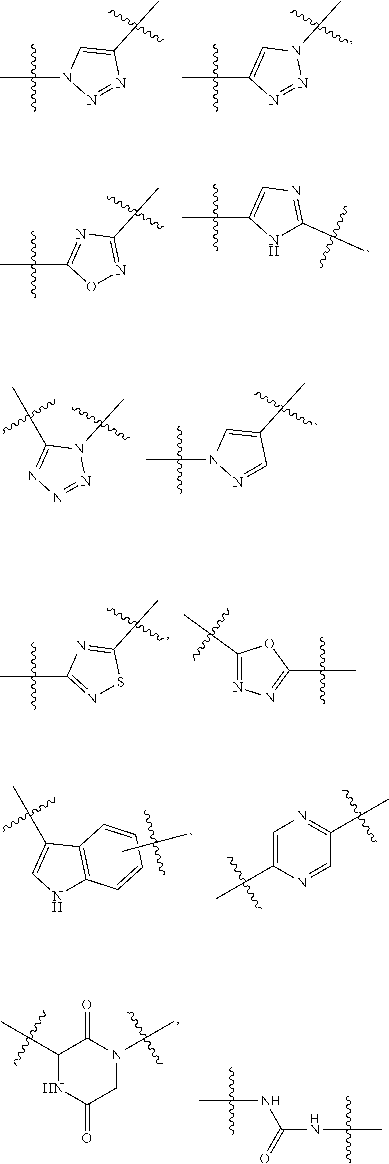

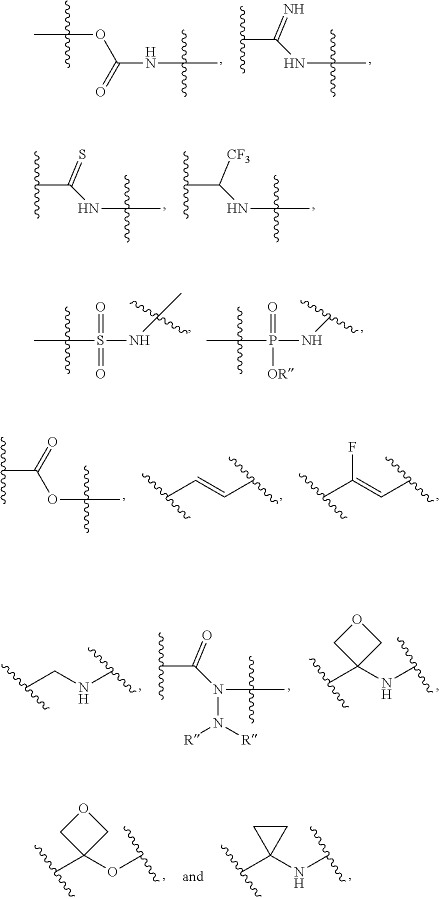

Where R″ is an optionally substituted (C₁-C₆)alkyl.

In certain cases, Z⁴ is a linking moiety selected from —CONR²¹—, —NR²¹—, —O—, —S—, optionally substituted aryl (e.g., 1,4-phenyl) and optionally substituted heteroaryl (e.g., oxadiazole or triazole), wherein R is selected from H, and optionally substituted (C₁-C₆)alkyl. In certain cases, R² is methyl. In certain cases, R is ethyl.

In some embodiments, Z⁴ is a linking group selected from:

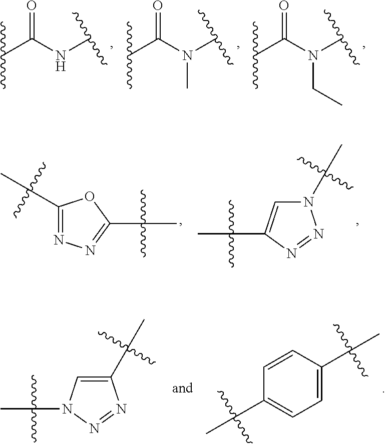

In some embodiments of formula (I) or (IIIA), —Z²CH(-T³-Z³)T⁴Z⁴— is selected from the following structures:

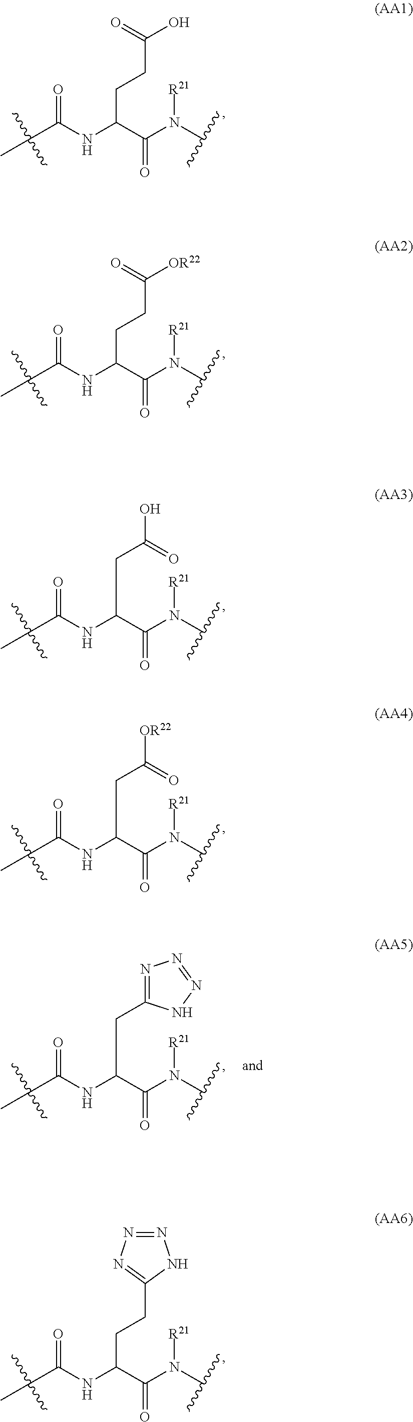

or a tautomer thereof, or a salt thereof.

In some embodiments of formula (I) or (IIIB), —Z²CH(-T³-Z³)T⁴Z⁴— of formula (I) is selected from the following structures:

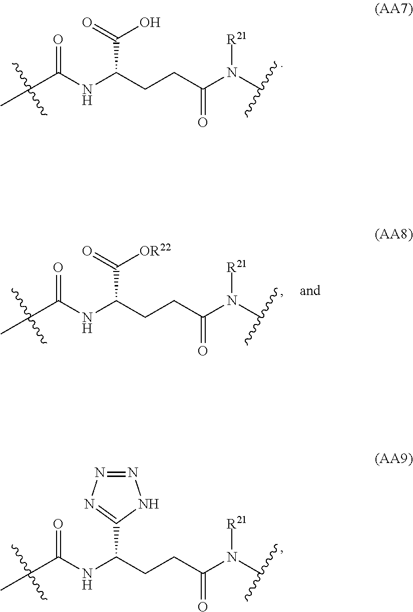

or a tautomer thereof, or a salt thereof.

In certain cases of (AA2), (AA4) or (AA8), R²² is optionally substituted (C₁-C₆)alkyl. In certain cases, R²² is methyl. In certain cases, R²² is ethyl. In some cases, R²² is propyl. In certain cases, R²² is substituted (C₁-C₆)alkyl. In certain cases, R²² is of the formula —(CH₂)mCH₂N(R²⁴)(R²¹), where R²⁴ and R²⁵ are independently selected from H and optionally substituted (C₁-C₆)alkyl, or R²⁴ and R²⁵ are cyclically linked to provide an optionally substituted 5 or 6-membered heterocycle; and m is 1 to 5. In certain cases, R²⁴ and R²⁵ are H. In certain embodiments, R²⁴ and R²⁵ is optionally substituted (C₁₋₃)alkyl. In certain cases, R²⁴ and R²⁵ are cyclically linked to provide an optionally substituted 5-membered heterocycle. In certain other cases, R²⁴ and R²⁵ are cyclically linked to provide an optionally substituted 6-membered heterocycle. In certain cases, R²² is of the following structure:

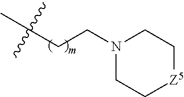

wherein
Z⁵ is O, NH or NR²¹; and R²¹ is (C₁-C₆)alkyl. In certain cases, Z⁵ is O and m is 1. In certain cases, Z⁵ is NH, and m is 1. In certain cases, Z⁵ is NCH₃, and m is 1.

In certain embodiments of any one of (AA1)-(AA9), R²¹ is H. In certain cases, R²¹ is methyl. In certain cases, R²¹ is ethyl. In certain cases, R²¹ is propyl. In certain cases, R²¹ is propargyl.

In some embodiments of formula (I) or (IIIA), —Z²CH(-T³-Z³)T⁴Z⁴— is of the structure (AA1). In certain cases, —Z²CH(-T³-Z³)T⁴Z⁴— is of the structure (AA2). In certain cases, —Z²CH(-T³-Z³)T⁴Z⁴— is of the structure (AA3). In certain cases, —Z²CH(-T³-Z³)T⁴Z⁴— is of the structure (AA4). In certain cases, —Z²CH(-T³-Z³)T⁴Z⁴— is of the structure (AA5). In certain cases, —Z²CH(-T³-Z³)T⁴Z⁴— is of the structure (AA6).

In certain embodiments of formula (I) or (IIIB), —Z²CH(-T³-Z³)T⁴Z⁴— is of the structure (AA7). In certain cases, —Z²CH(-T³-Z³)T⁴Z⁴— is of the structure (AA8). In certain other cases, —Z²CH(-T³-Z³)T⁴Z⁴— is of the structure (AA9).

In certain embodiments of the subject compounds, A1 of ring system A is selected from —N═CR³—, —CR³═N—, or —CR³═CR³—. In certain cases, A1 of ring system A is N═CR³—. In certain cases, A1 of ring system A is —CR³═N—. In certain other cases, A1 of ring system A is —CR³═CR³—.

In some embodiments of the subject compounds, A is of formula (IIA):

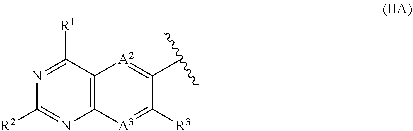

or a tautomer thereof, or a salt thereof, wherein:
A² is selected from N, and CR³;
A³ is independently selected from N, and CR^{2′}.

In certain embodiments of formula (IIA), A² and A³ are each N. In certain embodiments, A² is N and A³ is CR²¹. In certain cases, A² is CR³ and A³ is N. In certain other embodiments, A² and A³ are each independently CR³.

In certain embodiments of formula (IIA), each R³ is H. In certain other embodiments, R³ is halogen. In certain cases, the halogen is fluoride. In certain cases, R³ is OH. In certain cases, R³ is optionally substituted (C₁-C₆)alkyl. In certain cases, R³ is optionally substituted (C₁-C₆)alkoxy. In certain cases, R³ is COOH. In certain cases, R³ is NO₂. In certain cases, R³ is CN. In certain cases, R³ is NH₂, or —N(R²¹)₂. In certain cases, R³ is —OCOR²¹ or —COOR²¹. In certain other cases, R³ is —CONHR²¹, or —NHCOR²¹.

In certain embodiments of formula (IIA), R² is —NH₂. In certain embodiments, R² is optionally substituted (C₁-C₆)alkyl. In certain embodiments, R² is —CH₃. In certain embodiments, R² is —CH₂OH. In certain other embodiments, R² is H.

In certain embodiments of formula (IIA), R¹ is OH. In certain embodiments, R² is NH₂.

In certain embodiments of the subject compounds, A is selected from:

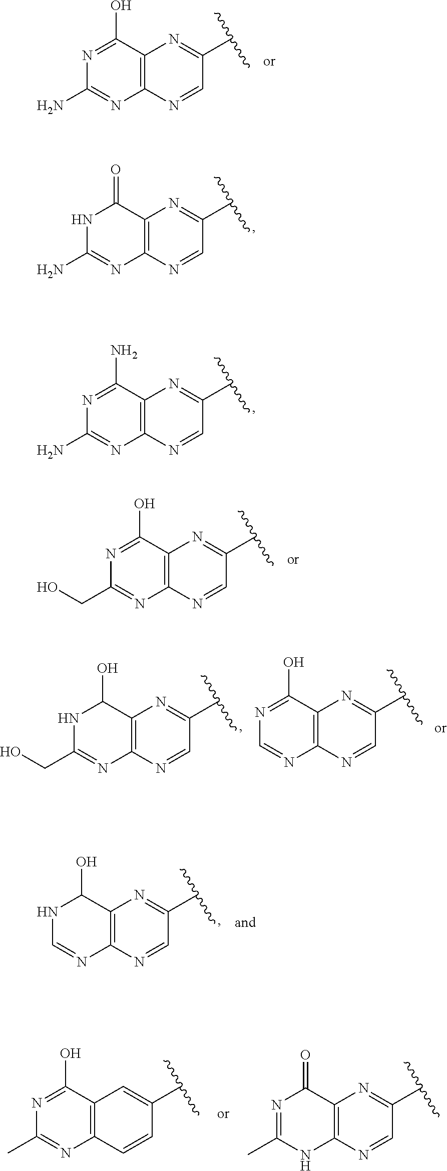

or a tautomer thereof.

In certain embodiments of the subject compounds, A¹ of ring system A is selected from —NR²¹—, —S—, —O— or —C(R²¹)₂—. In certain cases, A¹ of ring system A is —NR²¹—. In certain cases, A¹ of ring system A is —S—. In certain cases, A¹ of the ring system A is —O—. In certain other cases, A¹ of ring system A is —C(R²¹)₂—.

In some embodiments of the subject compounds, A is of formula (IIB) or (IIC):

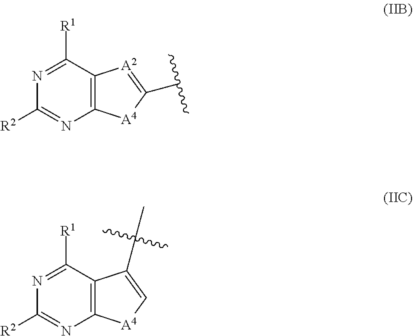

or a tautomer thereof, or a salt thereof, wherein A⁴ is selected from NR²¹, S, and O.

In certain embodiments of formula (IIB), A² is CR³. In certain cases, A² is N. In certain cases of formula (IIB), A⁴ is NR²¹. In certain cases, A⁴ is S. In certain other embodiments, A⁴ is O. In certain embodiments, A² is CR³ and A⁴ is NR²¹.

In certain embodiments of formula (IIB), each R³ is H. In certain other embodiments, R³ is halogen. In certain cases, the halogen is fluoride. In certain cases, R³ is OH. In certain cases, R³ is optionally substituted (C₁-C₆)alkyl. In certain cases, R³ is optionally substituted (C₁-C₆)alkoxy. In certain cases, R³ is COOH. In certain cases, R³ is NO₂. In certain cases, R³ is CN. In certain cases, R³ is NH₂, or —N(R²¹)₂. In certain cases, R³ is —OCOR²¹ or —COOR²¹. In certain other cases, R³ is —CONHR²¹, or —NHCOR²¹.

In certain embodiments of formula (IIB), R² is —NH₂. In certain embodiments, R² is optionally substituted (C₁-C₆)alkyl. In certain embodiments, R² is —CH₃. In certain embodiments, R² is —CH₂OH. In certain other embodiments, R² is H.

In certain embodiments of formula (IIB), R¹ is OH. In certain embodiments, R² is NH₂.

In certain embodiments of the subject compounds, A is selected from:

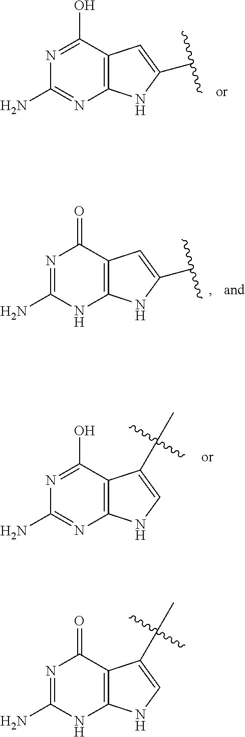 [SMILES: *c1cc2c(O)nc(N)nc2[nH]1 ; *c1cc2c(=O)nc(N)[nH]c2[nH]1 ; *c1c[nH]c2nc(N)nc(O)c12 ; *c1c[nH]c2[nH]c(N)nc(=O)c12]

or a tautomer thereof.

In certain embodiments of any one of formulae (I), (IIIA) or (IIIB), T¹ is CH₂. In certain embodiments, T¹ is CH₂CH₂. In certain other embodiments, T¹ is CH₂CH₂CH₂.

In certain embodiments of any one of formulae (I), (IIIA) or (IIIB), Z¹ is NR¹. In certain cases, R²¹ is H. In certain cases, R²¹ is methyl. In certain cases, R²¹ is ethyl. In certain cases, R²¹ is propyl. In certain cases, R²¹ is propargyl.

In certain cases of any one of formulae (I), (IIIA) or (IIIB), Z¹ is O. In certain other cases, Z¹ is S.

In certain cases of any one of formulae (I), (IIIA) or (IIIB), Z¹ is substituted methylene. In certain cases of any one of formulae (I), (IIIA) or (IIIB), Z¹ is methylene substituted with propargyl (i.e., —CH(propargyl)-. In certain cases of any one of formulae (I), (IIIA) or (IIIB), Z¹ is methylene substituted with (C₁-C₃)alkyl.

In certain embodiments of any one of formulae (I), (IIIA) or (IIIB), T¹-Z¹ is optionally substituted (C₁-C₆)alkylene. In certain cases, T¹-Z¹ is —CH₂CH₂—. In certain cases, T¹-Z¹ is —CH₂CH₂CH₂CH₂—. In certain cases, T¹-Z¹ is —CH₂CH₂CH₂—. In certain embodiments of any one of formulae (I), (IIIA) or (IIIB), T¹-Z¹ is —CH₂CH(propargyl)-.

In certain embodiments of the subject compounds, the B ring system is an optionally substituted aryl. In certain cases, the B ring system is an optionally substituted heteroaryl. In certain cases, the B ring system is an optionally substituted heterocycle. In certain cases, the B ring system is an optionally substituted cycloalkyl. In certain other cases, the B ring system is an optionally substituted bridged bicycle.

In certain embodiments of the subject compounds, the B ring system is selected from optionally substituted phenyl, optionally substituted pyridyl, optionally substituted pyrimidine, optionally substituted thiophene, optionally substituted pyrrole, optionally substituted furan, optionally substituted oxazole, optionally substituted thiazole, optionally substituted cyclohexyl, optionally substituted cyclopentyl, optionally substituted indole, and optionally substituted bicycloalkyl (e.g., bicyclo[1.1.1]pentane).

In certain embodiments of the subject compounds, the B ring system is selected from optionally substituted 1,4-phenylene, optionally substituted 1,3-phenylene, optionally substituted 2,5-pyridylene, optionally substituted 2,5-thiophene, optionally substituted 1,4-cyclohexyl, and optionally substituted 1,3-bicyclo[1.1.1]pentane.

In certain embodiments of the subject compounds, B—Z² is selected from any one of formulae (BZ1)-(BZ8):

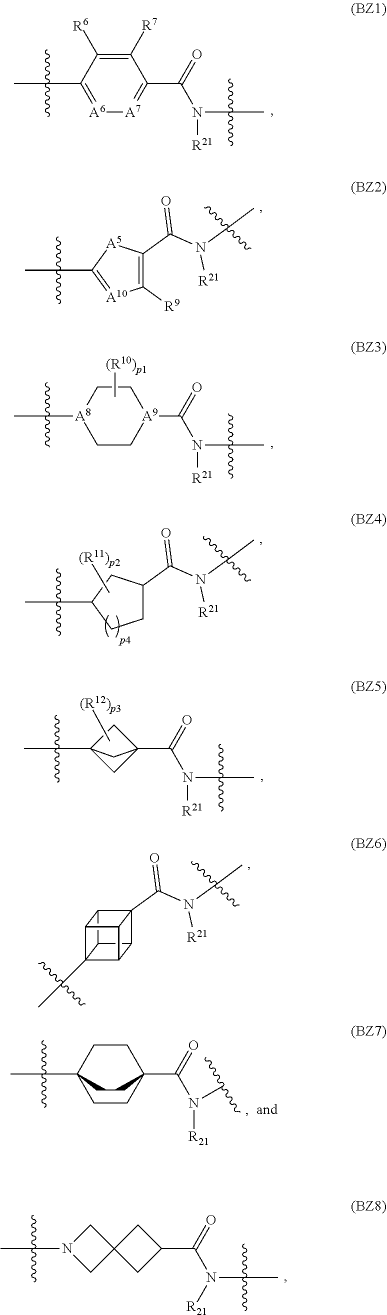

wherein:
A⁵ is selected from NR²¹, S, O, C(R)₂;
A⁶-A⁹ are independently selected from N, and CR⁵; A¹⁰ is selected from N, and CR;
R²¹ is selected from H, and optionally substituted (C₁-C₆)alkyl; each R⁵ to R¹² is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
p1 is 0 to 10;
p2 is 0 to 14;
p3 is 0 to 4; and
p4 0 to 4.

In certain embodiments of the subject compounds, B—Z² is of formula (BZ1). In certain embodiments of formula (BZ1), each A⁶ and A⁷ is CR⁵. In certain cases, at least one of A⁶ and A⁷ is N. In certain cases, A⁶ is CR⁵ and A⁷ is N. In certain other cases, A⁶ is N and A⁷ is CR⁵. In certain cases, R⁵ is H. In certain cases, R⁵ is halogen. In certain cases, the halogen is F or Cl. In certain cases, R⁵ is (C₁-C₃)alkyl. In certain cases, R⁵ is methyl. In certain cases, each of R⁶ and R⁷ is H. In certain other cases, at least one of R⁶ and R⁷ is a substituent other than H. In certain cases, at least one of R⁶ and R⁷ is halogen. In certain cases, the halogen is F or Cl. In certain cases, at least one of R⁶ and R⁷ is (C₁-C₃)alkyl. In certain cases, at least one of R⁶ and R⁷ is methyl. In certain embodiments of formula (BZ1), R²¹ is H. In certain other embodiments, R²¹ is (C₁-C₃)alkyl. In certain cases, R²¹ is methyl.

In certain embodiments of the subject compounds, B—Z² is of formula (BZ2). In certain embodiments of formula (BZ2), A⁵ is NR²¹, where R²¹ is selected from H or (C₁-C₃)alkyl, e.g., methyl. In certain cases, A⁵ is S. In certain cases, A⁵ is O. In certain other cases, A⁵ is C(R⁵)₂. In certain cases, R⁵ is H. In certain cases, R⁵ is halogen. In certain cases, the halogen is F or Cl. In certain cases, R⁵ is (C₁-C₃)alkyl. In certain cases, R⁵ is methyl. In certain cases, A¹⁰ is CR⁸ and each of R⁸ and R⁹ is H. In certain other cases, A¹⁰ is CR⁸ and at least one of R⁸ and R⁹ is a substituent other than H. In certain cases, A¹⁰ is CR⁸ and at least one of R⁸ and R⁹ is halogen. In certain cases, the halogen is F or Cl. In certain cases, at least one of R⁸ and R⁹ is (C₁-C₃)alkyl. In certain cases, A¹⁰ is CR⁸ and at least one of R⁸ and R⁹ is methyl. In certain embodiments of formula (BZ2), R²¹ is H. In certain other embodiments, R²¹ is (C₁-C₃)alkyl. In certain cases, R²¹ is methyl. In certain embodiments of formula (BZ2), A¹⁰ is CR⁸, where R⁸ is selected from H or (C₁-C₃)alkyl, e.g., methyl. In certain embodiments of formula (BZ2), A¹⁰ is CH. In cases of formula (BZ2), A¹⁰ is N. In certain embodiments of formula (BZ2), A⁵ is NR²¹ and A¹⁰ is CR⁸, where R²¹ and R⁸ are independently selected from H or (C₁-C₃)alkyl, e.g., methyl. In certain embodiments of formula (BZ2), A⁵ is NR²¹ and A¹⁰ is N. In certain embodiments of formula (BZ2), A⁵ is S and A¹⁰ is N.

In certain embodiments of the subject compounds, B—Z² is of formula (BZ3). In certain embodiments of formula (BZ3), each A⁸ and A⁹ is CR⁵. In certain cases, at least one of A⁸ and A⁹ is N. In certain cases, A⁸ is CR⁵ and A⁹ is N. In certain other cases, A⁸ is N and A⁹ is CR⁵. In certain cases, both of A⁸ and A⁹ are N. In certain cases, R⁵ is H. In certain cases, R⁵ is halogen. In certain cases, the halogen is F or Cl. In certain cases, R⁵ is (C₁-C₃)alkyl. In certain cases, R⁵ is methyl. In certain cases, each R¹⁰ is H (or p1 is 0). In certain other cases, p1 is 1 to 10 and at least one R¹⁰ group is a substituent other than H. In certain cases, at least one R¹⁰ group is halogen. In certain cases, the halogen is F or Cl. In certain cases, at least one R¹⁰ group is (C₁-C₃)alkyl. In certain cases, at least one of R¹⁰ group is methyl. In certain embodiments of formula (BZ3), R²¹ is H. In certain other embodiments, R²¹ is (C₁-C₃)alkyl. In certain cases, R²¹ is methyl.

In certain embodiments of the subject compounds, B—Z² is of formula (BZ4). In certain embodiments of formula (BZ4), p4 is 0, such that the B ring system is cyclobutyl. In certain cases, p4 is 1, such that the B ring system is a cyclopentyl. In certain cases, p4 is 2, such that the B ring system is cyclohexyl. In certain cases, p4 is 3, such that the B ring system is cycloheptyl. In certain other cases, p4 is 4, such that the B ring system is cyclooctyl. In certain cases, each R¹¹ is H (or p2 is 0). In certain other cases, p2 is 1 to 14 and at least one R¹¹ group is a substituent other than H. In certain cases, at least one R¹¹ group is halogen. In certain cases, the halogen is F or Cl. In certain cases, at least one R¹¹ group is (C₁-C₃)alkyl. In certain cases, at least one of R¹¹ group is methyl. In certain embodiments of formula (BZ4), R²¹ is H. In certain other embodiments, R²¹ is (C₁-C₃)alkyl. In certain cases, R²¹ is methyl.

In certain embodiments of the subject compounds, B—Z² comprises a bicycloalkyl group and is of any of formulae (BZ5)-(BZ8). In certain embodiments of formula (BZ5), each R¹² is H (or p3 is 0). In certain other cases, p3 is 1 to 4 and at least one R² group is a substituent other than H. In certain cases, at least one R¹² group is halogen. In certain cases, the halogen is F or Cl. In certain cases, at least one R¹² group is (C₁-C₃)alkyl. In certain cases, at least one of R¹² group is methyl. In certain embodiments of formula (BZ5), R²¹ is H. In certain other embodiments, R²¹ is (C₁-C₃)alkyl. In certain cases, R²¹ is methyl. In certain embodiments of any of formulae (BZ6)-(BZ8), R²¹ is H. In certain other embodiments, R²¹ is (C₁-C₃)alkyl. In certain cases, R²¹ is methyl.

In certain embodiments of the subject compounds, B—Z² is:

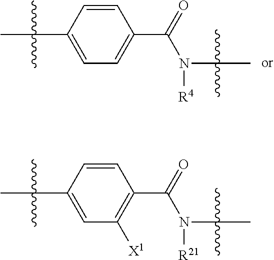

wherein X¹ is halogen. In certain cases, the halogen is F. In certain cases, the halogen is Cl. In certain cases, the halogen is bromide.

In certain embodiments of the subject compounds, T¹-Z¹—B is selected from:

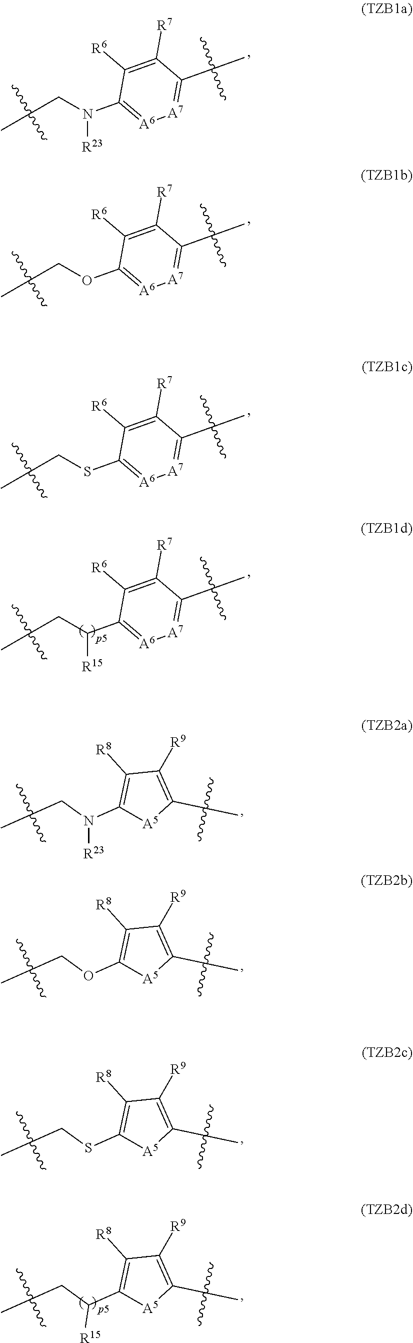

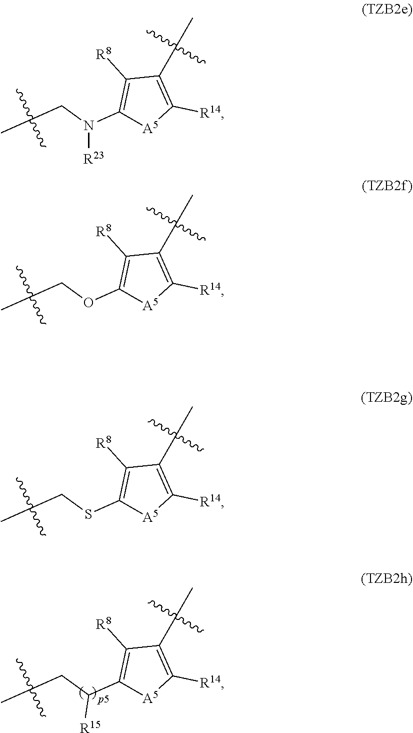

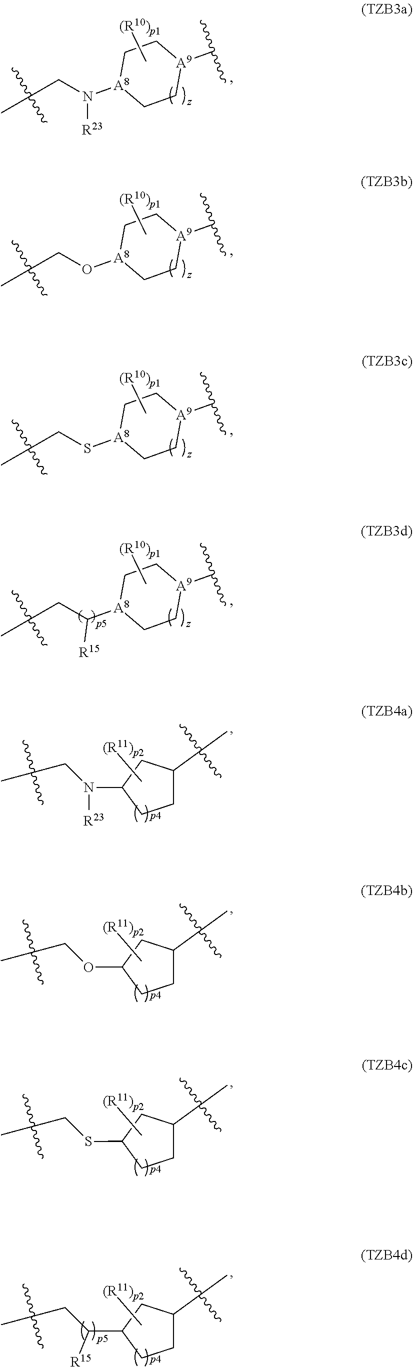

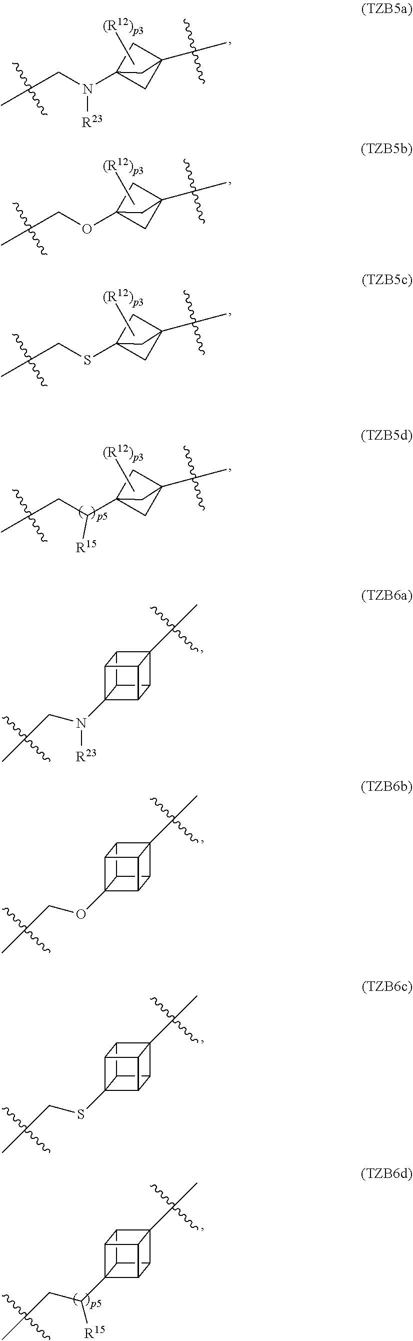

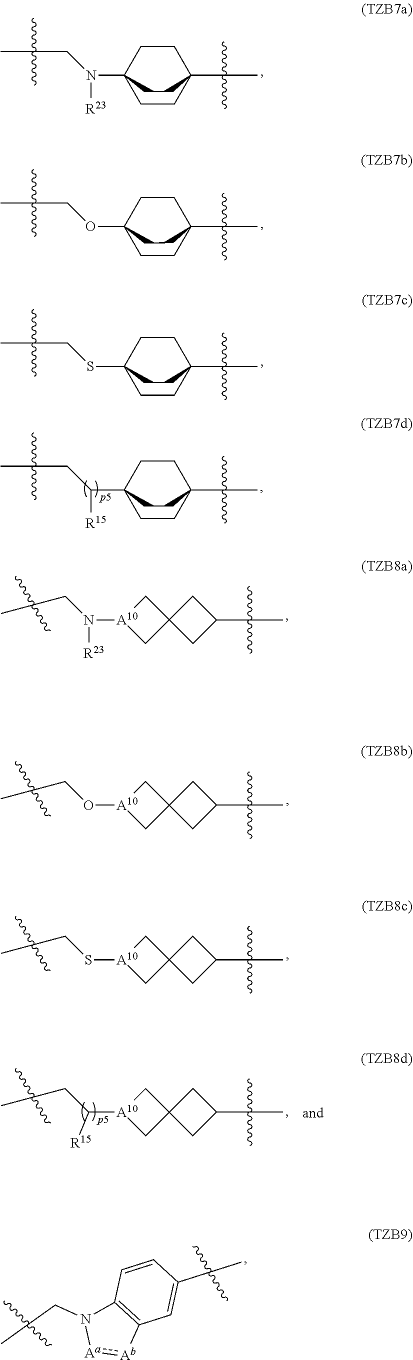

wherein:
A⁵ is selected from NR²¹, S, O, C(R)₂;
A⁶-A¹⁰ are independently selected from N, and CR⁵;
R²³ is H, optionally substituted (C₁-C₆)alkyl, or R²³ forms a 5 or 6 membered cycle together with an atom of the adjacent cycle;
each R⁵ to R¹² and R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR⁵, —CONHR²⁵, and —NHCOR²⁵;
R¹⁵ is H, optionally substituted (C₁-C₆)alkyl, or R¹⁵ forms a 5 or 6 membered cycle together with an atom of the adjacent cycle;
[symbol] is a single bond or a double bond;
wherein when [symbol] is a single bond A^{a} is selected from C(R⁵)₂, and C═O, and A^{b} is selected from C(R⁵)₂, and NR²¹; and
when [symbol] is a double bond A^{a} is CR⁵, and A^{b} is selected from CR⁵ and N
p1 is 0 to 10;
p2 is 0 to 14;
p3 is 0 to 4;
p4 0 to 4; and
p5 is 1 to 3.

In certain embodiments of the subject compounds, T¹-Z¹—B is of any one of formulae (TZB1a)-(TZB1d), and each of A⁶-A⁷, and R⁶-R⁷ are as defined for formula (BZ1). In certain embodiments of formula (TZB1a) or (TZB1d), R²³ or R⁵ is H. In certain other embodiments, R²³ or R¹⁵ is optionally substituted (C₁-C₃)alkyl. In certain cases, R²³ or R¹⁵ is methyl. In certain embodiments, R²³ or R¹⁵ is an alkyne moiety of formula —(CH₂)nCCH, where n is 1 or 2. In certain embodiments R²³ or R⁵ forms a fused 5-membered cycle with an atom of the adjacent aryl or heteroaryl ring. In certain embodiments R²³ or R⁵ forms a fused 6-membered cycle with an atom of the adjacent aryl or heteroaryl ring. In certain embodiments of formula (TZB1d), p5 is 1. In certain embodiments, p5 is 2. In certain other embodiments, p5 is 3.

In certain embodiments of the subject compounds, T¹-Z¹—B is of any one of formulae (TZB2a)-(TZB2h), and each of A⁵, and R⁸-R⁹ are as defined for formula (BZ2). In certain embodiments, R²³ or R¹⁵ is H. In certain other embodiments, R²³ or R¹⁵ is optionally substituted (C₁-C₃)alkyl. In certain cases, R²³ or R⁵ is methyl. In certain embodiments, R²³ or R¹⁵ is an alkyne moiety of formula —(CH₂)nCCH, where n is 1 or 2. In certain embodiments R²³ or R¹⁵ forms a fused 5-membered cycle with an atom of the adjacent 5-membered ring. In certain embodiments R²³ or R¹⁵ forms a fused 6-membered cycle with an atom of the adjacent 5-membered ring. In certain embodiments of formula (TZB2d) or (TZB2h), p5 is 1. In certain embodiments, p5 is 2. In certain other embodiments, p5 is 3.

In certain embodiments of the subject compounds, T¹-Z¹—B is of any one of formulae (TZB3a)-(TZB3d), and each of A⁸-A⁹, R¹⁰, z and p1 are as defined for formula (BZ3). In certain embodiments of formula (TZB3a) or (TZB3d), R²³ or R¹⁵ is H. In certain other embodiments, R²³ or R¹⁵ is optionally substituted (C₁-C₃)alkyl. In certain cases, R²³ or R¹⁵ is methyl. In certain embodiments, R²³ or R¹⁵ is an alkyne moiety of formula —(CH₂)nCCH, where n is 1 or 2. In certain embodiments R²³ or R⁵ forms a fused 5-membered cycle with an atom of the adjacent 6-membered ring. In certain embodiments R²³ or R¹⁵ forms a fused 6-membered cycle with an atom of the adjacent 6-membered ring. In certain embodiments of formula (TZB3d), p5 is 1. In certain embodiments, p5 is 2. In certain other embodiments, p5 is 3.

In certain embodiments of the subject compounds, T¹-Z¹—B is of any one of formulae (TZB4a)-(TZB4d), and each of R¹, p2 and p4 are as defined for formula (BZ4). In certain embodiments of formula (TZB4a) or (TZB4d), R²³ or R⁵ is H. In certain other embodiments, R²³ or R¹⁵ is optionally substituted (C₁-C₃)alkyl. In certain cases, R²³ or R¹⁵ is methyl. In certain embodiments, R²³ or R¹⁵ is an alkyne moiety of formula —(CH₂)nCCH, where n is 1 or 2. In certain embodiments R²³ or R⁵ forms a fused 5-membered cycle with an atom of the adjacent ring. In certain embodiments R²³ or R⁵ forms a fused 6-membered cycle with an atom of the adjacent ring. In certain embodiments of formula (TZB4d), p5 is 1. In certain embodiments, p5 is 2. In certain other embodiments, p5 is 3.

In certain embodiments of the subject compounds, T¹-Z¹—B is selected from any one of formulae (TZB5a)-(TZB5d), (TZB6a)-(TZB6d), (TZB7a)-(TZB7d), and (TZB8a)-(TZB8d), and each of R², and p3 are as defined for formula (BZ5). In certain embodiments R²³ or R¹⁵ is H. In certain other embodiments, R²³ or R¹⁵ is optionally substituted (C₁-C₃)alkyl. In certain cases, R²³ or R¹⁵ is methyl. In certain embodiments, R²³ or R¹⁵ is an alkyne moiety of formula —(CH₂)nCCH, where n is 1 or 2. In certain embodiments of formula (TZB4d), (TZB6d), (TZB7d), or (TZB8d), p5 is 1. In certain embodiments, p5 is 2. In certain other embodiments, p5 is 3.

In certain embodiments of the subject compounds, T¹-Z¹—B is of formula (TZB9). In certain cases, the compound of formula (TZB9) is of any one of the following structures:

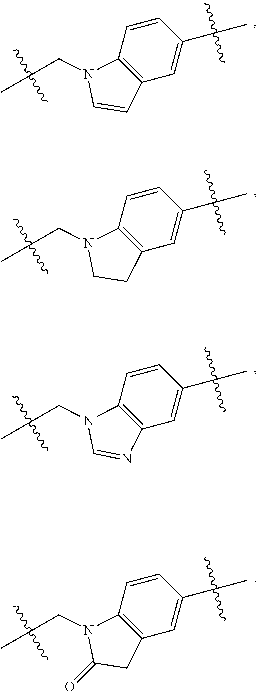 [SMILES: *Cn1ccc2cc(*)ccc21 ; *CN1CCc2cc(*)ccc21 ; *Cn1cnc2cc(*)ccc21 ; *CN1C(=O)Cc2cc(*)ccc21]

In certain embodiments of the subject compounds, T¹-Z¹ is optionally substituted (C₁-C₆)alkylene, and A-T¹-Z¹—B— is selected from one of formulae (AB1)-(AB6):

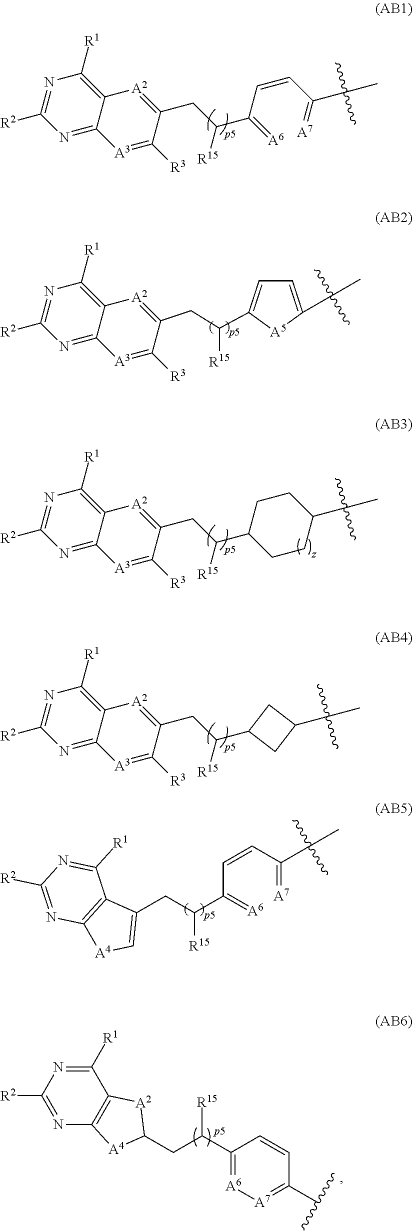

or a tautomer thereof, wherein:
A²-A⁷, R¹-R³ and z are as described herein above; each R¹⁵ is independently selected from H, halogen, OH, optionally substituted (C₁-C₆)alkyl,
optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
each p5 is independently 1 to 3.

In certain embodiments of the subject compounds, A-T¹-Z¹—B— is of formula (AB1), and each of A²-A³, A⁶-A⁷, and R¹-R³ are as described herein. In certain instances, R¹ is OH or NH₂. In certain instances, R² is NH₂, CH₃, or CH₂OH. In certain instances, R³ is H. In certain instances, both A² and A³ are N. In certain other instances, both A² and A³ are CH. In certain instances, both A⁶ and A⁷ are CH. In certain embodiments of formula (AB1), R⁵ is H. In certain other embodiments, R¹⁵ is optionally substituted (C₁-C₃)alkyl. In certain cases, R⁵ is methyl. In certain embodiments, R¹⁵ is an alkyne moiety of formula —(CH₂)nCCH, where n is 1 or 2. In certain embodiments R⁵ forms a fused 5-membered cycle with an atom of the adjacent aryl or heteroaryl ring. In certain embodiments R¹⁵ forms a fused 6-membered cycle with an atom of the adjacent aryl or heteroaryl ring. In certain embodiments of formula (AB1), p5 is 1. In certain embodiments, p5 is 2. In certain other embodiments, p5 is 3.

In certain embodiments of formula (AB1), the compound is selected from one of the following:

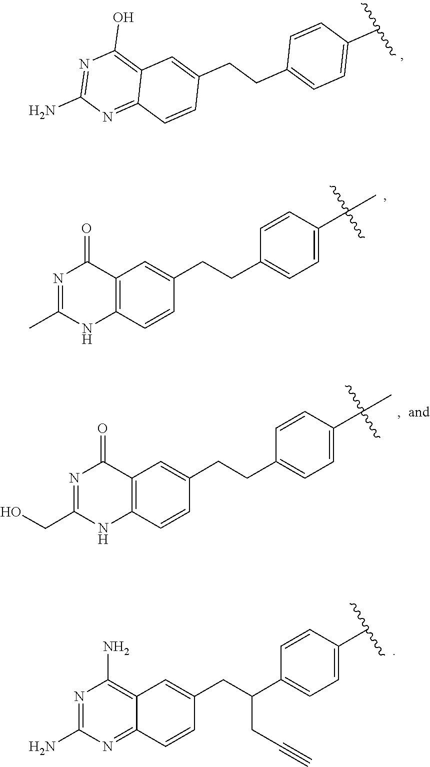 [SMILES: *c1ccc(CCc2ccc3nc(N)nc(O)c3c2)cc1 ; *c1ccc(CCc2ccc3[nH]c(C)nc(=O)c3c2)cc1 ; *c1ccc(CCc2ccc3[nH]c(CO)nc(=O)c3c2)cc1 ; *c1ccc(C(CC#C)Cc2ccc3nc(N)nc(N)c3c2)cc1]

In certain embodiments of the subject compounds, A-T¹-Z¹—B— is of formula (AB2), and each of A²-A³, A⁵, and R¹-R³ are as described herein. In certain instances, R¹ is OH or NH₂. In certain instances, R² is NH₂, CH₃, or CH₂OH. In certain instances, R³ is H. In certain instances, both A² and A³ are N. In certain other instances, both A² and A³ are CH. In certain instances, A⁵ is S or O. In certain embodiments of formula (AB2), R⁵ is H. In certain other embodiments, R¹⁵ is optionally substituted (C₁-C₃)alkyl. In certain cases, R¹⁵ is methyl. In certain embodiments, R⁵ is an alkyne moiety of formula —(CH₂)nCCH, where n is 1 or 2. In certain embodiments R¹⁵ forms a fused 5-membered cycle with an atom of the adjacent 5-membered ring. In certain embodiments R¹⁵ forms a fused 6-membered cycle with an atom of the adjacent 5-membered ring. In certain embodiments of formula (AB2), p5 is 1. In certain embodiments, p5 is 2. In certain other embodiments, p5 is 3.

In certain embodiments of the subject compounds, A-T¹-Z¹—B— is of formula (AB3), and each of A²-A³, R¹-R³ and z are as described herein. In certain instances, R¹ is OH or NH₂. In certain instances, R² is NH₂, CH₃, or CH₂OH. In certain instances, R³ is H. In certain instances, both A² and A³ are N. In certain other instances, both A² and A³ are CH. In certain instances, z is 1. In certain embodiments of formula (AB3), R¹⁵ is H. In certain other embodiments, R¹⁵ is optionally substituted (C₁-C₃)alkyl. In certain cases, R⁵ is methyl. In certain embodiments, R¹⁵ is an alkyne moiety of formula —(CH₂)nCCH, where n is 1 or 2. In certain embodiments R⁵ forms a fused 5-membered cycle with an atom of the adjacent cycloalkyl ring. In certain embodiments R⁵ forms a fused 6-membered cycle with an atom of the adjacent cycloalkyl ring. In certain embodiments of formula (AB1), p5 is 1. In certain embodiments, p5 is 2. In certain other embodiments, p5 is 3.

In certain embodiments of formula (AB3), the compound is of the following structure:

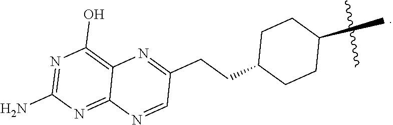 [SMILES: C[C@H]1CC[C@H](CCc2cnc3nc(N)nc(O)c3n2)CC1]

In certain embodiments of the subject compounds, A-T¹-Z¹—B— is of formula (AB4), and each of A²-A³, and R¹-R³ are as described herein. In certain instances, R¹ is OH or NH₂. In certain instances, R² is NH₂, CH₃, or CH₂OH. In certain instances, R³ is H. In certain instances, both A² and A³ are N. In certain other instances, both A² and A³ are CH. In certain embodiments of formula (AB4), R⁵ is H. In certain other embodiments, R¹⁵ is optionally substituted (C₁-C₃)alkyl. In certain cases, R⁵ is methyl. In certain embodiments, R¹⁵ is an alkyne moiety of formula —(CH₂)nCCH, where n is 1 or 2. In certain embodiments of formula (AB4), p5 is 1. In certain embodiments, p5 is 2. In certain other embodiments, p5 is 3.

In certain embodiments of the subject compounds, A-T¹-Z¹—B— is of formula (AB5) or (AB6), and each of A², A⁴, A⁶-A⁷, and R¹-R² are as described herein. In certain instances, R¹ is OH or NH₂. In certain instances, R² is NH₂, CH₃, or CH₂OH. In certain instances of formula (AB6), A² is CH. In certain other instances of formula (AB5) and (AB6), A⁴ is NH. In certain instances, both A⁶ and A⁷ are CH. In certain instances, A⁶ is CH and A⁷ are N. In certain embodiments of formula (AB5) or (AB6), R¹⁵ is H. In certain other embodiments, R¹ is optionally substituted (C₁-C₃)alkyl. In certain cases, R¹⁵ is methyl. In certain embodiments, R⁵ is an alkyne moiety of formula —(CH₂)nCCH, where n is 1 or 2. In certain embodiments R¹⁵ forms a fused 5-membered cycle with an atom of the adjacent aryl or heteroaryl ring. In certain embodiments R¹⁵ forms a fused 6-membered cycle with an atom of the adjacent aryl or heteroaryl ring. In certain embodiments of formula (AB5) or (AB6), p5 is 1. In certain embodiments, p5 is 2. In certain other embodiments, p5 is 3.

In certain embodiments, the compound of formula (AB5) or (AB6) is selected from the following structures:

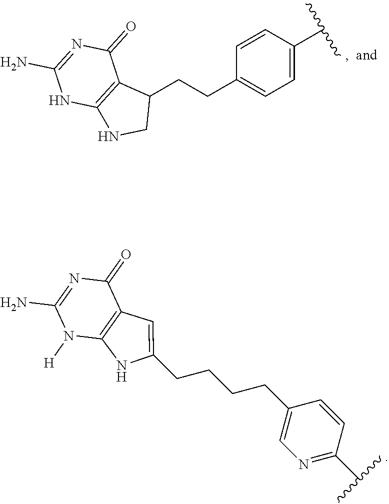 [SMILES: *c1ccc(CCC2CNc3[nH]c(N)nc(=O)c32)cc1 ; *c1ccc(CCCCc2cc3c(=O)nc(N)[nH]c3[nH]2)cn1]

In certain embodiments of the subject compounds, A-T¹-Z¹—B— is selected from one of formulae (AB7)-(AB12):

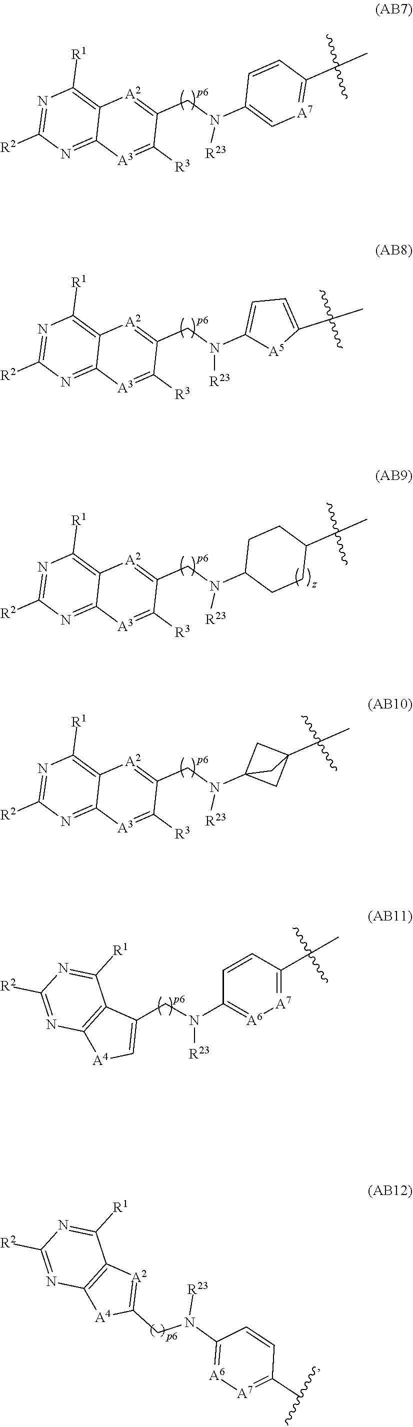

or a tautomer thereof, wherein:
A²-A⁷, R¹-R³ and z are as described herein above;
R²³ is H, optionally substituted (C₁-C₆)alkyl, or R²³ forms a 5 or 6 membered cycle together with an atom of the adjacent cycle;
each p6 is independently 1 to 3.

In certain embodiments of formula (AB7) to (AB12), R²³ is H. In certain other embodiments, R²³ is optionally substituted (C₁-C₃)alkyl. In certain cases, R²³ is methyl. In certain embodiments, R²³ is an alkyne moiety of formula —(CH₂)nCCH, where n is 1 or 2. In certain embodiments R²³ forms a fused 5-membered cycle with an atom of the adjacent aryl or heteroaryl ring. In certain embodiments R²³ forms a fused 6-membered cycle with an atom of the adjacent aryl or heteroaryl ring. In certain embodiments of formula (AB7) to (AB12), p6 is 1. In certain embodiments, p6 is 2. In certain other embodiments, p6 is 3.

In certain embodiments of the subject compounds, A-T¹-Z¹—B— is selected from one of formulae (AB13)-(AB18):

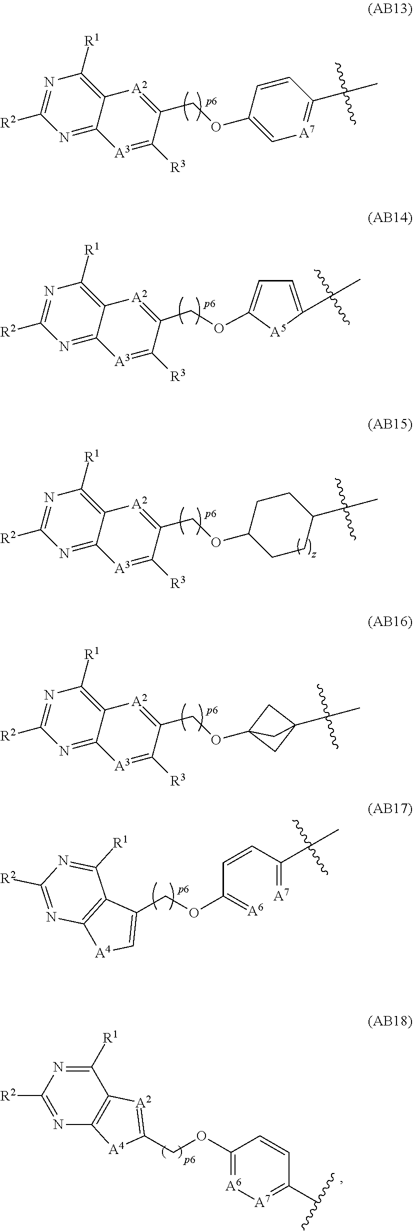

or a tautomer thereof, wherein:
A²-A⁷, R¹-R³ and z are as described herein above; and
each p6 is independently 1 to 3.

In certain embodiments of formula (AB13) to (AB18), p6 is 1. In certain embodiments, p6 is 2. In certain other embodiments, p6 is 3.

In certain embodiments of the subject compounds, A-T¹-Z¹—B— is selected from one of formulae (AB19)-(AB24):

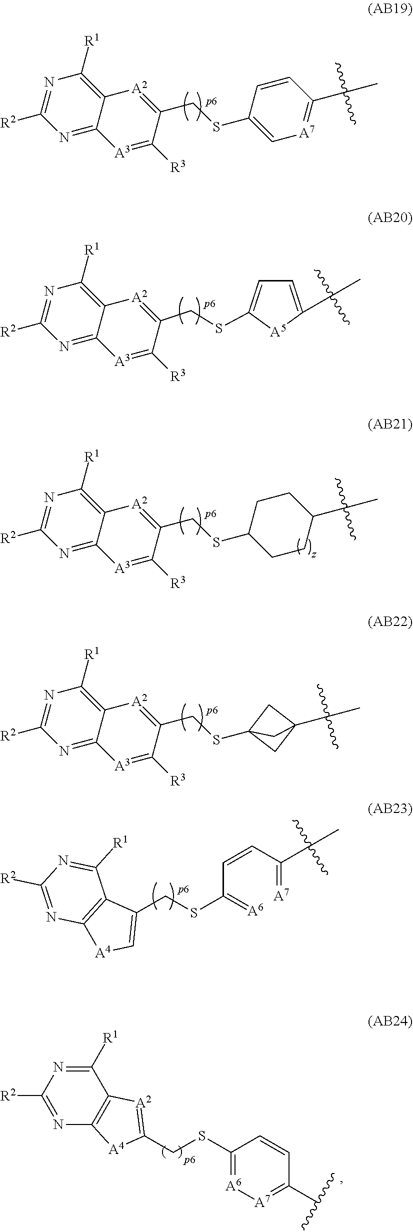

or a tautomer thereof, wherein:
A²-A⁷, R¹-R³ and z are as described herein above; and
each p6 is independently 1 to 3.

In certain embodiments of formula (AB19) to (AB24), p6 is 1. In certain embodiments, p6 is 2. In certain other embodiments, p6 is 3.

In some embodiments, the subject compound comprises a cell surface folate receptor ligand selected from one of the following structures:

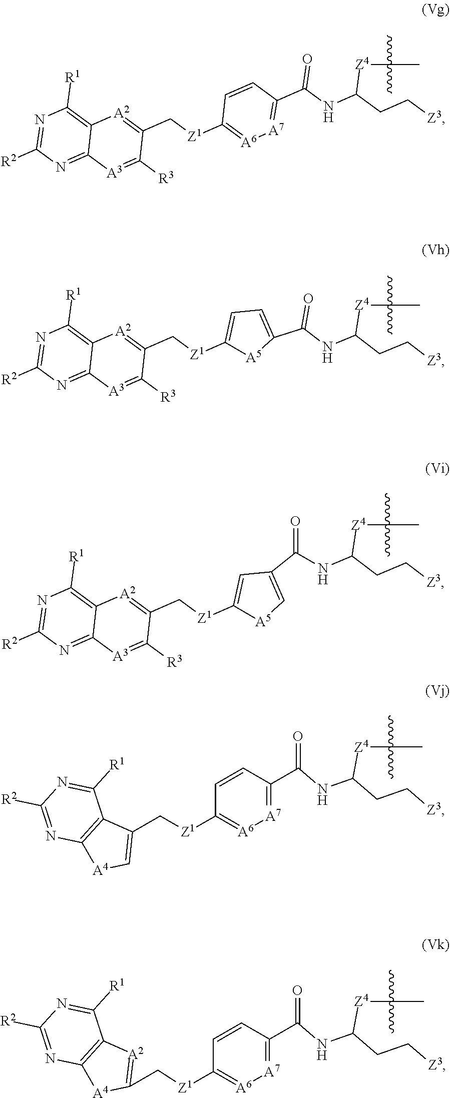

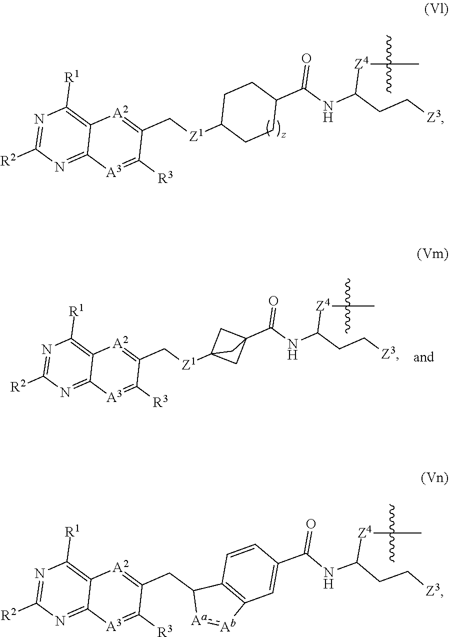

wherein:
A⁵ is selected from NR²¹, S, O, C(R)₂;
A⁶ and A⁷ are independently selected from N, and, CR⁵;
z is 0 to 3;
[symbol] is a single bond or a double bond;
wherein when [symbol] is a single bond A^{a} is selected from C(R)₂, and C═O, and A^{b} is selected from C(R⁵)₂, and NR²¹; and
when [symbol] is a double bond A^{a} is CR⁵, and A^{b} is selected from CR⁵ and N; and
wherein each R⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵.

In certain embodiments, the subject compound comprises a cell surface folate receptor ligand selected from one of the following structures:

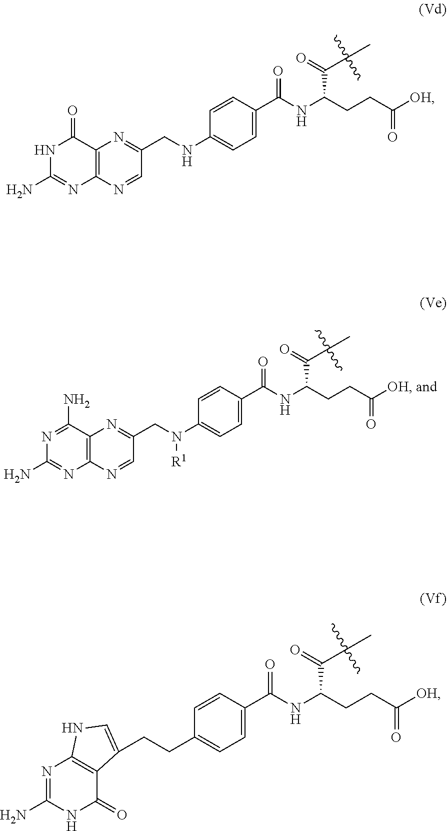

wherein R¹ is —H or —CH₃.

In certain embodiments, the subject compound comprises a cell surface folate receptor ligand is of formula (Vg) and each of R¹-R³, A²-A³, A⁶-A⁷, Z¹ and Z³-Z⁴ are as described herein above.

In certain embodiments, the subject compound comprises a cell surface folate receptor ligand is of formula (Vh) or (Vi) and each of R¹-R³, A²-A³, A⁵, Z¹ and Z³-Z⁴ are as described herein above.

In certain embodiments, the subject compound comprises a cell surface folate receptor ligand is of formula (Vj) or (Vk) and each of R¹-R², A², A⁴, A⁶-A⁷, Z¹ and Z³-Z⁴ are as described herein above.

In certain embodiments, the subject compound comprises a cell surface folate receptor ligand is of formula (Vl) and each of R¹-R³, A²-A³, z, Z¹ and Z³-Z⁴ are as described herein above.

In certain embodiments, the subject compound comprises a cell surface folate receptor ligand is of formula (Vm) and each of R¹-R³, A²-A³, Z¹ and Z³-Z⁴ are as described herein above.

In certain embodiments, the subject compound comprises a cell surface folate receptor ligand is of formula (Vn) and each of R¹-R³, A²-A³, A^{a}-A^{b}, and Z³-Z⁴ are as described herein above.

In some embodiments, the subject compound comprises a cell surface folate receptor ligand selected from one of the following structures:

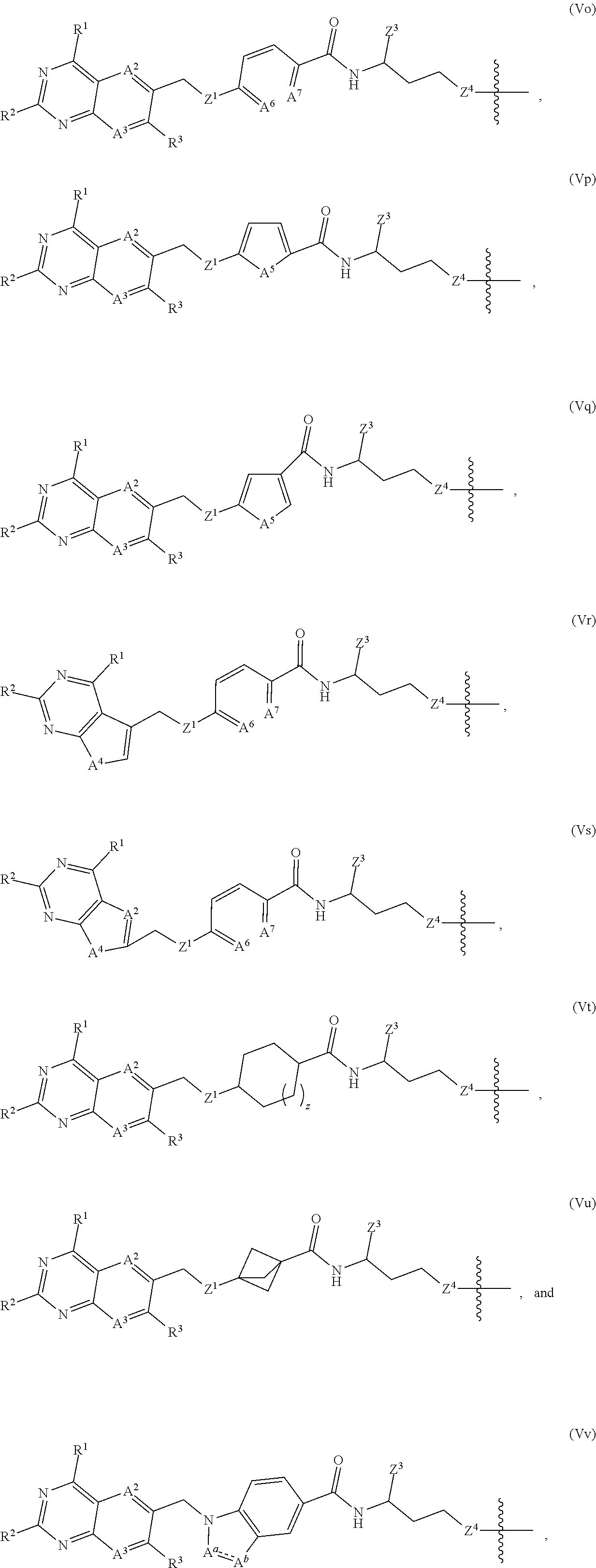

wherein:
A⁵ is selected from NR²¹, S, O, C(R²¹)₂;
A⁶ and A⁷ are each independently selected from N, and, CR²¹;
z is 0 to 3;
[symbol] is a single bond or a double bond;
wherein when [symbol] is a single bond A^{a} is selected from C(R²¹)₂, and C═O, and A^{b} is selected from C(R²¹)₂, and NR²¹; and
when [symbol] is a double bond A^{a} is CR²¹; and A^{b} is selected from CR²¹ and N.

In certain embodiments, the subject compound comprises a cell surface folate receptor ligand selected from one of the following structures:

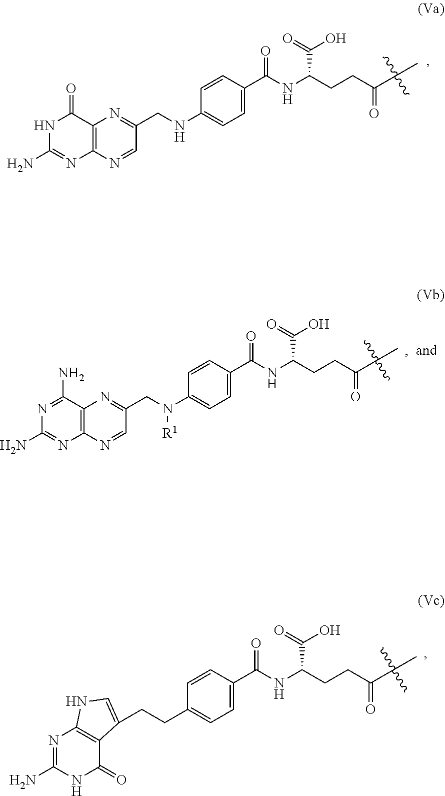

wherein R¹ is —H or —CH₃.

In certain embodiments, the subject compound comprises a cell surface folate receptor ligand is of formula (Vo) and each of R¹-R³, A²-A³, A⁶-A⁷, Z¹ and Z³-Z⁴ are as described herein above.

In certain embodiments, the subject compound comprises a cell surface folate receptor ligand is of formula (Vp) or (Vq) and each of R¹-R³, A²-A³, As, Z¹ and Z³-Z⁴ are as described herein above.

In certain embodiments, the subject compound comprises a cell surface folate receptor ligand is of formula (Vr) or (Vs) and each of R¹-R², A², A⁴, A⁶-A⁷, Z¹ and Z³-Z⁴ are as described herein above.

In certain embodiments, the subject compound comprises a cell surface folate receptor ligand is of formula (Vt) and each of R¹-R³, A²-A³, z, Z¹ and Z³-Z⁴ are as described herein above.

In certain embodiments, the subject compound comprises a cell surface folate receptor ligand is of formula (Vu) and each of R¹-R³, A²-A³, Z¹ and Z³-Z⁴ are as described herein above.

In certain embodiments, the subject compound comprises a cell surface folate receptor ligand is of formula (Vv) and each of R¹-R³, A²-A³, A^{a}-A^{b}, and Z³-Z⁴ are as described herein above.

In certain embodiments, the subject compound comprises a cell surface folate receptor ligand which can be utilized in the preparation of compounds of this disclosure are shown in tables 1-2.

TABLE 1
Exemplary cell surface folate receptor binding moieties

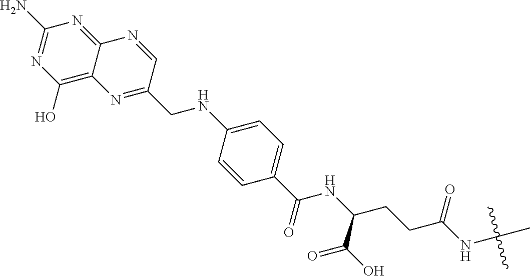 [SMILES: *NC(=O)CC[C@H](NC(=O)c1ccc(NCc2cnc3nc(N)nc(O)c3n2)cc1)C(=O)O]

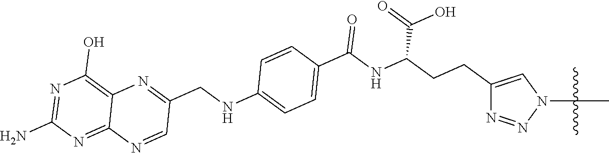 [SMILES: *n1cc(CC[C@H](NC(=O)c2ccc(NCc3cnc4nc(N)nc(O)c4n3)cc2)C(=O)O)nn1]

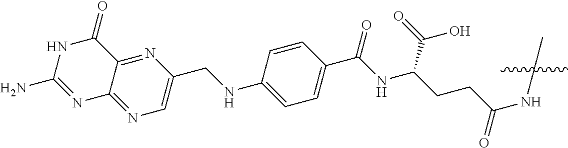 [SMILES: *NC(=O)CC[C@H](NC(=O)c1ccc(NCc2cnc3nc(N)[nH]c(=O)c3n2)cc1)C(=O)O]

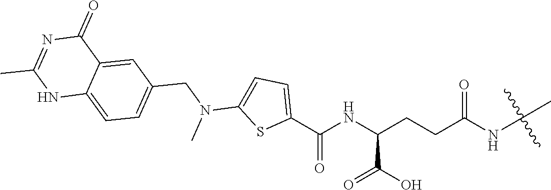 [SMILES: *NC(=O)CC[C@H](NC(=O)c1ccc(N(C)Cc2ccc3[nH]c(C)nc(=O)c3c2)s1)C(=O)O]

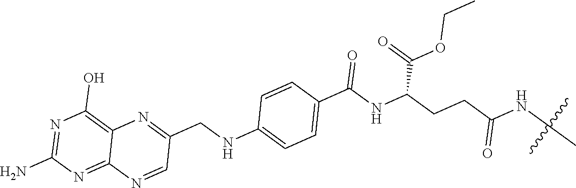 [SMILES: *NC(=O)CC[C@H](NC(=O)c1ccc(NCc2cnc3nc(N)nc(O)c3n2)cc1)C(=O)OCC]

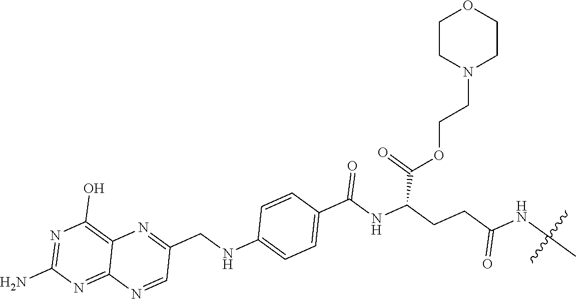 [SMILES: *NC(=O)CC[C@H](NC(=O)c1ccc(NCc2cnc3nc(N)nc(O)c3n2)cc1)C(=O)OCCN1CCOCC1]

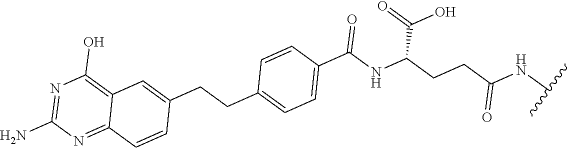 [SMILES: *NC(=O)CC[C@H](NC(=O)c1ccc(CCc2ccc3nc(N)nc(O)c3c2)cc1)C(=O)O]

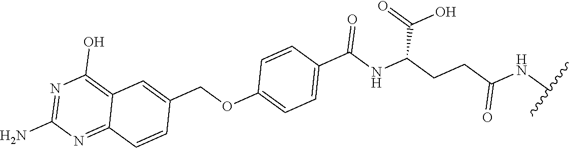 [SMILES: *NC(=O)CC[C@H](NC(=O)c1ccc(OCc2ccc3nc(N)nc(O)c3c2)cc1)C(=O)O]

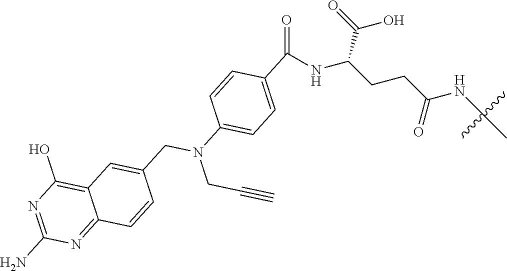 [SMILES: *NC(=O)CC[C@H](NC(=O)c1ccc(N(CC#C)Cc2ccc3nc(N)nc(O)c3c2)cc1)C(=O)O]

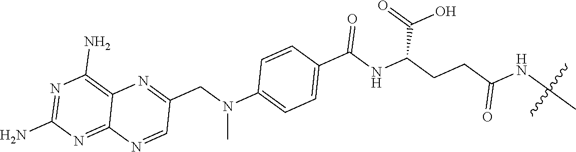 [SMILES: *NC(=O)CC[C@H](NC(=O)c1ccc(N(C)Cc2cnc3nc(N)nc(N)c3n2)cc1)C(=O)O]

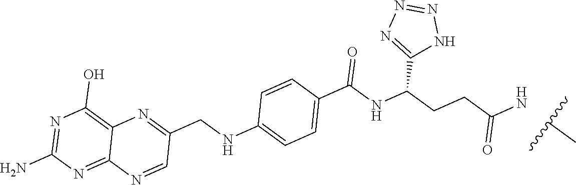

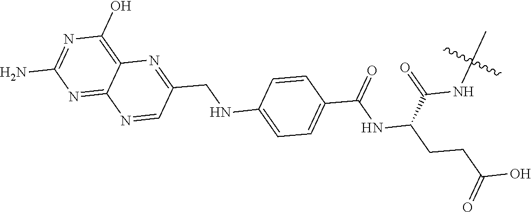 [SMILES: *NC(=O)[C@H](CCC(=O)O)NC(=O)c1ccc(NCc2cnc3nc(N)nc(O)c3n2)cc1]

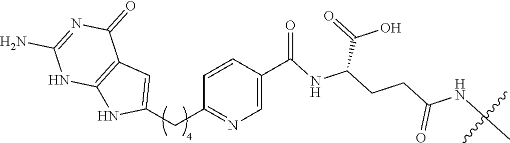

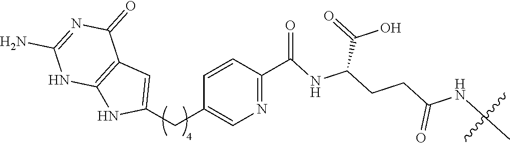

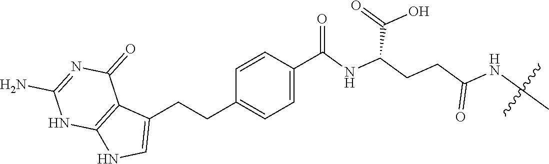 [SMILES: *NC(=O)CC[C@H](NC(=O)c1ccc(CCc2c[nH]c3[nH]c(N)nc(=O)c23)cc1)C(=O)O]

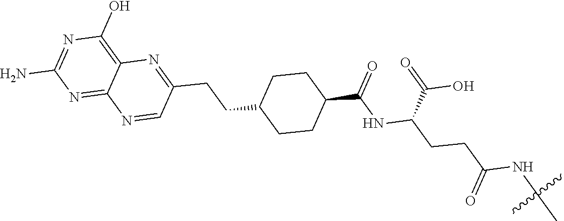

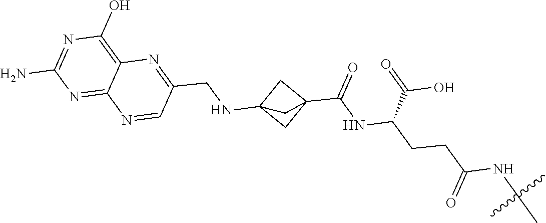 [SMILES: *NC(=O)CC[C@H](NC(=O)C12CC(NCc3cnc4nc(N)nc(O)c4n3)(C1)C2)C(=O)O]

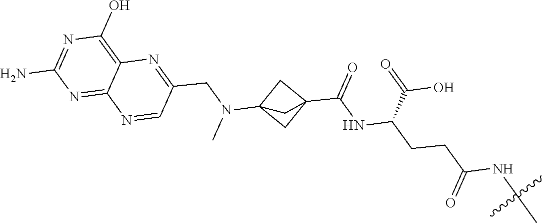 [SMILES: *NC(=O)CC[C@H](NC(=O)C12CC(N(C)Cc3cnc4nc(N)nc(O)c4n3)(C1)C2)C(=O)O]

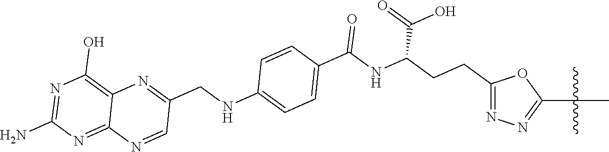 [SMILES: *c1nnc(CC[C@H](NC(=O)c2ccc(NCc3cnc4nc(N)nc(O)c4n3)cc2)C(=O)O)o1]

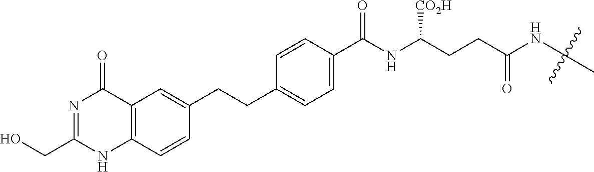 [SMILES: *NC(=O)CC[C@H](NC(=O)c1ccc(CCc2ccc3[nH]c(CO)nc(=O)c3c2)cc1)C(=O)O]

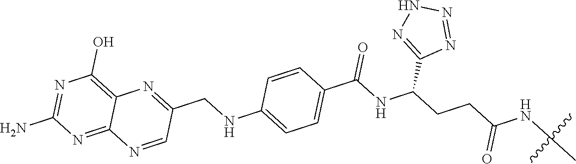 [SMILES: *NC(=O)CC[C@H](NC(=O)c1ccc(NCc2cnc3nc(N)nc(O)c3n2)cc1)c1nn[nH]n1]

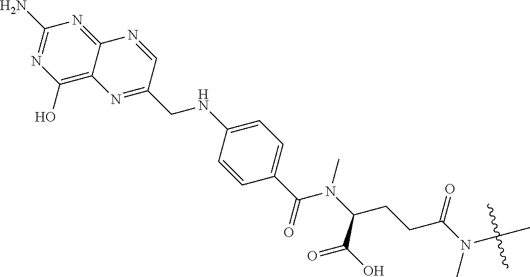 [SMILES: *N(C)C(=O)CC[C@@H](C(=O)O)N(C)C(=O)c1ccc(NCc2cnc3nc(N)nc(O)c3n2)cc1]

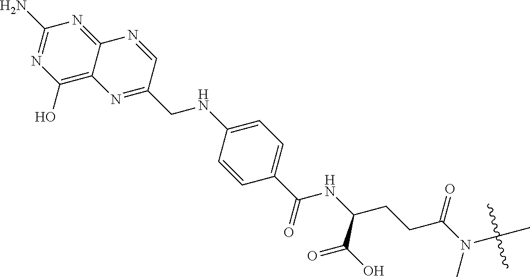 [SMILES: *N(C)C(=O)CC[C@H](NC(=O)c1ccc(NCc2cnc3nc(N)nc(O)c3n2)cc1)C(=O)O]

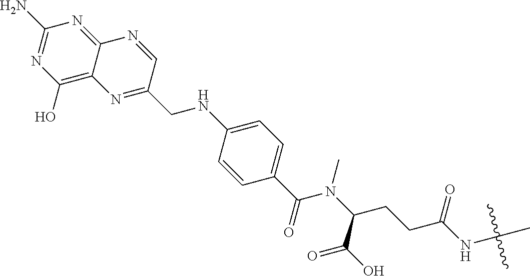 [SMILES: *NC(=O)CC[C@@H](C(=O)O)N(C)C(=O)c1ccc(NCc2cnc3nc(N)nc(O)c3n2)cc1]

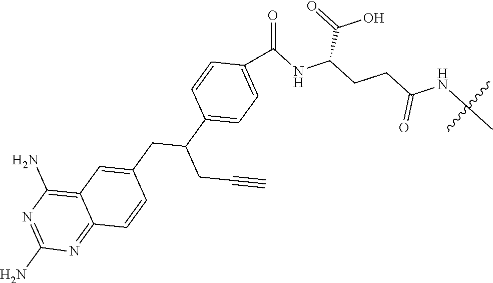 [SMILES: *NC(=O)CC[C@H](NC(=O)c1ccc(C(CC#C)Cc2ccc3nc(N)nc(N)c3c2)cc1)C(=O)O]

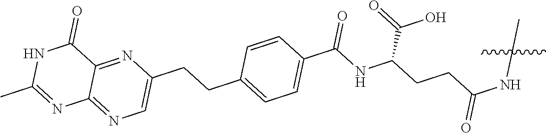 [SMILES: *NC(=O)CC[C@H](NC(=O)c1ccc(CCc2cnc3nc(C)[nH]c(=O)c3n2)cc1)C(=O)O]

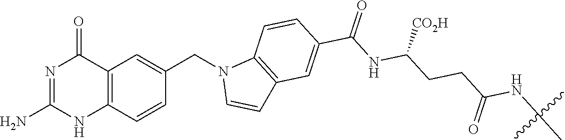 [SMILES: *NC(=O)CC[C@H](NC(=O)c1ccc2c(ccn2Cc2ccc3[nH]c(N)nc(=O)c3c2)c1)C(=O)O]

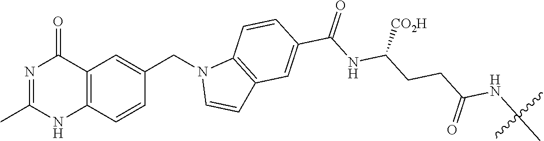 [SMILES: *NC(=O)CC[C@H](NC(=O)c1ccc2c(ccn2Cc2ccc3[nH]c(C)nc(=O)c3c2)c1)C(=O)O]

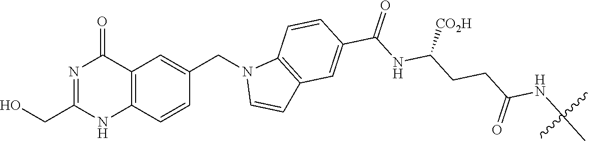 [SMILES: *NC(=O)CC[C@H](NC(=O)c1ccc2c(ccn2Cc2ccc3[nH]c(CO)nc(=O)c3c2)c1)C(=O)O]

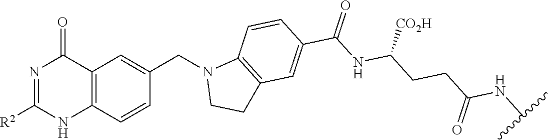

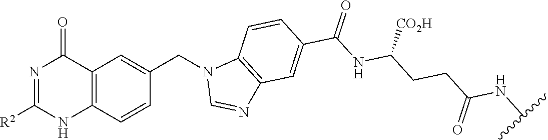

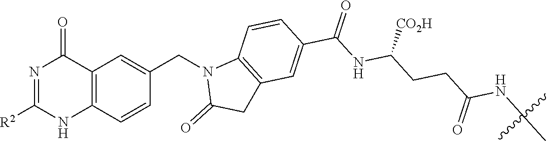

TABLE 2
cell surface folate receptor binding moieties

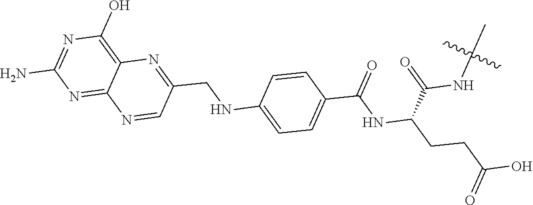 [SMILES: *NC(=O)[C@H](CCC(=O)O)NC(=O)c1ccc(NCc2cnc3nc(N)nc(O)c3n2)cc1]

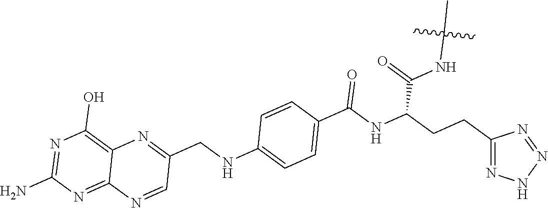 [SMILES: *NC(=O)[C@H](CCc1nn[nH]n1)NC(=O)c1ccc(NCc2cnc3nc(N)nc(O)c3n2)cc1]

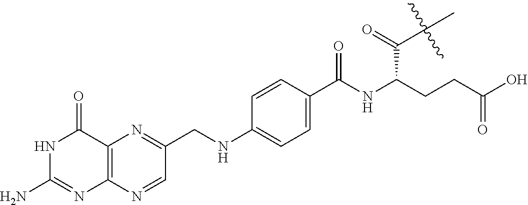 [SMILES: *C(=O)[C@H](CCC(=O)O)NC(=O)c1ccc(NCc2cnc3nc(N)[nH]c(=O)c3n2)cc1]

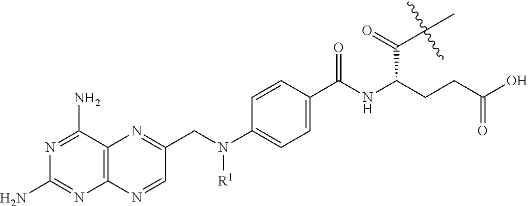

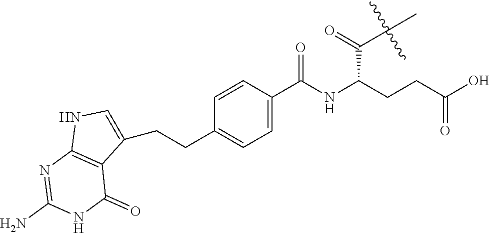 [SMILES: *C(=O)[C@H](CCC(=O)O)NC(=O)c1ccc(CCc2c[nH]c3nc(N)[nH]c(=O)c23)cc1]

In Tables 1 or 2, the [symbol] can represent the point of attachment to -L-Y.

In certain embodiments of the compound of formula (I), (IIIA), or (IIIB), n is 1. In certain cases, n is at least 2. In certain other cases, n is 2 to 20, such as 2 to 15, 2 to 10, 2 to 8, 2 to 6, or 2 to 4. In certain cases, n is 2 to 6. In certain other cases, n is 2 or 3.

Example compounds of formula (I), (IIIA) and (IIIB) are shown in tables 5-9.

### 5.1.1. Linkers

The terms “linker”, “linking moiety” and “linking group” are used interchangeably and refer to a linking moiety that covalently connects two or more moieties or compounds, such as ligands and other moieties of interest. In some cases, the linker is divalent and connects two moieties. In certain cases, the linker is a branched linking group that is trivalent or of a higher multivalency. In some cases, the linker that connects the two or more moieties has a linear or branched backbone of 500 atoms or less (such as 400 atoms or less, 300 atoms or less, 200 atoms or less, 100 atoms or less, 80 atoms or less, 60 atoms or less, 50 atoms or less, 40 atoms or less, 30 atoms or less, or even 20 atoms or less) in length, e.g., as measured between the two or more moieties. A linking moiety may be a covalent bond that connects two groups or a linear or branched chain of between 1 and 500 atoms in length, for example of about 1, 2, 3, 4, 5, 6, 8, 10, 12, 14, 16, 18, 20, 30, 40, 50, 100, 150, 200, 300, 400 or 500 carbon atoms in length, where the linker may be linear, branched, cyclic or a single atom. In certain cases, one, two, three, four, five or more, ten or more, or even more carbon atoms of a linker backbone may be optionally substituted with heteroatoms, e.g., sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, one or more of the following: oligo(ethylene glycol), ether, thioether, disulfide, amide, carbonate, carbamate, tertiary amine, alkyl which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle, a cycloalkyl group or a heterocycle group, where 2 or more atoms, e.g., 2, 3 or 4 atoms, of the cyclic group are included in the backbone.

In some embodiments, a “linker” or linking moiety is derived from a molecule with two reactive termini, one for conjugation to a moiety of interest (Y), e.g., a biomolecule (e.g., an antibody) and the other for conjugation to a moiety (noted as X) that binds to a cell surface folate receptor. For example, the moiety may be folic acid or an analog of a folic acid or folate moiety. When Y is a polypeptide, the polypeptide conjugation reactive terminus of the linker is in some cases a site that is capable of conjugation to the polypeptide through a cysteine thiol or lysine amine group on the polypeptide, and so is can be a thiol-reactive group such as a maleimide or a dibromomaleimide, or as defined herein, or an amine-reactive group such as an active ester (e.g., perfluorophenyl ester or tetrafluorophenyl ester), or as defined herein.

In certain embodiments of the formula described herein, the linker L comprises one or more straight or branched-chain carbon moieties and/or polyether (e.g., ethylene glycol) moieties (e.g., repeating units of —CH₂CH₂O—), and combinations thereof. In certain embodiments, these linkers optionally have amide linkages, urea or thiourea linkages, carbamate linkages, ester linkages, amino linkages, ether linkages, thioether linkages, sulfhydryl linkages, or other hetero functional linkages. In certain embodiments, the linker comprises one or more of carbon atoms, nitrogen atoms, sulfur atoms, oxygen atoms, and combinations thereof. In certain embodiments, the linker comprises one or more of an ether bond, thioether bond, amine bond, amide bond, carbon-carbon bond, carbon-nitrogen bond, carbon-oxygen bond, carbon-sulfur bond, and combinations thereof. In certain embodiments, the linker comprises a linear structure. In certain embodiments, the linker comprises a branched structure. In certain embodiments, the linker comprises a cyclic structure.

In certain embodiments, L is between about 10 Å and about 20 Å in length. In certain embodiments, L is between about 15 Å and about 20 Å in length. In certain embodiments, L is about 15 Å in length. In certain embodiments, L is about 16 Å in length. In certain embodiments, L is about 17 Å in length.

In certain embodiments, L is a linker between about 5 Å and about 500 Å. In certain embodiments, L is between about 10 Å and about 400 Å. In certain embodiments, L is between about 10 Å and about 300 Å. In certain embodiments, L is between about 10 Å and about 200 Å. In certain embodiments, L is between about 10 Å and about 100 Å. In certain embodiments, L is between about 10 Å and about 20 Å, between about 20 Å and about 30 Å, between about 30 Å and about 40 Å, between about 40 Å and about 50 Å, between about 50 Å and about 60 Å, between about 60 Å and about 70 Å, between about 70 Å and about 80 Å, between about 80 Å and about 90 Å, or between about 90 Å and about 100 Å. In certain embodiments, L is a linker between about 5 Å and about 500 Å, which comprises an optionally substituted arylene linked to a cell surface folate receptor binding moiety (X), optionally substituted heteroarylene linked to X, optionally substituted heterocyclene linked to X, or optionally substituted cycloalkylene linked to X. In certain embodiments, L is a linker between about 10 Å and about 500 Å, which comprises an optionally substituted arylene linked to X, optionally substituted heteroarylene linked to X, optionally substituted heterocyclene linked to X, or optionally substituted cycloalkylene linked to X. In certain embodiments, L is a linker between about 10 Å and about 400 Å, which comprises an optionally substituted arylene linked to X, optionally substituted heteroarylene linked to X, optionally substituted heterocyclene linked to X, or optionally substituted cycloalkylene linked to X. In certain embodiments, L is a linker between about 10 Å and about 200 Å, which comprises an optionally substituted arylene linked to X, optionally substituted heteroarylene linked to X, optionally substituted heterocyclene linked to X, or optionally substituted cycloalkylene linked to X.

In certain embodiments, L separates cell surface folate receptor binding moiety (Y) and Y (or Z) by a backbone comprising at least 10 consecutive atoms. In certain cases, the backbone is at least 12 consecutive atoms. In certain cases, the backbone is at least 14 consecutive atoms. In certain cases, the backbone is at least 16 consecutive atoms. In certain cases, the backbone is at least 18 consecutive atoms. In certain cases, the backbone is at least 20 consecutive atoms. In certain cases, the backbone is at least 22 consecutive atoms. In certain cases, the backbone is at least 24 consecutive atoms. In certain cases, the backbone is at least 26 consecutive atoms. In certain cases, the backbone is at least 28 consecutive atoms. In certain cases, the backbone is at least 30 consecutive atoms. In certain cases, the backbone is at least 32 consecutive atoms. In certain cases, the backbone is at least 34 consecutive atoms. In certain cases, the backbone is at least 36 consecutive atoms. In certain cases, the backbone is at least 38 consecutive atoms. In certain cases, the backbone is at least 40 consecutive atoms. In certain cases, the backbone is up to 50 consecutive atoms. In certain cases, the backbone is up to 60 consecutive atoms. In certain cases, the backbone is up to 70 consecutive atoms. In certain cases, the backbone is up to 80 consecutive atoms. In certain cases, the backbone is up to 90 consecutive atoms. In certain cases, the backbone is up to 100 consecutive atoms.

In certain embodiments, linker L separates cell surface folate receptor binding moiety (X) and Y (or Z) by a chain of 4 to 500 consecutive atoms. In certain embodiments, linker L separates X and Y (or Z) by a chain of 4 to 50 consecutive atoms. In certain embodiments, linker L separates X and Y (or Z) by a chain of 6 to 50 consecutive atoms, by a chain of 11 to 50 consecutive atoms, by a chain of 16 to 50 consecutive atoms, by a chain of 21 to 50 consecutive atoms, by a chain of 26 to 50 consecutive atoms, by a chain of 31 to 50 consecutive atoms, by a chain of 36 to 50 consecutive atoms, by a chain of 41 to 50 consecutive atoms, or by a chain of 46 to 50 consecutive atoms. In certain embodiments, linker L separates X and Y (or Z) by a chain of 6 to 50 consecutive atoms. In certain embodiments, linker L separates X and Y (or Z) by a chain of 11 to 50 consecutive atoms. In certain embodiments, linker L separates X and Y (or Z) by a chain of 16 to 50 consecutive atoms. In certain embodiments, linker L separates X and Y (or Z) by a chain of 21 to 50 consecutive atoms. In certain embodiments, linker L separates X and Y (or Z) by a chain of 26 to 50 consecutive atoms. In certain embodiments, linker L separates X and Y (or Z) by a chain of 31 to 50 consecutive atoms. In certain embodiments, linker L separates X and Y (or Z) by a chain of 36 to 50 consecutive atoms. In certain embodiments, linker L separates X and Y (or Z) by a chain of 41 to 50 consecutive atoms. In certain embodiments, linker L separates X and Y (or Z) by a chain of 46 to 50 consecutive atoms.

In certain embodiments, linker L separates X and Y (or Z) by a chain of 4 or 5 consecutive atoms, by a chain of 6 to 10 consecutive atoms, by a chain of 11 to 15 consecutive atoms, by a chain of 16 to 20 consecutive atoms, by a chain of 21 to 25 consecutive atoms, by a chain of 26 to 30 consecutive atoms, by a chain of 31 to 35 consecutive atoms, by a chain of 36 to 40 consecutive atoms, by a chain of 41 to 45 consecutive atoms, or by a chain of 46 to 50 consecutive atoms.

In certain embodiments, linker L separates X and Y (or Z) by a chain of 50 or 55 consecutive atoms, by a chain of 56 to 60 consecutive atoms, by a chain of 61 to 65 consecutive atoms, by a chain of 66 to 70 consecutive atoms, by a chain of 71 to 75 consecutive atoms, by a chain of 76 to 80 consecutive atoms, by a chain of 81 to 85 consecutive atoms, by a chain of 86 to 90 consecutive atoms, by a chain of 91 to 95 consecutive atoms, or by a chain of 96 to 100 consecutive atoms.

In certain embodiments, linker L is a chain of 5 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 7 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 10 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 15 to 400 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X.

In certain embodiments, linker L is a chain of 5 to 500 consecutive atoms separating X and Y (or Z) and which comprises an optionally substituted arylene linked to X or optionally substituted heteroarylene linked to X. In certain embodiments, linker L is a chain of 7 to 500 consecutive atoms separating X and Y (or Z) and which comprises an optionally substituted arylene linked to X or optionally substituted heteroarylene linked to X. In certain embodiments, linker L is a chain of 10 to 500 consecutive atoms separating X and Y (or Z) and which comprises an optionally substituted arylene linked to X or optionally substituted heteroarylene linked to X. In certain embodiments, linker L is a chain of 15 to 400 consecutive atoms separating X and Y (or Z) and which comprises an optionally substituted arylene linked to X or optionally substituted heteroarylene linked to X.

In certain embodiments, linker L is a chain of 5 to 500 consecutive atoms separating X and Y (or Z) and which comprises an optionally substituted phenylene linked to X. In certain embodiments, linker L is a chain of 7 to 500 consecutive atoms separating X and Y (or Z) and which comprises an optionally substituted phenylene linked to X. In certain embodiments, linker L is a chain of 10 to 500 consecutive atoms separating X and Y (or Z) and which comprises an optionally substituted phenylene linked to X. In certain embodiments, linker L is a chain of 15 to 400 consecutive atoms separating X and Y (or Z) and which comprises an optionally phenylene linked to X.

In certain embodiments, linker L is a chain of 16 to 400 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X.

It is understood that the linker may be considered as connecting directly to a Z⁴ group of a folate binding moiety (X) (e.g., as described herein). In some embodiments of formula (I), the linker may be considered as connecting directly to the Z³ group. Alternatively, the —Z²CH(-T³-Z³)T⁴Z⁴— group of formula (I) (e.g., as described herein) can be considered part of a linking moiety that connects Z⁴ to Y. The disclosure is meant to include all such configurations of folate binding moiety (X) and linker (L).

In certain embodiments of the subject compounds, L comprises one or more linking moieties independently selected from —C₁₋₆-alkylene-, —NHCO—C₁₋₆-alkylene-, —CONH—C₁₋₆-alkylene-, —NHC₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, pyrrolidine-2,5-dione, —NH—, and —NMe-, wherein p is 1 to 10.

In certain embodiments of the subject compounds, L comprises one or more —C₁₋₆-alkylene- linking moieties. In certain cases, L comprises one or more —NHCO—C₁₋₆-alkylene- linking moieties. In certain cases, L comprises one or more —CONH—C₁₋₆-alkylene- linking moieties. In certain cases, L comprises one or more —NHC₁₋₆-alkylene-linking moieties. In certain cases, L comprises one or more —NHCONH—C₁₋₆-alkylene- linking moieties. In certain cases, L comprises one or more —NHCSNH—C₁₋₆-alkylene- linking moieties. In certain cases, L comprises one or more —C₁₋₆-alkylene-NHCO— linking moieties. In certain cases, L comprises one or more —C₁₋₆-alkylene-CONH— linking moieties. In certain cases, L comprises one or more —C₁₋₆-alkylene-NH— linking moieties. In certain cases, L comprises one or more —C₁₋₆-alkylene-NHCONH— linking moieties. In certain cases, L comprises one or more —C₁₋₆-alkylene-NHCSNH— linking moieties. In certain cases, L comprises one or more —O(CH₂)ₚ— linking moieties. In certain cases, L comprises one or more —(OCH₂CH₂)ₚ— linking moieties. In certain cases, L comprises one or more —NHCO— linking moieties. In certain cases, L comprises one or more —CONH— linking moieties. In certain cases, L comprises one or more —NHSO₂— linking moieties. In certain cases, L comprises one or more —SO₂NH— linking moieties. In certain cases, L comprises one or more —CO— linking moieties. In certain cases, L comprises one or more —SO₂— linking moieties. In certain cases, L comprises one or more —O— linking moieties. In certain cases, L comprises one or more —S— linking moieties. In certain cases, L comprises one or more pyrrolidine-2,5-dione linking moieties. In certain cases, L comprises one or more —NH— linking moieties. In certain cases, L comprises one or more —NMe- linking moieties.

In certain embodiments of the subject compounds, L comprises repeating ethylene glycol moieties (e.g., —CH₂CH₂O— or —OCH₂CH₂—). In certain case, L comprises 1 to 20 ethylene glycol moieties. In certain cases, L comprise 2 to 18 ethylene glycol moieties. In certain cases, L comprise 2 to 16 ethylene glycol moieties. In certain cases, L comprises 2 to 14 ethylene glycol moieties. In certain cases, L comprises 2 to 12 ethylene glycol moieties. In certain cases, L comprises 2 to 10 ethylene glycol moieties. In certain cases, L comprises 2 to 8 ethylene glycol moieties. In certain cases, L comprises 2 to 8 ethylene glycol moieties. In certain cases, L comprises 2 to 6 ethylene glycol moieties.

In certain embodiments, L is of formula (IV):

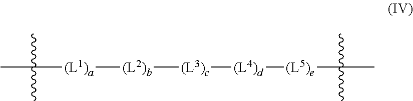

wherein
each L¹ to L⁵ is independently a linking moiety which together provide a linear or branched linker between Z⁴ and Y;
a is 1 or 2;
b, c, d, and e are each independently 0, 1, or 2.

In certain embodiments of formula (IV), -(L¹)ₐ- comprises an optionally substituted alkyl or ethylene glycol linking moiety. In certain cases, L¹ comprises an optionally substituted —C₁₋₆-alkylene-. In certain cases, L¹ comprises an ethylene glycol linking moiety.

In certain embodiments of formula (IV), L¹ is independently selected from: —C₁₋₆-alkylene-, —(CH₂CH₂O)ₜ—, —C₁₋₆-alkylene-NR⁴CO—, —C₁₋₆-alkyleneCONH—, or OCH₂, wherein t is 1 to 20; and R⁴ is independently selected from H, and optionally substituted (C₁-C₆)alkyl. In certain cases, L¹ is —C₁₋₆-alkylene-, such as —C₁₋₃-alkylene-. In certain cases, L¹ is —(CH₂CH₂O)ₜ—, where t is 1 to 20, such as 1 to 15, 1 to 10, 1 to 8, 1 to 6, or 1 to 4. In certain cases, L¹ is —C₁₋₆-alkylene-NR⁴CO—. In certain cases, L¹ is —C₁₋₆-alkyleneCONH—. In certain cases, L¹ is or OCH₂.

In certain embodiments of formula (IV), L² is independently selected from: —NR⁴CO—C₁₋₆-alkylene-, —CONR⁴—C₁₋₆-alkylene,

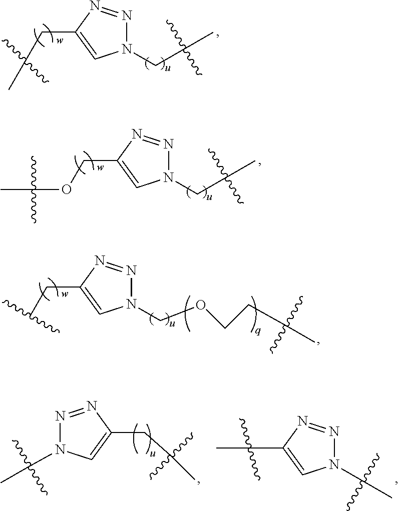

—OCH₂—, and —(OCH₂CH₂)_{q}—, wherein q is 1 to 10, u is 0 to 10, w is 1 to 10, and R⁴ is independently selected from H, and optionally substituted (C₁-C₆)alkyl. In certain cases, L² is —NR⁴CO—C₁₋₆-alkylene-. In certain cases, L² is —CONR⁴—C₁₋₆-alkylene.

In certain cases, L² is

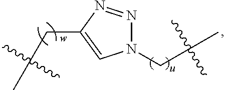

where w is 1 and u is 0 or 1.

In certain cases, L² is

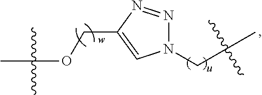

where w is 1 and u is 0 or 1.

In certain cases, L² is

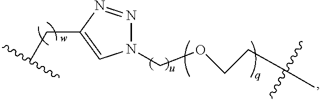

where w is 1, u is 0 or 1, and q is 1.

In certain cases, L² is

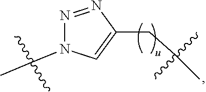

where u is 0 or 1.

In certain cases, L² is

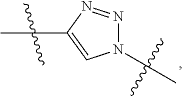

In certain embodiments, L² is —OCH₂—. In certain other embodiments, L² is (OCH₂CH₂)_{q}—, and q is 1 to 10, 1 to 9, 1 to 8, 1 to 7, 1 to 6, 1 to 5, 1 to 4, 1 to 3 or 1 to 2. In certain cases, q is 2 to 8, such as 2 to 6, 4 to 6, or 2 to 4.

In certain embodiments of formula (IV), L⁴ is absent or independently selected from —C₁₋₆-alkylene-, —(CH₂CH₂O)ₜ—, —C₁₋₆-alkylene-NHCO—, —C₁₋₆-alkyleneCONH—, or OCH₂, wherein t is 1 to 20. In certain cases, L⁴ is absent. In certain cases, L⁴ is —C₁₋₆-alkylene-. In certain cases, L⁴ is —(CH₂CH₂O)ₜ—, where t is 1 to 20, such as 1 to 15, 1 to 12, 1 to 10, 1 to 8, 1 to 6, 1 to 4 or 1 to 3. In certain cases, L⁴ is —C₁₋₆-alkylene-NHCO—. In certain cases, L⁴ is —C₁₋₆-alkyleneCONH—. In certain cases, L⁴ is OCH₂,

In some embodiments of formula (IV), each L³ is a linear or branched linking moiety. In certain cases, L³ is a linear linking moiety. In certain cases, L³ is —OCH₂CH₂—.

In certain embodiments of the subject compounds, n is 2 or more, at least one L³ is present and is a branched linking moiety.

Accordingly, in some embodiments of formula (IV), L³ is a branched linking moiety, e.g., a trivalent linking moiety. For example, an L³ linking moiety can be of the one of the following general formula:

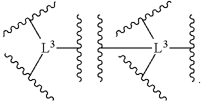

In some embodiments of formula (IV), the branched linking moiety can be of higher valency and be described by one of the one of the following general formula:

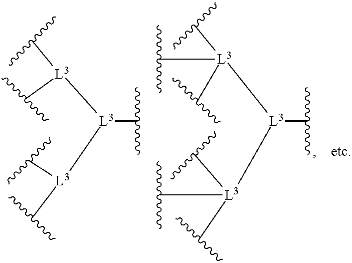

where any two L³ groups can be directed linked or connected via optional linear linking moieties (e.g., as described herein).

In some embodiments of formula (IV), the branched linking moiety can include one, two or more L³ linking moieties, each being trivalent moieties, which when linked together can provide for multiple branching points for covalent attachment of the ligands and be described by one of the one of the following general formula:

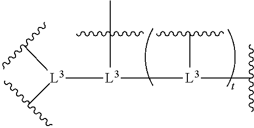

where t is 0 to 500, such as 0 to 100, 0 to 20, or 0 to 10.

In some embodiments, the branched linking moiety (e.g., L³) comprises one or more of: an amino acid residue (e.g., Asp, Lys, Orn, Glu), N-substituted amido (—N(—)C(═O)—), tertiary amino, polyol (e.g., O-substituted glycerol), and the like.

In some embodiments of formula (IV), one or more L³ is a branching moiety selected from

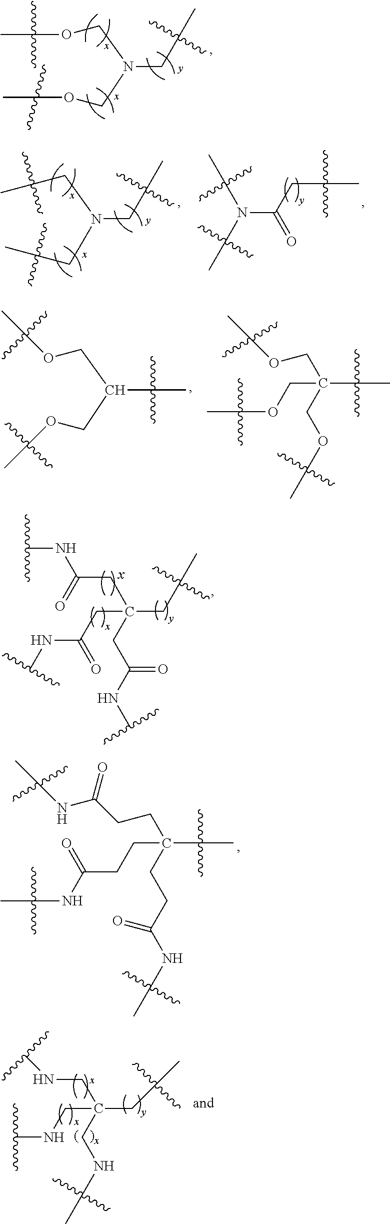

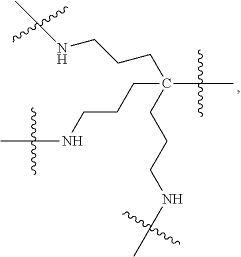

wherein each x and y are each independently 1 to 10, such as 1-6, 1-3, e.g., 1 or 2. In some cases, each x is 1, 2 or 3, e.g., 2.

In some embodiments of formula (IV) one or more L^{_}is independently —CH₂O—; —(CH₂CH₂O)ₜ—, —NR⁴CO—, —C₁₋₆-alkylene-,

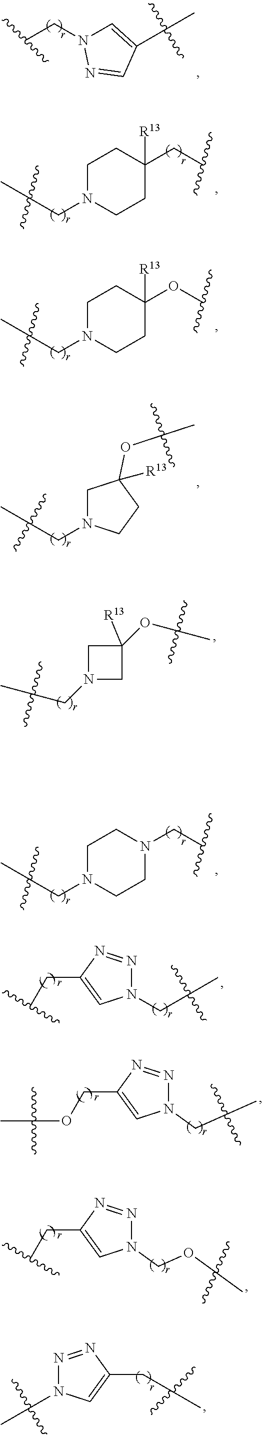

wherein:
R¹³ is selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; each r independently 0 to 20, and any of the L⁵ moieties are optionally further substituted.

In certain cases, L⁵ is —CH₂O—. In certain cases, L⁵ is —(CH₂CH₂O)ₜ—, where t is 1 to 20, such as 1-15, 1-12, 1-10, 1-8, 1-6, or 1 to 4. In certain cases, L⁵ is —NR⁴CO—, where R⁴ is H, or optionally substituted (C₁-C₆)alkyl. In certain cases, L⁵ is —C₁₋₆-alkylene-.

In certain cases, L⁵ is

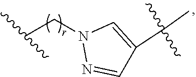

where r is 0 to 20, such as 0 to 15, 0 to 10, 0 to 8, or 0 to 5.

In certain cases, L⁵ is

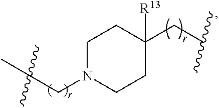

where each r is independently 0 to 20, such as 0 to 15, 0 to 10, 0 to 8, or 0 to 5 and R¹³ is H, or optionally substituted (C₁-C₆)alkyl.

In certain cases, L⁵ is

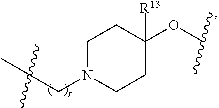

where r is 0 to 20, such as 0 to 15, 0 to 10, 0 to 8, or 0 to 5 and R¹³ is H, or optionally substituted (C₁-C₆)alkyl.

In certain cases, L⁵ is

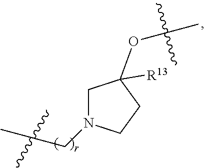

where r is 0 to 20, such as 0 to 15, 0 to 10, 0 to 8, or 0 to 5, and R¹³ is H, or optionally substituted (C₁-C₆)alkyl.

In certain cases, L⁵ is

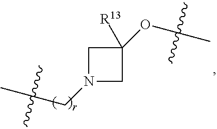

where r is 0 to 20, such as 0 to 15, 0 to 10, 0 to 8, or 0 to 5, and R¹³ is H, or optionally substituted (C₁-C₆)alkyl.

In certain cases, L⁵ is

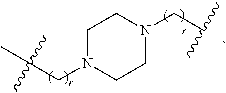

where each r is independently 0 to 20, such as 0 to 15, 0 to 10, 0 to 8, or 0 to 5.

In certain cases, L⁵ is

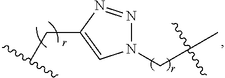

where each r is independently 0 to 20, such as 0 to 15, 0 to 10, 0 to 8, or 0 to 5.

In certain cases, L⁵ is

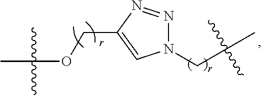

where each r is independently 0 to 20, such as 0 to 15, 0 to 10, 0 to 8, or 0 to 5.

In certain cases, L⁵ is

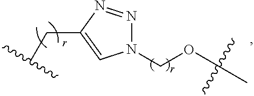

where each r is independently 0 to 20, such as 0 to 15, 0 to 10, 0 to 8, or 0 to 5.

In certain cases, L⁵ is

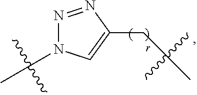

where r is 0 to 20, such as 0 to 15, 0 to 10, 0 to 8, or 0 to 5.

In certain embodiments of formula (IV), a is 1. In certain cases, at least one of b, c, d, and e is not 0. In certain cases, b is 1 or 2. In certain cases, c is 1 or 2. In certain cases, e is 1 or 2. In certain cases, b, d and e are independently 1 or 2. In certain cases, a, b, d, and e are each 1, and c is 0.

In certain embodiments of formula (IV), the linker comprises one or more of: an amino acid residue (e.g., Asp, Lys, Orn, Glu), an amino acid analogue, N-substituted amido (—N(—)C(═O)—), tertiary amino, polyol (e.g., O-substituted glycerol), and the like. Analogs of an amino acid, include but not limited to, unnatural amino acids, as well as other modifications known in the art. The amino acid includes L-amino acids, D-amino acids, or both, and may contain any of a variety of amino acid modifications or analogs known in the art.

In some embodiments of formula (IV), L comprises one or more of the following units:

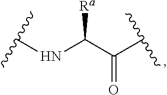

where R^{a} is (C₁-C₆)alkyl or substituted (C₁-C₆)alkyl, e.g., a (C₁-C₆)alkyl optionally substituted with amine, a tertiary amine, optionally substituted alkoxy, optionally substituted carboxyl, optionally substituted aryl, or optionally substituted heteroaryl.

In some embodiments of formula (IV), L has or comprises the following structure:

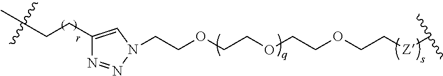

wherein:
r is 0 to 10;
q is 0 to 20 (e.g., 1, 2, 3, 4, 5, 6, 7, 8, 9, or 10);
s is 0 or 1;
Z′ is CO, NHCO, CONH or NH.

In some embodiments of formula (IV), L has or comprises the following structure:

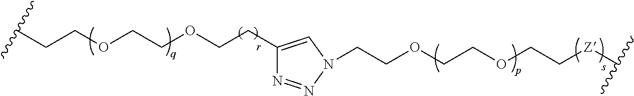

wherein:
r is 0 to 10;
p and q are 0 to 20;
s is 0 or 1; and
Z′ is CO, NHCO, CONH or NH.

In some embodiments of formula (IV), L has or comprises one of the following structures:

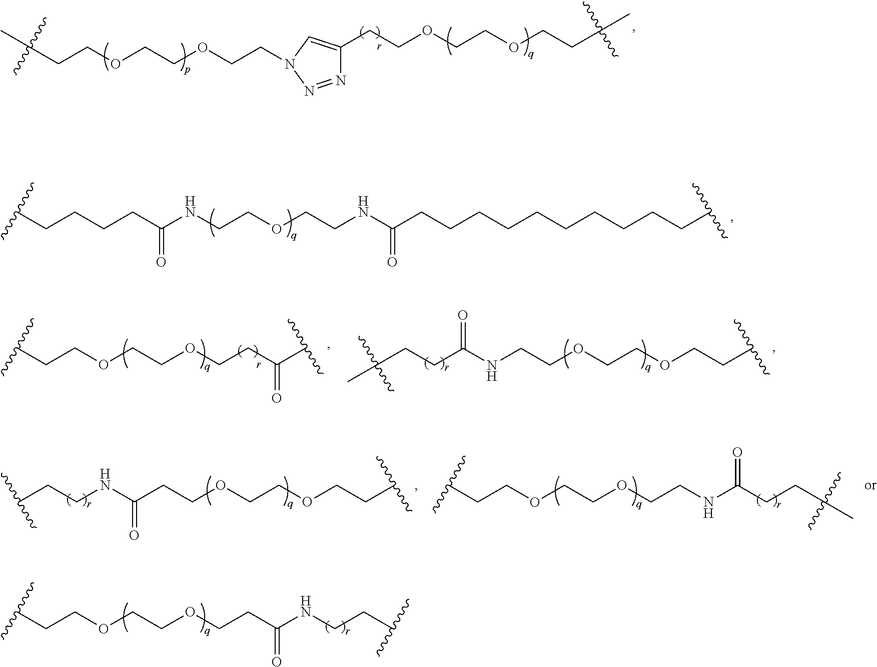

wherein r is 0 to 10, and p and q are independently 0 to 20.

Table 3 shows a variety of example linkers or linking moieties that find use in the compounds described herein. In some embodiments of formula (I), (IIIA) or (IIIB), the compound includes any one of the linkers or linking moieties set forth in Table 3.

TABLE 3
Example linkers of formula (IV)
Compound structure

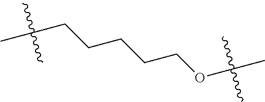 [SMILES: *CCCCCO*]

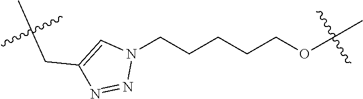 [SMILES: *Cc1cn(CCCCCO*)nn1]

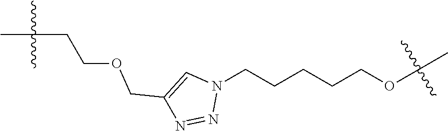 [SMILES: *CCOCc1cn(CCCCCO*)nn1]

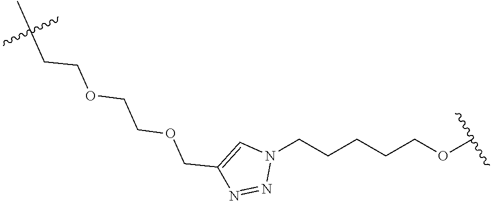 [SMILES: *CCOCCOCc1cn(CCCCCO*)nn1]

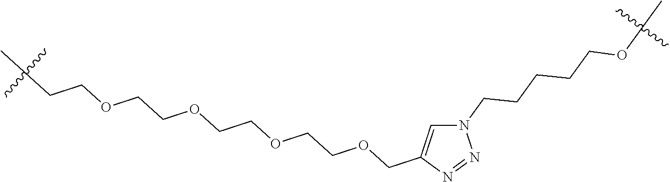 [SMILES: *CCOCCOCCOCCOCc1cn(CCCCCO*)nn1]

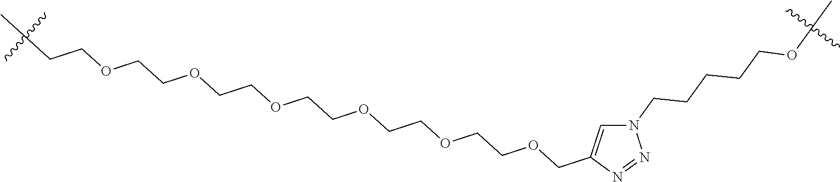 [SMILES: *CCOCCOCCOCCOCCOCCOCc1cn(CCCCCO*)nn1]

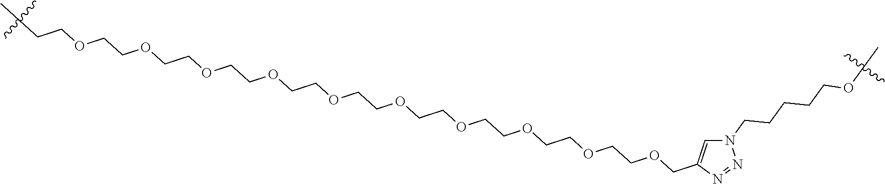 [SMILES: *CCOCCOCCOCCOCCOCCOCCOCCOCCOCCOCc1cn(CCCCCO*)nn1]

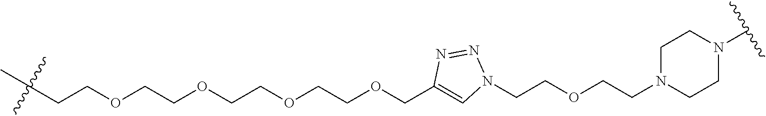

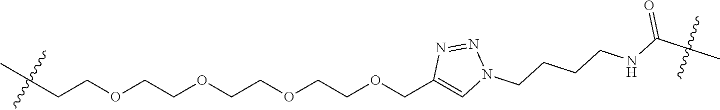

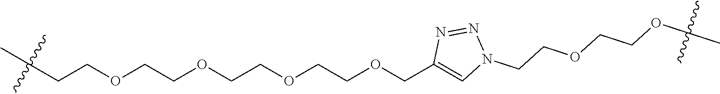

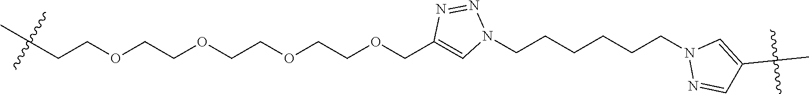

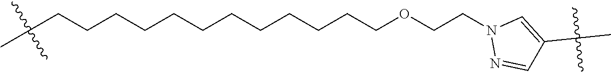 [SMILES: *CCCCCCCCCCCCOCCn1cc(*)cn1]

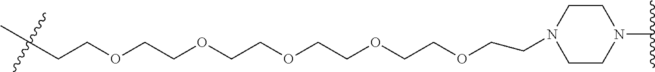 [SMILES: *CCOCCOCCOCCOCCOCCN1CCN(*)CC1]

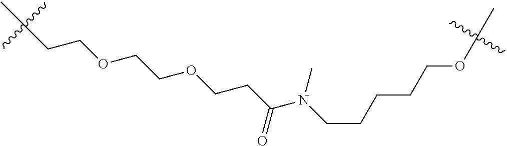 [SMILES: *CCOCCOCCC(=O)N(C)CCCCCO*]

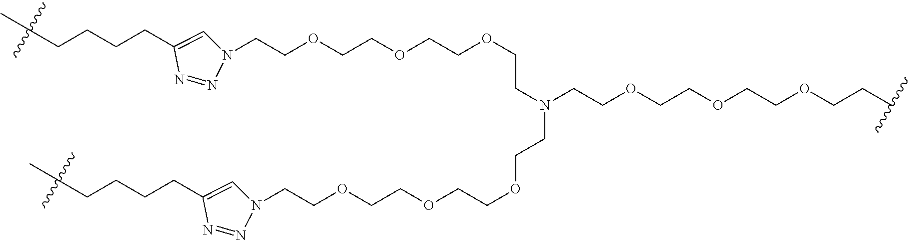 [SMILES: *CCCCc1cn(CCOCCOCCOCCN(CCOCCOCCOCC*)CCOCCOCCOCCn2cc(CCCC*)nn2)nn1]

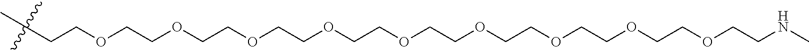 [SMILES: *CCOCCOCCOCCOCCOCCOCCOCCOCCOCCNC]

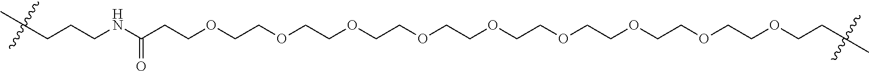 [SMILES: *CCCNC(=O)CCOCCOCCOCCOCCOCCOCCOCCOCCOCC*]

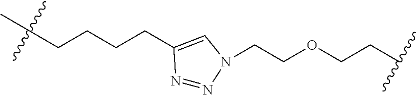 [SMILES: *CCCCc1cn(CCOCC*)nn1]

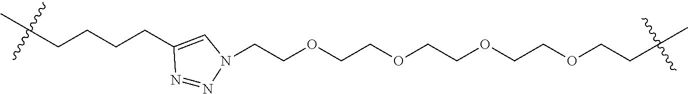 [SMILES: *CCCCc1cn(CCOCCOCCOCCOCC*)nn1]

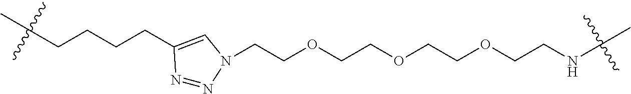 [SMILES: *CCCCc1cn(CCOCCOCCOCCN*)nn1]

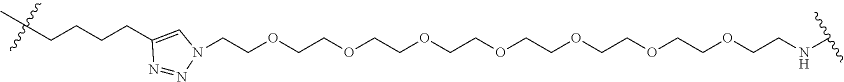 [SMILES: *CCCCc1cn(CCOCCOCCOCCOCCOCCOCCOCCN*)nn1]

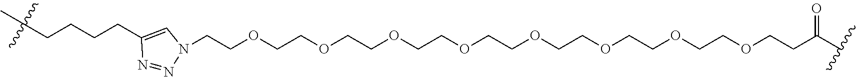 [SMILES: *CCCCc1cn(CCOCCOCCOCCOCCOCCOCCOCCOCCC(*)=O)nn1]

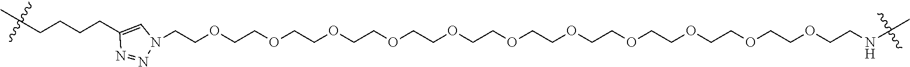 [SMILES: *CCCCc1cn(CCOCCOCCOCCOCCOCCOCCOCCOCCOCCOCCOCCN*)nn1]

In Table 3, the [symbol] can represent the point of attachment to X (on the left hand terminus) and Y (on the right hand terminus). It is understood that in any of the linking groups shown in Table 3, additional terminal groups (e.g., deriving from a functional group linkage) can be incorporated, such as —NH—, —CO—, —O—, —S—S—, —S—, —CONH—, SO2NH—, —NHCO—, NHSO2-, —COO—, etc.

### 5.1.2. Moiety of Interest (Y)

As summarized above, the compounds of this disclosure generally include a linked moiety of interest Y. In some embodiments, the moiety of interest Y is a chemoselective ligation group or a precursor thereof, and the compound can find use in the preparation of a variety of conjugates via conjugation of the chemoselective ligation group to a compatible reactive group of another moiety or molecule of interest, e.g., as described herein.

In some embodiments, the moiety of interest Y to which the folate receptor ligand is linked is itself a target molecule whose delivery to the interior of a target cell is desired. In some embodiments, Y is a target molecule that is a diagnostic agent. In some embodiments, Y is a target molecule that is a therapeutic agent.

In certain other embodiments, the moiety of interest Y to which the folate receptor ligand is linked is a moiety that specifically binds to an extracellular target protein. In certain cases, the target protein is a membrane bound protein. In certain cases, the target protein is a soluble extracellular protein. In such cases, the compounds of this disclosure bind to the extracellular target protein and can provide for folate receptor mediated internalization into the cell. The extracellular target protein of interest can be sequestered and/or degraded in the cell's lysosome.

In certain embodiments, the compound is a conjugate where Y is selected from a small molecule, peptide, protein, a dye, a fluorophore, a monosaccharide, a polysaccharide (e.g., disaccharide, or trisaccharide), lipid, enzyme, enzyme substrate, and a chemoselective ligation group, or precursor thereof.

In certain embodiments of the subject compounds, Y is a target-binding small molecule. In certain cases, Y is a small molecule inhibitor or ligand of the target protein.

In certain embodiments, the target protein of interest is PCSK9. In certain embodiments, Y is a small molecule that binds to PCSK9, such as any binder recited in WO2018/057409, or WO2021072269.

In certain embodiments, the target protein is VEGF.

In certain embodiments, the target protein is TGF-beta.

In certain embodiments, the target protein is IgA. In certain embodiments, Y that binds to IgA includes a peptide binder, or a Fc-alpha receptor peptide mimetic.

In certain embodiments, the target protein is MIF.

In some embodiments, Y is a target molecule that is a therapeutic agent.

In certain embodiments, Y is a chemotherapeutic agent. In certain embodiments, Y is a cytotoxic anticancer agent. Anticancer agents of interest which can be adapted for use in the subject compounds and conjugates include but are not limited to, antimitotic agent containing an epothilone moiety, antimitotic agent, e.g., maytansinoid DM4, vinca alkaloid, vinblastine, mitomycin C, paclitaxel, taxol or taxol derivative, and the like.

In some embodiments, Y is an immunotherapeutic agent. In some embodiments, Y is a cancer immunotherapeutic.

In some embodiments, Y is a toll-like receptor (TLR) ligand, e.g., a TLR agonist or antagonist. Any convenient TLR can be targeted, including but not limited to TLR3, TLR4, TLR7, TLR8, and TLR9. Any convenient TLR ligands, e.g., agonists, can be adapted for use in the compounds and conjugates of this disclosure, such as the TLR ligands described in US 20180289789. The TLR ligand can be PAMP ligand (pathogen-associated molecular patterns), an endogenous ligand, or a synthetic ligand. In some embodiments, the target TLR is TLR4. In some embodiments, Y is a lipopolysaccharide (LPS). In some embodiments, Y is a TLR ligand selected from al-acid glycoprotein (AGP), monophosphoryl lipid A (MPLA), RC-529, MDF20, and complete Freund's adjuvant (CFA). In some embodiments, Y is a CpG oligonucleotide, e.g., a TLR9 binding oligonucleotide containing a CpG motifs.

In certain embodiments, Y is a target-binding biomolecule. In certain cases, the biomolecule is selected from peptide, protein, glycoprotein, polynucleotide, aptamer, and antibody or antibody fragment. In certain cases, Y is selected from an antibody or an antibody fragment (e.g., an antigen-binding fragment of an antibody), chimeric fusion protein, an engineered protein domain, and a D-protein binder of target protein.

### Chemoselective Ligation Groups

In certain embodiments of formula (I), Y is a chemoselective ligation group, or a precursor thereof. A chemoselective ligation group is a group having a reactive functionality or function group capable of conjugation to a compatible group of a second moiety. For example, chemoselective ligation groups (or a precursor thereof) may be one of a pair of groups associated with a conjugation chemistry such as azido-alkyne click chemistry, copper free click chemistry, Staudinger ligation, tetrazine ligation, hydrazine-iso-Pictet-Spengler (HIPS) ligation, cysteine-reactive ligation chemistry (e.g., thiol-maleimide, thiol-haloacetamide or alkyne hydrothiolation), amine-active ester coupling, reductive amination, dialkyl squarate chemistry, etc.

Table 4 illustrates exemplary synthetic precursors of linker components that are used to prepare compounds of this disclosure and which have various chemoselective ligation groups. A variety of other chemical functional groups can also be incorporated as needed to prepare a desired linker.

Chemoselective ligation groups that may be utilized in linking two moieties, include, but are not limited to, amino (e.g., a N-terminal amino or a lysine sidechain group of a polypeptide), azido, aryl azide, alkynyl (e.g., ethynyl or cyclooctyne or derivative), active ester (e.g., N-hydroxysuccinimide (NHS) ester, sulfo-NHS ester or PFP ester or thioester), haloacetamide (e.g., iodoacetamide or bromoacetamide), chloroacetyl, bromoacetyl, hydrazide, maleimide, vinyl sulfone, 2-sulfonyl pyridine, cyano-alkyne, thiol (e.g., a cysteine residue), disulfide or protected thiol, isocyanate, isothiocyanate, aldehyde, ketone, alkoxyamine, hydrazide, aminooxy, phosphine, HIPS hydrazinyl-indolyl group, or aza-HIPS hydrazinyl-pyrrolo-pyridinyl group, tetrazine, cyclooctene, squarate, and the like.

In some instances, chemoselective ligation group is capable of spontaneous conjugation to a compatible chemical group when the two groups come into contact under suitable conditions (e.g., copper free Click chemistry conditions). In some instances, the chemoselective ligation group is capable of conjugation to a compatible chemical group when the two groups come into contact in the presence of a catalyst or other reagent (e.g., copper catalyzed Click chemistry conditions).

In some embodiments, the chemoselective ligation group is a photoactive ligation group. For example, upon irradiation with ultraviolet light, a diazirine group can form reactive carbenes, which can insert into C—H, N—H, and O—H bonds of a second moiety.

In some instances, Y is a precursor of the reactive functionality or function group capable of conjugation to a compatible group of a second moiety. For example, a carboxylic acid is a precursor of an active ester chemoselective ligation group.

In certain embodiments of formula (I), Y is a reactive moiety capable forming a covalent bond to a polypeptide (e.g., with an amino acid sidechain of a polypeptide having a compatible reactive group). The reactive moiety can be referred to as a chemoselective ligation group.

Example chemoselective ligation groups, and synthetic precursors thereof, which may be adapted for use in the compounds of this disclosure are shown in Table 4.

TABLE 4
Exemplary chemoselective ligation groups and precursors

| Groups | Exemplary structures |
|---|---|
| carboxylic acid or active ester | 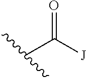 |
| | where J is selected from —OH, —Cl, —Br, —I, —F, |
| | —OH, —O—N-succinimide, —O-(4-nitrophenyl), |
| | —O-pentafluorophenyl, —O-tetrafluorophenyl, and |
| | —O—C(O)—OR^{J′}, and R^{J′} is —C₁-C₈ alkyl or -aryl, |
| | 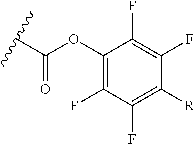 |
| | R is Hor F, |
| | 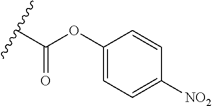 [SMILES: *C(=O)Oc1ccc([N+](=O)[O-])cc1] |
| | 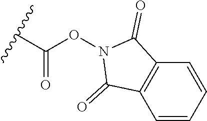 [SMILES: *C(=O)ON1C(=O)c2ccccc2C1=O] |
| | 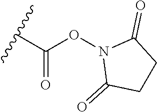 [SMILES: *C(=O)ON1C(=O)CCC1=O] |
| | 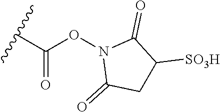 [SMILES: *C(=O)ON1C(=O)CC(S(=O)(=O)O)C1=O] |
| | 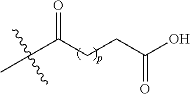 |
| | 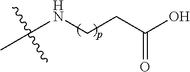 |
| | where p is 0 to 6 |
| maleimide | 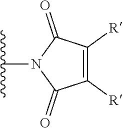 |
| | where each R′ is independently hydrogen or halogen (e.g., bromo) |
| isocyanate or | —NCS |
| isothiocyanate | —NCO |
| | 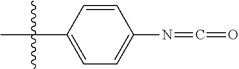 [SMILES: *c1ccc(N=C=O)cc1] |
| | 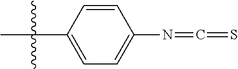 [SMILES: *c1ccc(N=C=S)cc1] |
| alkyl halide alkyl tosylate | 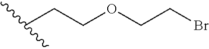 [SMILES: *CCOCCBr] |
| | 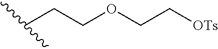 [SMILES: *CCOCCOS(=O)(=O)c1ccc(C)cc1] |
| aldehyde | 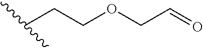 [SMILES: *CCOCC=O] |
| haloacetamide or alpha-leaving group acetamide | 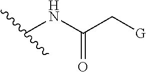 |
| | where G is selected from —F, —Cl, —Br, —I, |
| | —O-mesyl, and —O-tosyl |
| | 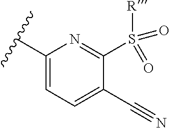 |
| | where R″′ is alkyl |
| diazirine | 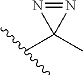 [SMILES: *C1(C)N=N1] |
| sulfonyl halide or vinyl sulfone | 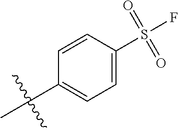 [SMILES: *c1ccc(S(=O)(=O)F)cc1] |
| | 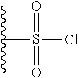 [SMILES: *S(=O)(=O)Cl] |
| | 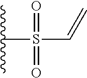 [SMILES: *S(=O)(=O)C=C] |
| hydrazide hydrazino hydroxylamino | 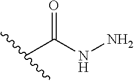 [SMILES: *C(=O)NN] |
| | 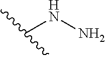 [SMILES: *NN] |
| | 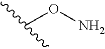 [SMILES: *ON] |
| pyridyl disulfide | 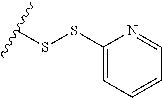 [SMILES: *SSc1ccccn1] |
| (HIPS) hydrazinyl- indolyl group, or (aza-HIPS) hydrazinyl- pyrrolo-pyridinyl group | 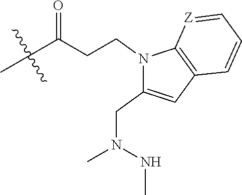 |
| | where Z is CH or N |
| alkyne or cyclooctyne | 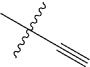 [SMILES: *C#C] |
| | 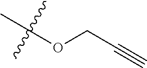 [SMILES: *OCC#C] |
| | 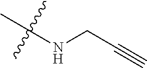 [SMILES: *NCC#C] |
| | 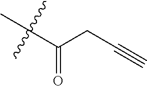 [SMILES: *C(=O)CC#C] |
| | 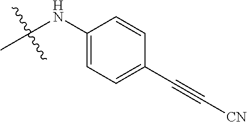 [SMILES: *Nc1ccc(C#CC#N)cc1] |
| | 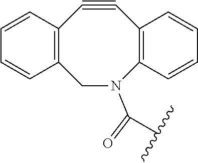 [SMILES: *C(=O)N1Cc2ccccc2C#Cc2ccccc21] |
| | 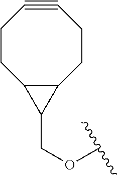 [SMILES: *OCC1C2CCC#CCCC21] |
| azide | 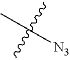 [SMILES: *N=[N+]=[N-]] |
| | 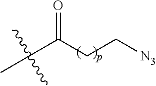 |
| | 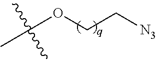 |
| | 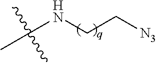 |
| | where p is 0 to 6 and where q is 1 to 6 |
| amine | 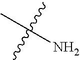 [SMILES: *N] |
| | 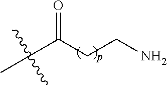 |
| | 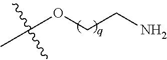 |
| | 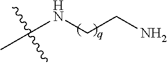 |
| | where p is 0 to 6 and where q is 1 to 6 |

In Table 4, the [symbol] can represent a point of attachment of Y to a linking moiety or a linked X moiety (e.g., FR binding moiety).

### Polypeptide Groups

In certain embodiments, Y is a polypeptide that binds to a soluble (e.g., secreted) polypeptide of interest. In certain embodiments, for example, the polypeptide of interest is a ligand that binds a cell surface receptor and Y is a polypeptide that comprises the ligand binding portion of the cell surface receptor, for example, the extracellular domain of the cell surface receptor, e.g., a ligand-binding domain of the extracellular domain of the cell surface receptor. In certain embodiments, polypeptide of interest is a cell surface receptor and Y is a polypeptide that comprises a ligand that binds the cell surface receptor or a receptor-binding portion of the ligand.

A Y group (e.g., a polypeptide) that binds to a polypeptide of interest binds as “binding” in this context is understood by one skilled in the art. For example, Y, e.g., a polypeptide, an antibody, or a conjugate as described herein comprising such Y groups, may bind to other polypeptides, generally with lower affinity as determined by, e.g., immunoassays or other assays known in the art. In a specific embodiment, Y, or a conjugate as described herein comprising such Y groups that specifically bind to a polypeptide of interest binds to the polypeptide of interest with an affinity that is at least 2 logs, 2.5 logs, 3 logs, 4 logs or greater than the affinity when Y or the conjugate bind to another polypeptide. In another specific embodiment, Y, or a conjugate as described herein comprising such Y groups, does not specifically bind a polypeptide other than the polypeptide of interest. In a specific embodiment, Y, or a conjugate as described herein comprising Y, specifically binds to a polypeptide of interest with an affinity (K_{d}) less than or equal to 20 mM. In particular embodiments, such binding is with an affinity (K_{d}) less than or equal to about 20 mM, about 10 mM, about 1 mM, about 100 μM, about 10 μM, about 1 μM, about 100 nM, about 10 nM, or about 1 nM. Unless otherwise noted, “binds,” “binds to,” “specifically binds” or “specifically binds to” in this context are used interchangeably.

In certain embodiments, for example, the polypeptide of interest is a cell surface receptor and Y comprises an antibody that binds to the cell surface protein, e.g., the extracellular domain of the cell surface receptor. In other embodiments, for example, the polypeptide of interest is a soluble, (e.g., secreted) polypeptide of interest, for example the ligand for a cell surface receptor polypeptide, and Y comprises an antibody that binds to the ligand.

Polypeptides may contain L-amino acids, D-amino acids, or both and may contain any of a variety of amino acid modifications or analogs known in the art. Useful modifications include, e.g., terminal acetylation, amidation, methylation, etc.

In certain embodiments, Y is a polypeptide that comprises about 10, about 20, about 30, about 40, about 50, about 100, about 150, about 200, about 250, about 300, about 350, about 400, about 450, about 500, about 550, about 600, about 650, about 700, about 750, about 800, about 850, about 900, or about 950 amino acids.

In certain embodiments, Y is a polypeptide comprises about 10-50, about 50-100, about 100-150, about 150-200, about 200-250, about 250-300, about 300-350, about 350-400, about 400-450, about 450-500, about 500-600, about 600-700, about 700-800, about 800-900, or about 900-1000 amino acids.

In certain embodiments, Y is an antibody (Ab). In certain embodiments, Ab is a monoclonal antibody. In certain embodiments, Ab is a human antibody. In certain embodiments, Ab is a humanized antibody. In certain embodiments, Ab is a chimeric antibody. In certain embodiments, Ab is a full-length antibody that comprises two heavy chains and two light chains. In particular embodiments, Ab is an IgG antibody, e.g., is an IgG1, IgG2, IgG3 or IgG4 antibody. In certain embodiments, Ab is a single chain antibody. In yet other embodiments, Ab is an antigen-binding fragment of an antibody, e.g., a Fab fragment.

In certain embodiments, the antibody specifically binds to a cancer antigen.

In certain embodiments, the antibody specifically binds to a hepatocyte antigen.

In certain embodiments, the antibody specifically binds to an antigen presented on a macrophage.

In certain embodiments, the antibody specifically binds to an intact complement or a fragment thereof. In certain embodiments, the antibody specifically binds to one or more immunodominant epitope(s) within intact complement or a fragment thereof.

In certain embodiments, the antibody specifically binds to a cell surface receptor. In certain embodiments, the antibody specifically binds to a cell surface receptor ligand.

In certain embodiments, the antibody specifically binds to an epidermal growth factor (EGF) protein, e.g., a human EGF. In certain embodiments, the antibody specifically binds to one or more immunodominant epitope(s) within an EGF protein.

In certain embodiments, the antibody specifically binds to an epidermal growth factor receptor (EGFR) protein, e.g., a human EGFR. In certain embodiments, the antibody specifically binds to one or more immunodominant epitope(s) within an EGFR protein. In a certain embodiment, the antibody comprises the CDRs present in cetuximab. In another certain embodiment, the antibody comprises the variable light chain and variable heavy chain present in cetuximab. In a particular embodiment, the antibody is cetuximab. In a certain embodiment, the antibody comprises the CDRs present in matuzumab. In another certain embodiment, the antibody comprises the variable light chain and variable heavy chain present in matuzumab. In a particular embodiment, the antibody is matuzumab.

In certain embodiments, the antibody specifically binds to vascular endothelial growth factor (VEGF) protein, e.g., human VEGF protein. In certain embodiments, the antibody specifically binds to one or more immunodominant epitope(s) within a VEGF protein.

In certain embodiments, the antibody specifically binds to a vascular endothelial growth factor receptor (VEGFR) protein, e.g., human VEGFR protein. In particular embodiments, the antibody specifically binds vascular endothelial growth factor receptor 2 (VEGFR2) protein, e.g., a human VEGFR2 protein. In other particular embodiments, the antibody specifically binds a vascular endothelial growth factor receptor 3 (VEGFR3) protein, e.g., a human VEGFR3 protein. In certain embodiments, the antibody specifically binds to one or more immunodominant epitope(s) within a VEGFR protein, a VEGFR2 protein or a VEGFR3 protein.

In certain embodiments, the antibody specifically binds to a fibroblast growth factor (FGF), e.g., a human FGF. In certain embodiments, the antibody specifically binds to one or more immunodominant epitope(s) within a FGF protein.

In certain embodiments, the antibody specifically binds to a fibroblast growth factor receptor (FGFR), e.g., a human FGFR. In particular embodiments, the antibody specifically binds fibroblast growth factor receptor 2 (FGFR2) protein, e.g., a human FGFR2 protein, for example, a FGFR2b protein. In other particular embodiments, the antibody specifically binds a fibroblast growth factor receptor 3 (FGFR3) protein, e.g., a human FGFR3 protein. In certain embodiments, the antibody specifically binds to one or more immunodominant epitope(s) within a FGFR protein, a FGFR2 protein or a FGFR3 protein. In a certain embodiment, the antibody comprises the CDRs present in vofatamab. In another certain embodiment, the antibody comprises the variable light chain and the variable heavy chain present in vofatamab. In a particular embodiment is vofatamab. In a certain embodiment, the antibody comprises the CDRs present in bemarituzumab. In another certain embodiment, the antibody comprises the variable light chain and the variable heavy chain present in bemarituzumab. In a particular embodiment is bemarituzumab.

In certain embodiments, the antibody specifically binds to a receptor tyrosine kinase cMET protein. In certain embodiments, the antibody specifically binds to one or more immunodominant epitope(s) within a receptor tyrosine kinase cMET protein. In certain embodiments, the antibody comprises the CDRs present in onartuzumab (MetMAb; see, e.g., CAS number 1133766-06-9). In certain embodiments, the antibody comprises the variable light chain and the heavy chain present in onartuzumab. In certain embodiments, the antibody is onartuzumab. In certain embodiments, the antibody comprises the CDRs present in emibetuzumab (LY2875358; see, e.g., CAS number 1365287-97-3). In certain embodiments, the antibody comprises the variable light chain and the heavy chain present in emibetuzumab. In certain embodiments, the antibody is emibetuzumab. In certain embodiments, the antibody specifically binds to a CD47 protein, e.g., a human CD47 protein. In certain embodiments, the antibody specifically binds to one or more immunodominant epitope(s) within a CD47 protein. In a certain embodiment, the antibody comprises the CDRs present in Hu5F9-G4 (5F9). In another certain embodiment, the antibody comprises the variable light chain and the variable heavy chain present in Hu5F9-G4 (5F9). In a particular embodiment is Hu5F9-G4 (5F9).

In certain embodiments, the antibody specifically binds to an immune checkpoint inhibitor. In certain embodiments, the antibody binds to one or more immunodominant epitope(s) within an immune checkpoint inhibitor.

In certain embodiments, the antibody specifically binds to a programmed death protein, e.g., a human PD-1. In certain embodiments, the antibody specifically binds to one or more immunodominant epitope(s) within PD-1 protein. In a certain embodiment, the antibody comprises the CDRs present in nivolumab. In another certain embodiment, the antibody comprises the variable light chain and variable heavy chain present in nivolumab. In a particular embodiment, the antibody is nivoumab. In a certain embodiment, the antibody comprises the CDRs present in pembrolizumab. In another certain embodiment, the antibody comprises the variable light chain and variable heavy chain present in pembrolizumab. In a particular embodiment, the antibody is pembrolizumab.

In certain embodiments, the antibody specifically binds to a programmed death ligand-1 (PD-L1) protein, e.g., a human PD-L1. In certain embodiments, the antibody specifically binds to one or more immunodominant epitope(s) within PD-L1 protein. In a certain embodiment, the antibody comprises the CDRs present in atezolizumab. In another certain embodiment, the antibody comprises the variable light chain and variable heavy chain present in atezolizumab. In a particular embodiment, the antibody is atezolizumab. In a certain embodiment, the antibody comprises the CDRs present in 29E.2A3 (BioXCell). In another certain embodiment, the antibody comprises the variable light chain and variable heavy chain present in 29E.2A3. In a particular embodiment, the antibody is 29E.2A3.

In certain embodiments, the antibody binds to TIM3. In certain embodiments, the antibody binds to one or more immunodominant epitope(s) within TIM3.

In certain embodiments, the antibody specifically binds to a lectin. In certain embodiments, the antibody specifically binds to one or more immunodominant epitope(s) within a lectin. In certain embodiments, the antibody binds to SIGLEC. In certain embodiments, the antibody binds to one or more immunodominant epitope(s) within SIGLEC. In certain embodiments, the antibody binds to a cytokine receptor. In certain embodiments, the antibody binds to a one or more immunodominant epitope(s) within cytokine receptor. In certain embodiments, the antibody binds to sIL6R. In certain embodiments, the antibody binds to one or more immunodominant epitope(s) within sIL6R. In certain embodiments, the antibody binds to a cytokine. In certain embodiments, the antibody binds to one or more immunodominant epitope(s) within a cytokine. In yet certain embodiments, the antibody binds to MCP-1, TNF (e.g., a TNFalpha), IL1a, IL1b, IL4, IL5, IL6, IL12/IL23, IL13, IL17 or p40. In yet certain embodiments, the antibody binds to one or more immunodominant epitope(s) within MCP-1, TNF (e.g., a TNFalpha), IL1a, IL1b, IL4, IL5, IL6, IL12/IL23, IL13, IL17 or p40.

In certain embodiments, the antibody binds to a major histocompatibility protein (e.g., a MHC class I or class II molecule). In certain embodiments, the antibody binds to one or more immunodominant epitope(s) within a major histocompatibility protein (e.g., a MHC class I or class II molecule). In certain embodiments, the antibody binds to beta 2 microglobulin. In certain embodiments, the antibody binds to one or more immunodominant epitope(s) within beta 2 microglobulin.

The heavy chain and light chain sequences of an exemplary anti-EGFR antibody (see, e.g., cetuximab, CAS number 205923-56-4) are shown in Table A.

TABLE A
Heavy chain
QVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARA
LTYYDYEFAYWGQGTLVTVSAASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQK
SLSLSPGK
(SEQ ID NO: 1)
Light chain
DILLTQSPVILSVSPGERVSFSCRASQSIGTNIHWYQQRTNGSPRLLIK
YASESISGIPSRFSGSGSGTDFTLSINSVESEDIADYYCQQNNNWPTTF
GAGTKLELKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
(SEQ ID NO: 2)

The heavy chain and light chain sequences of an exemplary Fab fragment of an anti-EGFR antibody (see, e.g., matuzumab, NCBI Accession Nos. 3C09H_H and 3C09_L, CAS number 339186-68-4) are shown in Table B.

TABLE B
Heavy chain Fab
QVQLVQSGAEVKKPGASVKVSCKASGYTFTSHWMHWVRQAPGQGLEWIG
EFNPSNGRTNYNEKFKSKATMTVDTSTNTAYMELSSLRSEDTAVYYCAS
RDYDYAGRYFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKS
(SEQ ID NO: 3)
Light chain
DIQMTQSPSSLSASVGDRVTITCSASSSVTYMYWYQQKPGKAPKLLIYD
TSNLASGVPSRFSGSGSGTDYTFTISSLQPEDIATYYCQQWSSHIFTFG
QGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGE
(SEQ ID NO: 4)

The heavy chain and light chain sequences of an exemplary anti-PD-L1 antibody (see, e.g., atezolizumab, CAS number 138723-44-3) are shown in Table C.

TABLE C
Heavy chain
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK
(SEQ ID NO: 5)
Light chain
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
(SEQ ID NO: 6)

### 5.2. Example Compounds

This disclosure includes compounds of formula (I) and (IIIA)-(IIIB) which can include:
(1) one or more particular folate binding ligand (X) of formula (Ia) (e.g., as described herein, such as ligands in Tables 1 or 2),
(2) a linker including one or more linking moieties (e.g., as described herein, such as any one or more of the linking moieties of Tables 3); and
(3) a moiety of interest (Y) e.g., as described herein, such as any one of the groups of (Table 4).

Tables 5-8 illustrate several example folate receptor binding compounds of this disclosure. It is understood that this disclosure includes Y (e.g., as described herein) conjugates of each of the exemplary compounds of Tables 5-8. For example, conjugates where the group Y has been conjugated to a different Y, such as a biomolecule or a small molecule ligand for a target protein.

The Y groups of such compounds can be utilized to connect to another Y moiety of interest (e.g., as described below). It is understood that any of these compounds can also be prepared de novo to include an alternative Y moiety of interest (e.g., as described herein below) rather than the Y groups depicted. In some embodiments, such compounds are referred to as a conjugate, e.g., a biomolecule conjugate that specifically binds a target protein.

TABLE 5
Example folate receptor binder-linker compounds

| # | Compound structure |
|---|---|
| I-1 | 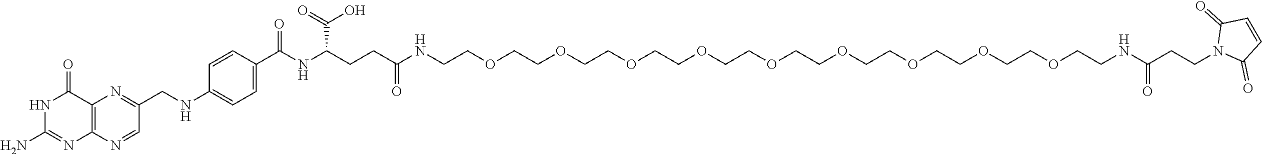 [SMILES: Nc1nc2ncc(CNc3ccc(C(=O)N[C@@H](CCC(=O)NCCOCCOCCOCCOCCOCCOCCOCCOCCOCCNC(=O)CCN4C(=O)C=CC4=O)C(=O)O)cc3)nc2c(=O)[nH]1] |
| I-2 | 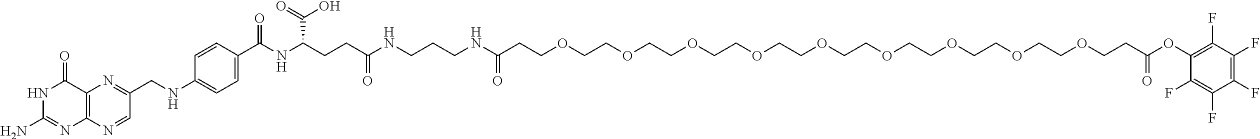 [SMILES: Nc1nc2ncc(CNc3ccc(C(=O)N[C@@H](CCC(=O)NCCCNC(=O)CCOCCOCCOCCOCCOCCOCCOCCOCCOCCC(=O)Oc4c(F)c(F)c(F)c(F)c4F)C(=O)O)cc3)nc2c(=O)[nH]1] |
| I-3 | 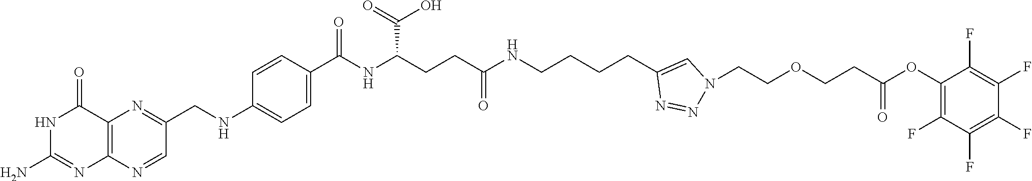 [SMILES: Nc1nc2ncc(CNc3ccc(C(=O)N[C@@H](CCC(=O)NCCCCc4cn(CCOCCC(=O)Oc5c(F)c(F)c(F)c(F)c5F)nn4)C(=O)O)cc3)nc2c(=O)[nH]1] |
| I-4 | 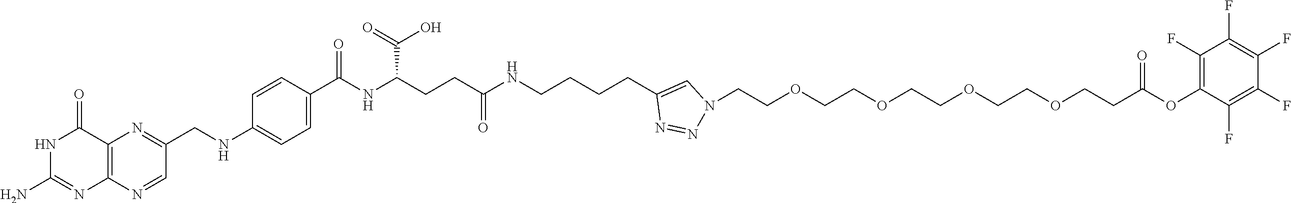 [SMILES: Nc1nc2ncc(CNc3ccc(C(=O)N[C@@H](CCC(=O)NCCCCc4cn(CCOCCOCCOCCOCCC(=O)Oc5c(F)c(F)c(F)c(F)c5F)nn4)C(=O)O)cc3)nc2c(=O)[nH]1] |
| I-4B | 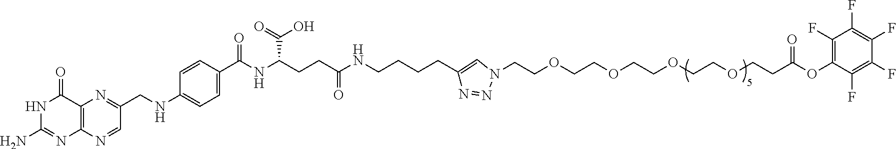 |
| I-4C | 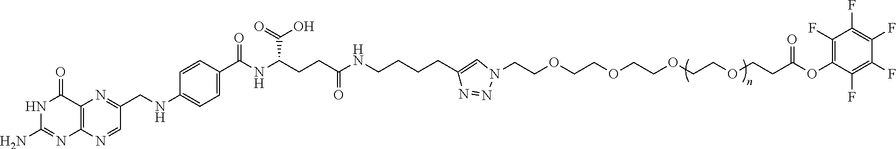 |
| | where n is 0, 1, 2, 3, 4, 5, or 6 |
| I-5 | 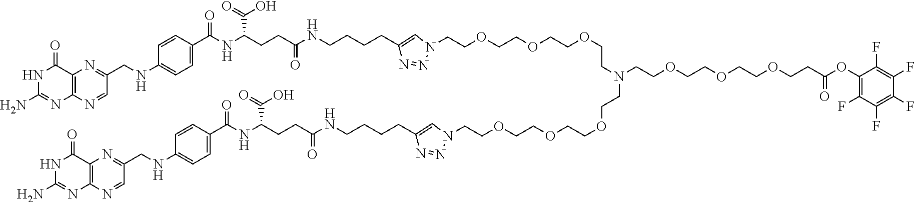 [SMILES: Nc1nc2ncc(CNc3ccc(C(=O)N[C@@H](CCC(=O)NCCCCc4cn(CCOCCOCCOCCN(CCOCCOCCOCCC(=O)Oc5c(F)c(F)c(F)c(F)c5F)CCOCCOCCOCCn5cc(CCCCNC(=O)CC[C@H](NC(=O)c6ccc(NCc7cnc8nc(N)[nH]c(=O)c8n7)cc6)C(=O)O)nn5)nn4)C(=O)O)cc3)nc2c(=O)[nH]1] |
| I-6 | 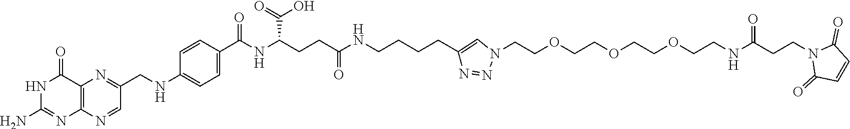 [SMILES: Nc1nc2ncc(CNc3ccc(C(=O)N[C@@H](CCC(=O)NCCCCc4cn(CCOCCOCCOCCNC(=O)CCN5C(=O)C=CC5=O)nn4)C(=O)O)cc3)nc2c(=O)[nH]1] |
| I-7 | 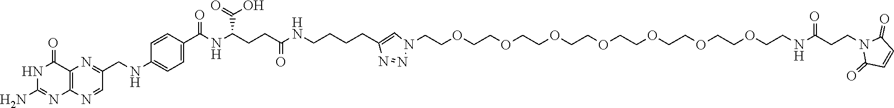 [SMILES: Nc1nc2ncc(CNc3ccc(C(=O)N[C@@H](CCC(=O)NCCCCc4cn(CCOCCOCCOCCOCCOCCOCCOCCNC(=O)CCN5C(=O)C=CC5=O)nn4)C(=O)O)cc3)nc2c(=O)[nH]1] |
| I-8 | 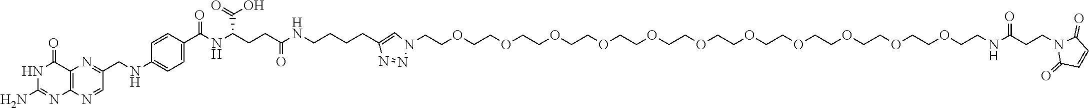 [SMILES: Nc1nc2ncc(CNc3ccc(C(=O)N[C@@H](CCC(=O)NCCCCc4cn(CCOCCOCCOCCOCCOCCOCCOCCOCCOCCOCCOCCNC(=O)CCN5C(=O)C=CC5=O)nn4)C(=O)O)cc3)nc2c(=O)[nH]1] |
| I-9 | 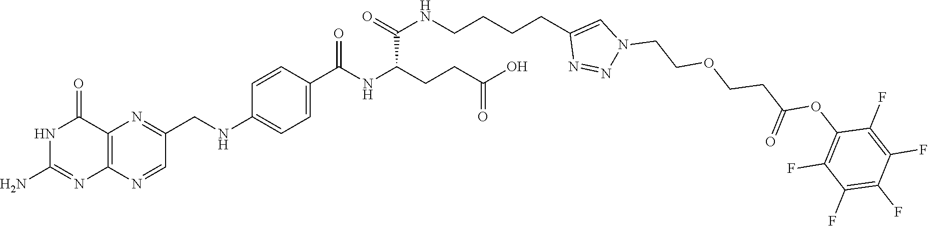 [SMILES: Nc1nc2ncc(CNc3ccc(C(=O)N[C@@H](CCC(=O)O)C(=O)NCCCCc4cn(CCOCCC(=O)Oc5c(F)c(F)c(F)c(F)c5F)nn4)cc3)nc2c(=O)[nH]1] |
| I-10 | 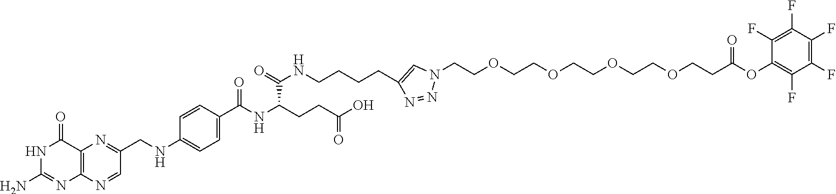 [SMILES: Nc1nc2ncc(CNc3ccc(C(=O)N[C@@H](CCC(=O)O)C(=O)NCCCCc4cn(CCOCCOCCOCCOCCC(=O)Oc5c(F)c(F)c(F)c(F)c5F)nn4)cc3)nc2c(=O)[nH]1] |
| I- 10B | 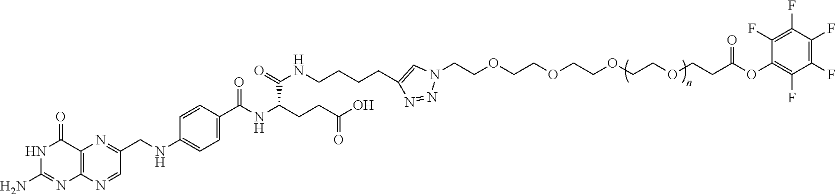 |
| | where n is 0, 1, 2, 3, 4, 5, or 6. |

TABLE 6
Exemplary folate receptor binding TNF-alpha targeting compounds of Formula (IIIB)

| # | Compound structure |
|---|---|
| I-11 | 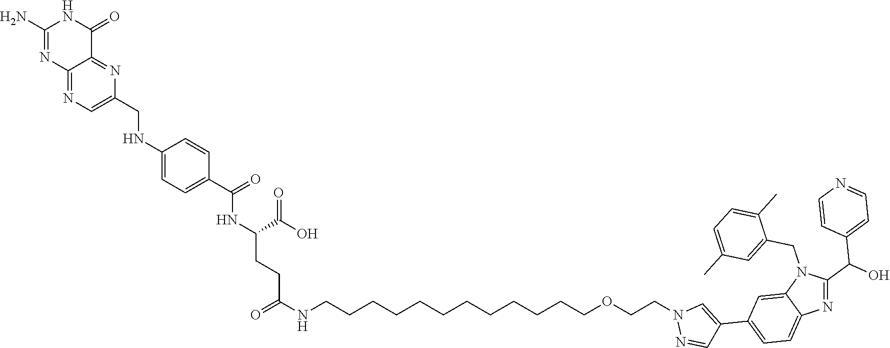 [SMILES: Cc1ccc(C)c(Cn2c(C(O)c3ccncc3)nc3ccc(-c4cnn(CCOCCCCCCCCCCCCNC(=O)CC[C@H](NC(=O)c5ccc(NCc6cnc7nc(N)[nH]c(=O)c7n6)cc5)C(=O)O)c4)cc32)c1] |
| I-12 | 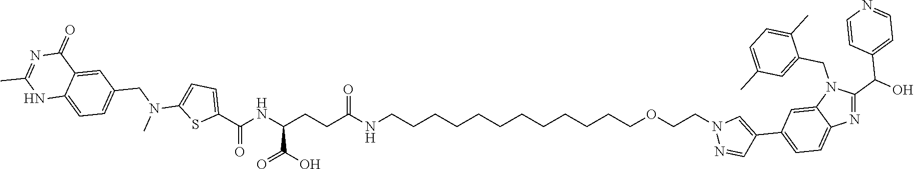 [SMILES: Cc1ccc(C)c(Cn2c(C(O)c3ccncc3)nc3ccc(-c4cnn(CCOCCCCCCCCCCCCNC(=O)CC[C@H](NC(=O)c5ccc(N(C)Cc6ccc7[nH]c(C)nc(=O)c7c6)s5)C(=O)O)c4)cc32)c1] |
| I-13 | 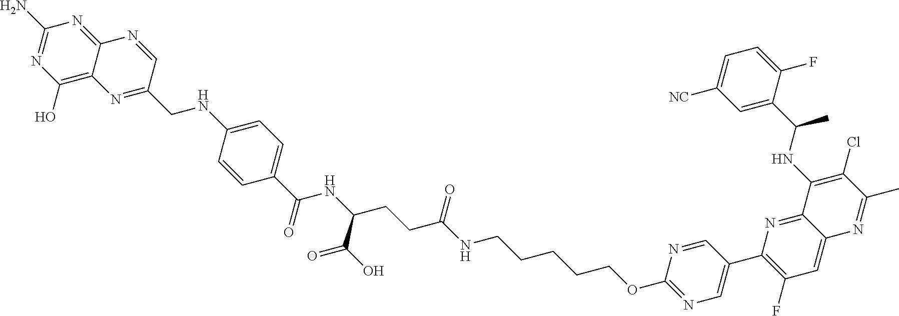 [SMILES: Cc1nc2cc(F)c(-c3cnc(OCCCCCNC(=O)CC[C@H](NC(=O)c4ccc(NCc5cnc6nc(N)nc(O)c6n5)cc4)C(=O)O)nc3)nc2c(N[C@H](C)c2cc(C#N)ccc2F)c1Cl] |
| I-14 | 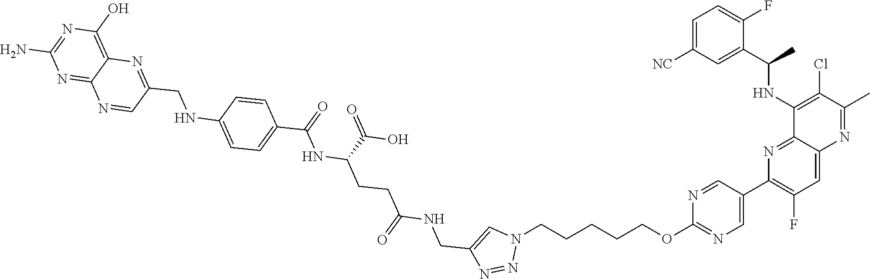 [SMILES: Cc1nc2cc(F)c(-c3cnc(OCCCCCn4cc(CNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I-15 | 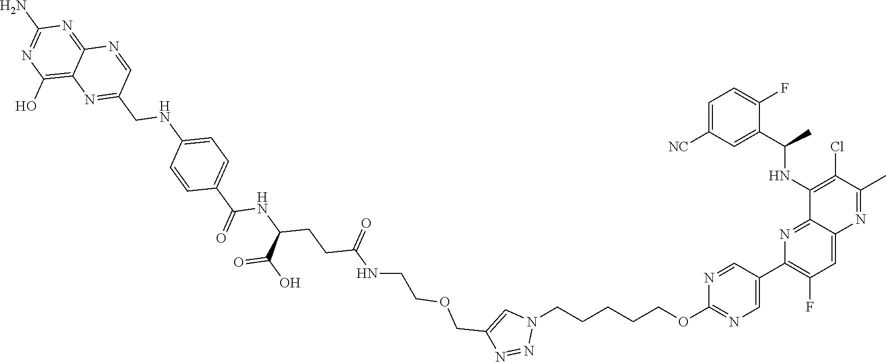 [SMILES: Cc1nc2cc(F)c(-c3cnc(OCCCCCn4cc(COCCNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I-16 | 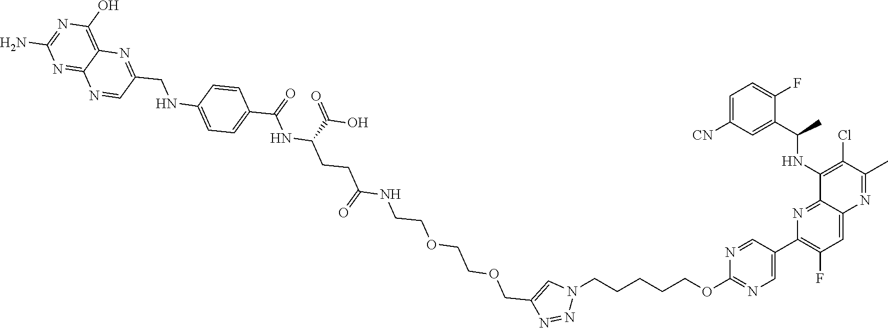 |
| I-17 | 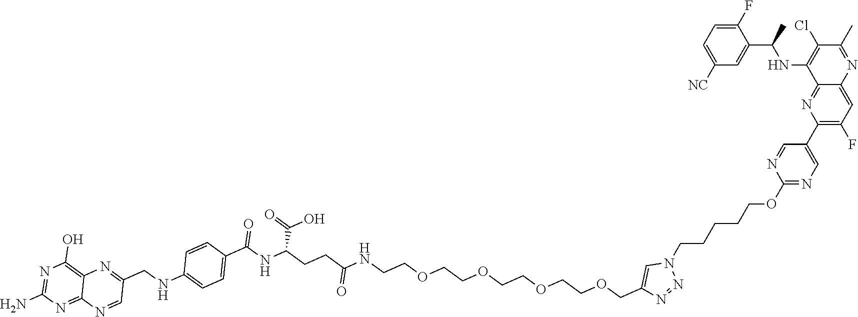 [SMILES: Cc1nc2cc(F)c(-c3cnc(OCCCCCn4cc(COCCOCCOCCOCCNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I-18 | 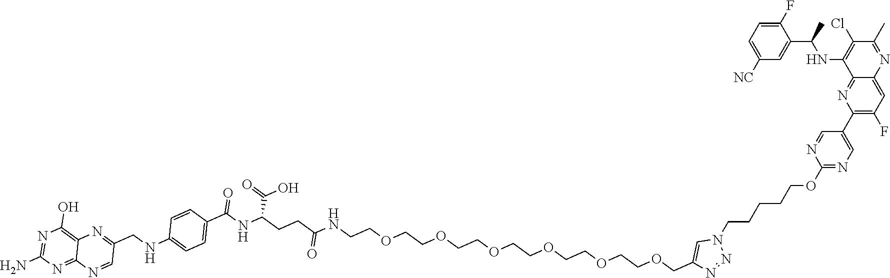 [SMILES: Cc1nc2cc(F)c(-c3cnc(OCCCCCn4cc(COCCOCCOCCOCCOCCOCCNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I-19 | 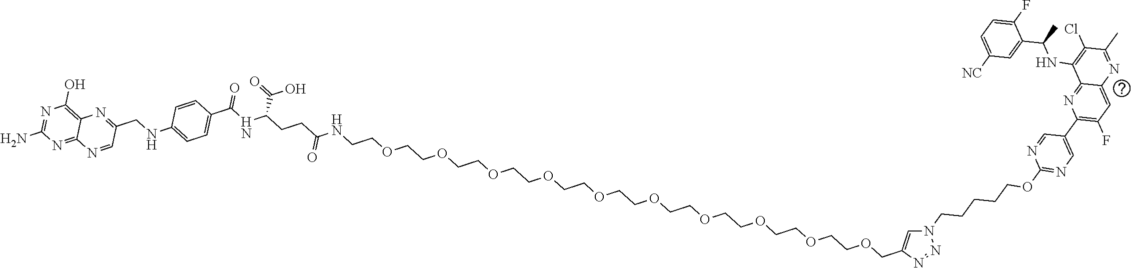 [SMILES: Cc1nc2cc(F)c(-c3cnc(OCCCCCn4cc(COCCOCCOCCOCCOCCOCCOCCOCCOCCOCCNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I-20 | 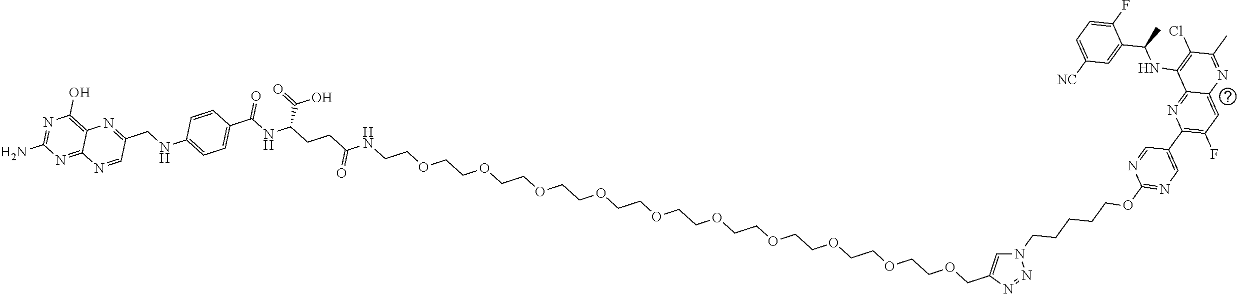 [SMILES: Cc1nc2cc(F)c(-c3cnc(OCCCCCn4cc(COCCOCCOCCOCCOCCOCCOCCOCCOCCOCCNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I-22 | 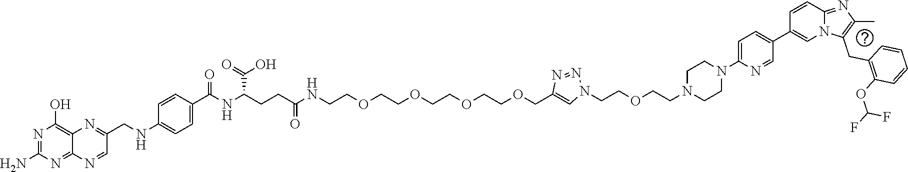 [SMILES: Cc1nc2ccc(-c3ccc(N4CCN(CCOCCn5cc(COCCOCCOCCOCCNC(=O)CC[C@H](NC(=O)c6ccc(NCc7cnc8nc(N)nc(O)c8n7)cc6)C(=O)O)nn5)CC4)nc3)cn2c1Cc1ccccc1OC(F)F] |
| I-23 | 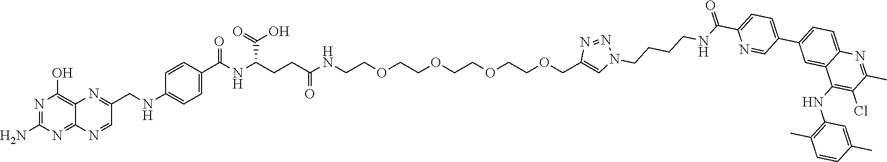 [SMILES: Cc1ccc(C)c(Nc2c(Cl)c(C)nc3ccc(-c4ccc(C(=O)NCCCCn5cc(COCCOCCOCCOCCNC(=O)CC[C@H](NC(=O)c6ccc(NCc7cnc8nc(N)nc(O)c8n7)cc6)C(=O)O)nn5)nc4)cc23)c1] |
| I-24 | 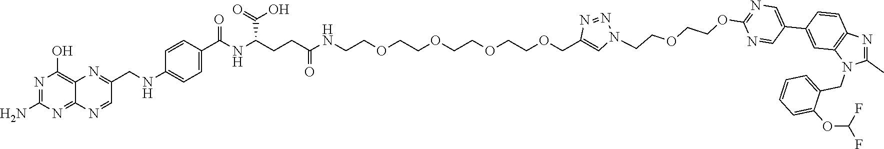 [SMILES: Cc1nc2ccc(-c3cnc(OCCOCCn4cc(COCCOCCOCCOCCNC(=O)CC[C@H](NC(=O)c5ccc(NCc6cnc7nc(N)nc(O)c7n6)cc5)C(=O)O)nn4)nc3)cc2n1Cc1ccccc1OC(F)F] |
| I-25 | 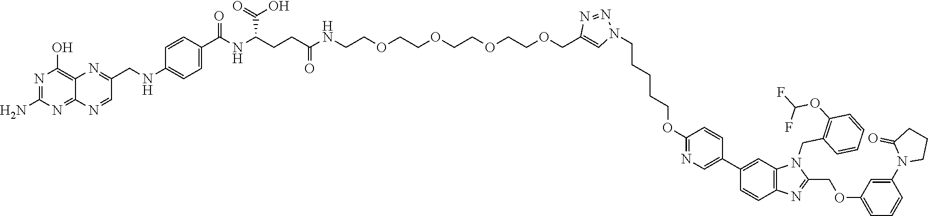 [SMILES: Nc1nc(O)c2nc(CNc3ccc(C(=O)N[C@@H](CCC(=O)NCCOCCOCCOCCOCc4cn(CCCCCOc5ccc(-c6ccc7nc(COc8cccc(N9CCCC9=O)c8)n(Cc8ccccc8OC(F)F)c7c6)cn5)nn4)C(=O)O)cc3)cnc2n1] |
| I-26 | 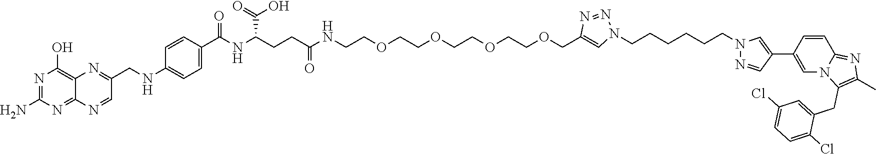 [SMILES: Cc1nc2ccc(-c3cnn(CCCCCCn4cc(COCCOCCOCCOCCNC(=O)CC[C@H](NC(=O)c5ccc(NCc6cnc7nc(N)nc(O)c7n6)cc5)C(=O)O)nn4)c3)cn2c1Cc1cc(Cl)ccc1Cl] |
| I-27 | 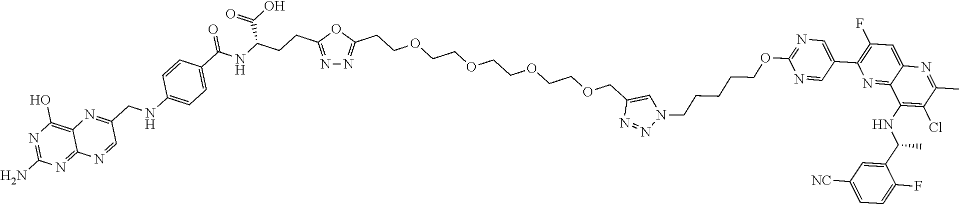 [SMILES: Cc1nc2cc(F)c(-c3cnc(OCCCCCn4cc(COCCOCCOCCOCCc5nnc(CC[C@H](NC(=O)c6ccc(NCc7cnc8nc(N)nc(O)c8n7)cc6)C(=O)O)o5)nn4)nc3)nc2c(N[C@H](C)c2cc(C#N)ccc2F)c1Cl] |
| I-28 | 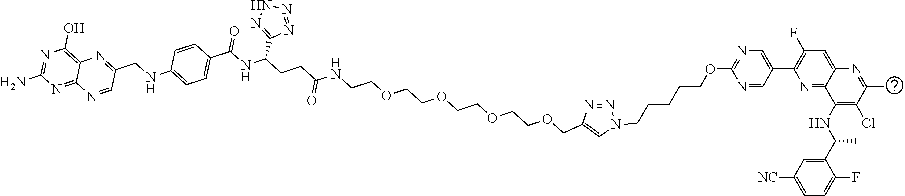 [SMILES: Cc1nc2cc(F)c(-c3cnc(OCCCCCn4cc(COCCOCCOCCOCCNC(=O)CC[C@H](NC(=O)c5ccc(NCc6cnc7nc(N)nc(O)c7n6)cc5)c5nn[nH]n5)nn4)nc3)nc2c(N[C@H](C)c2cc(C#N)ccc2F)c1Cl] |
| I-29 | 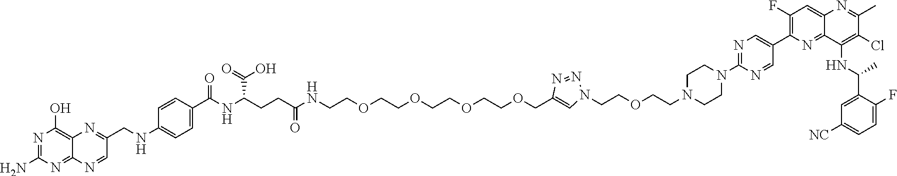 [SMILES: Cc1nc2cc(F)c(-c3cnc(N4CCN(CCOCCn5cc(COCCOCCOCCOCCNC(=O)CC[C@H](NC(=O)c6ccc(NCc7cnc8nc(N)nc(O)c8n7)cc6)C(=O)O)nn5)CC4)nc3)nc2c(N[C@H](C)c2cc(C#N)ccc2F)c1Cl] |
| I-30 | 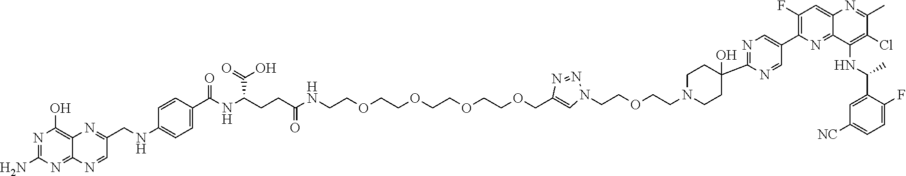 [SMILES: Cc1nc2cc(F)c(-c3cnc(C4(O)CCN(CCOCCn5cc(COCCOCCOCCOCCNC(=O)CC[C@H](NC(=O)c6ccc(NCc7cnc8nc(N)nc(O)c8n7)cc6)C(=O)O)nn5)CC4)nc3)nc2c(N[C@H](C)c2cc(C#N)ccc2F)c1Cl] |
| I-31 | 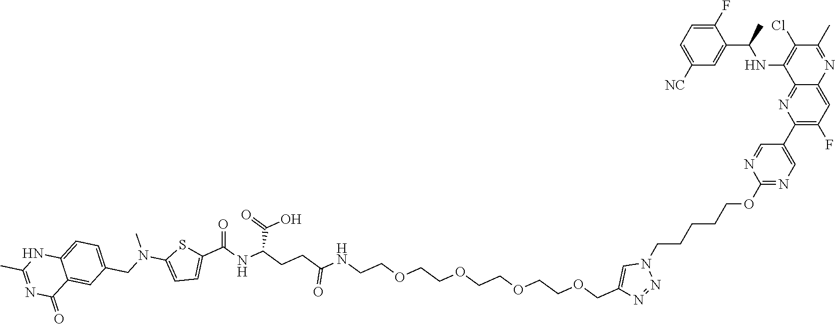 [SMILES: Cc1nc(=O)c2cc(CN(C)c3ccc(C(=O)N[C@@H](CCC(=O)NCCOCCOCCOCCOCc4cn(CCCCCOc5ncc(-c6nc7c(N[C@H](C)c8cc(C#N)ccc8F)c(Cl)c(C)nc7cc6F)cn5)nn4)C(=O)O)s3)ccc2[nH]1] |
| I-33 | 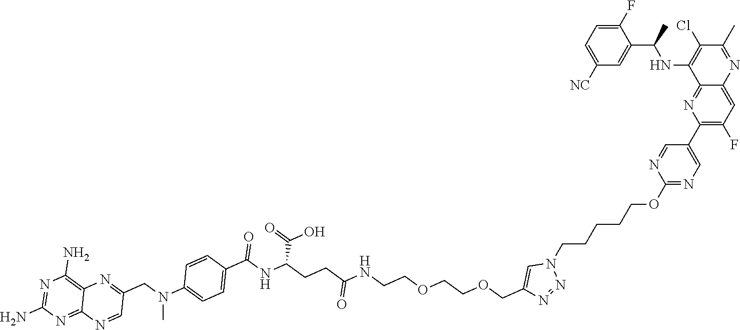 [SMILES: Cc1nc2cc(F)c(-c3cnc(OCCCCCn4cc(COCCOCCNC(=O)CC[C@H](NC(=O)c5ccc(N(C)Cc6cnc7nc(N)nc(N)c7n6)cc5)C(=O)O)nn4)nc3)nc2c(N[C@H](C)c2cc(C#N)ccc2F)c1Cl] |
| I-36 | 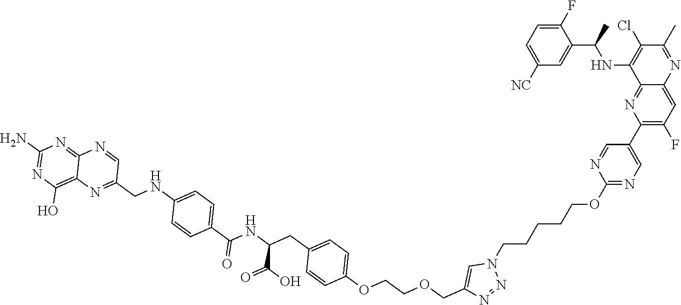 [SMILES: Cc1nc2cc(F)c(-c3cnc(OCCCCCn4cc(COCCOc5ccc(C[C@H](NC(=O)c6ccc(NCc7cnc8nc(N)nc(O)c8n7)cc6)C(=O)O)cc5)nn4)nc3)nc2c(N[C@H](C)c2cc(C#N)ccc2F)c1Cl] |
| I-40 | 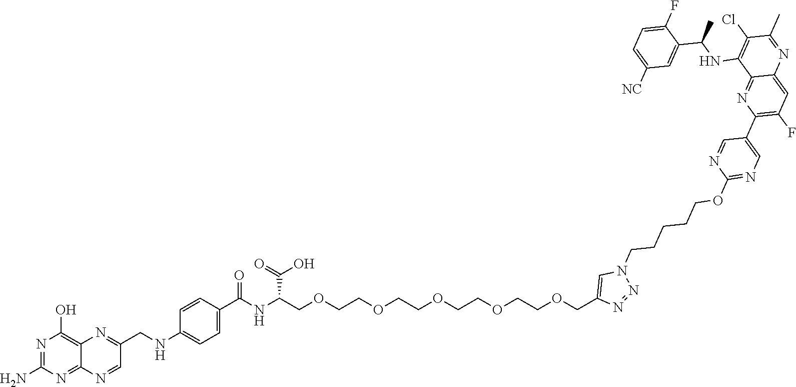 [SMILES: Cc1nc2cc(F)c(-c3cnc(OCCCCCn4cc(COCCOCCOCCOCCOC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I-41 | 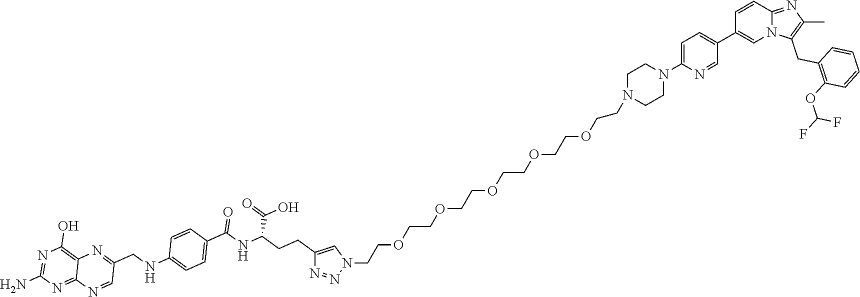 [SMILES: Cc1nc2ccc(-c3ccc(N4CCN(CCOCCOCCOCCOCCOCCn5cc(CC[C@H](NC(=O)c6ccc(NCc7cnc8nc(N)nc(O)c8n7)cc6)C(=O)O)nn5)CC4)nc3)cn2c1Cc1ccccc1OC(F)F] |
| I-42 | 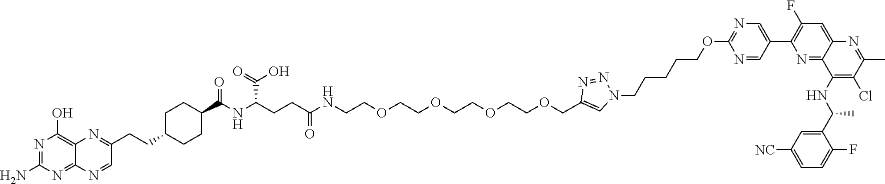 [SMILES: Cc1nc2cc(F)c(-c3cnc(OCCCCCn4cc(COCCOCCOCCOCCNC(=O)CC[C@H](NC(=O)[C@H]5CC[C@H](CCc6cnc7nc(N)nc(O)c7n6)CC5)C(=O)O)nn4)nc3)nc2c(N[C@H](C)c2cc(C#N)ccc2F)c1Cl] |
| I-43 | 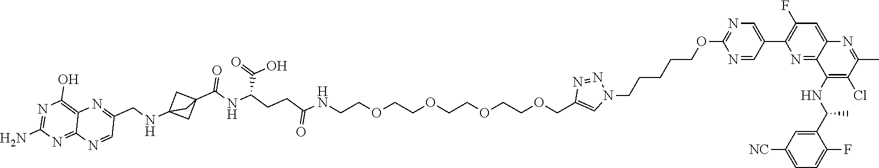 [SMILES: Cc1nc2cc(F)c(-c3cnc(OCCCCCn4cc(COCCOCCOCCOCCNC(=O)CC[C@H](NC(=O)C56CC(NCc7cnc8nc(N)nc(O)c8n7)(C5)C6)C(=O)O)nn4)nc3)nc2c(N[C@H](C)c2cc(C#N)ccc2F)c1Cl] |
| I-44 | 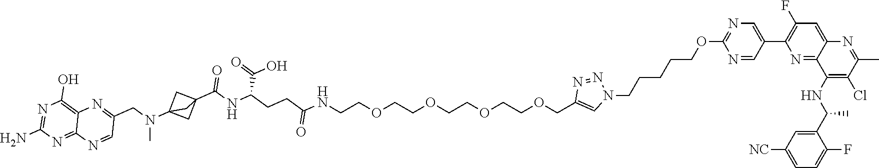 [SMILES: Cc1nc2cc(F)c(-c3cnc(OCCCCCn4cc(COCCOCCOCCOCCNC(=O)CC[C@H](NC(=O)C56CC(N(C)Cc7cnc8nc(N)nc(O)c8n7)(C5)C6)C(=O)O)nn4)nc3)nc2c(N[C@H](C)c2cc(C#N)ccc2F)c1Cl] |
| I-45 | 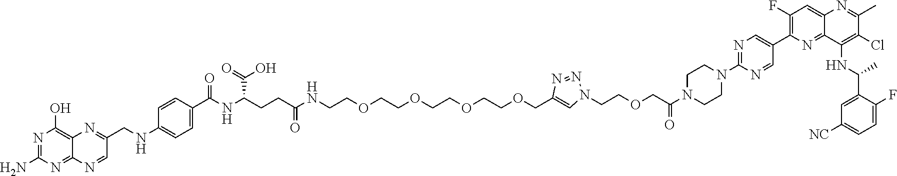 [SMILES: Cc1nc2cc(F)c(-c3cnc(N4CCN(C(=O)COCCn5cc(COCCOCCOCCOCCNC(=O)CC[C@H](NC(=O)c6ccc(NCc7cnc8nc(N)nc(O)c8n7)cc6)C(=O)O)nn5)CC4)nc3)nc2c(N[C@H](C)c2cc(C#N)ccc2F)c1Cl] |
| I-46 | 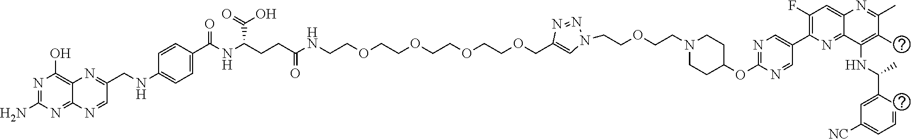 [SMILES: Cc1nc2cc(F)c(-c3cnc(OC4CCN(CCOCCn5cc(COCCOCCOCCOCCNC(=O)CC[C@H](NC(=O)c6ccc(NCc7cnc8nc(N)nc(O)c8n7)cc6)C(=O)O)nn5)CC4)nc3)nc2c(N[C@H](C)c2cccc(C#N)c2)c1C] |
| I-47 | 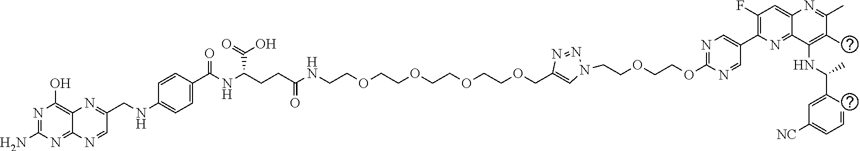 [SMILES: Cc1nc2cc(F)c(-c3cnc(OCCOCCn4cc(COCCOCCOCCOCCNC(=O)CC[C@H](NC(=O)c5ccc(NCc6cnc7nc(N)nc(O)c7n6)cc5)C(=O)O)nn4)nc3)nc2c(N[C@H](C)c2cccc(C#N)c2)c1C] |
| I-48 | 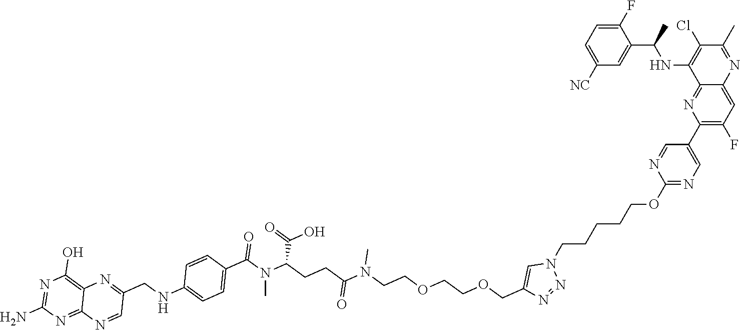 [SMILES: Cc1nc2cc(F)c(-c3cnc(OCCCCCn4cc(COCCOCCN(C)C(=O)CC[C@@H](C(=O)O)N(C)C(=O)c5ccc(NCc6cnc7nc(N)nc(O)c7n6)cc5)nn4)nc3)nc2c(N[C@H](C)c2cc(C#N)ccc2F)c1Cl] |
| I-49 | 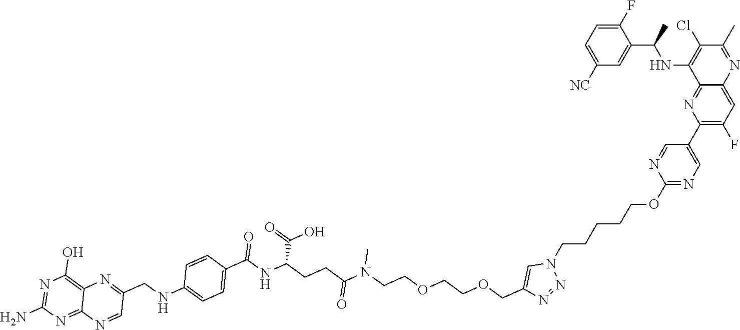 [SMILES: Cc1nc2cc(F)c(-c3cnc(OCCCCCn4cc(COCCOCCN(C)C(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I-50 | 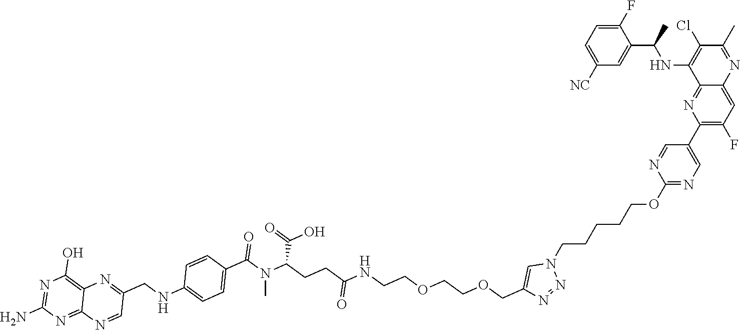 [SMILES: Cc1nc2cc(F)c(-c3cnc(OCCCCCn4cc(COCCOCCNC(=O)CC[C@@H](C(=O)O)N(C)C(=O)c5ccc(NCc6cnc7nc(N)nc(O)c7n6)cc5)nn4)nc3)nc2c(N[C@H](C)c2cc(C#N)ccc2F)c1Cl] |
| I-51 | 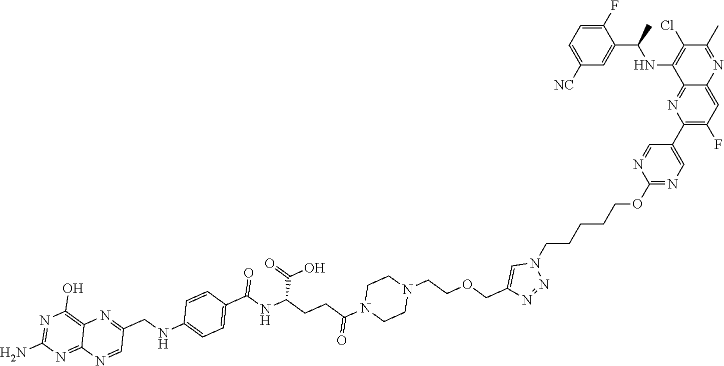 [SMILES: Cc1nc2cc(F)c(-c3cnc(OCCCCCn4cc(COCCN5CCN(C(=O)CC[C@H](NC(=O)c6ccc(NCc7cnc8nc(N)nc(O)c8n7)cc6)C(=O)O)CC5)nn4)nc3)nc2c(N[C@H](C)c2cc(C#N)ccc2F)c1Cl] |
| I-52 | 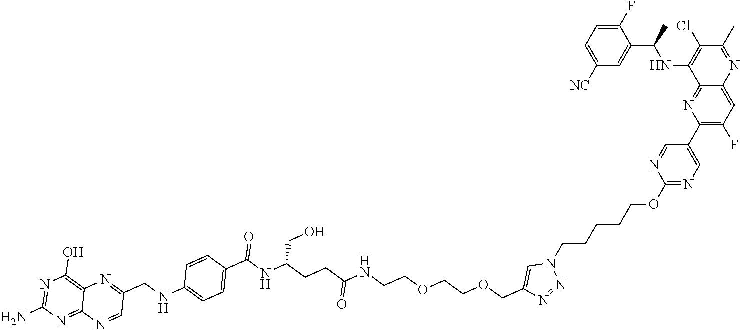 [SMILES: Cc1nc2cc(F)c(-c3cnc(OCCCCCn4cc(COCCOCCNC(=O)CC[C@@H](CO)NC(=O)c5ccc(NCc6cnc7nc(N)nc(O)c7n6)cc5)nn4)nc3)nc2c(N[C@H](C)c2cc(C#N)ccc2F)c1Cl] |
| I-53 | 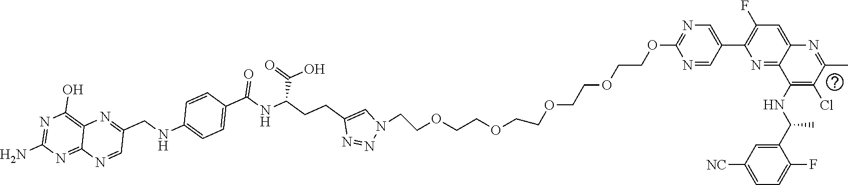 [SMILES: Cc1nc2cc(F)c(-c3cnc(OCCOCCOCCOCCOCCn4cc(CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I-54 | 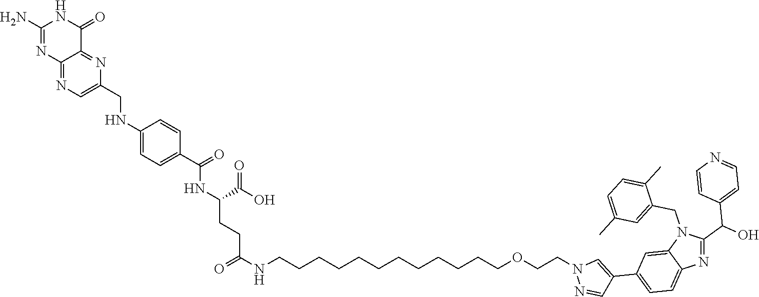 [SMILES: Cc1ccc(C)c(Cn2c(C(O)c3ccncc3)nc3ccc(-c4cnn(CCOCCCCCCCCCCCCNC(=O)CC[C@H](NC(=O)c5ccc(NCc6cnc7nc(N)[nH]c(=O)c7n6)cc5)C(=O)O)c4)cc32)c1] |
| I-55 | 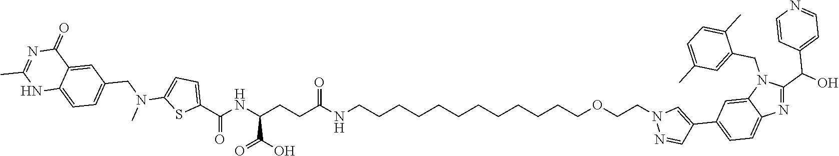 [SMILES: Cc1ccc(C)c(Cn2c(C(O)c3ccncc3)nc3ccc(-c4cnn(CCOCCCCCCCCCCCCNC(=O)CC[C@H](NC(=O)c5ccc(N(C)Cc6ccc7[nH]c(C)nc(=O)c7c6)s5)C(=O)O)c4)cc32)c1] |
| I-56 | 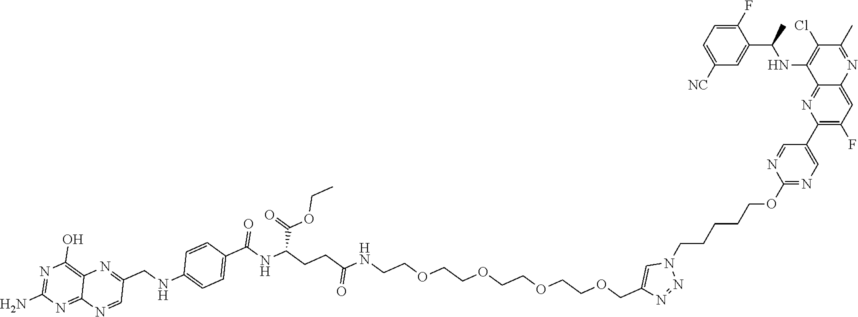 [SMILES: CCOC(=O)[C@H](CCC(=O)NCCOCCOCCOCCOCc1cn(CCCCCOc2ncc(-c3nc4c(N[C@H](C)c5cc(C#N)ccc5F)c(Cl)c(C)nc4cc3F)cn2)nn1)NC(=O)c1ccc(NCc2cnc3nc(N)nc(O)c3n2)cc1] |
| I-57 | 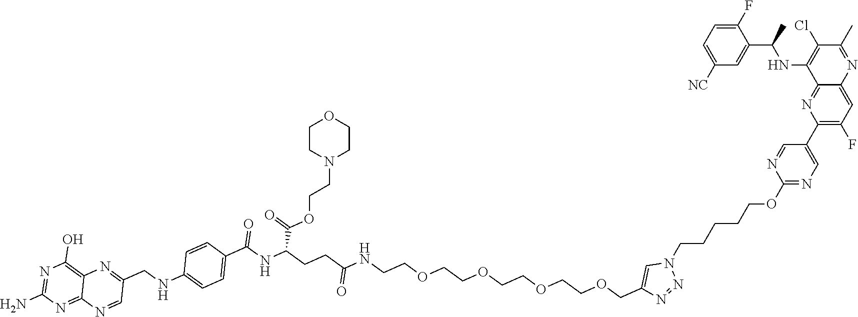 [SMILES: Cc1nc2cc(F)c(-c3cnc(OCCCCCn4cc(COCCOCCOCCOCCNC(=O)CC[C@H](NC(=O)c5ccc(NCc6cnc7nc(N)nc(O)c7n6)cc5)C(=O)OCCN5CCOCC5)nn4)nc3)nc2c(N[C@H](C)c2cc(C#N)ccc2F)c1Cl] |
| I-58 | 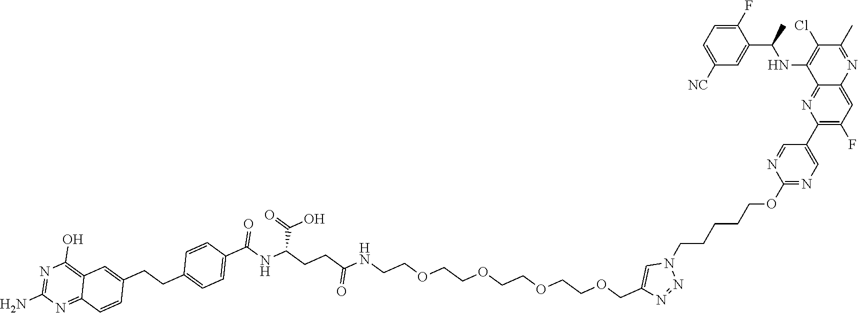 [SMILES: Cc1nc2cc(F)c(-c3cnc(OCCCCCn4cc(COCCOCCOCCOCCNC(=O)CC[C@H](NC(=O)c5ccc(CCc6ccc7nc(N)nc(O)c7c6)cc5)C(=O)O)nn4)nc3)nc2c(N[C@H](C)c2cc(C#N)ccc2F)c1Cl] |
| I-59 | 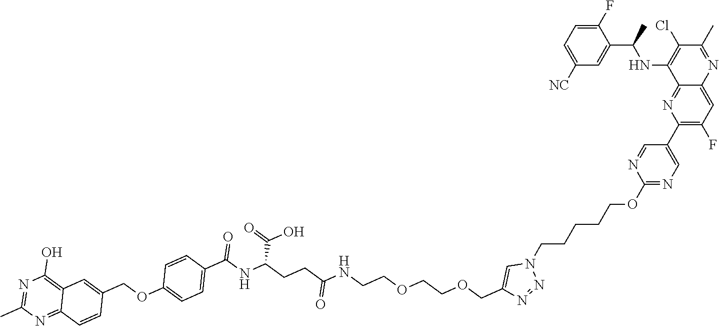 [SMILES: Cc1nc(O)c2cc(COc3ccc(C(=O)N[C@@H](CCC(=O)NCCOCCOCc4cn(CCCCCOc5ncc(-c6nc7c(N[C@H](C)c8cc(C#N)ccc8F)c(Cl)c(C)nc7cc6F)cn5)nn4)C(=O)O)cc3)ccc2n1] |
| I-60 | 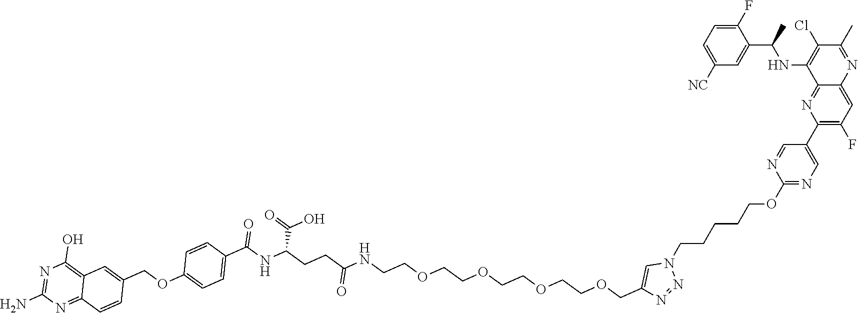 [SMILES: Cc1nc2cc(F)c(-c3cnc(OCCCCCn4cc(COCCOCCOCCOCCNC(=O)CC[C@H](NC(=O)c5ccc(OCc6ccc7nc(N)nc(O)c7c6)cc5)C(=O)O)nn4)nc3)nc2c(N[C@H](C)c2cc(C#N)ccc2F)c1Cl] |
| I-61 | 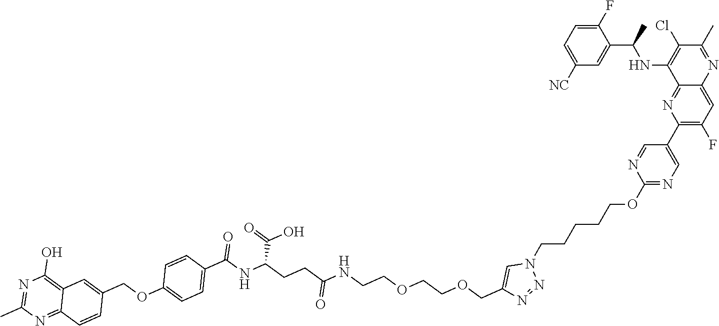 [SMILES: Cc1nc(O)c2cc(COc3ccc(C(=O)N[C@@H](CCC(=O)NCCOCCOCc4cn(CCCCCOc5ncc(-c6nc7c(N[C@H](C)c8cc(C#N)ccc8F)c(Cl)c(C)nc7cc6F)cn5)nn4)C(=O)O)cc3)ccc2n1] |
| I-62 | 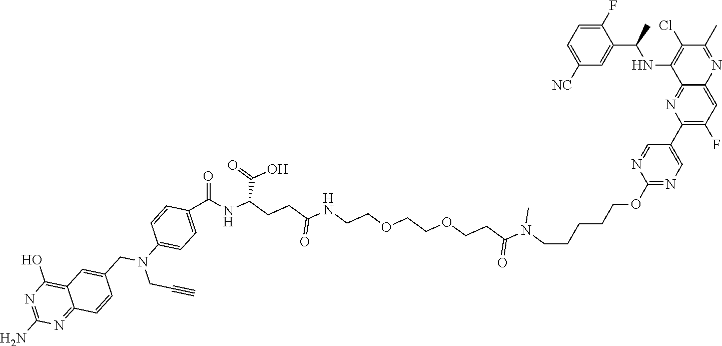 [SMILES: C#CCN(Cc1ccc2nc(N)nc(O)c2c1)c1ccc(C(=O)N[C@@H](CCC(=O)NCCOCCOCCC(=O)N(C)CCCCCOc2ncc(-c3nc4c(N[C@H](C)c5cc(C#N)ccc5F)c(Cl)c(C)nc4cc3F)cn2)C(=O)O)cc1] |
| I-63 | 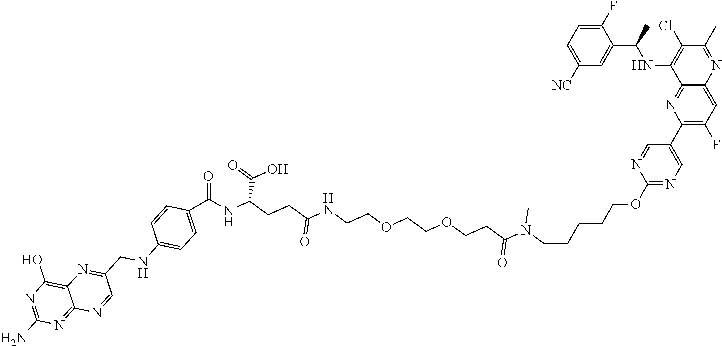 [SMILES: Cc1nc2cc(F)c(-c3cnc(OCCCCCN(C)C(=O)CCOCCOCCNC(=O)CC[C@H](NC(=O)c4ccc(NCc5cnc6nc(N)nc(O)c6n5)cc4)C(=O)O)nc3)nc2c(N[C@H](C)c2cc(C#N)ccc2F)c1Cl] |
| I-64 | 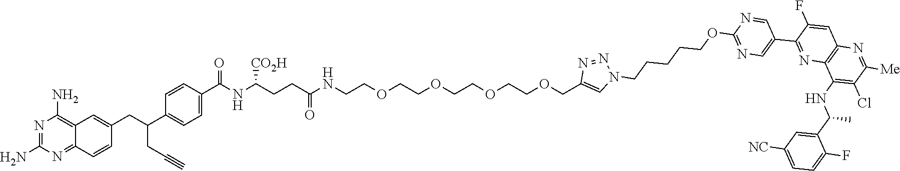 |
| I-65 | 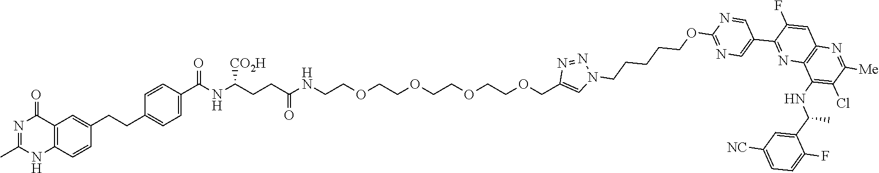 |
| I-66 | 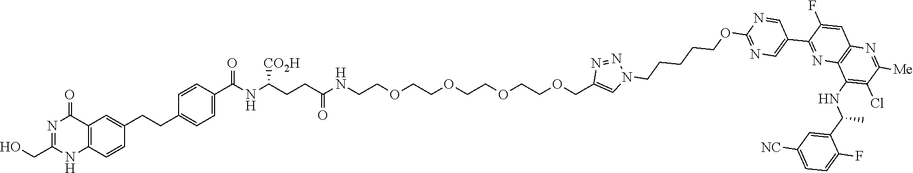 |
| I-67 | 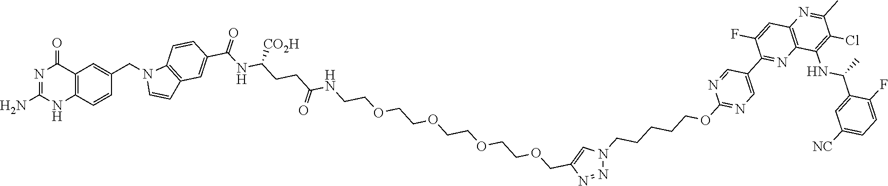 [SMILES: Cc1nc2cc(F)c(-c3cnc(OCCCCCn4cc(COCCOCCOCCOCCNC(=O)CC[C@H](NC(=O)c5ccc6c(ccn6Cc6ccc7[nH]c(N)nc(=O)c7c6)c5)C(=O)O)nn4)nc3)nc2c(N[C@H](C)c2cc(C#N)ccc2F)c1Cl] |
| I-68 | 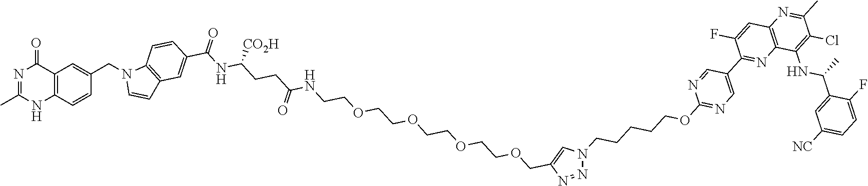 [SMILES: Cc1nc(=O)c2cc(Cn3ccc4cc(C(=O)N[C@@H](CCC(=O)NCCOCCOCCOCCOCc5cn(CCCCCOc6ncc(-c7nc8c(N[C@H](C)c9cc(C#N)ccc9F)c(Cl)c(C)nc8cc7F)cn6)nn5)C(=O)O)ccc43)ccc2[nH]1] |
| I-69 | 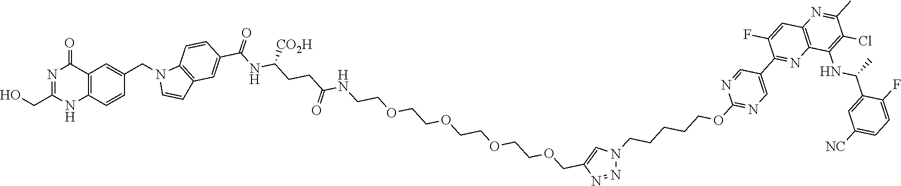 [SMILES: Cc1nc2cc(F)c(-c3cnc(OCCCCCn4cc(COCCOCCOCCOCCNC(=O)CC[C@H](NC(=O)c5ccc6c(ccn6Cc6ccc7[nH]c(CO)nc(=O)c7c6)c5)C(=O)O)nn4)nc3)nc2c(N[C@H](C)c2cc(C#N)ccc2F)c1Cl] |
| I-70 | 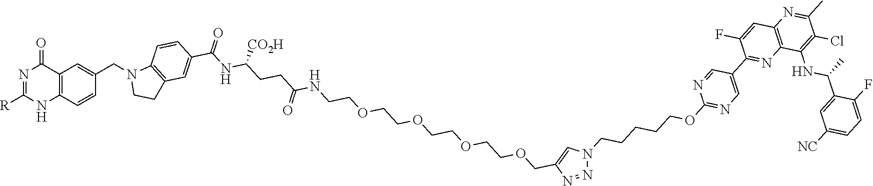 |
| | where R is —NH₂, —Me, or —CH₂—OH (3 separate compounds) |
| I-71 | 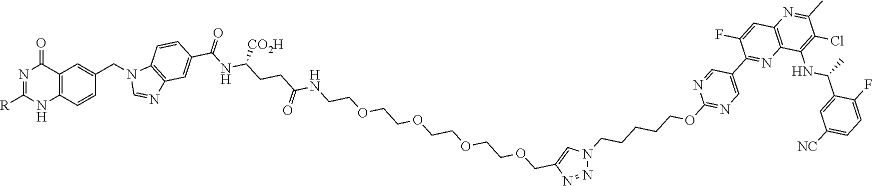 |
| | where R is —NH₂, —Me, or —CH₂—OH (3 separate compounds) |
| I-72 | 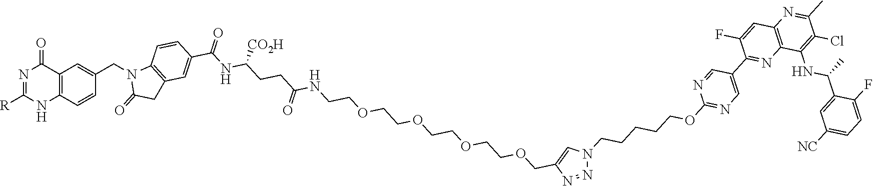 |
| | where R is —NH₂, —Me, or —CH₂—OH (3 separate compounds) |
| I-73 | 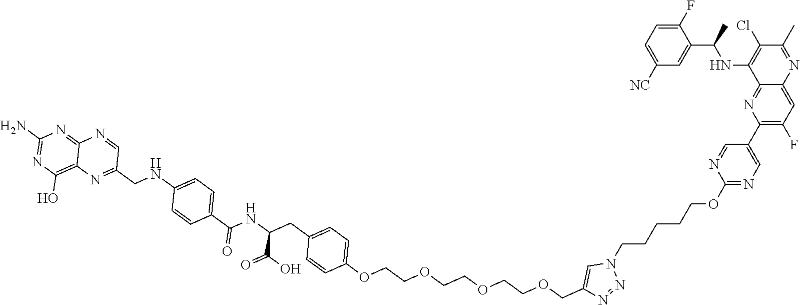 [SMILES: Cc1nc2cc(F)c(-c3cnc(OCCCCCn4cc(COCCOCCOCCOc5ccc(C[C@H](NC(=O)c6ccc(NCc7cnc8nc(N)nc(O)c8n7)cc6)C(=O)O)cc5)nn4)nc3)nc2c(N[C@H](C)c2cc(C#N)ccc2F)c1Cl] |

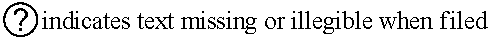

TABLE 7
Exemplary folate receptor binding TNF-alpha targeting compounds of Formula (IIIA)

| # | Compound structure |
|---|---|
| I- 21 | 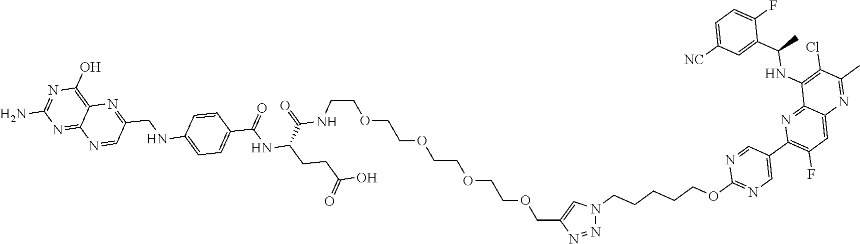 [SMILES: Cc1nc2cc(F)c(-c3cnc(OCCCCCn4cc(COCCOCCOCCOCCNC(=O)[C@H](CCC(=O)O)NC(=O)c5ccc(NCc6cnc7nc(N)nc(O)c7n6)cc5)nn4)nc3)nc2c(N[C@H](C)c2cc(C#N)ccc2F)c1Cl] |
| I- 35 | 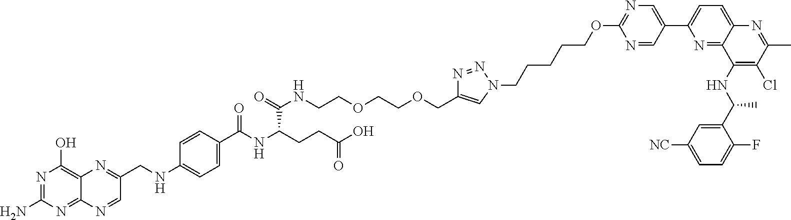 [SMILES: Cc1nc2ccc(-c3cnc(OCCCCCn4cc(COCCOCCNC(=O)[C@H](CCC(=O)O)NC(=O)c5ccc(NCc6cnc7nc(N)nc(O)c7n6)cc5)nn4)nc3)nc2c(N[C@H](C)c2cc(C#N)ccc2F)c1Cl] |
| I- 34 | 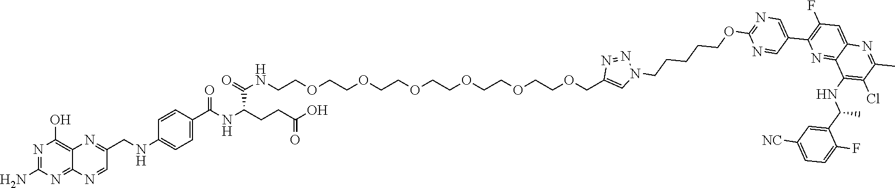 |
| I- 74 | 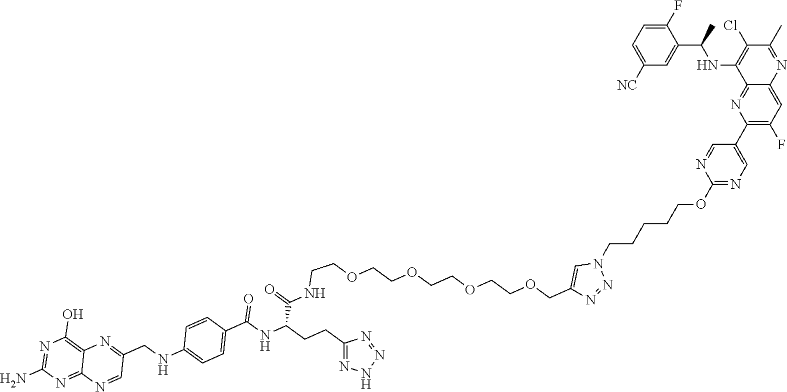 [SMILES: Cc1nc2cc(F)c(-c3cnc(OCCCCCn4cc(COCCOCCOCCOCCNC(=O)[C@H](CCc5nn[nH]n5)NC(=O)c5ccc(NCc6cnc7nc(N)nc(O)c7n6)cc5)nn4)nc3)nc2c(N[C@H](C)c2cc(C#N)ccc2F)c1Cl] |

TABLE 8
Exemplary folate receptor binding TNF-alpha targeting compounds of Formula (IIIB)

| # | Compound structure |
|---|---|
| I- 75 | 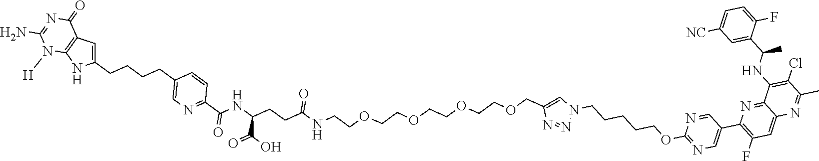 |
| I- 32 | 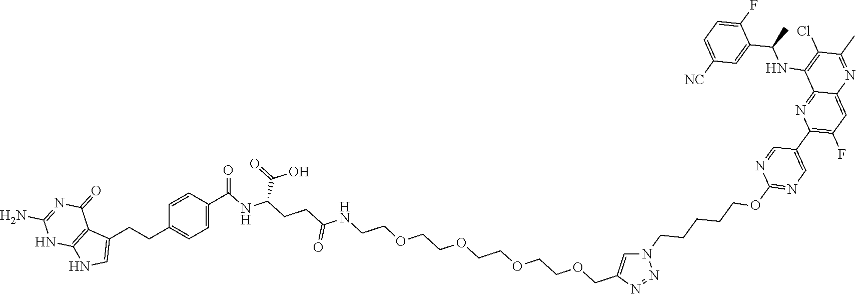 [SMILES: Cc1nc2cc(F)c(-c3cnc(OCCCCCn4cc(COCCOCCOCCOCCNC(=O)CC[C@H](NC(=O)c5ccc(CCc6c[nH]c7[nH]c(N)nc(=O)c67)cc5)C(=O)O)nn4)nc3)nc2c(N[C@H](C)c2cc(C#N)ccc2F)c1Cl] |

TABLE 9
Other example folate receptor binder bifunctional compounds of formula (IIIB)

| # | Compound structure |
|---|---|
| I-37 | 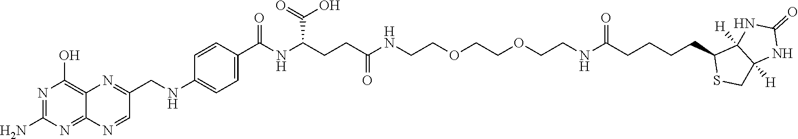 [SMILES: Nc1nc(O)c2nc(CNc3ccc(C(=O)N[C@@H](CCC(=O)NCCOCCOCCNC(=O)CCCC[C@@H]4SC[C@@H]5NC(=O)N[C@@H]54)C(=O)O)cc3)cnc2n1] |
| I-38 | 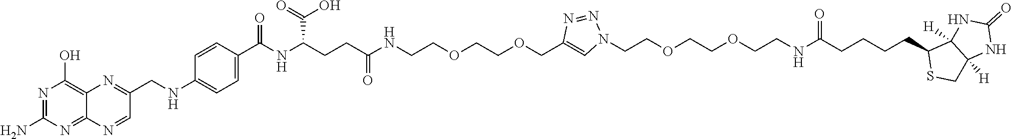 |
| I-39 | 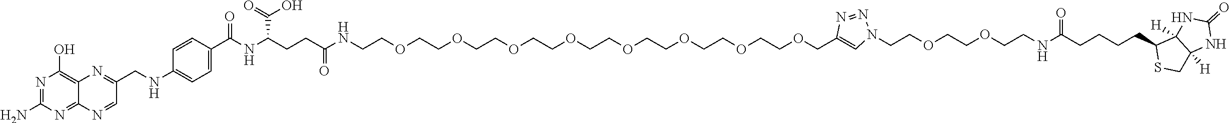 |
| I-40 | 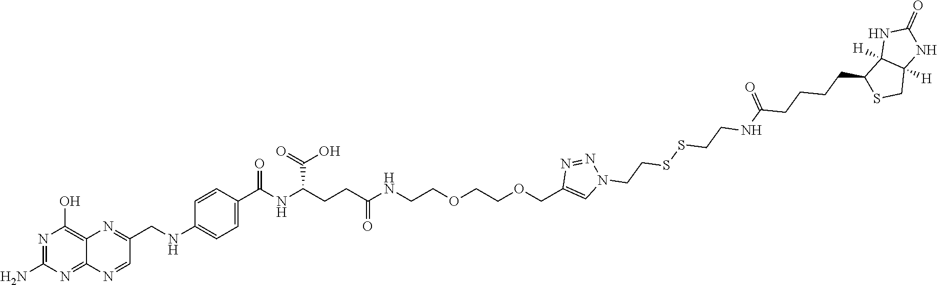 |

### 5.3. Conjugates

The compounds of this disclosure can be referred to as a conjugate, e.g., when the moiety of interest (Y) is a molecule (e.g., as described herein). Such conjugates can be prepared by conjugation of a chemoselective ligation group of any one of the compounds described herein with a compatible reactive group of a molecule Y. The compatible group of the molecule Y can be introduced by modification prior to conjugation, or can be a group present in the molecule. Alternatively, such conjugates can be prepared de novo, e.g., via modification of a Y molecule of interest starting material to introduce a linker, e.g., to which a ligand X can be attached.

Aspects of this disclosure include compounds of formula (I) where the moiety of interest Y is a selected from small molecule, dye, fluorophore, monosaccharide, disaccharide, trisaccharide, and biomolecule.

In some embodiments of the compounds of this disclosure, Y is a biomolecule. In some embodiments, the biomolecule is selected from protein, polynucleotide, polysaccharide, peptide, glycoprotein, lipid, enzyme, antibody, and antibody fragment.

In some embodiments, Y is a molecule that specifically binds to a target molecule, such as an extracellular target protein.

In some embodiments, Y is a molecule that is itself targeted for intracellular delivery.

The compounds of this disclosure can, in some cases, be referred to as a conjugate, e.g., when the moiety of interest (Y) is a molecule such as a biomolecule, where the conjugate can derived from a conjugation or coupling reaction between a chemoselective ligation group and a compatible group on the biomolecule. In some embodiments, the biomolecule is conjugated via a naturally occurring group of the biomolecule. In some embodiments, the biomolecule is conjugated via a compatible functional group that is introduced into the biomolecule prior to chemoselective conjugation. In such cases, the linking moiety between the folate binding moiety (X) and Y incorporates the residual group (e.g., Z) that is the product of the chemoselective ligation chemistry.

5.3.1. Target Binding Conjugates

Aspects of this disclosure include compounds of formula (IIIA) or (IIIB) where the moiety of interest Y is a moiety that specifically binds to a target molecule, such as a target protein. The target protein can be the target protein is a membrane bound protein or an extracellular protein. In some embodiments, Y is a small molecule that specifically binds to a target molecule, such as a target protein. In some embodiments of the compounds of this disclosure, Y is a biomolecule that specifically binds to a target protein. This disclosure provides conjugates of the particular folate binding compounds and conjugates. In some embodiments, the conjugate includes a moiety of interest Y that specifically binds a target protein, and can find use in methods of cell uptake or internalization of the target protein via binding to the cell surface receptor, and eventual degradation of the target protein.

In some embodiments, Y is an aptamer that specifically binds to a target molecule, such as a target protein. In some embodiments, Y is a peptide or protein (e.g., peptidic binding motif, protein domain, seered polypeptide, or glycoprotein) that specifically binds to a target molecule, such as a target protein. In some embodiments, Y is an antibody or antibody fragment that specifically binds to a target molecule, such as a target protein. In some embodiments, Y is a polynucleotide or oligonucleotide that specifically binds to a target molecule, such as a target protein or a target nucleic acid.

In some embodiments, one Y biomolecule is conjugated to a single moiety (X) that specifically binds to the cell surface folate receptor via a linker L. In some embodiments, one Y biomolecule is conjugated to one (Xₙ-L)- group, wherein when n=1 the (Xₙ-L)- group is referred to as monovalent, and when n>1 the (Xₙ-L)- group is referred to as multivalent (e.g., bivalent, trivalent, etc.). It is understood that in some embodiments of formula (IIIA) or (IIIB), where Y is a biomolecule, Y can be conjugated to two or more (Xₙ-L)- groups, wherein each (Xₙ-L)- group may itself be monovalent or multivalent (e.g., bivalent, trivalent, etc.). In such cases, the ratio of linked (Xₙ-L)- groups to biomolecule can be referred to as 2 or more.

In some embodiments, Y is an antibody. Accordingly, provided herein are conjugates of the following formula (VIIIa):

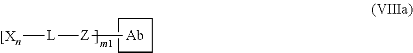

or a pharmaceutically acceptable salt thereof, wherein:
n is 1 to 20;
m1 is an average loading of 1 to 80;
each X is a moiety that binds to a cell surface folate receptor;
each L is a linker;
each Z is a residual moiety resulting from the covalent linkage of a chemoselective ligation group to a compatible group of Ab; and
Ab is the antibody or antibody fragment that specifically binds the target protein.

In some embodiments of formula (VIIIa), L is a linker of formula (IV) (e.g., as described herein). In some embodiments of formula (VIIIa), Xn-L-Z is derived from a compound of formula (I), (IIIA) and (IIIB) (e.g., as described herein), where Y is a chemoselective ligation group.

In some embodiments of formula (VIIIa), L is a linker of formula (IV):

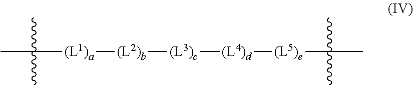

wherein L¹, L², L³, L⁴, L⁵, a, b, c, d, e, and n are defined herein.

In certain embodiments, L is selected from the linkers of Table 3.

In formula (VIIIa), Z can be any convenient residual moiety that results from the covalent linkage or conjugation of a chemoselective ligation group (Y) to a compatible reactive group of an antibody (Ab). In some instances, the compatible reactive group of antibody (Ab) is a group that can naturally be part on the biomolecule. In some instances, the compatible reactive group of antibody (Ab) is one that is introduced or incorporated into the biomolecule prior to conjugation. In such cases, the antibody (Ab) can be a modified version of a biomolecule. For example, a functional group (e.g., an amino group, a carboxylic acid group or a thiol group) of a biomolecule can be modified (e.g., using a chemical reagent such as 2-haloacetyl reagent, or 2-iminothiolane, or the like, or via coupling of a linker group including a chemoselective ligation group, such as an azide, alkyne, or the like) to introduce a compatible chemoselective ligation group.

In some embodiments of formula (VIIIa), Z is selected from the group consisting of

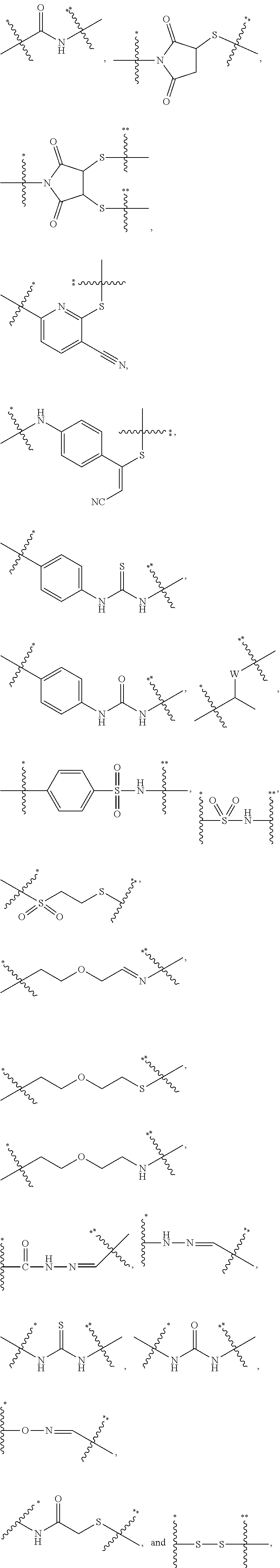

wherein [symbol] represents the point of attachment to the linker L,
wherein [symbol] represents the point of attachment to Ab,
W is CH₂, N, O or S; and
Ab is an antibody.

In certain embodiments of formula (VIIIa), Z is selected from the group consisting of

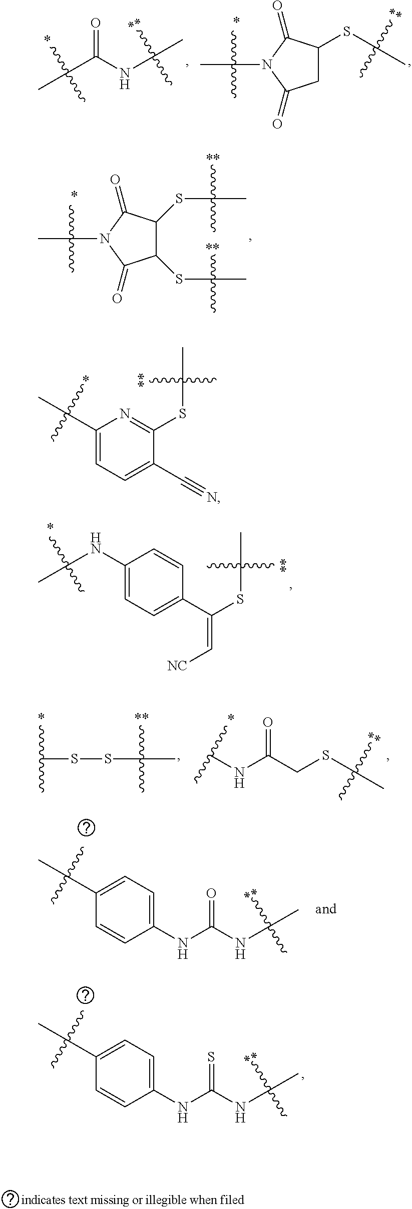

wherein [symbol] represents the point of attachment to L,
wherein [symbol] represents the point of attachment to Ab; and
Ab is an antibody.

In certain embodiments of formula (VIII), Z is selected from the group consisting of

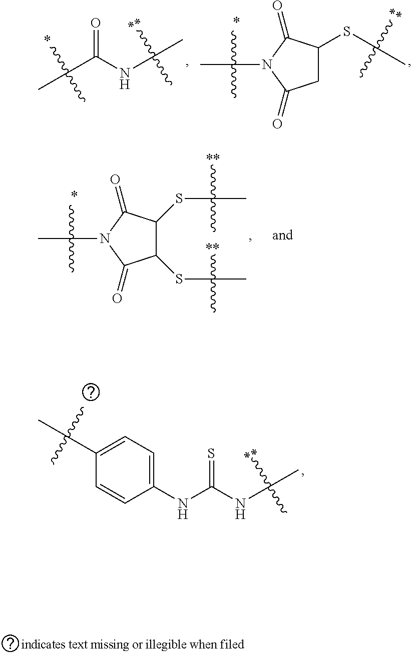

wherein [symbol] represents the point of attachment to L, wherein [symbol] represents the point of attachment to Ab.

In certain embodiments, Z is selected from the moieties of Table 4.

In certain embodiments, n is 1. In certain embodiments, n is 2. In certain embodiments, n is 3. In certain embodiments, n is 4. In certain embodiments, n is 5.

In certain embodiments of formula (VIIIa), n is 1. In certain embodiments, n is 2. In certain embodiments, n is 3. In certain embodiments, n is 4. In certain embodiments, n is 5.

In certain embodiments of formula (VIIIa), the cell surface folate receptor is folate receptor 1 (FRα).

In certain embodiments of formula (VIIIa), the cell surface folate receptor is folate receptor 2 (FRβ).

In certain embodiments of formula (VIIIa), the folate binding moiety X, is of formula (Ia):

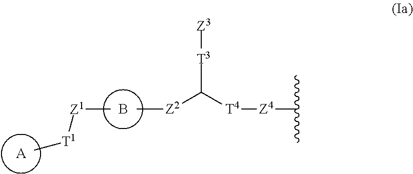

wherein:
A is a ring system of formula (XII):

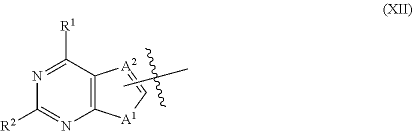

or a tautomer thereof, wherein:
   R¹ and R² are independently selected from OH, NR²¹, and optionally substituted (C₁-C₆)alkyl (e.g., —CH₃ or —CH₂OH);
   A¹ is selected from —N═CR³—, —CR³═N—, —CR³═CR³—, NR²¹, S, O, and C(R⁴)₂;
   A² is selected from N, and CR³;
   each R³ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
   each R⁴ is independently selected from H, halogen (e.g., F), and optionally substituted (C₁-C₆)alkyl
   T¹ is an optionally substituted (C₁-C₃)alkylene;
   Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;
   B is a ring system selected from optionally substituted aryl, optionally substituted heteroaryl, optionally substituted heterocycle, optionally substituted cycloalkyl, and optionally substituted bridged bicycle;
   Z² is absent, or a linking moiety selected from optionally substituted amide, optionally substituted sulfonamide, optionally substituted urea, optionally substituted thiourea, —NR²¹—, —O—, —S—, and optionally substituted (C₁-C₆)alkylene;
   Z³ is carboxyl or carboxyl bioisostere, or a prodrug thereof;
   T³ is absent, or is selected from optionally substituted (C₁-C₆)alkylene;
   T⁴ is optionally substituted (C₁-C₆)alkylene (e.g., —CH₂CH₂—), or is absent;
   Z⁴ is a linking moiety (e.g., a linking moiety selected from ester, amide, urea, thiourea, amine, sulfonamide, ether, optionally substituted aryl, optionally substituted heterocycle, and optionally substituted heteroaryl);
   each R²¹ is independently selected from H, and optionally substituted (C₁-C₆)alkyl;
   and [symbol] represents the point of attachment to -L-Y (e.g., as described herein).

In certain embodiments of formula (VIIIa), X is not folic acid, methotrexate, or pemetrexed.

In certain embodiments of formula (VIIIa), each X is independently of formula (Va), (Vb), (Vc), (Vd), (Ve), or (Vf):

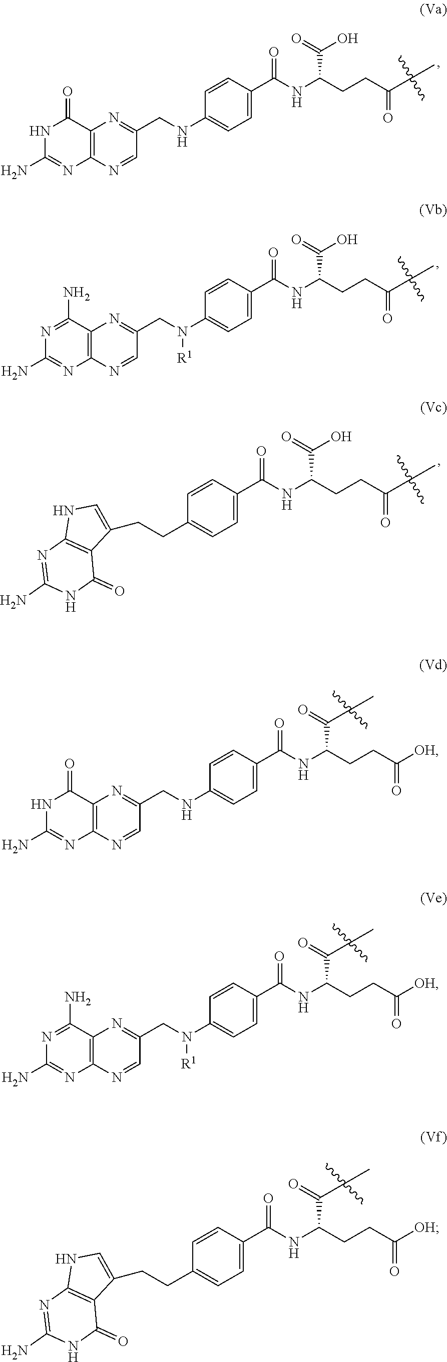

wherein R¹ is —H or —CH₃.

In certain embodiments of formula (VIIIa), each X is independently of formula (Vg)-(Vn):

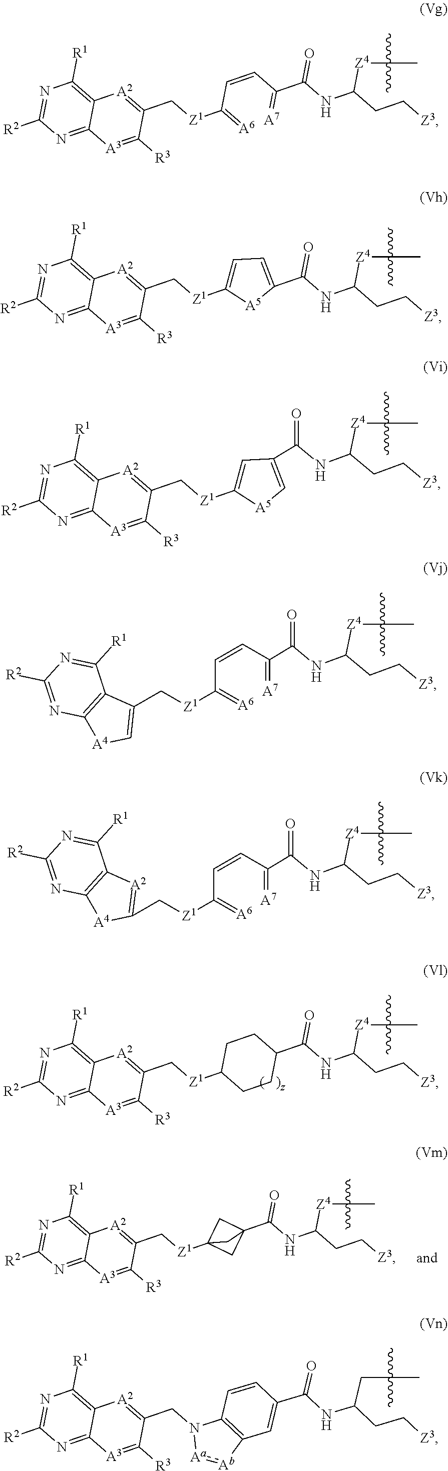

wherein R¹-R³, A²-A⁷, A^{a}-A^{b}, Z¹, Z³-Z⁴ and z are as described herein.

In certain embodiments of formula (VIIIa), each X is independently of formula (Vl), (Vm), (Vn), (Vo), or (Vp):

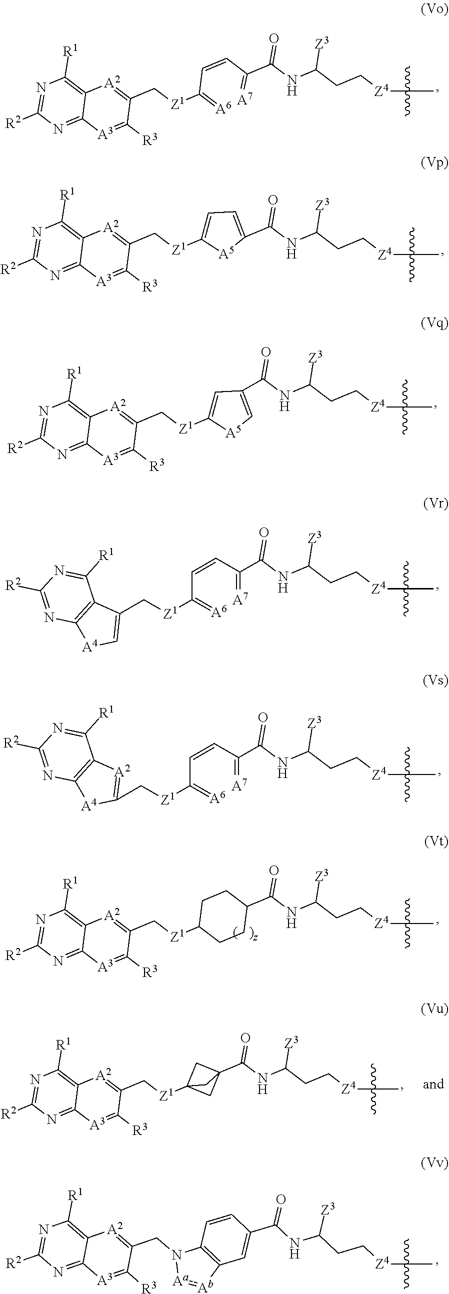

wherein R¹-R³, A²-A⁷, A^{a}-A^{b}, Z¹, Z³-Z⁴ and z are as described herein.

In certain embodiments of formula (VIIIa), each X is independently selected from a compound of Tables 2-3.

In certain embodiments of formula (VIIIa), n is 1 to 6, such as 1 to 5, 1 to 4, 1 to 3, or 1 to 2. In certain cases, n is 2 or less. In certain embodiments, n is 1. In certain embodiments, n is at least 2. In certain instances, n is 2. In certain embodiments, n is 3. In certain embodiments, n is 4. In certain embodiments, n is 5.

In certain embodiments of formula (VIIIa), m1 is 1 to 20, such as 1 to 18, 1 to 16, 1 to 14, 1 to 12, 1 to 10, 1 to 8, 1 to 6, or 1 to 4. In certain instances, m1 is 1 to 12, such as 1 to 10, 1 to 8, 1 to 6, or 1 to 4. In certain instances, m1 is at least about 2. In certain cases, m1 is at least about 3. In certain cases, m1 is at least about 4.

In certain embodiments of formula (VIIIa), Z is a residual moiety resulting from the covalent linkage of a thiol-reactive chemoselective ligation group to one or more cysteine residue(s) of Ab.

In certain embodiments of formula (VIIIa), Z is a residual moiety resulting from the covalent linkage of an amine-reactive chemoselective ligation group to one or more lysine residue(s) of Ab.

In certain embodiments, the conjugates with their linker structures described herein have weaker binding affinity to cell surface receptors. Without being bound to any particular mechanism or theory, such weaker binding affinity may be corrected to longer half-life of the conjugates, and may be useful for tuning (e.g., modifying) the pharmacokinetic properties of the conjugates described herein. In certain embodiments, such weaker binding conjugates still have sufficiently robust uptake.

The term “pharmaceutically acceptable” means being approved by a regulatory agency of the Federal or a state government, or listed in the U.S. Pharmacopeia, European Pharmacopeia or other generally recognized Pharmacopeia for use in animals, and, more particularly in humans.

The term “pharmaceutically acceptable salt” refers to those salts of the conjugate provided herein, which are formed by the process of the present application which are suitable for use in contact with the tissues of humans and lower animals without undue toxicity, irritation, allergic response and the like. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the conjugate compounds, or separately by reacting the free base function or group of a compound with a suitable organic acid. Examples of pharmaceutically acceptable salts include, but are not limited to, nontoxic acid addition salts, or salts of an amino group formed with inorganic acids such as hydrochloric acid, hydrobromic acid, phosphoric acid, etc., or with organic acids such as acetic acid, maleic acid, tartaric acid, citric acid, succinic acid or malonic acid. Other pharmaceutically acceptable salts include, but are not limited to, adipate, alginate, ascorbate, benzenesulfonate, benzoate, bisulfate, citrate, digluconate, dodecylsulfate, ethanesulfonate, formate, fumarate, gluconate, 2-hydroxy-ethanesulfonate, lactate, laurate, malate, maleate, malonate, methanesulfonate, oleate, oxalate, palmitate, phosphate, propionate, stearate, succinate, sulfate, tartrate, p-toluenesulfonate, valerate salts, and the like. Representative alkali or alkaline earth metal salts include sodium, lithium, potassium, calcium, or magnesium salts, and the like. Further pharmaceutically acceptable salts include, nontoxic ammonium, quaternary ammonium, and amine cations formed using counterions such as halide, hydroxide, carboxylate, sulfate, phosphate, nitrate, alkyl groups having from 1 to 6 carbon atoms (e.g., C₁₋₆ alkyl), sulfonate and aryl sulfonate.

Conjugates of the polypeptide (P), e.g., an antibody (Ab) and compound (Xn-L-Y) may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that the conjugates described herein may be prepared using any suitable methods as disclosed in the art (see, e.g., Bioconjugate Techniques (Hermanson ed., 2d ed. 2008)).

In certain embodiments of the conjugates described herein, L is bonded through an amide bond to a lysine residue of P. In certain embodiments of the conjugates described herein, L is bonded through a thioether bond to a cysteine residue of P. In certain embodiments of the conjugates described herein, L is bonded through an amide bond to a lysine residue of Ab, as depicted above. In certain embodiments of the conjugates described herein, L is bonded through a thioether bond to a cysteine residue of Ab, as depicted above. In certain embodiments of the conjugates described herein, L is bonded through two thioether bonds to two cysteine residues of Ab, wherein the two cysteine residues are from an opened cysteine-cysteine disulfide bond in Ab, as depicted above. In certain embodiments, the opened cysteine-cysteine disulfide bond is an interchain disulfide bond.

In certain embodiments of the conjugates described herein, when L is bonded through an amide bond to a lysine residue of P, m is an integer from 1 to 80. In certain embodiments of the conjugates described herein, when L is bonded through a thioether bond to a cysteine residue of P, m is an integer from 1 to 8.

In certain embodiments, conjugation to the polypeptide P or the antibody Ab may be via site-specific conjugation. Site-specific conjugation may, for example, result in homogeneous loading and minimization of conjugate subpopulations with potentially altered antigen-binding or pharmacokinetics. In certain embodiments, for example, conjugation may comprise engineering of cysteine substitutions at positions on the polypeptide or antibody, e.g., on the heavy and/or light chains of an antibody that provide reactive thiol groups and do not disrupt polypeptide or antibody folding and assembly or alter polypeptide or antigen binding (see, e.g., Junutula et al., J. Immunol. Meth. 2008; 332: 41-52; and Junutula et al., Nature Biotechnol. 2008; 26: 925-32; see also WO2006/034488 (herein incorporated by reference in its entirety)). In another non-limiting approach, selenocysteine is cotranslationally inserted into a polypeptide or antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., Proc. Natl. Acad. Sci. USA 2008; 105: 12451-56; and Hofer et al., Biochemistry 2009; 48(50): 12047-57). Yet other non-limiting techniques that allow for site-specific conjugation to polypeptides or antibodies include engineering of non-natural amino acids, including, e.g., p-acetylphenylalanine (p-acetyl-Phe), p-azidomethyl-N-phenylalanine (p-azidomethyl-Phe), and azidolysine (azido-Lys) at specific linkage sites, and can further include engineering unique functional tags, including, e.g., LPXTG, LLQGA, sialic acid, and GlcNac, for enzyme mediated conjugation. See Jackson, Org. Process Res. Dev. 2016; 20: 852-866; and Tsuchikama and An, Protein Cell 2018; 9(1):33-46, the contents of each of which is incorporated by reference in its entirety. See also US 2019/0060481 A1 & US 2016/0060354 A1, the contents of each of which is incorporated by reference in its entirety. All such methodologies are contemplated for use in connection with making the conjugates described herein.

Loading of the compounds of formula (I) to the polypeptides (e.g., antibodies) described herein is represented by “m1” in formula (VIIIa), and is the average number of units of “Xn-L-” or “Xn-” per conjugate molecule. As used herein, the term “DAR” refers to the average value of “m” or the loading of the conjugate. The number of “X” moieties (e.g., folate moieties) per each unit of “Xn-L-” or “Xn-” is represented by “n” in formula (IIIa). As used herein, the term “valency” or “valencies” refers to the number of “X” moieties per unit (“n”). It will be understood that loading, or DAR, is not necessarily equivalent to the number of “X” moieties per conjugate molecule. By means of example, where there is one “X” moiety per unit (n=1; valency is “1”), and one “Xn-L-” unit per conjugate (m=1), there will be 1×1=1 “X” moiety per conjugate. However, where there are two “X” moieties per unit (n=2; valency is “2”), and four “Xn-L-” units per conjugate (m=4), there will be 2×4=8 “X” moieties per conjugate. Accordingly, for the conjugates described herein, the total number of “X” moieties per conjugate molecule will be n×m. As used herein, the term “total valency” or “total valencies” refers to the total number of “X” moieties per conjugate molecule (n×m; total valency).

DAR (loading) may range from 1 to 80 units per conjugate. The conjugates provided herein may include collections of polypeptides, antibodies or antigen binding fragments conjugated with a range of units, e.g., from 1 to 80. The average number of units per polypeptide or antibody in preparations of the conjugate from conjugation reactions may be characterized by conventional means such as mass spectroscopy. The quantitative distribution of DAR (loading) in terms of m may also be determined. In some instances, separation, purification, and characterization of homogeneous conjugate where m is a certain value may be achieved by means such as electrophoresis.

In certain embodiments, the DAR for a conjugate provided herein ranges from 1 to 80. In certain embodiments, the DAR for a conjugate provided herein ranges from 1 to 70. In certain embodiments, the DAR for a conjugate provided herein ranges from 1 to 60. In certain embodiments, the DAR for a conjugate provided herein ranges from 1 to 50. In certain embodiments, the DAR for a conjugate provided herein ranges from 1 to 40. In certain embodiments, the DAR for a conjugate provided herein ranges from 1 to 35. In certain embodiments, the DAR for a conjugate provided herein ranges from 1 to 30. In certain embodiments, the DAR for a conjugate provided herein ranges from 1 to 25. In certain embodiments, the DAR for a conjugate provided herein ranges from 1 to 20. In certain embodiments, the DAR for a conjugate provided herein ranges from 1 to 18. In certain embodiments, the DAR for a conjugate provided herein ranges from 1 to 15. In certain embodiments, the DAR for a conjugate provided herein ranges from 1 to 12. In certain embodiments, the DAR for a conjugate provided herein ranges from 1 to 10. In certain embodiments, the DAR for a conjugate provided herein ranges from 1 to 9. In certain embodiments, the DAR for a conjugate provided herein ranges from 1 to 8. In certain embodiments, the DAR for a conjugate provided herein ranges from 1 to 7. In certain embodiments, the DAR for a conjugate provided herein ranges from 1 to 6. In certain embodiments, the DAR for a conjugate provided herein ranges from 1 to 5. In certain embodiments, the DAR for a conjugate provided herein ranges from 1 to 4. In certain embodiments, the DAR for a conjugate provided herein ranges from 1 to 3. In certain embodiments, the DAR for a conjugate provided herein ranges from 2 to 12. In certain embodiments, the DAR for a conjugate provided herein ranges from 2 to 10. In certain embodiments, the DAR for a conjugate provided herein ranges from 2 to 9. In certain embodiments, the DAR for a conjugate provided herein ranges from 2 to 8. In certain embodiments, the DAR for a conjugate provided herein ranges from 2 to 7. In certain embodiments, the DAR for a conjugate provided herein ranges from 2 to 6. In certain embodiments, the DAR for a conjugate provided herein ranges from 2 to 5. In certain embodiments, the DAR for a conjugate provided herein ranges from 2 to 4. In certain embodiments, the DAR for a conjugate provided herein ranges from 3 to 12. In certain embodiments, the DAR for a conjugate provided herein ranges from 3 to 10. In certain embodiments, the DAR for a conjugate provided herein ranges from 3 to 9. In certain embodiments, the DAR for a conjugate provided herein ranges from 3 to 8. In certain embodiments, the DAR for a conjugate provided herein ranges from 3 to 7. In certain embodiments, the DAR for a conjugate provided herein ranges from 3 to 6. In certain embodiments, the DAR for a conjugate provided herein ranges from 3 to 5. In certain embodiments, the DAR for a conjugate provided herein ranges from 3 to 4.

In certain embodiments, the DAR for a conjugate provided herein ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7.

In certain embodiments, the DAR for a conjugate provided herein is about 1, about 2, about 3, about 4, about 5, about 6, about 7, about 8, about 9, about 10, about 11, about 12, or more. In some embodiments, the DAR for a conjugate provided herein is about 3.1, about 3.2, about 3.3, about 3.4, about 3.5, about 3.6, about 3.7, about 3.8, or about 3.9.

In some embodiments, the DAR for a conjugate provided herein ranges from 2 to 20, 2 to 19, 2 to 18, 2 to 17, 2 to 16, 2 to 15, 2 to 14, or 2 to 13. In some embodiments, the DAR for a conjugate provided herein ranges from 3 to 20, 3 to 19, 3 to 18, 3 to 17, 3 to 16, 3 to 15, 3 to 14, or 3 to 13. In some embodiments, the DAR for a conjugate provided herein is about 1. In some embodiments, the DAR for a conjugate provided herein is about 2. In some embodiments, the DAR for a conjugate provided herein is about 3. In some embodiments, the DAR for a conjugate provided herein is about 4. In some embodiments, the DAR for a conjugate provided herein is about 3.8. In some embodiments, the DAR for a conjugate provided herein is about 5. In some embodiments, the DAR for a conjugate provided herein is about 6. In some embodiments, the DAR for a conjugate provided herein is about 7. In some embodiments, the DAR for a conjugate provided herein is about 8. In some embodiments, the DAR for a conjugate provided herein is about 9. In some embodiments, the DAR for a conjugate provided herein is about 10. In some embodiments, the DAR for a conjugate provided herein is about 11. In some embodiments, the DAR for a conjugate provided herein is about 12. In some embodiments, the DAR for a conjugate provided herein is about 13. In some embodiments, the DAR for a conjugate provided herein is about 14. In some embodiments, the DAR for a conjugate provided herein is about 15. In some embodiments, the DAR for a conjugate provided herein is about 16. In some embodiments, the DAR for a conjugate provided herein is about 17. In some embodiments, the DAR for a conjugate provided herein is about 18. In some embodiments, the DAR for a conjugate provided herein is about 19. In some embodiments, the DAR for a conjugate provided herein is about 20.

In some embodiments, the DAR for a conjugate provided herein is about 25. In some embodiments, the DAR for a conjugate provided herein is about 30. In some embodiments, the DAR for a conjugate provided herein is about 35. In some embodiments, the DAR for a conjugate provided herein is about 40. In some embodiments, the DAR for a conjugate provided herein is about 50. In some embodiments, the DAR for a conjugate provided herein is about 60. In some embodiments, the DAR for a conjugate provided herein is about 70. In some embodiments, the DAR for a conjugate provided herein is about 80.

In certain embodiments, fewer than the theoretical maximum of units are conjugated to the polypeptide, e.g., antibody, during a conjugation reaction. A polypeptide may contain, for example, lysine residues that do not react with the compound or linker reagent. Generally, for example, antibodies do not contain many free and reactive cysteine thiol groups which may be linked to a drug unit; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the compound is conjugated via a lysine residue on the antibody. In some embodiments, the linker unit or a drug unit is conjugated via a cysteine residue on the antibody.

In certain embodiments, the amino acid that attaches to a unit is in the heavy chain of an antibody. In certain embodiments, the amino acid that attaches to a unit is in the light chain of an antibody. In certain embodiments, the amino acid that attaches to a unit is in the hinge region of an antibody. In certain embodiments, the amino acid that attaches to a unit is in the Fc region of an antibody. In certain embodiments, the amino acid that attaches to a unit is in the constant region (e.g., CH1, CH2, or CH3 of a heavy chain, or CH1 of a light chain) of an antibody. In yet other embodiments, the amino acid that attaches to a unit or a drug unit is in the VH framework regions of an antibody. In yet other embodiments, the amino acid that attaches to unit is in the VL framework regions of an antibody.

The DAR (loading) of a conjugate may be controlled in different ways, e.g., by: (i) limiting the molar excess of compound or conjugation reagent relative to polypeptide, (ii) limiting the conjugation reaction time or temperature, (iii) partial or limiting reductive conditions for cysteine thiol modification, (iv) engineering by recombinant techniques the amino acid sequence of the polypeptide, such that the number and position of cysteine residues is modified for control of the number and/or position of linker-drug attachments (such as for thiomabs prepared as disclosed in WO2006/034488 (herein incorporated by reference in its entirety)).

It is to be understood that the preparation of the conjugates described herein may result in a mixture of conjugates with a distribution of one or more units attached to a polypeptide, for example, an antibody. Individual conjugate molecules may be identified in the mixture by mass spectroscopy and separated by HPLC, e.g. hydrophobic interaction chromatography, including such methods known in the art. In certain embodiments, a homogeneous conjugate with a single DAR (loading) value may be isolated from the conjugation mixture by electrophoresis or chromatography.

### 5.4. Pharmaceutical Compositions

In another embodiment, provided herein are pharmaceutical compositions comprising one or more conjugates disclosed herein and a pharmaceutically acceptable carrier.

In certain embodiments, the pharmaceutical compositions provided herein contain therapeutically effective amounts of one or more of the conjugates provided herein, and optionally one or more additional prophylactic or therapeutic agents, in a pharmaceutically acceptable carrier.

Pharmaceutical carriers suitable for administration of the conjugates provided herein include any such carriers known to those skilled in the art to be suitable for the particular mode of administration.

The conjugates described herein can be formulated as the sole pharmaceutically active ingredient in the composition or can be combined with other active ingredients.

In certain embodiments, the conjugate is formulated into one or more suitable pharmaceutical preparations, such as solutions, suspensions, powders, sustained release formulations or elixirs in sterile solutions or suspensions for parenteral administration, or as transdermal patch preparation and dry powder inhalers.

In compositions provided herein, a conjugate described herein may be mixed with a suitable pharmaceutical carrier. The concentration of the conjugate in the compositions can, for example, be effective for delivery of an amount, upon administration, that treats, prevents, or ameliorates a condition or disorder described herein or a symptom thereof.

In certain embodiments, the pharmaceutical compositions provided herein are formulated for single dosage administration. To formulate a composition, the weight fraction of conjugate is dissolved, suspended, dispersed or otherwise mixed in a selected carrier at an effective concentration such that the treated condition is relieved, prevented, or one or more symptoms are ameliorated.

Concentrations of the conjugate in a pharmaceutical composition provided herein will depend on, e.g., the physicochemical characteristics of the conjugate, the dosage schedule, and amount administered as well as other factors known to those of skill in the art.

Pharmaceutical compositions described herein are provided for administration to a subject, for example, humans or animals (e.g., mammals) in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals in unit dosage form, including oral or nasal solutions or suspensions and oil-water emulsions containing suitable quantities of a conjugate or pharmaceutically acceptable derivatives thereof. The conjugate is, in certain embodiments, formulated and administered in unit-dosage forms or multiple-dosage forms. Unit-dose forms as used herein refers to physically discrete units suitable for human or animal (e.g., mammal) subjects and packaged individually as is known in the art. Each unit-dose contains a predetermined quantity of a conjugate sufficient to produce the desired therapeutic effect, in association with the required pharmaceutical carrier, vehicle or diluent. Examples of unit-dose forms include ampoules and syringes and individually packaged capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of capsules or bottles. Hence, in specific aspects, multiple dose form is a multiple of unit-doses which are not segregated in packaging.

In certain embodiments, the conjugates herein are in a liquid pharmaceutical formulation. Liquid pharmaceutically administrable formulations can, for example, be prepared by dissolving, dispersing, or otherwise mixing a conjugate and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington: The Science and Practice of Pharmacy (2012) 22nd ed., Pharmaceutical Press, Philadelphia, PA Dosage forms or compositions containing antibody in the range of 0.005% to 100% with the balance made up from non-toxic carrier can be prepared.

Parenteral administration, in certain embodiments, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. Other routes of administration may include, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

In certain embodiments, intravenous or intraarterial infusion of a sterile aqueous solution containing a conjugate described herein is an effective mode of administration. Another embodiment is a sterile aqueous or oily solution or suspension containing a conjugate described herein injected as necessary to produce the desired pharmacological effect.

In certain embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving a conjugate provided herein, in a suitable solvent. In some embodiments, the lyophilized powder is sterile. Suitable solvents can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. A suitable solvent can also contain a buffer, such as citrate, sodium or potassium phosphate or other such buffer known to those of skill in the art at, in certain embodiments, about neutral pH. Subsequent sterile filtration of the solution followed by lyophilization under standard conditions known to those of skill in the art provides an example of a formulation. In certain embodiments, the resulting solution will be apportioned into vials for lyophilization. Lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier.

In certain embodiments, the conjugates provided herein can be formulated for local administration or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

### 5.5. Uses and Methods

In one aspect, provided herein are methods of using the conjugates described herein to remove a polypeptide of interest (a target protein) from a cell's surface. In one aspect, provided herein are methods of using the conjugates described herein to remove a polypeptide of interest (a target protein) from the extracellular milieu. For example, in one embodiment, provided herein are methods of using the conjugates described herein to remove a polypeptide of interest (a target protein) from the surface of a cell by sequestering the target protein in the cell's lysosome. In another embodiment, provided herein are methods of using the conjugates described herein to remove a polypeptide of interest (a target protein) from the extracellular space (the extracellular milieu) of a cell by sequestering the target protein in the cell's lysosome. In another embodiment, provided herein are methods of using the conjugates described herein to remove a polypeptide of interest (a target protein) from the surface of a cell by sequestering the target protein in the cell's lysosome and degrading the target protein. In another embodiment, provided herein are methods of using the conjugates described herein to remove a polypeptide of interest (a target protein) from the extracellular space (the extracellular milieu) of a cell by sequestering the target protein in the cell's lysosome and degrading the target protein.

Removal of a target protein may refer to reduction, or depletion, of the target protein from the cell surface or from the extracellular space, or the extracellular milieu, that is, a reduction, or depletion, of the amount of the target protein on the cell surface or in the extracellular milieu.

In one aspect, provided herein are methods of using the conjugates described herein to sequester a polypeptide of interest (a target protein) in a cell's lysosome. In one aspect, provided herein are methods of using the conjugates described herein to sequester a polypeptide of interest (a target protein) in a cell's lysosome and to degrade the polypeptide of interest.

In one aspect, provided herein are methods of using the conjugates described herein to degrade a polypeptide of interest (a target protein).

In one aspect, provided herein are methods of depleting a polypeptide of interest (a target protein) described herein by degradation through a cell's lysosomal pathway.

In another aspect, provided herein are methods of depleting a polypeptide of interest (a target protein) described herein by administering to a subject in need thereof an effective amount of a conjugate or pharmaceutically acceptable salt described herein, or a pharmaceutical composition described herein. In certain embodiments, the subject is a mammal (e.g., human).

In certain embodiments, the target protein is a VEGF protein, an EGFR protein, a VEGFR protein, a PD-L1 protein, an FGFR2 protein or an FGFR3 protein.

In another aspect, provided herein are methods of treating a disease or disorder by administering to a subject, e.g., a human, in need thereof an effective amount of a conjugate or pharmaceutically acceptable salt described herein, or a pharmaceutical composition described herein.

The terms “administer”, “administration”, or “administering” refer to the act of injecting or otherwise physically delivering a substance (e.g., a conjugate or pharmaceutical composition provided herein) to a subject or a patient (e.g., human), such as by mucosal, topical, intradermal, parenteral, intravenous, intramuscular delivery and/or any other method of physical delivery described herein or known in the art. In a particular embodiment, administration is by intravenous infusion.

The terms “effective amount” or “therapeutically effective amount” refer to an amount of a therapeutic (e.g., a conjugate or pharmaceutical composition provided herein) which is sufficient to treat, diagnose, prevent, delay the onset of, reduce and/or ameliorate the severity and/or duration of a given condition, disorder or disease and/or a symptom related thereto. These terms also encompass an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy or to serve as a bridge to another therapy. In some embodiments, “effective amount” as used herein also refers to the amount of a conjugate described herein to achieve a specified result.

In certain embodiments, when the disorder or disease is cancer, “effective amount” or “therapeutically effective amount” mean that amount of a conjugate or pharmaceutical composition provided herein which, when administered to a human suffering from a cancer, is sufficient to effect treatment for the cancer. “Treating” or “treatment” of the cancer includes one or more of:
(1) limiting/inhibiting growth of the cancer, e.g. limiting its development;
(2) reducing/preventing spread of the cancer, e.g. reducing/preventing metastases;
(3) relieving the cancer, e.g. causing regression of the cancer,
(4) reducing/preventing recurrence of the cancer; and
(5) palliating symptoms of the cancer.

The terms “subject” and “patient” are used interchangeably. A subject can be a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder provided herein. In another embodiment, the subject is a mammal, e.g., a human, at risk of developing a disease or disorder provided herein. In a specific embodiment, the subject is human.

The terms “therapies” and “therapy” can refer to any protocol(s), method(s), compositions, formulations, and/or agent(s) that can be used in the prevention, treatment, management, or amelioration of a disease or disorder or symptom thereof (e.g., a disease or disorder provided herein or one or more symptoms or condition associated therewith). In certain embodiments, the terms “therapies” and “therapy” refer to drug therapy, adjuvant therapy, radiation, surgery, biological therapy, supportive therapy, and/or other therapies useful in treatment, management, prevention, or amelioration of a disease or disorder or one or more symptoms thereof. In certain embodiments, the term “therapy” refers to a therapy other than a conjugate described herein or pharmaceutical composition thereof.

In certain embodiments, the disease or disorder is treated by depletion of the target protein by degradation through the lysosomal pathway.

In certain embodiments, the disease or disorder is treated by depletion of certain proteins, for example, soluble proteins, e.g., secreted proteins, cell surface proteins (for example, cell surface receptor proteins, e.g., tyrosine kinase receptors, soluble cytokine receptors, and immune checkpoint receptors, e.g., EGFR, VEGFR, FGFR, and PD-L1), lectins, complements, lipoproteins, transport proteins, MHC class I and class II molecules, cytokines, chemokines, and/or receptors, or fragments or subunits of any of the foregoing.

In certain embodiments, the disease or disorder is a cancer.

In certain embodiments, the cancer is selected from the group consisting of bladder cancer, breast cancer, cervical cancer, cholangiocarcinoma, endometrial cancer, hepatocellular carcinoma, kidney cancer, melanoma, myeloid neoplasms, non-small cell lung cancer (NSCLC), Ewing's sarcoma, and Hodgkin's Lymphoma.

In certain embodiments, the cancer is a solid tumor.

In certain embodiments, the disease or disorder is an inflammatory or autoimmune disease.

In certain embodiments, the disease or disorder is an inflammatory disease.

In certain embodiments, the disease or disorder is an autoimmune disease.

### 5.6. Definitions

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

Unless specifically stated otherwise herein, references made in the singular may also include the plural. For example, “a” and “an” may refer to either one, or one or more.

The term “compounds” refers to at least one compound. For example, a compound of Formula (I) or (X) includes a compound of the formula, and/or two or more compounds of Formula (I).

Unless otherwise indicated, any heteroatom with unsatisfied valences is assumed to have hydrogen atoms sufficient to satisfy the valences.

Throughout the specification, groups and substituents thereof may be chosen by one skilled in the field to provide stable moieties and compounds.

The terms “halo” and “halogen,” refer to F, Cl, Br, and I.

The term “cyano” refers to the group —CN.

The term “amino” refers to the group —NH₂.

The term “hydroxy” refers to the group —OH.

The term “nitro” refers to the group —NO₂.

The term “oxo” refers to the group ═O.

The term “alkyl” refers to both branched and straight-chain saturated aliphatic hydrocarbon groups containing, for example, from 1 to 12 carbon atoms, from 1 to 6 carbon atoms, and from 1 to 4 carbon atoms. Examples of alkyl groups include, but are not limited to, methyl (Me), ethyl (Et), propyl (e.g., n-propyl and i-propyl), butyl (e.g., n-butyl, i-butyl, sec-butyl, and t-butyl), and pentyl (e.g., n-pentyl, isopentyl, neopentyl), n-hexyl, 2-methylpentyl, 2-ethylbutyl, 3-methylpentyl, and 4-methylpentyl. When numbers appear in a subscript after the symbol “C”, the subscript defines with more specificity the number of carbon atoms that a particular group may contain. For example, “C₁₋₆ alkyl” denotes straight and branched chain alkyl groups with one to six carbon atoms.

The term “haloalkyl” is intended to include both branched and straight-chain saturated aliphatic hydrocarbon groups substituted with one or more halogen atoms. For example, “C₁₋₄ haloalkyl” is intended to include C₁, C₂, C₃, and C₄ alkyl groups substituted with one or more halogen atoms. Representative examples of haloalkyl groups include, but are not limited to, —CF₃, —CCl₃, —CFCl₂, and —CH₂CF₃.

The term “fluoroalkyl” is intended to include both branched and straight-chain saturated aliphatic hydrocarbon groups substituted with one or more fluorine atoms. For example, “C₁₋₄ fluoroalkyl” is intended to include C₁, C₂, C₃, and C₄ alkyl groups substituted with one or more fluorine atoms. Representative examples of fluoroalkyl groups include, but are not limited to, —CF₃ and —CH₂CF₃.

The term “hydroxyalkyl” includes both branched and straight-chain saturated alkyl groups substituted with one or more hydroxyl groups. For example, “hydroxyalkyl” includes —CH₂OH, —CH₂CH₂OH, and C₁₋₄ hydroxyalkyl.

The term “aminoalkyl” includes both branched and straight-chain saturated alkyl groups substituted with one or more amine groups. For example, “aminoalkyl” includes —CH₂NH₂, —CH₂CH₂NH₂, and C₁₋₄ aminoalkyl.

The term “alkenyl” refers to a straight or branched chain hydrocarbon radical containing from 2 to 12 carbon atoms and at least one carbon-carbon double bond.

Exemplary such groups include ethenyl or allyl. For example, “C₂₋₆ alkenyl” denotes straight and branched chain alkenyl groups with two to six carbon atoms.

The term “alkynyl” refers to a straight or branched chain hydrocarbon radical containing from 2 to 12 carbon atoms and at least one carbon to carbon triple bond.

Exemplary such groups include ethynyl. For example, “C₂₋₆ alkynyl” denotes straight and branched chain alkynyl groups with two to six carbon atoms.

The term “cycloalkyl,” as used herein, refers to a group derived from a saturated monocyclic or polycyclic hydrocarbon molecule by removal of one hydrogen atom from a saturated ring carbon atom. Representative examples of cycloalkyl groups include, but are not limited to, cyclopropyl, cyclopentyl, and cyclohexyl. When numbers appear in a subscript after the symbol “C”, the subscript defines with more specificity the number of carbon atoms that a particular cycloalkyl group may contain. For example, “C₃₋₆ cycloalkyl” denotes cycloalkyl groups with three to six carbon atoms.

The term “cycloalkenyl,” as used herein, refers to a group derived from a non-aromatic monocyclic or polycyclic hydrocarbon molecule having at least one carbon-carbon double bond, by removal of one hydrogen atom from a saturated ring carbon atom. Representative examples of cycloalkenyl groups include, but are not limited to, cyclobutenyl, cyclopentenyl, and cyclohexenyl. When numbers appear in a subscript after the symbol “C”, the subscript defines with more specificity the number of carbon atoms that a particular cycloalkyl group may contain. For example, “C₄₋₆ cycloalkenyl” denotes cycloalkenyl groups with four to six carbon atoms.

The term “alkoxy,” as used herein, refers to an alkyl group attached to the parent molecular moiety through an oxygen atom, for example, methoxy group (—OCH₃). For example, “C₁₋₃ alkoxy” denotes alkoxy groups with one to three carbon atoms.

The terms “haloalkoxy” and “—O(haloalkyl)” represent a haloalkyl group as defined above attached through an oxygen linkage (—O—). For example, “C₁₋₄ haloalkoxy” is intended to include C₁, C₂, C₃, and C₄ haloalkoxy groups.

The terms “fluoroalkoxy” and “—O(fluoroalkyl)” represent a fluoroalkyl group as defined above attached through an oxygen linkage (—O—). For example, “C₁₋₄ fluoroalkoxy” is intended to include C₁, C₂, C₃, and C₄ fluoroalkoxy groups.

The terms “hydroxyalkoxy” and “—O(hydroxyalkyl)” represent a hydroxyalkyl group as defined above attached through an oxygen linkage (—O—). For example, “C₁₋₄ hydroxyalkoxy” is intended to include C₁, C₂, C₃, and C₄ hydroxyalkoxy groups.

The term “alkylthio,” refers to an alkyl group attached to the parent molecular moiety through a sulfur atom, for example, methylthio group (—SCH₃). For example, “C₁₋₃ alkylthio” denotes alkylthio groups with one to three carbon atoms.

The term “arylthio,” refers to an aryl group attached to the parent molecular moiety through a sulfur atom, for example, phenylthio group (—S(phenyl)).

The terms “carbocycle”, “carbocyclo”, “carbocyclic” or “carbocyclyl” are used interchangeably and refer to cyclic groups having at least one saturated or partially saturated non-aromatic ring wherein all atoms of all rings are carbon. The carbocyclyl ring may be unsubstituted or may contain one or more substituents as valence allows. Thus, the term includes nonaromatic rings such as for example, cycloalkyl, cycloalkenyl, and cycloalkynyl rings. Exemplary bicyclic carbocyclyl groups include, indanyl, indenyl, dihydronaphthalenyl, tetrahydronaphthenyl, hexahydronaphthalenyl, octahydronaphthalenyl, decahydronaphthalenyl, bicycloheptanyl, bicyclooctanyl, and bicyclononanyl.

The term “aryl” refers to a group of atoms derived from a molecule containing aromatic ring(s) by removing one hydrogen that is bonded to the aromatic ring(s). Heteroaryl groups that have two or more rings must include only aromatic rings. Representative examples of aryl groups include, but are not limited to, phenyl and naphthyl. The aryl ring may be unsubstituted or may contain one or more substituents as valence allows.

The term “benzyl” refers to a methyl group in which one of the hydrogen atoms is replaced by a phenyl group. The phenyl ring may be unsubstituted or may contain one or more substituents as valence allows.

The term “aryloxy” refers to an aryl group attached to the parent molecular moiety through an oxygen atom, for example, phenoxy group (—O(phenyl)).

The term “heteroatom” refers to oxygen (O), sulfur (S), and nitrogen (N).

The terms “heterocycle”, “heterocyclo”, “heterocyclic”, and “heterocyclyl” are used interchangeably and refer to cyclic groups having at least saturated or partially saturated non-aromatic ring and wherein one or more of the rings have at least one heteroatom (O, S or N), said heteroatom containing ring preferably having 1 to 3 heteroatoms independently selected from O, S, and/or N. The ring of such a group containing a heteroatom can contain one or two oxygen or sulfur atoms and/or from one to four nitrogen atoms provided that the total number of heteroatoms in each ring is four or less, and further provided that the ring contains at least one carbon atom. The nitrogen and sulfur atoms may optionally be oxidized and the nitrogen atoms may optionally be quaternized. The heterocyclo group may be attached at any available nitrogen or carbon atom. The heterocyclo ring may be unsubstituted or may contain one or more substituents as valence allows.

Exemplary monocyclic heterocyclyl groups include pyrrolidinyl, imidazolinyl, oxazolidinyl, isoxazolinyl, thiazolidinyl, isothiazolidinyl, tetrahydrofuranyl, piperidinyl, piperazinyl, 2-oxopiperazinyl, 2-oxopiperidinyl, 2-oxopyrrolodinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane, tetrahydro-1,1-dioxothienyl, dihydroisoindolyl, and tetrahydroquinolinyl.

The term “heteroaryl” refers to substituted and unsubstituted aromatic 5- or 6-membered monocyclic groups and 9- or 10-membered bicyclic groups that have at least one heteroatom (0, S or N) in at least one of the rings, said heteroatom-containing ring preferably having 1, 2, or 3 heteroatoms independently selected from O, S, and/or N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic group are aromatic and may contain only carbon atoms. The nitrogen and sulfur atoms may optionally be oxidized and the nitrogen atoms may optionally be quaternized. Bicyclic heteroaryl groups must include only aromatic rings. The heteroaryl group may be attached at any available nitrogen or carbon atom of any ring. The heteroaryl ring system may be unsubstituted or may contain one or more substituents.

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thiophenyl, oxadiazolyl, pyridinyl, pyrazinyl, pyrimidinyl, pyridazinyl, and triazinyl.

Exemplary bicyclic heteroaryl groups include indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, and pyrrolopyridyl.

The term “spirocarbocyclo”, “spirocarbocyclic”, or “spirocarbocyclyl” refers to a carbocyclyl ring attached to the molecular moiety by a carbon atom in the carbocyclyl ring that is shared with the molecular moiety.

The term “spiroheterocyclo”, “spiroheterocyclic”, or “spiroheterocyclyl” refers to a heterocyclyl ring attached to the molecular moiety by a carbon atom in the heterocyclyl ring that is shared with the molecular moiety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds of Formula (I) or (X) can be provided as amorphous solids or crystalline solids. Lyophilization can be employed to provide the compounds as amorphous solids.

It should further be understood that solvates (e.g., hydrates) of the compounds of Formula (I) or (X) are also within the scope of the present disclosure. The term “solvate” means a physical association of a compound of Formula (I) or (X) with one or more solvent molecules, whether organic or inorganic. This physical association includes hydrogen bonding. In certain instances, the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

The terms “binds,” “binds to,” “specifically binds” or “specifically binds to” in the context of antibody binding refer to antibody binding to an antigen (e.g., epitope) as such binding is understood by one skilled in the art. For example, a molecule that specifically binds to an antigen may bind to other polypeptides, generally with lower affinity as determined by, e.g., immunoassays, Biacore™, KinExA 3000 instrument (Sapidyne Instruments, Boise, ID), or other assays known in the art. In a specific embodiment, molecules that specifically bind to an antigen bind to the antigen with an affinity (K_{d}) that is at least 2 logs, 2.5 logs, 3 logs, 4 logs lower (higher affinity) than the K_{d} when the molecules bind to another antigen. In another specific embodiment, molecules that specifically bind to an antigen do not cross react with other proteins. In another specific embodiment, where EGFR is the protein of interest, molecules that specifically bind to an antigen do not cross react with other non-EGFR proteins.

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. In certain embodiments, where an integer is required, the term “about” means within plus or minus 10% of a given value or range, rounded either up or down to the nearest integer.

In the description herein, if there is any discrepancy between a chemical name and chemical structure, the chemical structure shall prevail.

The terms “protein” and “polypeptide” are used interchangeably. Proteins may include moieties other than amino acids (e.g., may be glycoproteins, etc.) and/or may be otherwise processed or modified. Those of ordinary skill in the art will appreciate that a “protein” can be a complete protein chain as produced by a cell (with or without a signal sequence), or can be a protein portion thereof. Those of ordinary skill will appreciate that a protein can sometimes include more than one protein chain, for example non-covalently or covalently attached, e.g., linked by one or more disulfide bonds or associated by other means. Polypeptides may contain 1-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof.

The terms “antibody” and “immunoglobulin” are terms of art and can be used interchangeably herein, and refer to a molecule with an antigen binding site that specifically binds an antigen.

In a certain embodiments, an isolated antibody (e.g., monoclonal antibody) described herein, or an antigen-binding fragment thereof, which specifically binds to a protein of interest, for example, EGFR, is conjugated to one or more lysosomal targeting moieties, for example, via a linker.

An “antigen” is a moiety or molecule that contains an epitope to which an antibody can specifically bind. As such, an antigen is also is specifically bound by an antibody. In a specific embodiment, the antigen, to which an antibody described herein binds, is a protein of interest, for example, EGFR (e.g., human EGFR), or a fragment thereof, or for example, an extracellular domain of EGFR (e.g., human EGFR).

An “epitope” is a term known in the art and refers to a localized region of an antigen to which an antibody can specifically bind. An epitope can be a linear epitope of contiguous amino acids or can comprise amino acids from two or more non-contiguous regions of the antigen.

The terms “binds,” “binds to,” “specifically binds” or “specifically binds to” in the context of antibody binding refer to antibody binding to an antigen (e.g., epitope) as such binding is understood by one skilled in the art. For example, a molecule that specifically binds to an antigen may bind to other polypeptides, generally with lower affinity as determined by, e.g., immunoassays, Biacore™, KinExA 3000 instrument (Sapidyne Instruments, Boise, ID), or other assays known in the art. In a specific embodiment, molecules that specifically bind to an antigen bind to the antigen with an affinity (K_{d}) that is at least 2 logs, 2.5 logs, 3 logs, 4 logs lower (higher affinity) than the K_{d} when the molecules bind to another antigen. In another specific embodiment, molecules that specifically bind to an antigen do not cross react with other proteins. In another specific embodiment, where EGFR is the protein of interest, molecules that specifically bind to an antigen do not cross react with other non-EGFR proteins.

Antibodies can include, for example, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain/antibody heavy chain pair, an antibody with two light chain/heavy chain pairs (e.g., identical pairs), intrabodies, heteroconjugate antibodies, single domain antibodies, monovalent antibodies, bivalent antibodies (including monospecific or bispecific bivalent antibodies), single chain antibodies, or single-chain Fvs (scFv), camelized antibodies, affybodies, Fab fragments, F(ab′) fragments, F(ab′)₂ fragments, disulfide-linked Fvs (sdFv), anti-idiotypic (anti-Id) antibodies (including, e.g., anti-anti-Id antibodies), and epitope-binding fragments of any of the above.

Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies (e.g., human IgG), or a class (e.g., human IgG1, IgG2, IgG3 or IgG4) or subclass thereof.

In a particular embodiment, an antibody is a 4-chain antibody unit comprising two heavy (H) chain/light (L) chain pairs, wherein the amino acid sequences of the H chains are identical and the amino acid sequences of the L chains are identical. In a specific embodiment, the H and L chains comprise constant regions, for example, human constant regions. In a yet more specific embodiment, the L chain constant region of such antibodies is a kappa or lambda light chain constant region, for example, a human kappa or lambda light chain constant region. In another specific embodiment, the H chain constant region of such antibodies comprise a gamma heavy chain constant region, for example, a human gamma heavy chain constant region. In a particular embodiment, such antibodies comprise IgG constant regions, for example, human IgG constant regions.

The term “constant region” or “constant domain” is a well-known antibody term of art (sometimes referred to as “Fc”), and refers to an antibody portion, e.g., a carboxyl terminal portion of a light and/or heavy chain which is not directly involved in binding of an antibody to antigen but which can exhibit various effector functions, such as interaction with the Fc receptor. The terms refer to a portion of an immunoglobulin molecule having a generally more conserved amino acid sequence relative to an immunoglobulin variable domain.

The term “heavy chain” when used in reference to an antibody can refer to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

The term “light chain” when used in reference to an antibody can refer to any distinct types, e.g., kappa (κ) of lambda (λ) based on the amino acid sequence of the constant domains. Light chain amino acid sequences are well known in the art. In specific embodiments, the light chain is a human light chain.

The term “monoclonal antibody” is a well-known term of art that refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single cell (e.g., hybridoma or host cell producing a recombinant antibody), wherein the antibody specifically binds to an epitope as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody. In particular embodiments, a monoclonal antibody is a monospecific or multispecific antibody (e.g., bispecific antibody).

The terms “variable region” or “variable domain” refer to a portion of an antibody, generally, a portion of a light or heavy chain, typically about the amino-terminal 110 to 120 amino acids in the mature heavy chain and about 90 to 100 amino acids in the mature light chain. Variable regions comprise complementarity determining regions (CDRs) flanked by framework regions (FRs). Generally, the spatial orientation of CDRs and FRs are as follows, in an N-terminal to C-terminal direction: FR1-CDR1-FR2-CDR2-FR3-CDR3-FR4. Without wishing to be bound by any particular mechanism or theory, it is believed that the CDRs of the light and heavy chains are primarily responsible for the interaction of the antibody with antigen and for the specificity of the antibody for an epitope. In a specific embodiment, numbering of amino acid positions of antibodies described herein is according to the EU Index, as in Kabat et al. (1991) Sequences of Proteins of Immunological Interest, Fifth Edition, U.S. Department of Health and Human Services, NIH Publication No. 91-3242. In certain embodiments, the variable region is a human variable region.

In certain aspects, the CDRs of an antibody can be determined according to (i) the Kabat numbering system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196: 901-917; Al-Lazikani et al., 1997, J. Mol. Biol., 273: 927-948; Chothia et al., 1992, J. Mol. Biol., 227: 799-817; Tramontano et al., 1990, J. Mol. Biol. 215(1):175-82; U.S. Pat. No. 7,709,226; and Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001)); or (iii) the ImMunoGeneTics (IMGT) numbering system, for example, as described in Lefranc, 1999, The Immunologist, 7: 132-136 and Lefranc et al., 1999, Nucleic Acids Res., 27: 209-212 (“IMGT CDRs”); or (iv) the AbM numbering system, which will be referred to herein as the “AbM CDRs”, for example as described in MacCallum et al., 1996, J. Mol. Biol., 262: 732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001); or (v) the Contact numbering system, which will be referred to herein as the “Contact CDRs” (the Contact definition is based on analysis of the available complex crystal structures (bioinf.org.uk/abs) (see, e.g., MacCallum et al., 1996, J. Mol. Biol., 262:732-745)).

The terms “full length antibody,” “intact antibody” and “whole antibody” are used herein interchangeably to refer to an antibody in its substantially intact form, and are not antibody fragments as defined below. The terms particularly refer to an antibody with heavy chains that contain the Fc region.

“Antibody fragments” comprise only a portion of an intact antibody, wherein the portion retains at least one, two, three and as many as most or all of the functions normally associated with that portion when present in an intact antibody. In one aspect, an antibody fragment comprises an antigen binding site of the intact antibody and thus retains the ability to bind antigen. In another aspect, an antibody fragment, such as an antibody fragment that comprises the Fc region, retains at least one of the biological functions normally associated with the Fc region when present in an intact antibody. Such functions may include FcRn binding, antibody half-life modulation, conjugate function and complement binding. In another aspect, an antibody fragment is a monovalent antibody that has an in vivo half-life substantially similar to an intact antibody. For example, such an antibody fragment may comprise on antigen binding arm linked to an Fc sequence capable of conferring in vivo stability to the fragment.

Unless specifically stated otherwise, where a compound may assume alternative tautomeric, regioisomeric and/or stereoisomeric forms, all alternative isomers, are intended to be encompassed within the scope of the claimed subject matter. For example, when a compound is described as a particular optical isomer D- or L-, it is intended that both optical isomers be encompassed herein. For example, where a compound is described as having one of two tautomeric forms, it is intended that both tautomers be encompassed herein. Thus, the compounds provided herein may be enantiomerically pure, or be stereoisomeric or diastereomeric mixtures. The compounds provided herein may contain chiral centers. Such chiral centers may be of either the (R) or (S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O, ¹⁸F, ³⁶Cl, ⁸²Br, ¹²³I, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with ³H, ¹⁴C and/or ¹⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

Thus, any of the embodiments described herein are meant to include a salt, a single stereoisomer, a mixture of stereoisomers and/or an isotopic form of the compounds.

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. In certain embodiments, where an integer is required, the term “about” means within plus or minus 10% of a given value or range, rounded either up or down to the nearest integer.

In the description herein, if there is any discrepancy between a chemical name and chemical structure, the chemical structure shall prevail.

### 5.7. Exemplary Embodiments

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

Notwithstanding the appended claims, aspects of the present disclosure are illustrated by the following clauses.

Clause 1. A compound of the following formula (Ia):

Xₙ-L-Y (Ia);

or a salt, a single stereoisomer, a mixture of stereoisomers or an isotopic form thereof,
wherein:
X is a moiety that binds to a cell surface folate receptor;
L is a linker of the following formula (IIa):

-[(L¹)ₐ-(L²)_{b}-(L³)_{c}]ₙ-(L⁴)_{d}-(L⁵)ₑ-(L⁶)_{f}-(L⁷)_{g}- (IIa); and

wherein
each L¹ is independently —NH—C₁₋₆-alkylene;
each L² is independently —C₁₋₆-alkylene-, —NHCO—C₁₋₆-alkylene-, —CONH—C₁₋₆-alkylene-, —(OCH₂)ₚ—, or —(OCH₂CH₂)ₚ—;
each L³ is independently

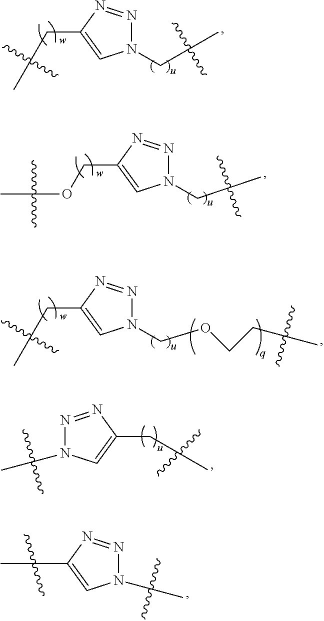

or —(OCH₂CH₂)_{q}—;
each L⁴ is independently —OCH₂CH₂—,

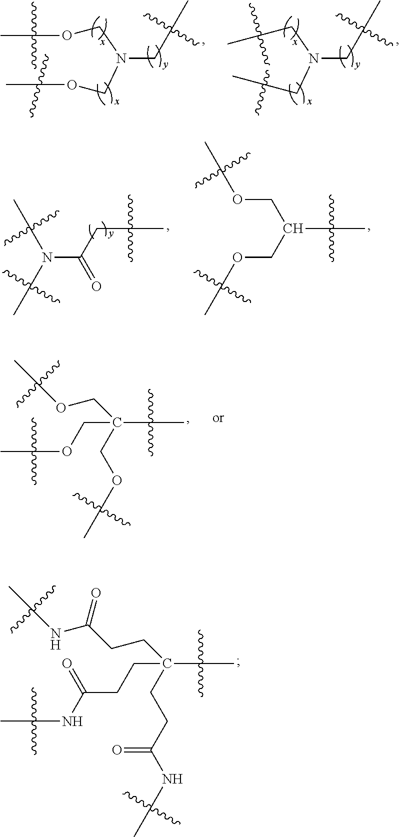

each L⁵ is independently —NHCO—C₁₋₆-alkylene-, —CONH—C₁₋₆-alkylene-, —C₁₋₆-alkylene-,

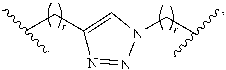

or —(OCH₂CH₂)ᵣ—;
each L⁶ is independently —NHCO—C₁₋₆-alkylene-, —CONH—C₁₋₆-alkylene-, —C₁₋₆-alkylene-, or —(OCH₂CH₂)ₛ—;
each L⁷ is independently —NHCO—C₁₋₆-alkylene-, —CONH—C₁₋₆-alkylene-, —C₁₋₆-alkylene-, —(OCH₂CH₂)ₜ—, or —OCH₂—;
p, q, r, s, and t are each independently an integer of 1 to 20; a is 1 or 2; b, c, d, e, f, and g are each independently 0, 1, or 2; u, v, w, x, y, and z are each independently an integer of 1 to 10;
n is an integer of 1 to 5; wherein when d is 0, n is 1, when d is 1, n is an integer of 1 to 3, and when d is 2, n is an integer of 1 to 5;
Y is a moiety selected from the group consisting of

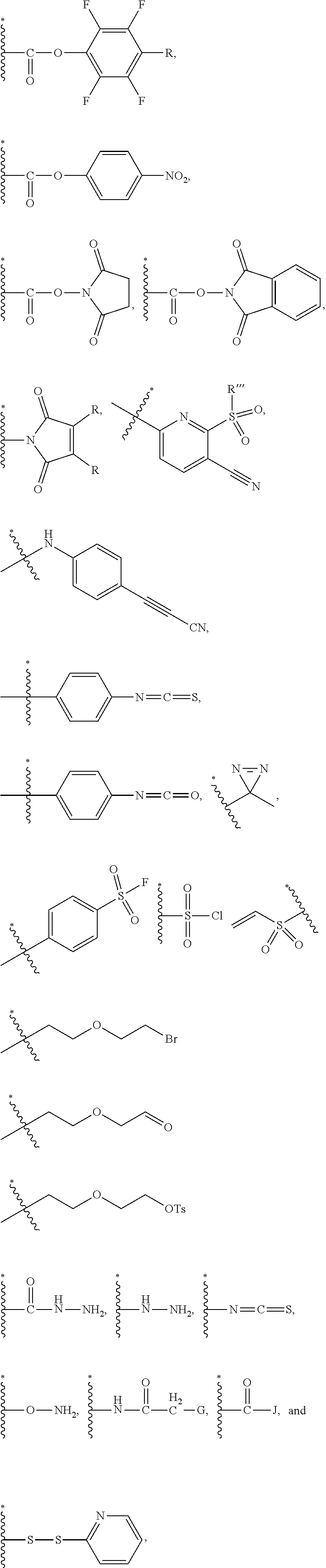

[symbol]

R is hydrogen or fluorine;
each R′ is independently hydrogen or halo;
G is selected from —F, —Cl, —Br, —I, —O-mesyl, and —O-tosyl;
J is selected from —Cl, —Br, —I, —F, —OH, —O—N-succinimide, —O-(4-nitrophenyl), —O-pentafluorophenyl, —O-tetrafluorophenyl, and —O—C(O)—OR^{J′}; and
R^{J′} is —C₁-C₈ alkyl or -aryl.

Clause 2. The compound of clause 1, wherein a is 1.

Clause 3. The compound of clause 1, wherein at least one of b, c, e, f, and g is not 0.

Clause 4. The compound of clause 1, wherein at least one of b or c is not 0 and at least one of e, f, and g is not 0.

Clause 5. The compound of clause 1, wherein a, b, and c are each independently 1 or 2.

Clause 6. The compound of any one of clauses 1-5, wherein the cell surface folate receptor is folate receptor 1 (FRα).

Clause 7. The compound of any one of clauses 1-5, wherein the cell surface folate receptor is folate receptor 2 (FRβ).

Clause 8. The compound of any one of clauses 1-5, wherein the cell surface folate receptor is folate receptor 3 (FRγ).

Clause 9. The compound of any one of clauses 1-8, wherein X is of formula (IIIa), (IIIb), (IIIc), (IIId), (IIIe) or (IIf).

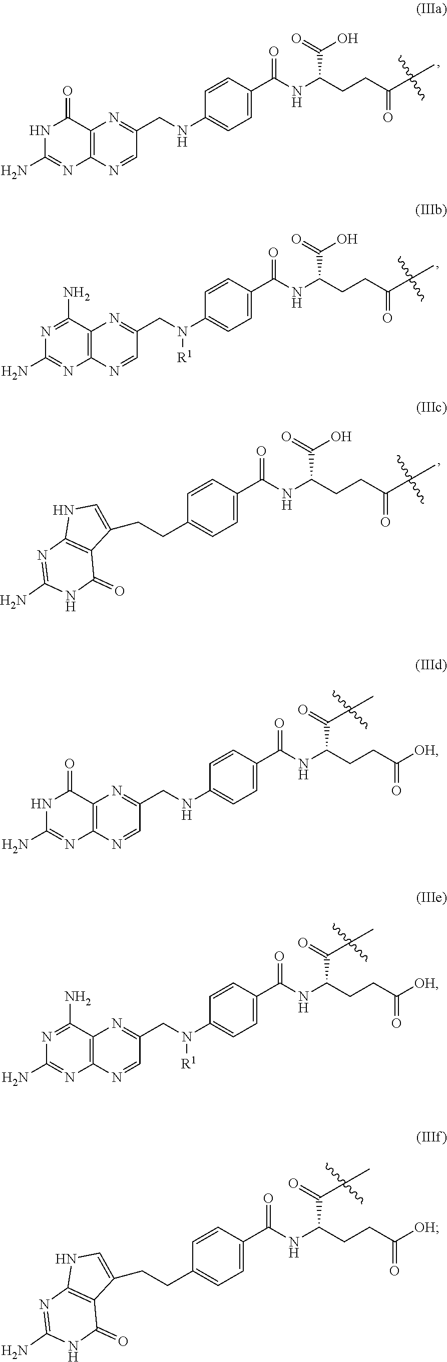

wherein R¹ is —H or —CH₃.

Clause 10. The compound of clause 1, wherein the compound is

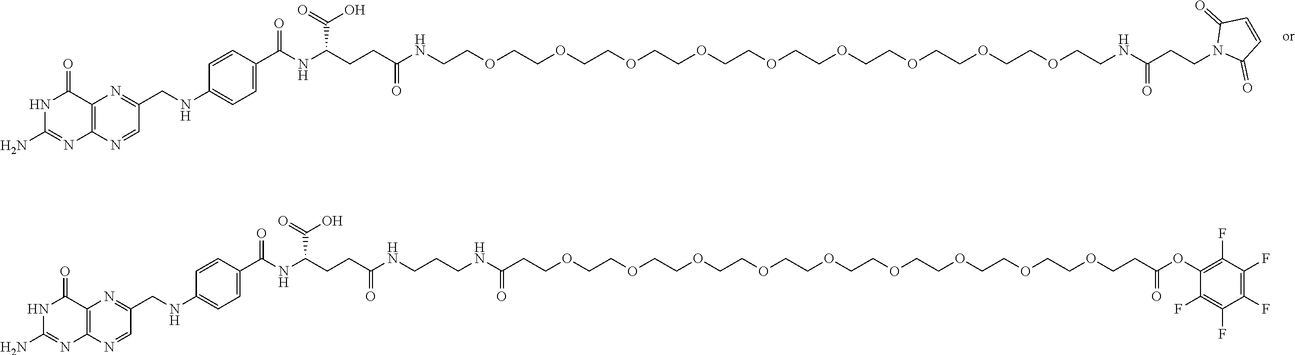 [SMILES: Nc1nc2ncc(CNc3ccc(C(=O)N[C@@H](CCC(=O)NCCOCCOCCOCCOCCOCCOCCOCCOCCOCCNC(=O)CCN4C(=O)C=CC4=O)C(=O)O)cc3)nc2c(=O)[nH]1 ; Nc1nc2ncc(CNc3ccc(C(=O)N[C@@H](CCC(=O)NCCCNC(=O)CCOCCOCCOCCOCCOCCOCCOCCOCCOCCC(=O)Oc4c(F)c(F)c(F)c(F)c4F)C(=O)O)cc3)nc2c(=O)[nH]1]

Clause 11. The compound of clause 1, selected from the group consisting of

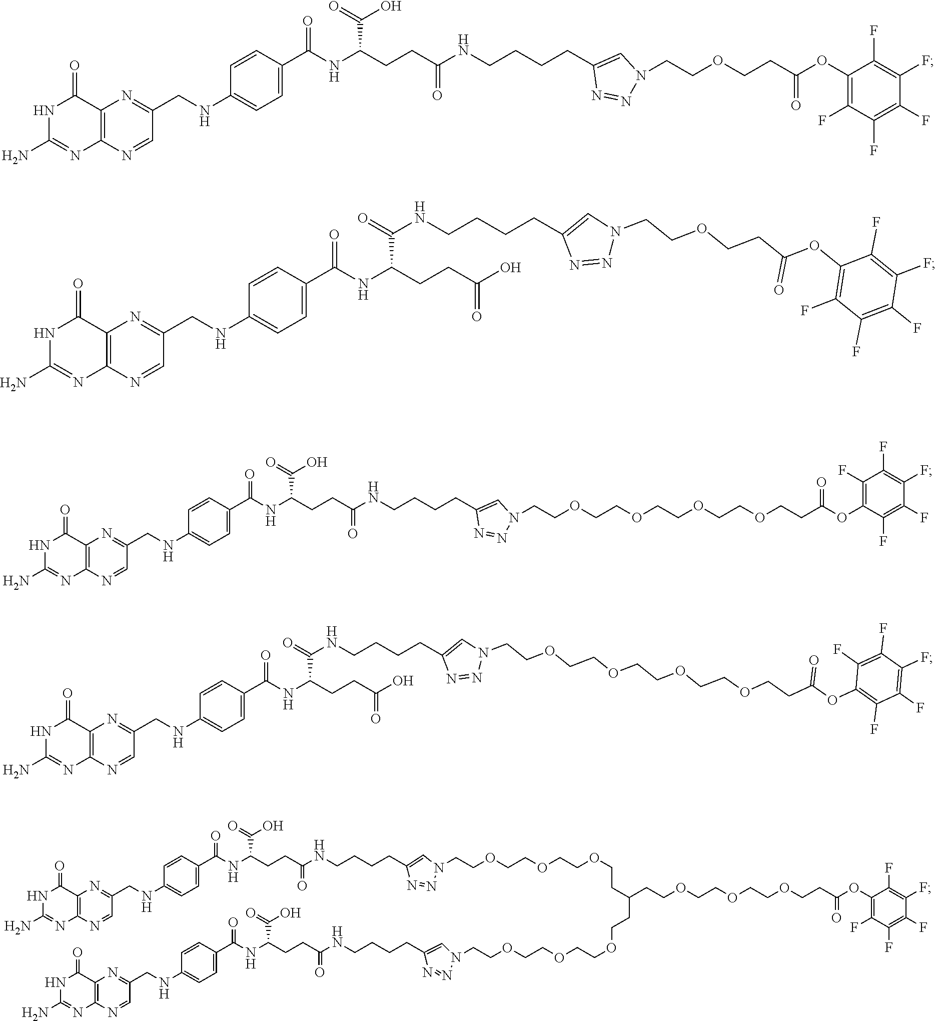

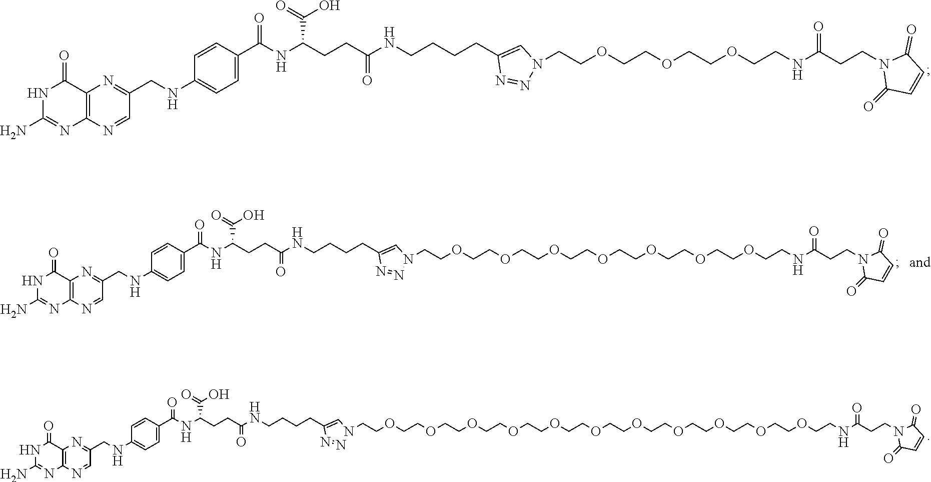 [SMILES: Nc1nc2ncc(CNc3ccc(C(=O)N[C@@H](CCC(=O)NCCCCc4cn(CCOCCOCCOCCNC(=O)CCN5C(=O)C=CC5=O)nn4)C(=O)O)cc3)nc2c(=O)[nH]1 ; Nc1nc2ncc(CNc3ccc(C(=O)N[C@@H](CCC(=O)NCCCCc4cn(CCOCCOCCOCCOCCOCCOCCOCCNC(=O)CCN5C(=O)C=CC5=O)nn4)C(=O)O)cc3)nc2c(=O)[nH]1 ; Nc1nc2ncc(CNc3ccc(C(=O)N[C@@H](CCC(=O)NCCCCc4cn(CCOCCOCCOCCOCCOCCOCCOCCOCCOCCOCCOCCNC(=O)CCN5C(=O)C=CC5=O)nn4)C(=O)O)cc3)nc2c(=O)[nH]1]

Clause 12. The compound of cause 1, selected from the group consisting of

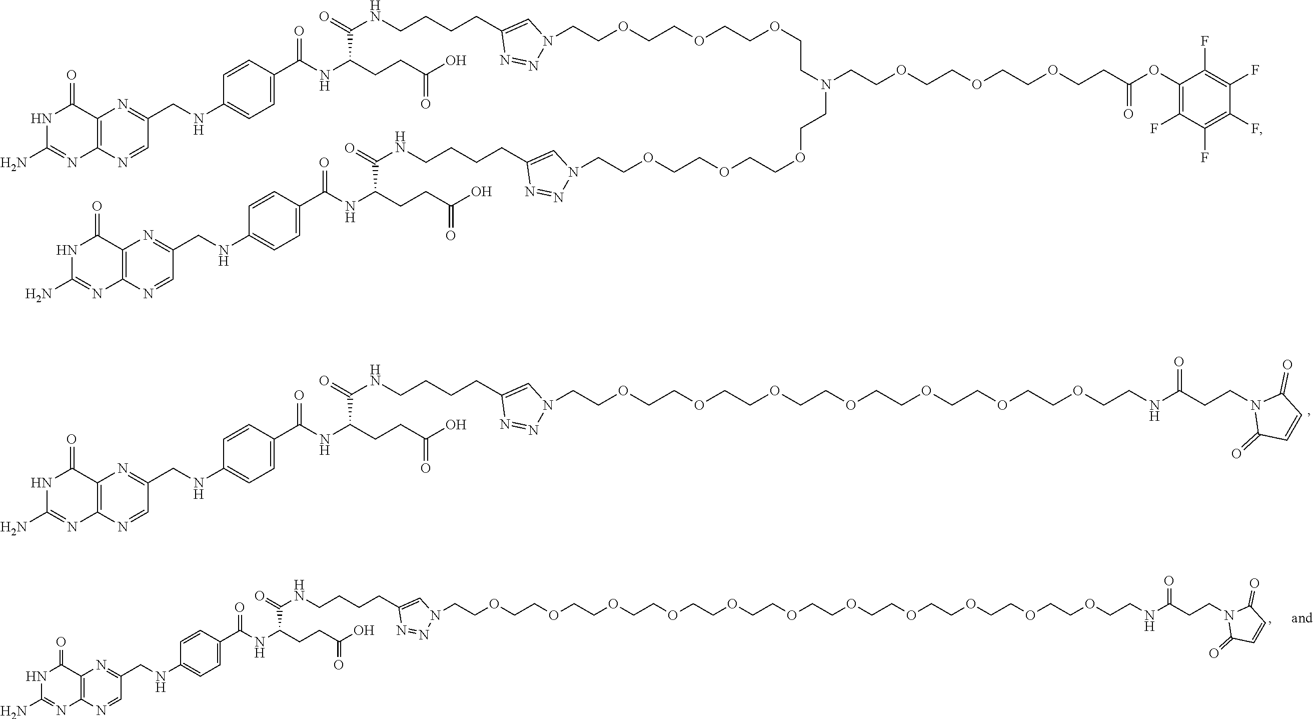 [SMILES: Nc1nc2ncc(CNc3ccc(C(=O)N[C@@H](CCC(=O)O)C(=O)NCCCCc4cn(CCOCCOCCOCCN(CCOCCOCCOCCC(=O)Oc5c(F)c(F)c(F)c(F)c5F)CCOCCOCCOCCn5cc(CCCCNC(=O)[C@H](CCC(=O)O)NC(=O)c6ccc(NCc7cnc8nc(N)[nH]c(=O)c8n7)cc6)nn5)nn4)cc3)nc2c(=O)[nH]1 ; Nc1nc2ncc(CNc3ccc(C(=O)N[C@@H](CCC(=O)O)C(=O)NCCCCc4cn(CCOCCOCCOCCOCCOCCOCCOCCNC(=O)CCN5C(=O)C=CC5=O)nn4)cc3)nc2c(=O)[nH]1 ; Nc1nc2ncc(CNc3ccc(C(=O)N[C@@H](CCC(=O)O)C(=O)NCCCCc4cn(CCOCCOCCOCCOCCOCCOCCOCCOCCOCCOCCOCCNC(=O)CCN5C(=O)C=CC5=O)nn4)cc3)nc2c(=O)[nH]1]

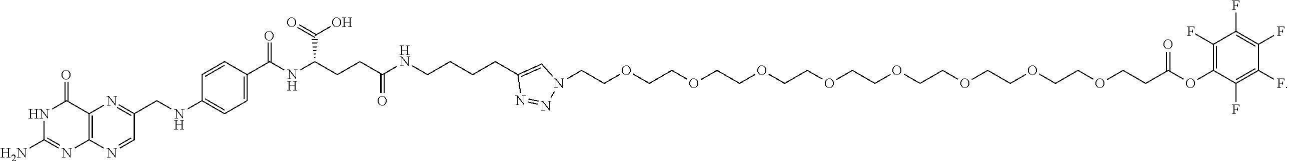 [SMILES: Nc1nc2ncc(CNc3ccc(C(=O)N[C@@H](CCC(=O)NCCCCc4cn(CCOCCOCCOCCOCCOCCOCCOCCOCCC(=O)Oc5c(F)c(F)c(F)c(F)c5F)nn4)C(=O)O)cc3)nc2c(=O)[nH]1]

Clause 13. A conjugate of the following formula (IVa):

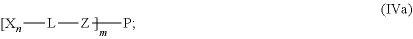

or a pharmaceutically acceptable salt thereof,
wherein:
X is a moiety that binds to a cell surface folate receptor;
L is a linker of the following formula (IIa):

-[(L¹)ₐ-(L²)_{b}-(L³)_{c}]ₙ-(L⁴)_{d}-(L⁵)ₑ-(L⁶)_{f}-(L⁷)_{g}- (IIa); and
wherein
each L¹ is independently —NH—C₁₋₆-alkylene;
each L² is independently —C₁₋₆-alkylene-, —NHCO—C₁₋₆-alkylene-, —CONH—C₁₋₆-alkylene-, —(OCH₂)ₚ—, or —(OCH₂CH₂)ₚ—;
each L³ is independently

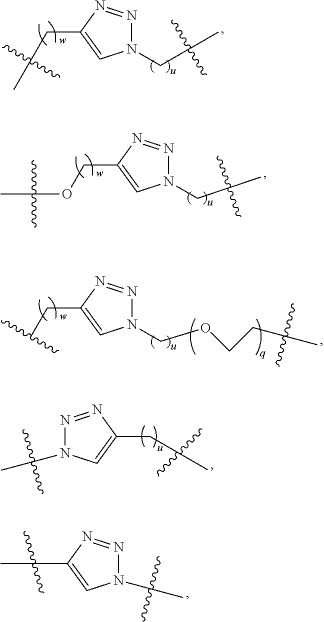

or —(OCH₂CH₂)_{q}—;
each L⁴ is independently —OCH₂CH₂—,

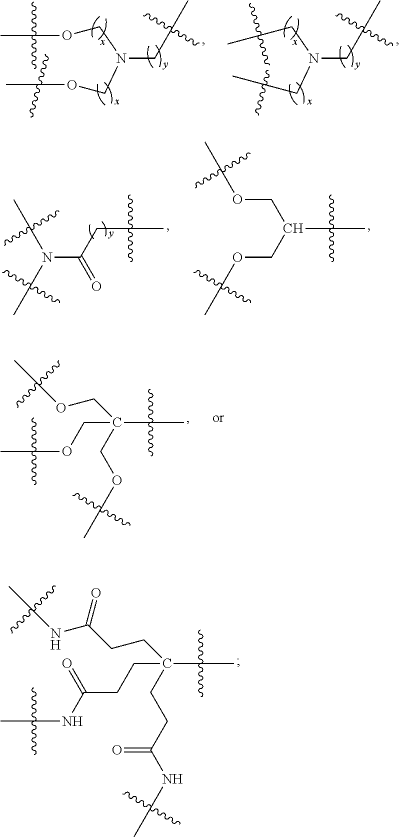

each L⁵ is independently —NHCO—C₁₋₆-alkylene-, —CONH—C₁₋₆-alkylene-, —C₁₋₆-alkylene-,

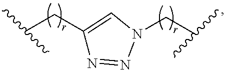

or —(OCH₂CH₂)ᵣ—;
each L⁶ is independently —NHCO—C₁₋₆-alkylene-, —CONH—C₁₋₆-alkylene-, —C₁₋₆-alkylene-, or —(OCH₂CH₂)ₛ—;
each L⁷ is independently —NHCO—C₁₋₆-alkylene-, —CONH—C₁₋₆-alkylene-, —C₁₋₆-alkylene-, —(OCH₂CH₂)ₜ—, or —OCH₂—;
p, q, r, s, and t are each independently an integer of 1 to 20; a is 1 or 2; b, c, d, e, f, and g are each independently 0, 1, or 2; u, v, w, x, y, and z are each independently an integer of 1 to 10;
n is an integer of 1 to 5; wherein when d is 0, n is 1, when d is 1, n is an integer of 1 to 3, and when d is 2, n is an integer of 1 to 5;
m is an integer from 1 to 80;
Z is selected from the group consisting of

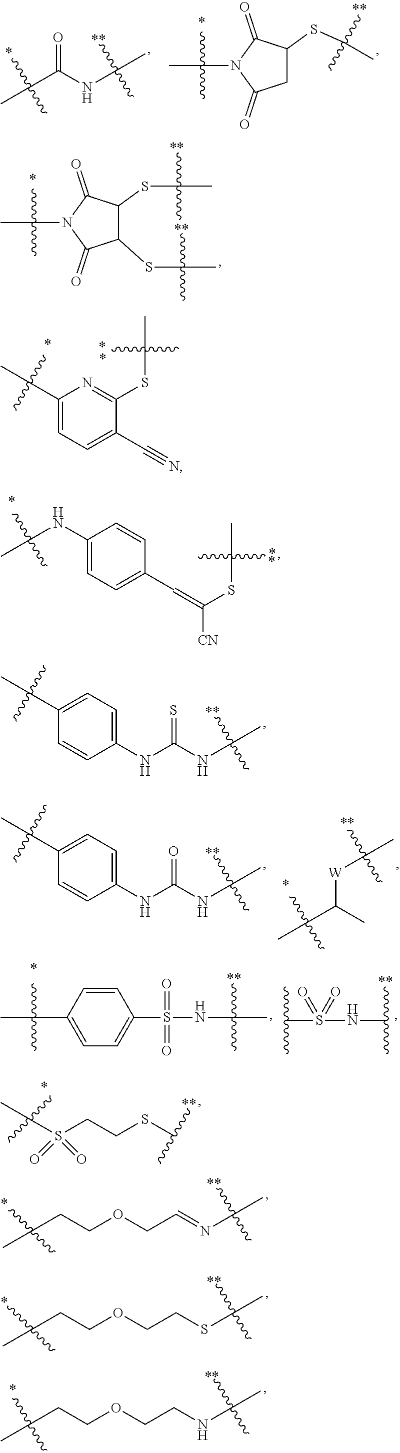

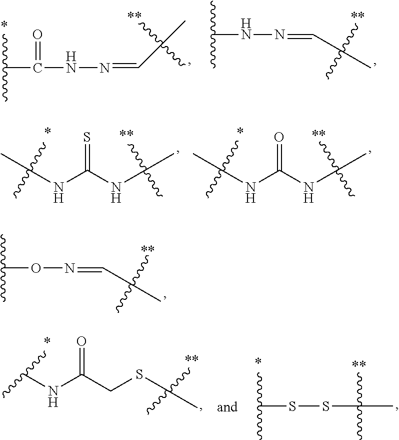

wherein [symbol] represents the point of attachment to L,
wherein [symbol] represents the point of attachment to P,
W is CH₂, N, O or S; and
P is a polypeptide.

Clause 14. The conjugate of clause 13, wherein P comprises an antibody or an antigen-binding fragment of an antibody.

Clause 15. The conjugate of clause 13, wherein the cell surface folate receptor is folate receptor 1 (FRα).

Clause 16. The conjugate of clause 13, wherein the cell surface folate receptor is folate receptor 2 (FRβ).

Clause 17. The conjugate of clause 13, wherein the cell surface folate receptor is folate receptor 3 (FRγ).

Clause 18. The conjugate of any one of clauses 11-15, wherein X is of formula (IIIa), (IIIb), (IIIc), (IIId), (IIIe), or (IIIf).

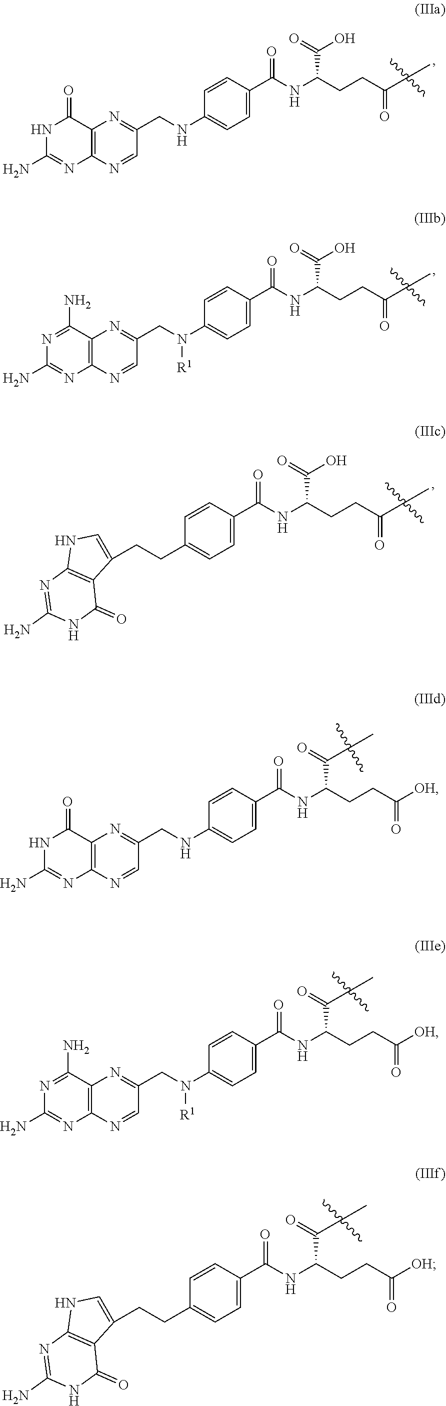

wherein R¹ is —H or —CH₃.

Clause 19. A conjugate of the following formula (Va):

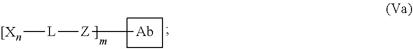

or a pharmaceutically acceptable salt thereof,
wherein:
X is a moiety that binds to a cell surface folate receptor;
L is a linker of the following formula (IIa):

-[(L¹)ₐ-(L²)_{b}-(L³)_{c}]ₙ-(L⁴)_{d}-(L⁵)ₑ-(L⁶)_{f}-(L⁷)_{g}- (IIa); and
wherein
each L¹ is independently —NH—C₁₋₆-alkylene;
each L² is independently —C₁₋₆-alkylene-, —NHCO—C₁₋₆-alkylene-, —CONH—C₁₋₆-alkylene-, —(OCH₂)ₚ—, or —(OCH₂CH₂)ₚ—;
each L³ is independently

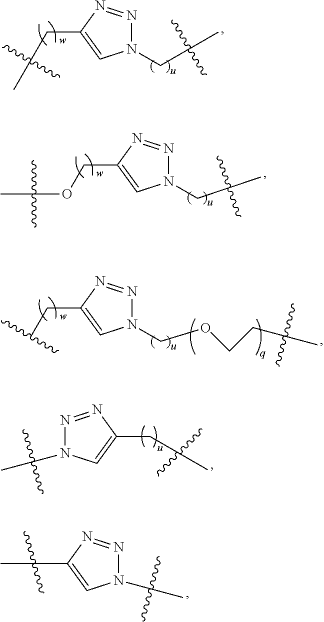

or —(OCH₂CH₂)_{q}—;
each L⁴ is independently —OCH₂CH₂—,

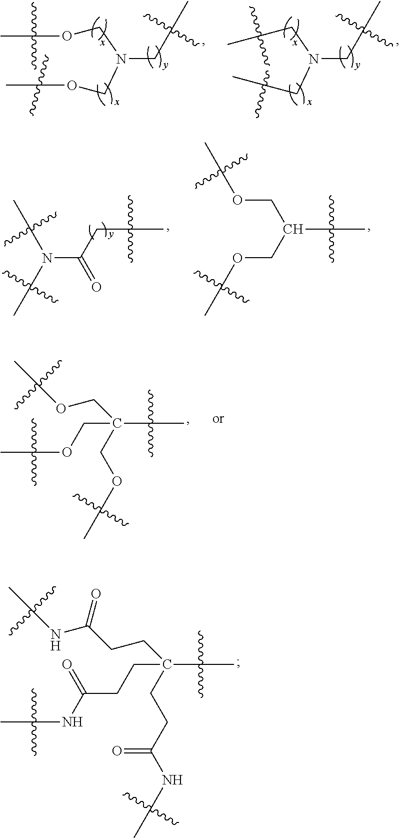

each L⁵ is independently —NHCO—C₁₋₆-alkylene-, —CONH—C₁₋₆-alkylene-, —C₁₋₆-alkylene-,

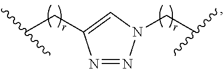

or —(OCH₂CH₂)ᵣ—;
each L⁶ is independently —NHCO—C₁₋₆-alkylene-, —CONH—C₁₋₆-alkylene-, —C₁₋₆-alkylene-, or —(OCH₂CH₂)ₛ—;
each L⁷ is independently —NHCO—C₁₋₆-alkylene-, —CONH—C₁₋₆-alkylene-, —C₁₋₆-alkylene-, —(OCH₂CH₂)ₜ—, or —OCH₂—;
p, q, r, s, and t are each independently an integer of 1 to 20; a is 1 or 2; b, c, d, e, f, and g are each independently 0, 1, or 2; u, v, w, x, y, and z are each independently an integer of 1 to 10;
n is an integer of 1 to 5; wherein when d is 0, n is 1, when d is 1, n is an integer of 1 to 3, and when d is 2, n is an integer of 1 to 5;
m is an integer from 1 to 80;
Z is selected from the group consisting of

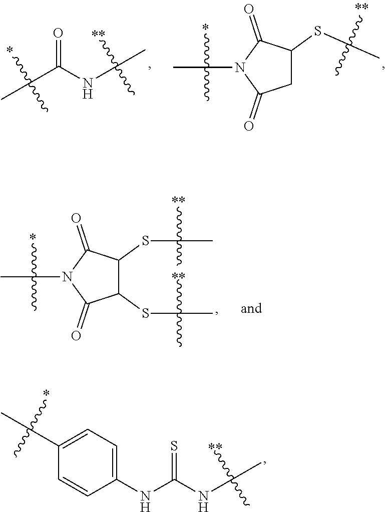

wherein [symbol] represents the point of attachment to L, wherein [symbol] represents the point of attachment to

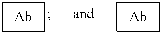

is an antibody.

Clause 20. The conjugate of clause 19, wherein the folate receptor is folate receptor 1 (FRα).

Clause 21. The conjugate of clause 19, wherein the folate receptor is folate receptor 2 (FRβ).

Clause 22. The conjugate of clause 19, wherein the folate receptor is folate receptor 3 (FRγ).

Clause 23. The conjugate of any one of clauses 19-22, wherein X is of formula (IIIa), (IIIb), (IIIc), (IIId), (IIIe) or (IIIf).

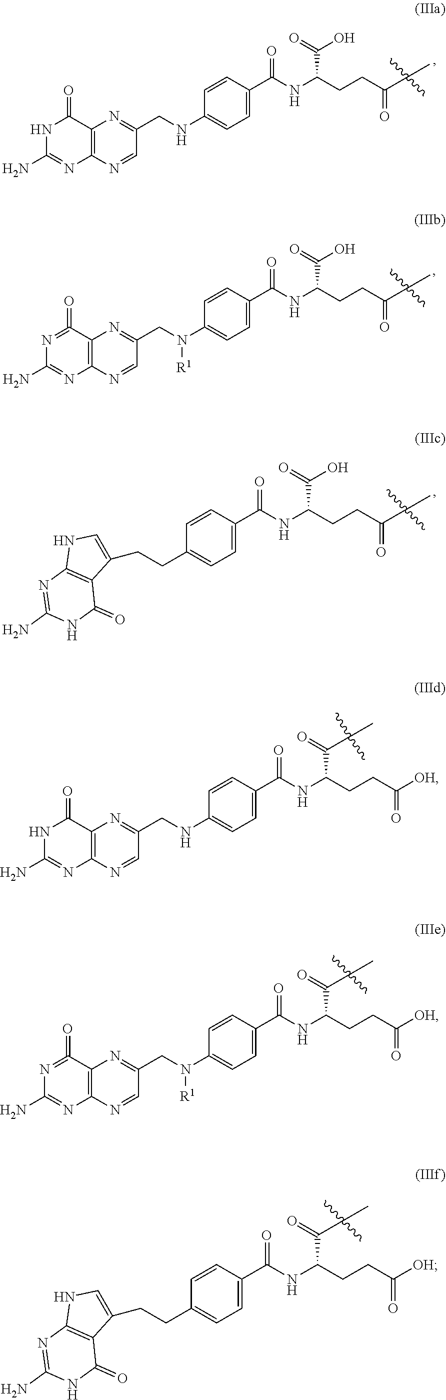

wherein R¹ is —H or —CH₃.

Clause 24. A pharmaceutical composition comprising the conjugate or pharmaceutically acceptable salt of any one of clauses 13-23, and a pharmaceutically acceptable carrier.

Clause 25. The pharmaceutical composition of clause 24, wherein m is an integer of 4 to 8.

Clause 26. The pharmaceutical composition comprising the conjugate or pharmaceutically acceptable salt of clause 25, wherein m is 4.

Clause 27. The conjugate of any one of clauses 19-26, wherein the antibody is an IgG antibody.

Clause 28. The conjugate of any one of clauses 19-26, wherein the antibody is a humanized antibody.

Clause 29. The conjugate of any one of clauses 19-26, wherein the antibody specifically binds to a secreted or soluble protein.

Clause 30. The conjugate of any one of clauses 19-26, wherein the antibody specifically binds to a cell surface receptor.

Clause 31. The conjugate of any one of clauses 19-26, wherein the antibody specifically binds to programmed death ligand-1 (PD-L1) protein.

Clause 32. The conjugate of any one of clauses 19-26, wherein the antibody specifically binds to Vascular Endothelial Growth Factor (VEGF) protein.

Clause 33. The conjugate of any one of clauses 19-26, wherein the antibody specifically binds to a Fibroblast Growth Factor Receptor 2 (FGFR2) protein or a Fibroblast Growth Factor Receptor 3 (FGFR3) protein.

Clause 34. The conjugate of any one of clauses 19-26, wherein the antibody is cetuximab.

Clause 35. The conjugate of any one of clauses 19-26, wherein the antibody is matuzumab.

Clause 36. The conjugate of any one of clauses 19-26, wherein the antibody is atezolizumab.

Clause 101. A cell surface folate receptor binding compound of formula (I):

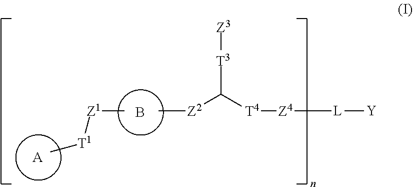

or a salt thereof,
wherein:
T¹ is an optionally substituted (C₁-C₃)alkylene;
Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;
B is a ring system selected from optionally substituted aryl, optionally substituted heteroaryl, optionally substituted heterocycle, optionally substituted cycloalkyl, and optionally substituted bridged bicycle;
Z² is absent, or a linking moiety selected from optionally substituted amide, optionally substituted sulfonamide, optionally substituted urea, optionally substituted thiourea, —NR²¹—, —O—, —S—, and optionally substituted (C₁-C₆)alkylene;
Z³ is carboxyl or carboxyl bioisostere, or a prodrug thereof;
T³ is absent, or is selected from optionally substituted (C₁-C₆)alkylene;
T⁴ is optionally substituted (C₁-C₆)alkylene (e.g., —CH₂CH₂—), or is absent;
Z⁴ is a linking moiety (e.g., a linking moiety selected from ester, amide, sulfonamide, urea, thiourea, amine, ether, optionally substituted aryl, optionally substituted heterocycle, and optionally substituted heteroaryl);
each R²¹ is independently selected from H, and optionally substituted (C₁-C₆)alkyl;
n is 1 to 100;
L is a linker;
Y is a moiety of interest; and
A is a ring system of formula (II):

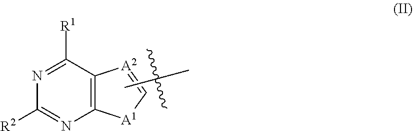

or a tautomer thereof, wherein:
R¹ and R² are independently selected from H, OH, NR²¹, and optionally substituted (C₁-C₆)alkyl (e.g., —CH₃ or —CH₂OH);
A¹ is selected from —N═CR³—, —CR³═N—, —CR³═CR³—, NR²¹, S, O, and C(R⁴)₂;
A² is selected from N, and CR³;
each R³ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each R⁴ is independently selected from H, halogen (e.g., F), and optionally substituted (C₁-C₆)alkyl;
with the proviso that at least one of following applies:
1) T³ is optionally substituted (C₁-C₆)alkylene (e.g., —CH₂CH₂—);
2) L is a non-cleavable linker and Y is an extracellular target-binding moiety;
3) when A is of formula (II-A) or (II-A′), or a tautomer thereof:

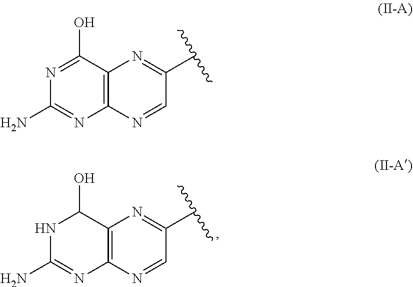 [SMILES: *c1cnc2nc(N)nc(O)c2n1 ; *c1cnc2c(n1)C(O)NC(N)=N2]

then Z¹ is not NR²¹, and/or B is not 1,4-linked phenyl;
4) when A is of formula (II-B), or a tautomer thereof:

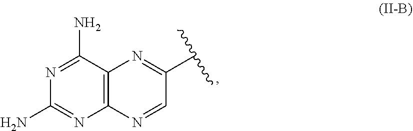 [SMILES: *c1cnc2nc(N)nc(N)c2n1]

then Z¹ is not NR²¹, and/or B is not 1,4-linked phenyl; and/or
5) when A is of formula (II-C) or (II-C′), or a tautomer thereof:

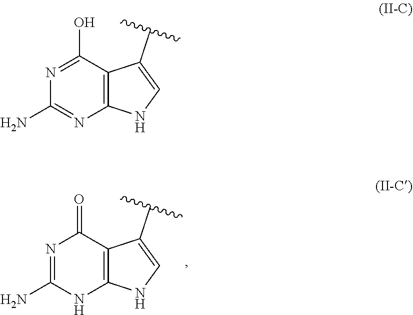 [SMILES: *c1c[nH]c2nc(N)nc(O)c12 ; *c1c[nH]c2[nH]c(N)nc(=O)c12]

then T¹-Z¹ is not —CH₂CH₂—, and/or B is not phenyl.

Clause 102. The compound of clause 101, wherein T³ is optionally substituted (C₁-C₆)alkylene.

Clause 103. The compound of clause 102, wherein T³ is (C₁-C₃)alkylene.

Clause 104. The compound of clause 103, wherein T³ is —CH₂CH₂—.

Clause 105. The compound of any one of clauses 101 to 104, wherein T⁴ is absent.

Clause 106. The compound of clause 105, wherein the compound is of formula (IIIA):

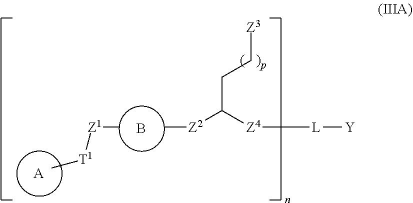

wherein p is 0 or 1.

Clause 107. The compound of clause 101, wherein T³ is absent.

Clause 108. The compound of clause 107, wherein T⁴ is optionally substituted (C₁-C₆)alkylene.

Clause 109. The compound of clause 108, wherein T⁴ is (C₁-C₃)alkylene.

Clause 110. The compound of clause 109, wherein T⁴ is —CH₂CH₂—.

Clause 111. The compound of any one of clauses 107 to 110, wherein the compound is of formula (IIIB):

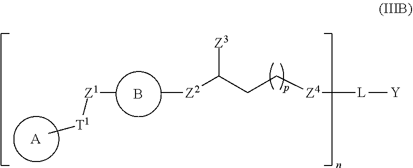

wherein p is 0 or 1.

Clause 112. The compound of any one of clauses 101 to 111, wherein Z³ is selected from —COOH, —COOR²², —CH₂OH, —CH₂OR²², —CN, and tetrazole, wherein R²² is optionally substituted (C₁-C₆)alkyl.

Clause 113. The compound of clause 112, wherein Z³ is selected from:

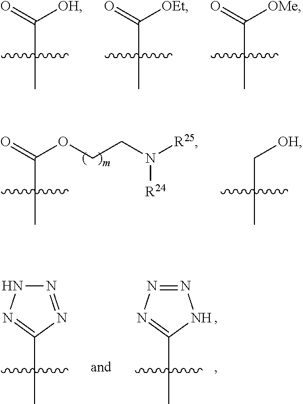

wherein:
R²⁴ and R²⁵ are independently selected from H and optionally substituted (C₁-C₆)alkyl, or R²⁴ and R²⁵ are cyclically linked to provide an optionally substituted 5 or 6-membered heterocycle; and
m is 1 to 5.

Clause 114. The compound of clause 113, wherein Z³ is COOH.

Clause 115. The compound of clause 113, wherein Z³ is

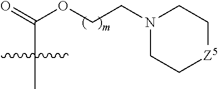

wherein
Z⁵ is O, NH or NR²¹; and
R²¹ is (C₁-C₆)alkyl.

Clause 116. The compound of clause 115, wherein Z⁵ is O, NH or NMe, and m is 1.

Clause 117. The compound of clause 110, wherein Z² is —CONR²¹—, wherein R²¹ is selected from H, and optionally substituted (C₁-C₆)alkyl.

Clause 118. The compound of any one of clauses 112 to 117, wherein Z² is —CONR²¹—, —NR²¹CO—, —SO₂NR²¹—, —NR²¹C(═O)NR²¹—, or —NR²¹C(═S)NR²¹, wherein each R²¹ is independently selected from H, and optionally substituted (C₁-C₆)alkyl.

Clause 119. The compound of any one of clauses 112 to 118, wherein Z⁴ is a linking moiety selected from —CONR²¹—, —NR²¹—, —O—, —S—, optionally substituted aryl (e.g., 1,4-phenyl) and optionally substituted heteroaryl (e.g., oxadiazole or triazole), wherein R²¹ is selected from H, and optionally substituted (C₁-C₆)alkyl.

Clause 120. The compound of clause 119, wherein Z⁴ is a linking group selected from:

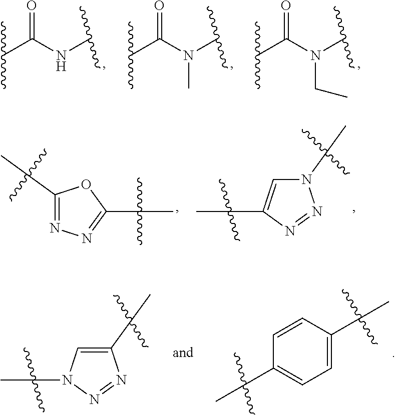

Clause 121. The compound of any one of clauses 112 to 120, wherein —Z²CH(-T³-Z³)T⁴Z⁴— of formula (I) is selected from the following structures:

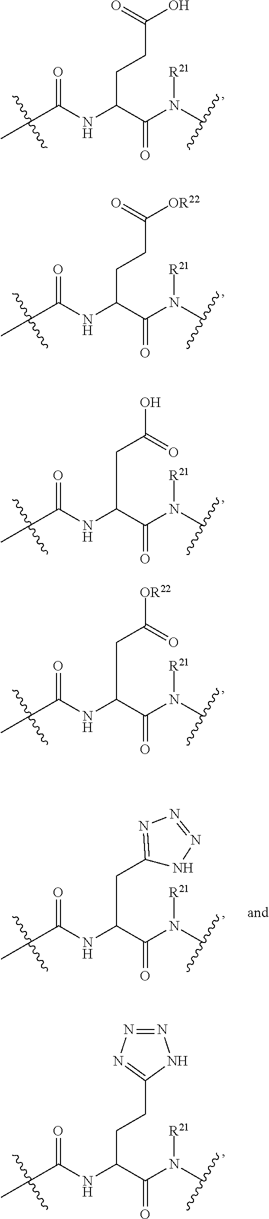

or a tautomer thereof, or a salt thereof.

Clause 122. The compound of any one of clauses 112 to 120, wherein —Z²CH(-T³-Z³)T⁴Z⁴— of formula (I) is selected from the following structures:

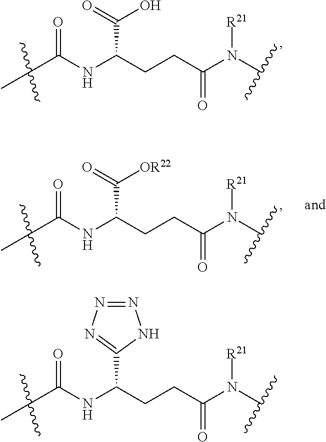

or a tautomer thereof, or a salt thereof.

Clause 123. The compound of any one of clauses 101 to 122, wherein A¹ of ring system A is independently —N═CR³—, —CR³═N—, or —CR³═CR³—.

Clause 124. The compound of clause 123, wherein A is of formula (IIA):

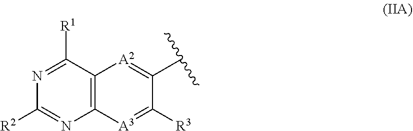

or a tautomer thereof, or a salt thereof, wherein:
A² is selected from N, and CR³;
A³ is independently selected from N, and CR²¹.

Clause 125. The compound of clause 124, wherein A² and A³ are each N.

Clause 126. The compound of clause 124, wherein A² and A³ are each independently CR³.

Clause 127. The compound of any one of clauses 123 to 126, wherein each R³ is H.

Clause 128. The compound of any one of clauses 123 to 127, wherein R² is —NH₂.

Clause 129. The compound of any one of clauses 123 to 126, wherein R² is optionally substituted (C₁-C₆)alkyl.

Clause 130. The compound of any one of clauses 123 to 129, wherein R² is —CH₃ or —CH₂OH.

Clause 131. The compound of any one of clauses 123 to 130, wherein R¹ is OH.

Clause 132. The compound of any one of clauses 123 to 130, wherein A is selected from:

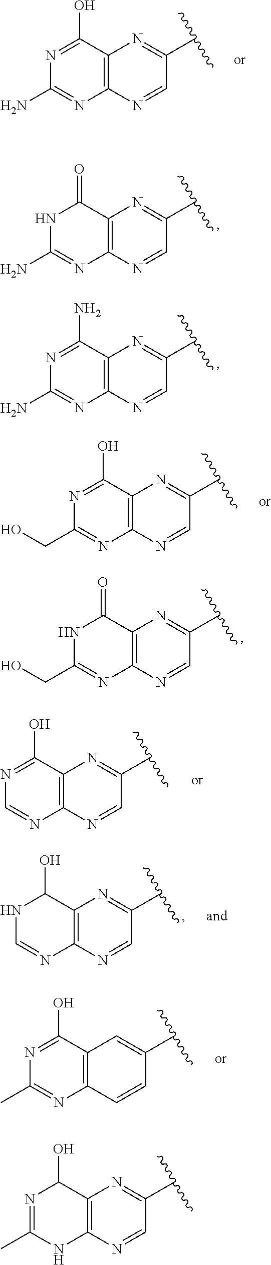

or a tautomer thereof.

Clause 133. The compound of any one of clauses 101 to 122, wherein A¹ of ring system A is NR²¹, S, O, or C(R²¹)₂.

Clause 134. The compound of clause 133, wherein A is of formula (IIB) or (IIC):

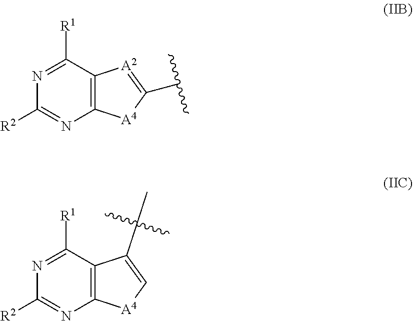

or a tautomer thereof, or a salt thereof, wherein A⁴ is selected from NR²¹, S, and O.

Clause 135. The compound of clause 134, wherein A⁴ is NR²¹.

Clause 136. The compound of clause 134 or 135, wherein A² is CR³.

Clause 137. The compound of any one of clauses 133 to 136, wherein R² is —NH₂.

Clause 138. The compound of any one of clauses 133 to 136, wherein R² is optionally substituted (C₁-C₆)alkyl (e.g., —CH₃ or —CH₂OH).

Clause 139. The compound of any one of clauses 133 to 138, wherein R¹ is OH.

Clause 140. The compound of any one of clauses 133 to 139, wherein A is:

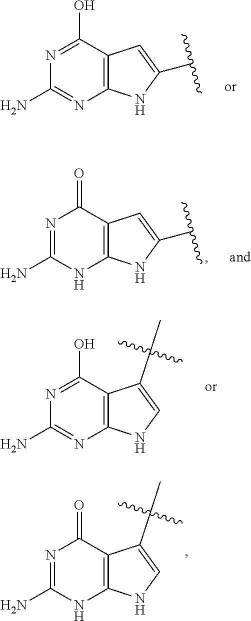 [SMILES: *c1cc2c(O)nc(N)nc2[nH]1 ; *c1cc2c(=O)nc(N)[nH]c2[nH]1 ; *c1c[nH]c2nc(N)nc(O)c12 ; *c1c[nH]c2[nH]c(N)nc(=O)c12]

or a tautomer thereof.

Clause 141. The compound of any one of clauses 123 to 140, wherein T¹ is CH₂.

Clause 142. The compound of any one of clauses 123 to 141, wherein Z¹ is NR²¹.

Clause 143. The compound of clause 142, wherein R²¹ is H.

Clause 144. The compound of clause 142, wherein R²¹ is methyl, ethyl, propyl, or propargyl.

Clause 145. The compound of any one of clauses 123 to 141, wherein Z¹ is O or S.

Clause 146. The compound of any one of clauses 123 to 141, wherein T¹-Z¹ is optionally substituted (C₁-C₆)alkylene.

Clause 147. The compound of clause 146, wherein T¹-Z¹ is —CH₂CH₂—.

Clause 148. The compound of clause 146, wherein T¹-Z¹ is —CH₂CH₂CH₂CH₂— or —CH₂CH₂CH₂—.

Clause 149. The compound of any one of clauses 101 to 148, wherein B is selected from optionally substituted phenyl, optionally substituted pyridyl, optionally substituted pyrimidine, optionally substituted thiophene, optionally substituted pyrrole, optionally substituted furan, optionally substituted oxazole, optionally substituted thiazole, optionally substituted cyclohexyl, optionally substituted cyclopentyl, optionally substituted indole, and optionally substituted bicycloalkyl (e.g., bicyclo[1.1.1]pentane).

Clause 150. The compound of clause 149, wherein B is selected from optionally substituted 1,4-phenylene, optionally substituted 1,3-phenylene, optionally substituted 2,5-pyridylene, optionally substituted 2,5-thiophene, optionally substituted 1,4-cyclohexyl, and optionally substituted 1,3-bicyclo[1.1.1]pentane.

Clause 151. The compound of clause 149 or 150, wherein —B—Z²— is selected from:

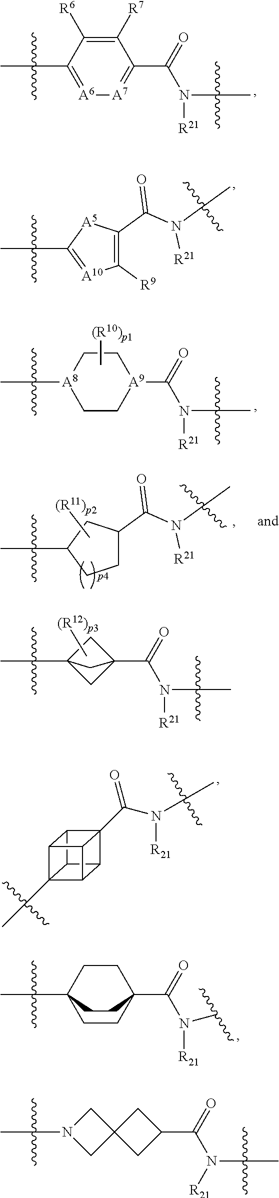

wherein:
A⁵ is selected from NR²¹, S, O, C(R)₂;
A⁶-A⁹ are independently selected from N, and CR⁵; R²¹ is selected from H, and optionally substituted (C₁-C₆)alkyl;
A¹⁰ is selected from N, and CR;
each R⁵ to R¹² is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
p1 is 0 to 10;
p2 is 0 to 14;
p3 is 0 to 4; and
p4 0 to 4.

Clause 152. The compound of clause 151, wherein B—Z² is:

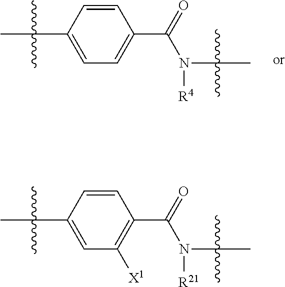

wherein X¹ is halogen.

Clause 153. The compound of clause 146, wherein A-T¹-Z¹—B— is selected from one of the following:

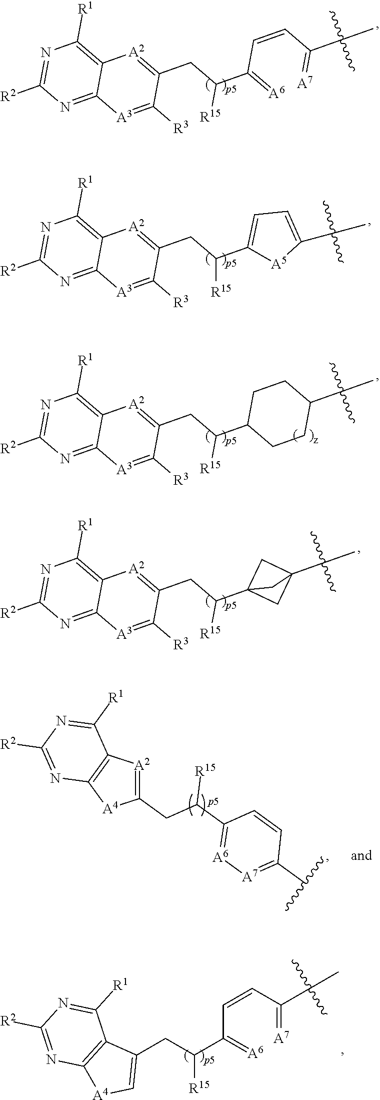

wherein:
A⁵ is selected from NR²¹, S, O, C(R)₂;
A⁶ and A⁷ are independently selected from N, and, CR⁵;
z is 0 to 3
each R⁵ and R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
each p5 is independently 1 to 3.

Clause 154. The compound of clause 101, wherein the compound comprises a cell surface folate receptor ligand selected from:

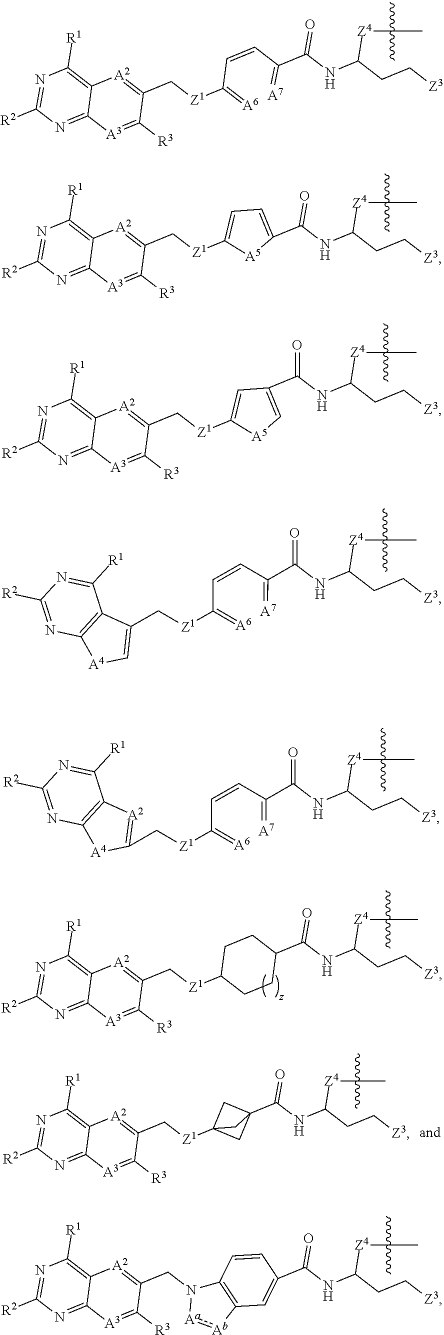

wherein:
A⁵ is selected from NR²¹, S, O, C(R)₂;
A⁶ and A⁷ are independently selected from N, and, CR⁵;
z is 0 to 3;
[symbol] is a single bond or a double bond;
wherein when [symbol] is a single bond A^{a} is selected from C(R)₂, and C═O, and A^{b} is selected from C(R⁵)₂, and NR²¹; and
when [symbol] is a double bond A^{a} is CR⁵, and A^{b} is selected from CR⁵ and N; and
wherein each R⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵.

Clause 155. The compound of clause 101, wherein the compound comprises a cell surface folate receptor ligand selected from:

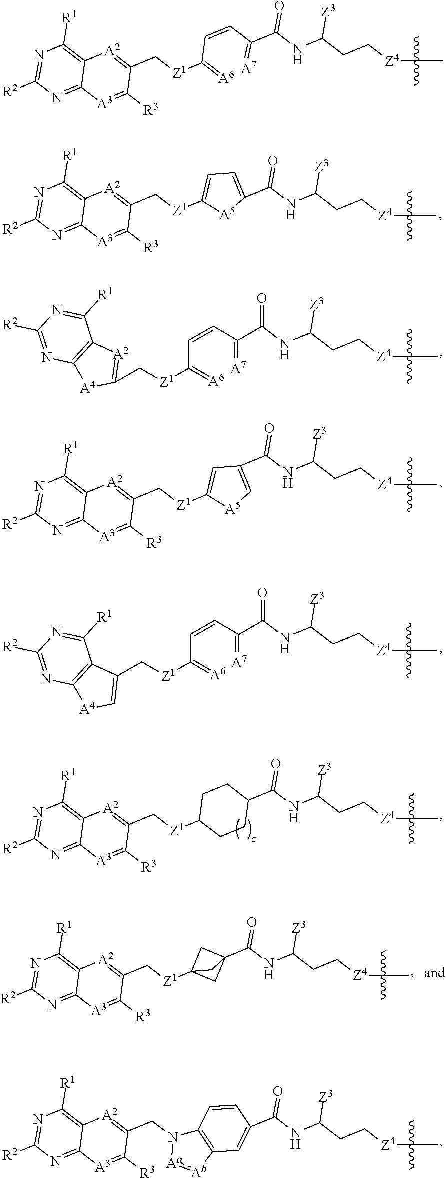

wherein:
A⁵ is selected from NR²¹, S, O, C(R⁵)₂;
A⁶ and A⁷ are each independently selected from N, and, CR⁵;
z is 0 to 3;
[symbol] is a single bond or a double bond;
wherein when [symbol] is a single bond A^{a} is selected from C(R⁵)₂, and C═O, and A^{b} is selected from C(R⁵)₂, and NR²¹; and
when [symbol] is a double bond A^{a} is CR⁵, and A^{b} is selected from CR⁵ and N; and
wherein each R⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵. The compound of any one of clauses 101 to 155, wherein n is 1.

Clause 156. The compound of any one of clauses 101 to 155, wherein n is at least 2.

Clause 157. The compound of clause 156, wherein n is 2 to 20 (e.g., n is 2 to 6, such as 2 or 3).

Clause 158. The compound of any one of clauses 101 to 157, wherein L comprises a backbone of at least 10 consecutive atoms (e.g., by a backbone of at least 12, at least 14, or at least 16 consecutive atoms, e.g., and wherein the backbone is up to 100 consecutive atoms).

Clause 159. The compound of any one of clauses 156 to 158, wherein L comprises one or more linking moieties independently selected from —C₁₋₆-alkylene-, —NHCO—C₁₋₆-alkylene-, —CONH—C₁₋₆-alkylene-, —NHC₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, pyrrolidine-2,5-dione, —NH—, and —NMe-, wherein p is 1 to 10.

Clause 160. The compound of clause 158, wherein L comprises repeating ethylene glycol moieties (e.g., —CH₂CH₂O— or —OCH₂CH₂—).

Clause 161. The compound of clause 158 or 159, wherein L comprises 1 to 20 ethylene glycol moieties (e.g., 2 to 10, or 4 to 6 ethylene glycol moieties).

Clause 162. The compound of any one of clauses 101 to 161, wherein L is of formula (IV):

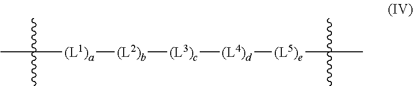

wherein
each L¹ to L⁵ is independently a linking moiety which together provide a linear or branched linker between Z⁴ and Y;
a is 1 or 2; and
b, c, d, and e are each independently 0, 1, or 2.

Clause 163. The compound of clause 162, wherein -(L¹)ₐ- comprises an optionally substituted alkyl or ethylene glycol linking moiety.

Clause 164. The compound of clause 162 or 163, wherein each L¹ is independently selected from: —C₁₋₆-alkylene-, —(CH₂CH₂O)ₜ—, —C₁₋₆-alkylene-NR⁴CO—, —C₁₋₆-alkyleneCONH-, or OCH₂, wherein t is 1 to 20; and R⁴ is independently selected from H, and optionally substituted (C₁-C₆)alkyl.

Clause 165. The compound of any one of clauses 162 to 164, wherein: each L² is independently selected from —NR⁴CO—C₁₋₆-alkylene-, —CONR⁴—C₁₋₆-alkylene,

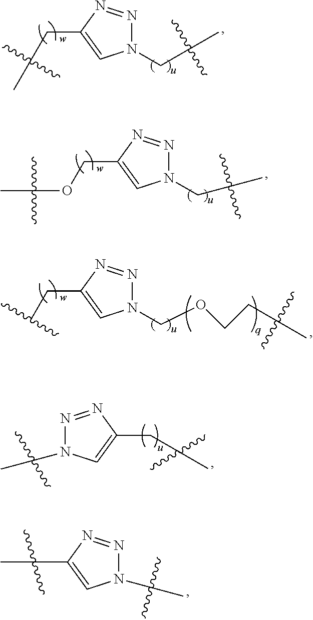

—OCH₂—, and —(OCH₂CH₂)_{q}—, wherein q is 1 to 10, u is 0 to 10, w is 1 to 10, and R⁴ is independently selected from H, and optionally substituted (C₁-C₆)alkyl; and
each L⁴ is absent or independently selected from —C₁₋₆-alkylene-, —(CH₂CH₂O)ₜ—, —C₁₋₆-alkylene-NHCO—, —C₁₋₆-alkyleneCONH-, or OCH₂,
wherein t is 1 to 20.

Clause 166. The compound of any one of clauses 162 to 165, wherein when n is 2 or more, at least one L³ is present and is a branched linking moiety.

Clause 167. The compound of any one of clauses 162 to 166, wherein each L³ is independently selected from:

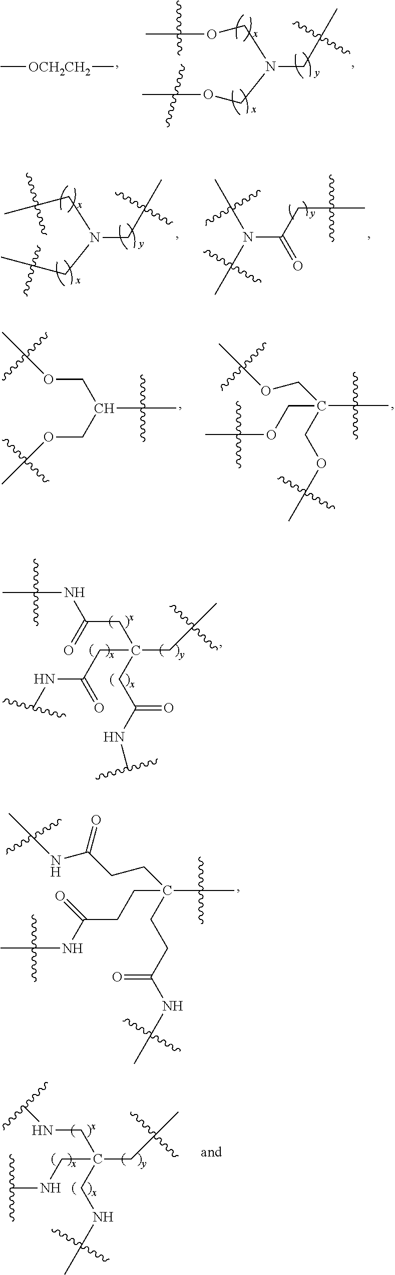

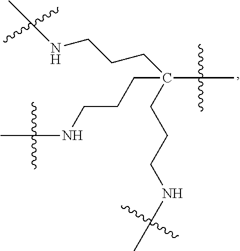

wherein each x and y are each independently 1 to 10.

Clause 168. The compound of any one of clauses 162 to 167, wherein:
each L⁵ is independently —CH₂O—; —(CH₂CH₂O)ᵣ—, —NR⁴CO—, —C₁₋₆-alkylene-,

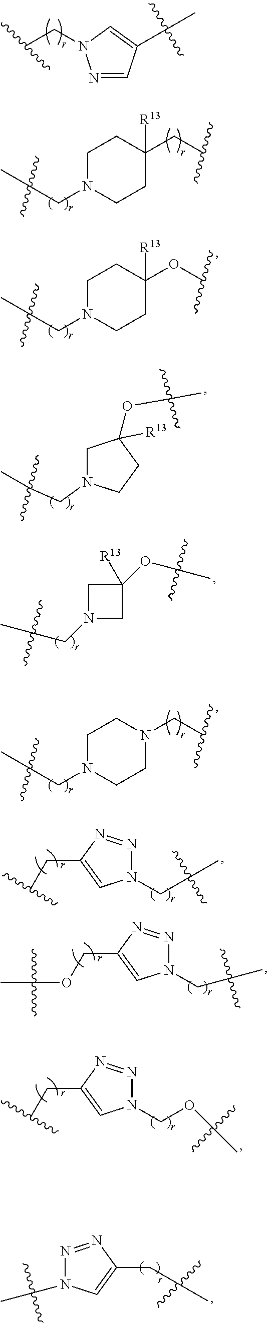

wherein:
R¹³ is selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each r independently 0 to 20.

Clause 169. The compound of any one of clauses 162 to 168, wherein a is 1.

Clause 170. The compound of any one of clauses 162 to 169, wherein at least one of b, c, d, and e is not 0.

Clause 171. The compound of any one of clauses 162 to 170, wherein b, d, and e are each independently 1 or 2.

Clause 172. The compound of any one of clauses 162 to 171, wherein a, b, d, and e are each 1, and c is 0.

Clause 173. The compound of any one of clauses 162 to 172, wherein the linker L is selected from any one of the structures of Table 3.

Clause 174. The compound of any one of clauses 101 to 173, wherein the compound comprises a cell surface folate receptor ligand of one of the structures of Tables 1 or 2.

Clause 175. The compound of any one of clauses 101 to 174, wherein Y is selected from small molecule, dye, fluorophore, monosaccharide, polysaccharide (e.g., disaccharide, or trisaccharide), lipid, enzyme, enzyme substrate and chemoselective ligation group or precursor thereof.

Clause 176. The compound of any one of clauses 101 to 175, wherein Y is a moiety that specifically binds an extracellular target protein.

Clause 177. The compound of clause 176, wherein the target protein is a membrane bound protein.

Clause 178. The compound of clause 177, wherein the target protein is a soluble extracellular protein.

Clause 179. The compound of any one of clauses 1 to 178, wherein Y is a target-binding small molecule.

Clause 180. The compound of clause 179, wherein Y is a small molecule inhibitor or ligand of the target protein.

Clause 181. The compound of any one of clauses 101 to 181, wherein Y is a target-binding biomolecule.

Clause 182. The compound of clause 181, wherein the biomolecule is selected from peptide, protein, glycoprotein, polynucleotide, aptamer, and antibody or antibody fragment.

Clause 183. The compound of clause 182, wherein Y is selected from antibody, antibody fragment (e.g., antigen-binding fragment of an antibody), chimeric fusion protein, an engineered protein domain, and D-protein binder of target protein.

Clause 184. The compound of clause 183, wherein Y is antibody or antibody fragment that specifically binds the target protein and the compound is of formula (VIIIa):

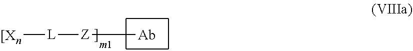

or a pharmaceutically acceptable salt thereof, wherein:
n is 1 to 20;
m1 is an average loading of 1 to 80;
each X is a moiety that binds to a cell surface folate receptor;
each L is a linker;
each Z is a residual moiety resulting from the covalent linkage of a chemoselective ligation group to a compatible group of Ab; and
Ab is the antibody or antibody fragment that specifically binds the target protein.

Clause 185. The compound of clause 184, wherein X is not folic acid, methotrexate, or pemetrexed.

Clause 186. The compound of any one of clauses 184 to 185, wherein X is selected from:

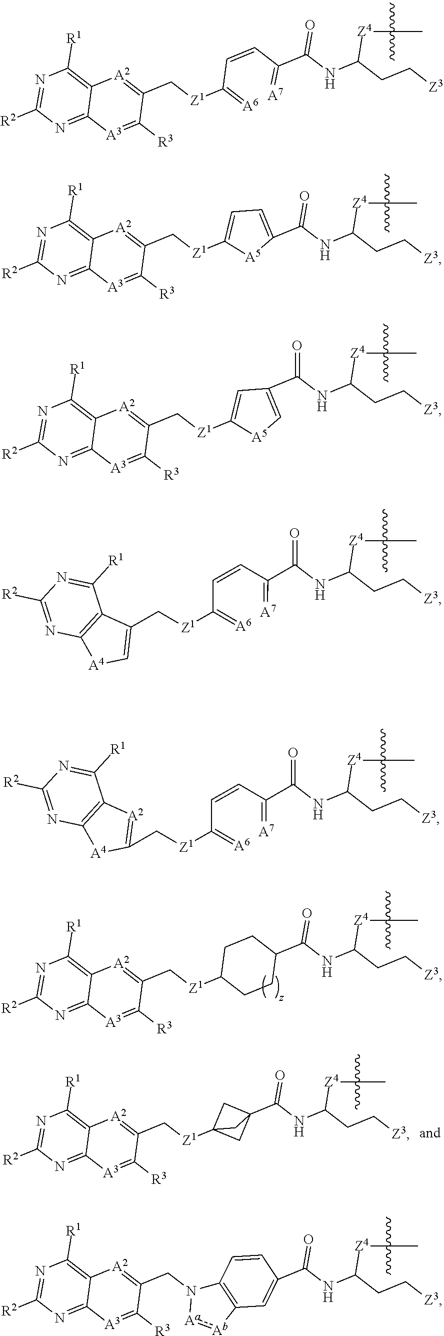

wherein:
A⁵ is selected from NR, S, O, C(R⁵)₂;
A⁶ and A⁷ are independently selected from N, and, CR⁵;
z is 0 to 3;
[symbol] is a single bond or a double bond;
wherein when [symbol] is a single bond A^{a} is selected from C(R⁵)₂, and C═O, and A^{b} is selected from C(R⁵)₂, and NR²¹;
when [symbol] is a double bond A^{a} is CR⁵, and A^{b} is selected from CR⁵ and N; and
wherein each R⁵ independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵.

Clause 187. The compound of any one of clauses 185 to 186, wherein X is selected from:

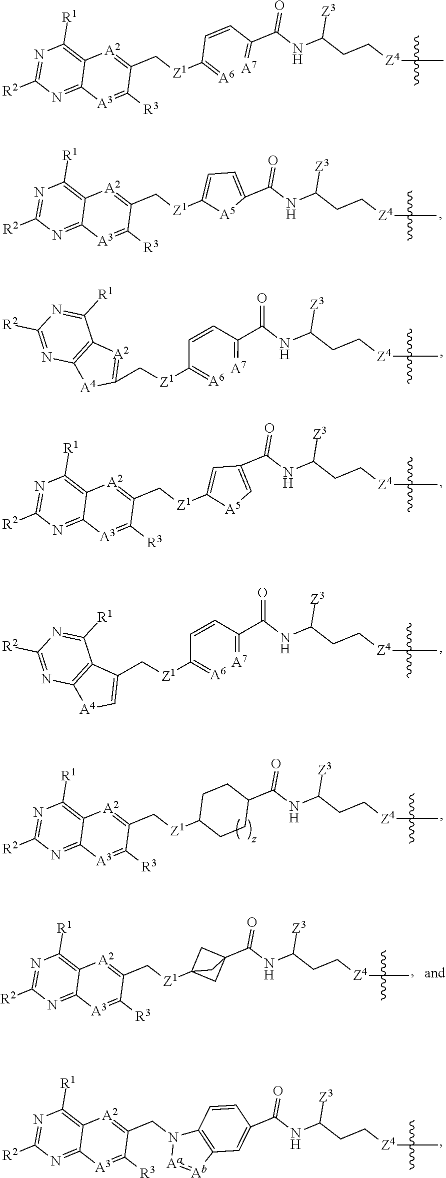

wherein:
A⁵ is selected from NR, S, O, C(R)₂;
A⁶ and A⁷ are each independently selected from N, and, CR⁵;
z is 0 to 3;
[symbol] is a single bond or a double bond;
wherein when [symbol] is a single bond A^{a} is selected from C(R⁵)₂, and C═O, and A^{b} is selected from C(R⁵)₂, and NR²¹;
when [symbol] is a double bond A^{a} is CR⁵, and A^{b} is selected from CR⁵ and N; and
wherein each R⁵ independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵. The compound of any one of clauses 85 to 88, wherein n is 1 to 6.

Clause 188. The compound of clause 187, wherein n is 2 or less.

Clause 189. The compound of clause 189, wherein n is 1.

Clause 190. The compound of any one of clauses 185 to 188, wherein n is at least 2.

Clause 191. The compound of clause 190, wherein n is 2.

Clause 192. The compound of clause 190, wherein n is 3.

Clause 193. The compound of clause 190, wherein n is 4.

Clause 194. The compound of any one of clauses 185 to 193, wherein m1 is 1 to 20.

Clause 195. The compound of clause 194, wherein m1 is 1 to 12.

Clause 196. The compound of clause 194 or 195, wherein m1 is at least about 2.

Clause 197. The compound of clause 194 or 195, wherein m1 is at least about 3.

Clause 198. The compound of clause 194 or 195, wherein m1 is at least about 4.

Clause 199. The compound of any one of clauses 185 to 198, wherein Z is a residual moiety resulting from the covalent linkage of a thiol-reactive chemoselective ligation group to one or more cysteine residue(s) of Ab.

Clause 200. The compound of any one of clauses 185 to 198, wherein Z is a residual moiety resulting from the covalent linkage of an amine-reactive chemoselective ligation group to one or more lysine residue(s) of Ab.

Clause 201. The compound of any one of clauses 185 to 200, wherein the antibody or antibody fragment is an IgG antibody.

Clause 202. The compound of any one of clauses 185 to 200, wherein the antibody or antibody fragment is a humanized antibody.

Clause 203. The compound of any one of clauses 185 to 202, wherein the antibody or antibody fragment specifically binds to a secreted or soluble protein.

Clause 204. The compound of any one of clauses 185 to 202, wherein the antibody or antibody fragment specifically binds to a cell surface receptor.

Clause 205. A method of internalizing a target protein in a cell comprising a cell surface folate receptor, the method comprising: contacting a cellular sample comprising the cell and the target protein with an effective amount of a compound according to any one of clauses 101 to 204, wherein the compound specifically binds the target protein and specifically binds the cell surface folate receptor to facilitate cellular uptake of the target protein.

Clause 206. The method of clause 205, wherein the target protein is a membrane bound protein.

Clause 207. The method of clause 205, wherein the target protein is an extracellular protein.

Clause 208. The method of any one of clauses 205 to 207, wherein the compound or conjugate comprises an antibody or antibody fragment (Ab) that specifically binds the target protein.

Clause 209. A method of reducing levels of a target protein in a biological system, the method comprising: contacting the biological system with an effective amount of a compound according to any one of clauses 1 to 104, wherein the compound specifically binds the target protein and specifically binds a cell surface receptor of cells in the biological system to facilitate cellular uptake and degradation of the target protein.

Clause 210. The method of clause 109, wherein the biological system comprises cells that comprise a folate cell surface receptor.

Clause 211. The method of clause 109 or 110, wherein the biological system is a human subject.

Clause 212. The method of any one of clauses 109 to 111, wherein the biological system is an in vitro cellular sample.

Clause 213. The method of any one of clauses 109 to 112, wherein the target protein is a membrane bound protein.

Clause 214. The method of any one of clauses 109 to 112, wherein the target protein is an extracellular protein.

Clause 215. A method of treating a disease or disorder associated with a target protein, the method comprising: administering to a subject in need thereof an effective amount of a compound according to any one of clauses 101 to 204, wherein the compound specifically binds the target protein.

Clause 216. The method of clause 215, wherein the disease or disorder is an inflammatory disease.

Clause 217. The method of clause 215, wherein the disease or disorder is an autoimmune disease.

Clause 218. The method of clause 215, wherein the disease or disorder is a cancer.

### 6. EXAMPLES

The following examples are offered to illustrate the present disclosure and are not to be construed in any way as limiting the scope of the present technology. Any methods that are functionally equivalent are within the scope of the present technology. Various modifications of the present technology in addition to those described herein will become apparent to those skilled in the art from the foregoing description and accompanying figures. Such modifications fall within the scope of the appended claims.

### 6.1. Preparation of Compounds

The following are illustrative schemes and examples of how the compounds described herein can be prepared and tested. Although the examples can represent only some embodiments, it should be understood that the following examples are illustrative and not limiting. All substituents, unless otherwise specified, are as previously defined. The reagents and starting materials are readily available to one of ordinary skill in the art. The specific synthetic steps for each of the routes described may be combined in different ways, or in conjunction with steps from different schemes, to prepare the compounds described herein.

### 6.1.1. Example 1: Compound I-1

### Example 1: Synthesis of (S)-38-(4-(((2-amino-4-oxo-3,4-dihydropteridin-6-yl)methyl)amino)benzamido)-1-(2,5-dioxo-2,5-dihydro-1H-pyrrol-1-yl)-3,35-dioxo-7,10,13,16,19,22,25,28,31-nonaoxa-4,34-diazanonatriacontan-39-oic acid (Compound I-1)

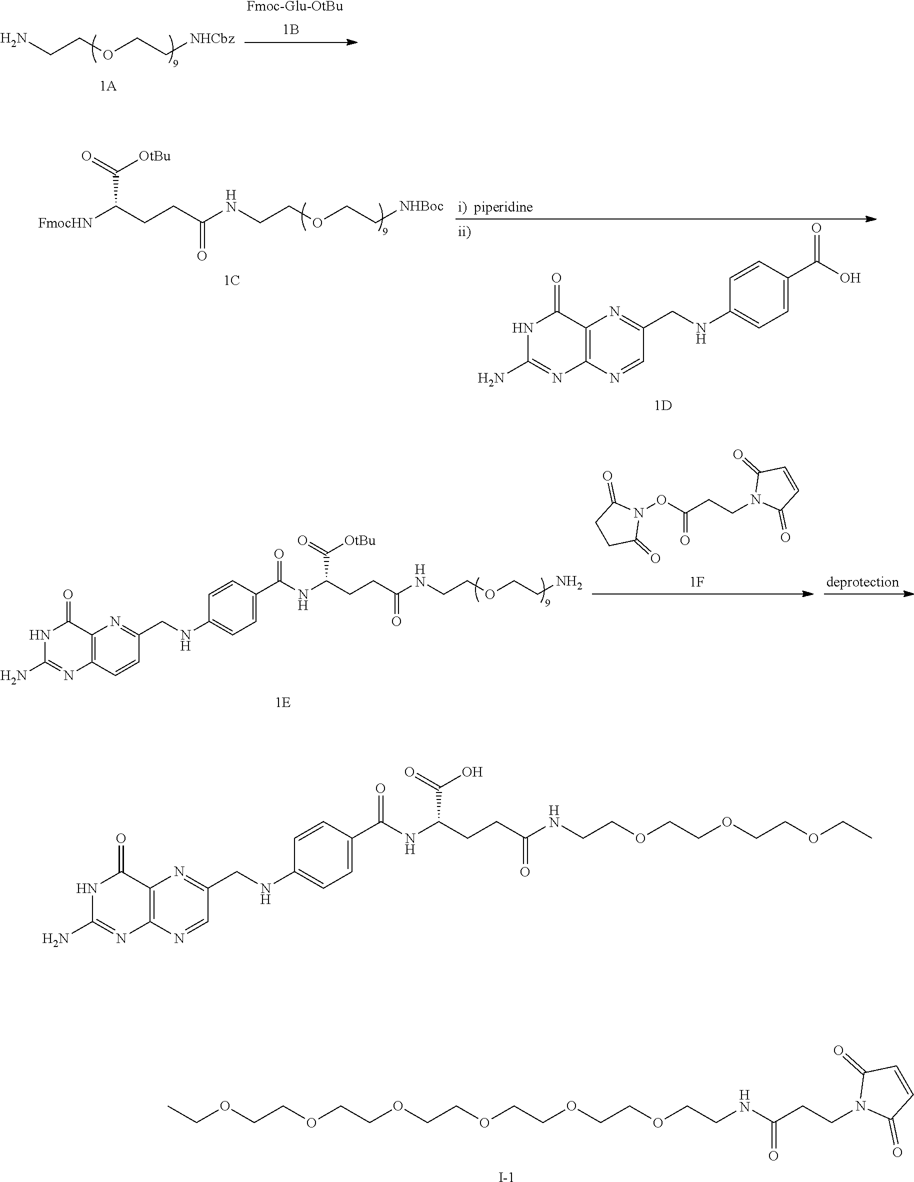

To a solution of Compound 1A and Fmoc-Glu-OtBu (1B) in DMF is added DIPEA, HOBt and HBTU in DMF. The reaction mixture is stirred at room temperature and monitored by TLC until ninhydrin test shows no free amine is observed. Upon completion, DMF is removed under vacuum and the reaction mixture is purified by preparatory HPLC. Fractions containing the desired product were combined and lyophilized to dryness to afford Compound 1C.

To a solution of Compound 1C in DMF is added piperidine, and the reaction mixture is stirred at room temperature for 1 h. A mixture of pteroic acid (1D), DIPEA, HOBt and HBTU in DMF/DMSO is added to the solution of Compound 1C. The reaction is monitored by LCMS until no free amine is observed. The reaction mixture is purified by preparatory HPLC. Fractions containing the coupled product were combined and lyophilized to dryness. The protected intermediate in DCM is treated with excess TFA and stirred at room temperature until deprotected. The mixture is concentrated to dryness to afford Compound 1E.

To a stirred solution of Compound 1E and Compound 1F in DMF, DIPEA is added and reaction mixture is stirred for 3 h. The progress of reaction is monitored by LC-MS and stirred until completion. The reaction mixture is purified by preparatory HPLC to afford Compound I-1.

### 6.1.2. Example 2: Compound I-2

### Synthesis of (S)-40-(4-(((2-amino-4-oxo-3,4-dihydropteridin-6-yl)methyl)amino)benzamido)-1,31,37-trioxo-1-(perfluorophenoxy)-4,7,10,13,16,19,22,25,28-nonaoxa-32,36-diazahentetracontan-41-oic acid (Compound I-2)

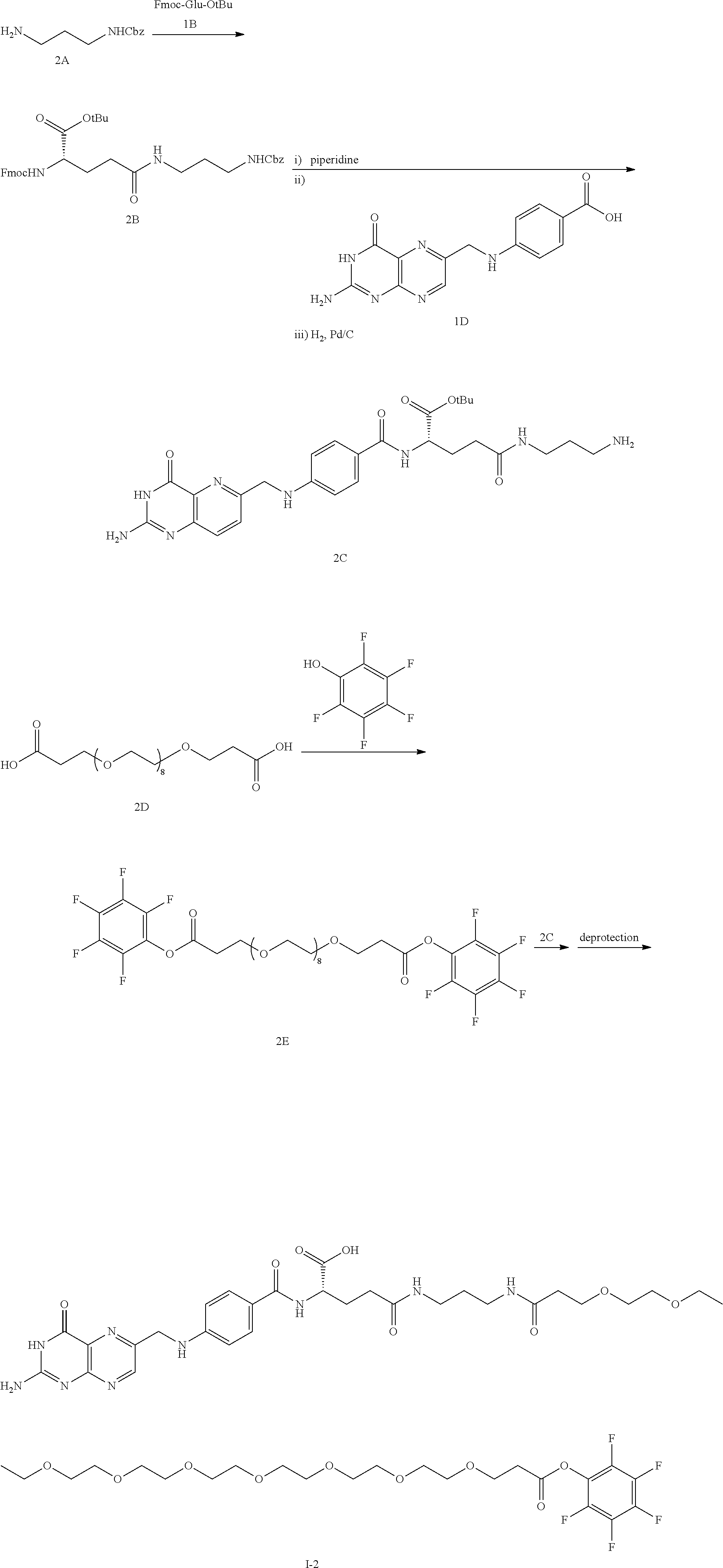

To a solution of Compound 2A and Fmoc-Glu-OtBu (1B) in DMF is added DIPEA, HOBt and HBTU in DMF. The reaction mixture is stirred at room temperature and monitored by TLC until ninhydrin test shows no free amine is observed. Upon completion, DMF is removed under vacuum and the reaction mixture is purified by preparatory HPLC. Fractions containing the desired product are combined and lyophilized to dryness to afford Compound 2B.

To a solution of Compound 2B in DMF is added piperidine, and the reaction mixture is stirred at room temperature for 1 h. A mixture of -pteroic acid (1D), DIPEA, HOBt and HBTU in DMF is added to the solution of Compound 1C. The reaction is monitored by LCMS until no free amine is detected. Upon completion, the reaction mixture is purified by preparatory HPLC. Fractions containing the desired product are combined and lyophilized to dryness. The protected intermediate is dissolved in EtOH/EtOAc and Pd/C is added and the mixture placed under a hydrogen atmosphere and stirred until the Cbz group is removed. The mixture is filtered and concentrated, and the crude material is purified by preparative HPLC to afford Compound 2C.

A solution of Compound 2D and pentafluorophenol in ethyl acetate is cooled at 0° C., N,N′-diisopropylcarbodiimide is added and reaction mixture is stirred at room temperature for 3 h. Reaction mixture is filtered through Celite bed and Celite bed was washed with ethyl acetate. The filtrate is concentrated to get crude product which is purified by column chromatography using silica gel (100-200 mesh) and 0-10% ethyl acetate in hexane to afford Compound 2E.

To a stirred solution of Compound 2C and Compound 2E in DMF, DIPEA is added and reaction mixture is stirred for 3 h. The progress of reaction was monitored by LC-MS. After the completion of reaction, a 95:2.5:2.5 mixture of TFA:TIPS:H₂O is added to the reaction mixture and the reaction mixture is stirred at room temperature for 30 min. The reaction mixture is concentrated under reduced pressure to afford crude. The crude is purified by preparatory HPLC to afford Compound I-2.

### 6.1.3. Example 3: Compound I-3

### Example 3: Synthesis of N-(4-(((2-amino-4-oxo-3,4-dihydropteridin-6-yl)methyl)amino)benzoyl)-N⁵-(4-(1-(2-(3-oxo-3-(perfluorophenoxy)propoxy)ethyl)-1H-1,2,3-triazol-4-yl)butyl)-L-glutamine (Compound I-3)

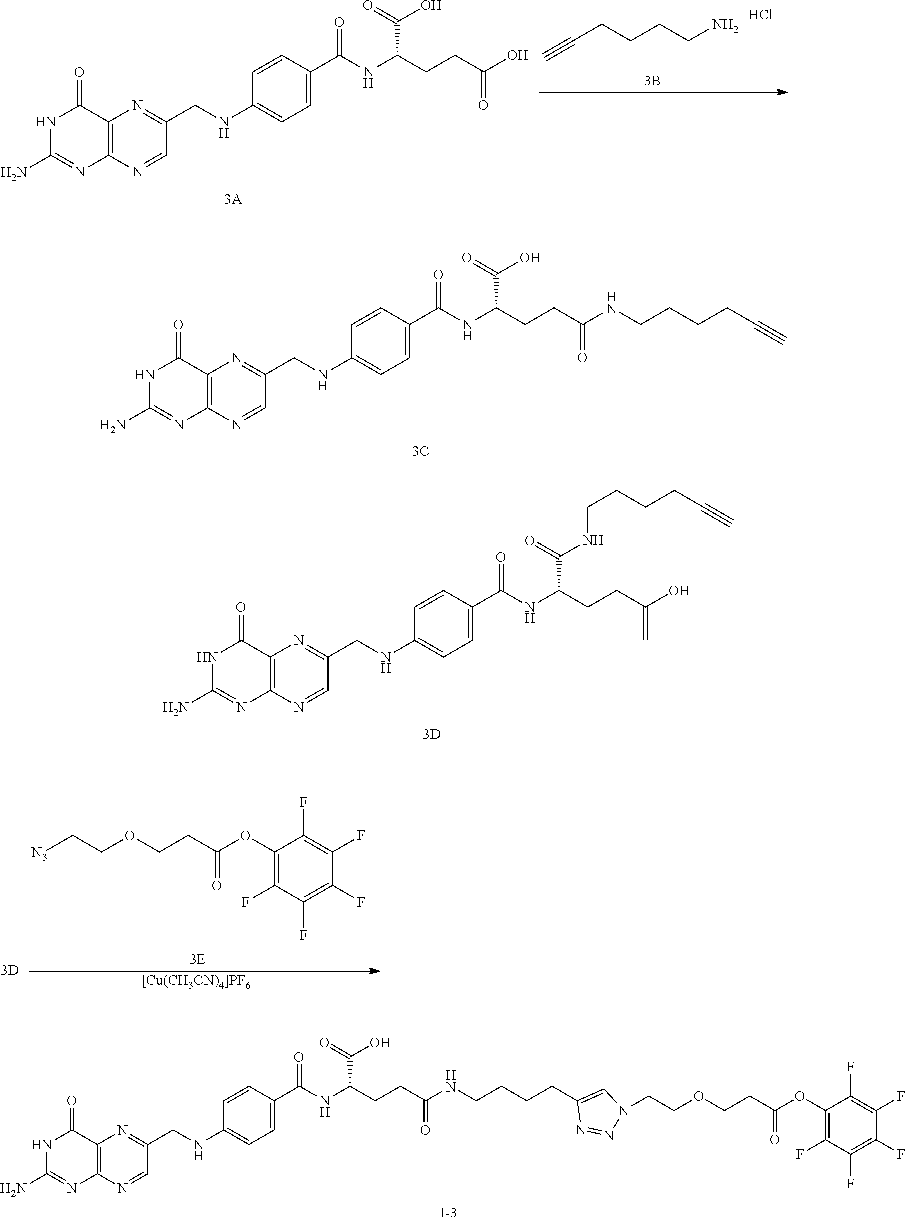

To a solution of (4-(((2-amino-4-oxo-3,4-dihydropteridin-6-yl)methyl)amino)benzoyl)-L-glutamic acid (3A) (1.0 eq, 1.0 g, 2.27 mmol) in N,N-dimethylformamide (20 mL) and dimethyl sulfoxide (20 mL), N-hydroxysuccinimide (1.1 eq, 0.287 g, 2.49 mmol), 1-(3-Dimethylaminopropyl)-3-ethylcarbodiimide hydrochloride (1.1 eq, 0.478 g, 2.49 mmol) and N,N-diisopropylethylamine (3.0 eq, 1.25 mL, 6.80 mmol) were added and reaction mixture was stirred at room temperature for 30 minutes. Then, hex-5-yn-1-amine hydrochloride (3B) (1.1 eq., 0.333 g, 2.49 mmol) was added and reaction mixture was stirred at room temperature for 16 h. After completion, reaction mixture was poured into 30% acetone in diethyl ether to get solid which was filtered off and dried to afford crude. Crude was purified by prep HPLC (22-35% acetonitrile in water with 0.1% TFA). Fractions containing the desired product were combined and lyophilized to dryness to afford a mixture of Compounds 3C and 3D, which was repurified by prep HPLC (20-40% acetonitrile in water with 0.1% acetic acid). Fractions containing the desired product were combined and lyophilized to dryness to afford Compound 3C as a yellow solid, Yield: 0.080 g, 6.72%; LC-MS m/z 521.0 [M+1]⁺ and Compound 3D as a yellow solid, Yield: 0.040 g, 2.78%; LC-MS m/z 521.0 [M+1]⁺.

To a solution of Compound 3C (1.0 eq., 0.025 g, 0.048 mmol) in dimethylsulfoxide (0.5 mL), perfluorophenyl 3-(2-azidoethoxy)propanoate (3E) (1.0 eq., 0.015 g, 0.048 mmol) was added and stirred for 5 minutes. Then, tetrakis(acetonitrile)copper(I) hexafluorophosphate (2.8 eq., 0.050 g, 0.134 mmol) was added and reaction mixture was stirred at room temperature for 30 minutes. After completion, reaction mixture was diluted with acetonitrile and purified by prep HPLC (40-55% acetonitrile in water with 0.1% acetic acid). Fractions containing the desired product were combined and lyophilized to dryness to afford Compound I-3 as a yellow solid. Yield: 0.0088 g, 20.7%; LC-MS m/z 846.18 [M+1]+; ¹H NMR (400 MHz, DMSO-d₆) δ 12.51 (bs, 1H), 11.42 (bs, 1H), 8.64 (s, 1H), 8.19 (d, J=8.0 Hz, 1H), 7.81 (t, J=5.6 Hz, 1H), 7.72 (s, 1H), 7.64 (d, J=8.4 Hz, 2H), 6.94 (bs, 3H), 6.63 (d, J=8.0 Hz, 2H), 4.48-4.44 (m, 4H), 4.25 (bs, 1H), 3.81 (t, J=5.6 Hz, 2H), 3.74 (t, J=6.0 Hz, 2H), 3.01 (t, J=5.2 Hz, 4H), 2.56-2.54 (m, 2H), 2.16-2.14 (m, 2H), 2.03-2.02 (m, 1H), 1.91-1.87 (m, 1H), 1.54-1.48 (m, 2H), 1.41-1.35 (m, 2H).

### 6.1.4. Example 4: Compound I-4

### Example 4: Synthesis of N²-(4-(((2-amino-4-oxo-3,4-dihydropteridin-6-yl)methyl)amino)benzoyl)-N⁵-(4-(1-(15-oxo-15-(perfluorophenoxy)-3,6,9,12-tetraoxapentadecyl)-1H-1,2,3-triazol-4-yl)butyl)-L-glutamine (Compound I-4)

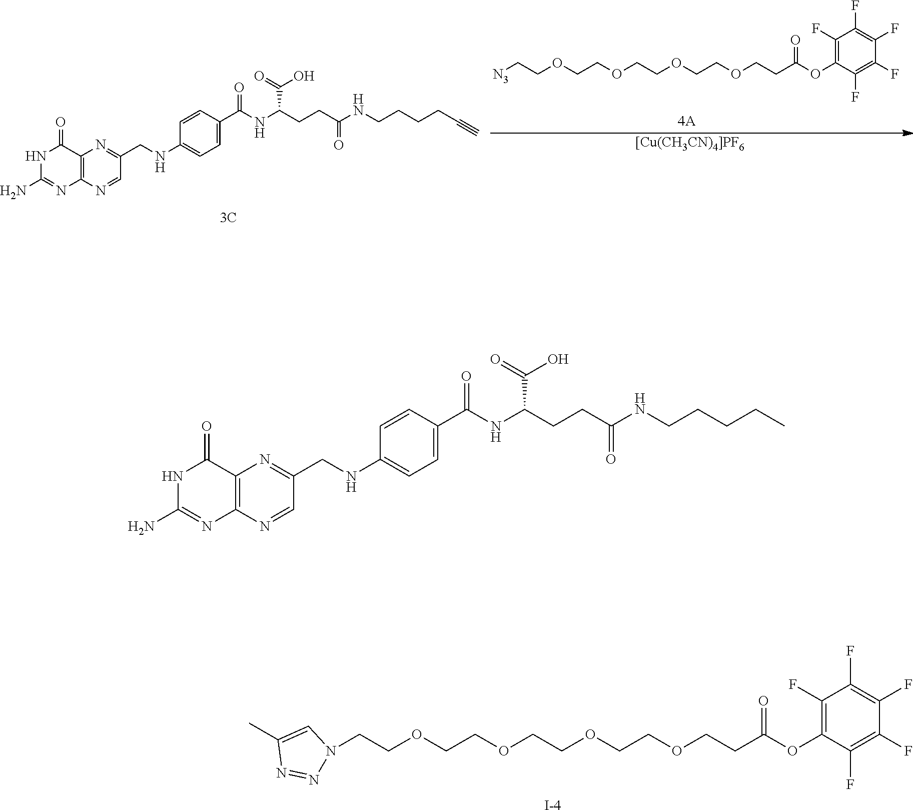

To a solution of Compound 3C (1.0 eq., 0.020 g, 0.038 mmol) in dimethylsulfoxide (0.5 mL), perfluorophenyl 1-azido-3,6,9,12-tetraoxapentadecan-15-oate (4A) (1.0 eq., 0.017 g, 0.038 mmol) was added and stirred for 5 minutes. Then, tetrakis(acetonitrile)copper(I) hexafluorophosphate (2.8 eq., 0.040 g, 0.108 mmol) was added and reaction mixture was stirred at room temperature for 30 minutes. After completion, reaction mixture was diluted with acetonitrile and purified by prep HPLC (35-48% acetonitrile in water with 0.1% TFA). Fractions containing the desired product were combined and lyophilized to dryness to afford Compound I-4 as a yellow solid. Yield: 0.0078 g, 20.4%; LC-MS m/z 978.2 [M+1]+; ¹H NMR (400 MHz, DMSO-d₆) δ 12.44 (bs, 1H), 11.42 (bs, 1H), 8.66 (s, 1H), 8.20 (d, J=7.6 Hz, 1H), 7.82-7.80 (m, 1H), 7.78 (s, 1H), 7.64 (d, J=8.0 Hz, 2H), 7.12 (bs, 2H), 6.63 (d, J=8.4 Hz, 2H), 4.49 (s, 2H), 4.43 (t, J=5.2 Hz, 2H), 4.26-4.23 (m, 2H), 3.78-3.73 (m, 4H), 3.52-3.47 (m, 11H), 3.05-2.99 (m, 4H), 2.58-2.55 (m, 2H), 2.16-2.15 (m, 2H), 2.07-2.02 (m, 1H), 1.93-1.85 (m, 2H), 1.56-1.50 (m, 2H), 1.40-1.37 (m, 2H).

### Synthesis of N2-(4-(((2-amino-4-oxo-3,4-dihydropteridin-6-yl)methyl)amino)benzoyl)-N5-(4-(1-(27-oxo-27-(perfluorophenoxy)-3,6,9,12,15,18,21,24-octaoxaheptacosyl)-1H-1,2,3-triazol-4-yl)butyl)-L-glutamine

A variety of compounds having different linker lengths are prepared using a similar procedure. Compound 3C was used to prepare Compound I-4B having a PEG8 linking group adjacent to the triazolyl ring.

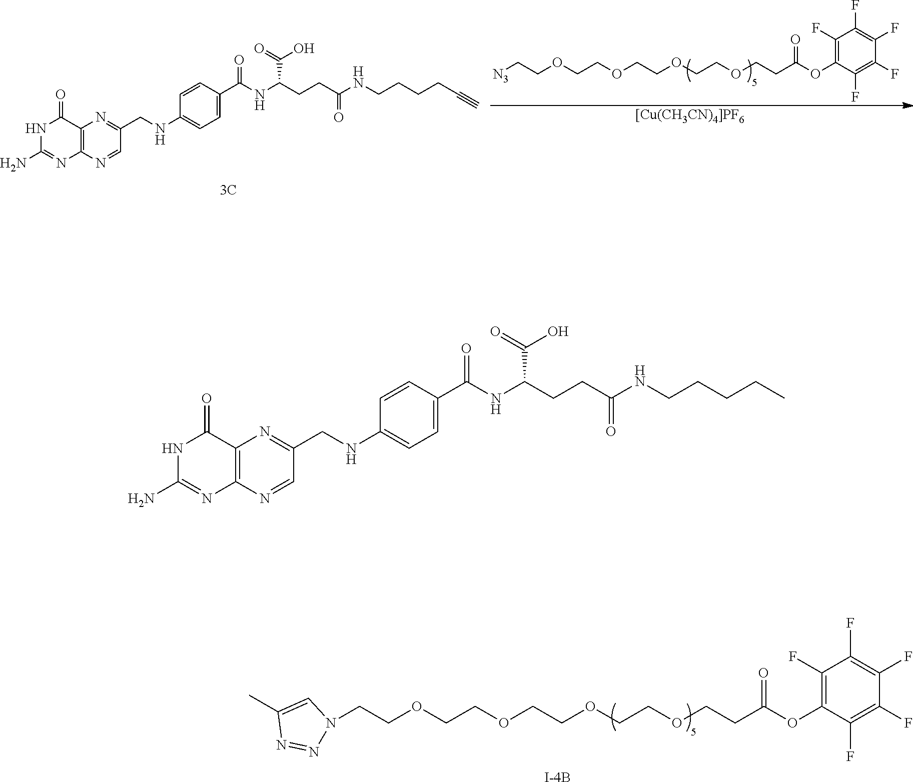

### 6.1.5. Example 5: Compound I-5

### Example 5: Synthesis of (2S,2′S)-5,5′-((((12-(2-(2-(2-(3-oxo-3-(perfluorophenoxy)propoxy)ethoxy)ethoxy)ethyl)-3,6,9,15,18,21-hexaoxa-12-azatricosane-1,23-diyl)bis(1H-1,2,3-triazole-1,4-diyl))bis(butane-4,1-diyl))bis(azanediyl))bis(2-(4-(((2-amino-4-oxo-3,4-dihydropteridin-6-yl)methyl)amino)benzamido)-5-oxopentanoic acid) (Compound I-5)

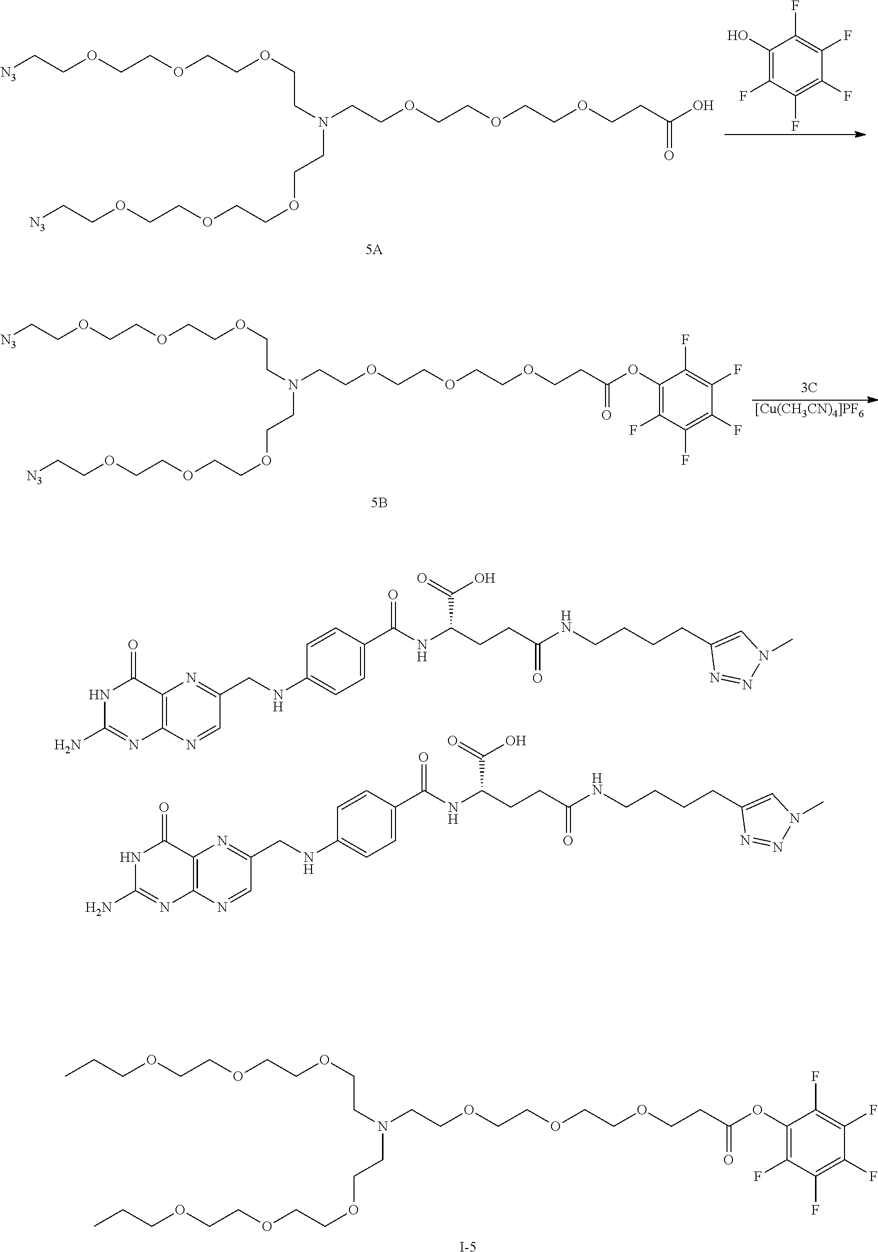

A solution of Compound 5A and pentafluorophenol in ethyl acetate is cooled at 0° C., N,N′-diisopropylcarbodiimide is added and reaction mixture is stirred at room temperature for 3 h. Reaction mixture is filtered through a Celite bed and the Celite bed is washed with ethyl acetate. The filtrate is concentrated to get crude product, which is purified by column chromatography using silica gel (100-200 mesh) and 0-10% ethyl acetate in hexane to afford Compound 5B.

To a solution of Compound 3C (1.0 eq.) in dimethylsulfoxide (0.5 mL), Compound 5B (1.0 eq.) is added and stirred for 5 minutes. Then, tetrakis(acetonitrile)copper(I) hexafluorophosphate (2.8 eq.) is added and reaction mixture is stirred at room temperature for 30 minutes. After completion, reaction mixture is diluted with acetonitrile and purified by prep HPLC (35-48% acetonitrile in water with 0.1% TFA). Fractions containing the desired product are combined and lyophilized to dryness to afford Compound I-5 as a solid. 6.1.6. Example 6: Compound I-6

### Example 6: Synthesis of N²-(4-(((2-amino-4-oxo-3,4-dihydropteridin-6-yl)methyl)amino)benzoyl)-N⁵-(4-(1-(15-(2,5-dioxo-2,5-dihydro-1H-pyrrol-1-yl)-13-oxo-3,6,9-trioxa-12-azapentadecyl)-1H-1,2,3-triazol-4-yl)butyl)-L-glutamine (Compound I-6)

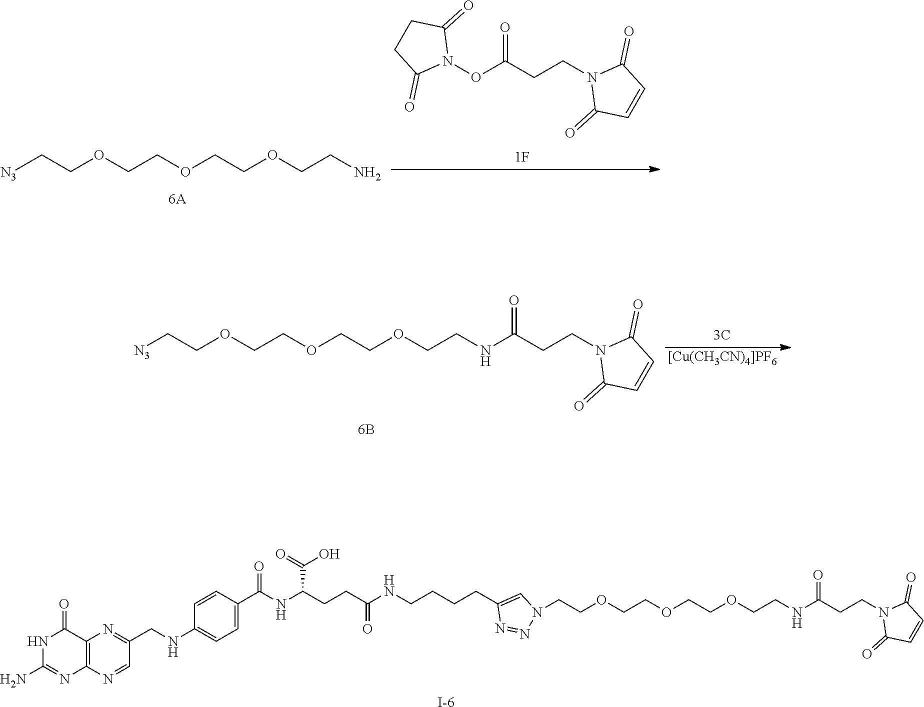

To a solution of 2-(2-(2-(2-azidoethoxy)ethoxy)ethoxy)ethan-1-amine (6A) (1.0 eq, 0.025 g, 0.115 mmol) in dimethyl sulfoxide (2 mL), 2,5-dioxopyrrolidin-1-yl 3-(2,5-dioxo-2,5-dihydro-1H-pyrrol-1-yl)propanoate (1F) (1.0 eq, 0.035 g, 0.115 mmol) was added and reaction mixture was stirred at room temperature for 30 minutes. Reaction was monitored by TLC. After completion of reaction, the solution of crude Compound 6B was used as such for next reaction. To this solution was added Compound 3C (0.6 eq, 0.0358 g, 0.0687 mmol) and stirred for 5 minutes. Tetrakis(acetonitrile)copper(I) hexafluorophosphate (2.5 eq, 0.0937 g, 0.286 mmol) was added and reaction mixture was stirred at room temperature for 1 h. The progress of reaction was monitored by LC-MS. After completion, reaction mixture was diluted with acetonitrile and purified by prep HPLC (13-22% acetonitrile in water with 0.1% TFA). Fractions containing the desired product were combined and lyophilized to dryness to afford Compound I-6 as a yellow solid. Yield: 0.015 g, 13.6%; LC-MS m/z 890.3 [M+1]+; ¹H NMR (400 MHz, DMSO-d₆) δ 12.5 (bs, 1H), 11.5 (bs, 1H), 8.64 (s, 1H), 8.20 (d, J=7.44 Hz, 1H), 8.03 (t, J=5.32 Hz, 1H), 7.82 (t, J=5.84 Hz, 1H), 7.78 (s, 1H), 7.64 (d, J=8.72 Hz, 2H), 6.99 (s, 2H), 6.63 (d, J=8.68 Hz, 2H), 4.48 (s, 2H), 4.44 (t, J=5.4 Hz, 2H), 4.30-4.20 (m, 1H), 3.77 (t, J=5.16 Hz, 2H), 3.59-3.45 (m, 10H), 3.34 (t, J=5.92 Hz, 2H), 3.15-3.12 (m, 2H), 3.03-3.00 (m, 2H), 2.57 (t, J=7.6 Hz, 2H), 2.33-2.31 (m, 2H), 2.17-2.15 (m, 2H), 2.10-2.00 (m, 1H), 1.90-1.80 (m, 1H), 1.56-1.52 (m, 2H), 1.40-1.36 (m, 2H).

### 6.1.7. Example 7: Compound I-7

### Example 7: Synthesis of N²-(4-(((2-amino-4-oxo-3,4-dihydropteridin-6-yl)methyl)amino)benzoyl)-N⁵-(4-(1-(27-(2,5-dioxo-2,5-dihydro-1H-pyrrol-1-yl)-25-oxo-3,6,9,12,15,18,21-heptaoxa-24-azaheptacosyl)-1H-1,2,3-triazol-4-yl)butyl)-L-glutamine (Compound I-7)

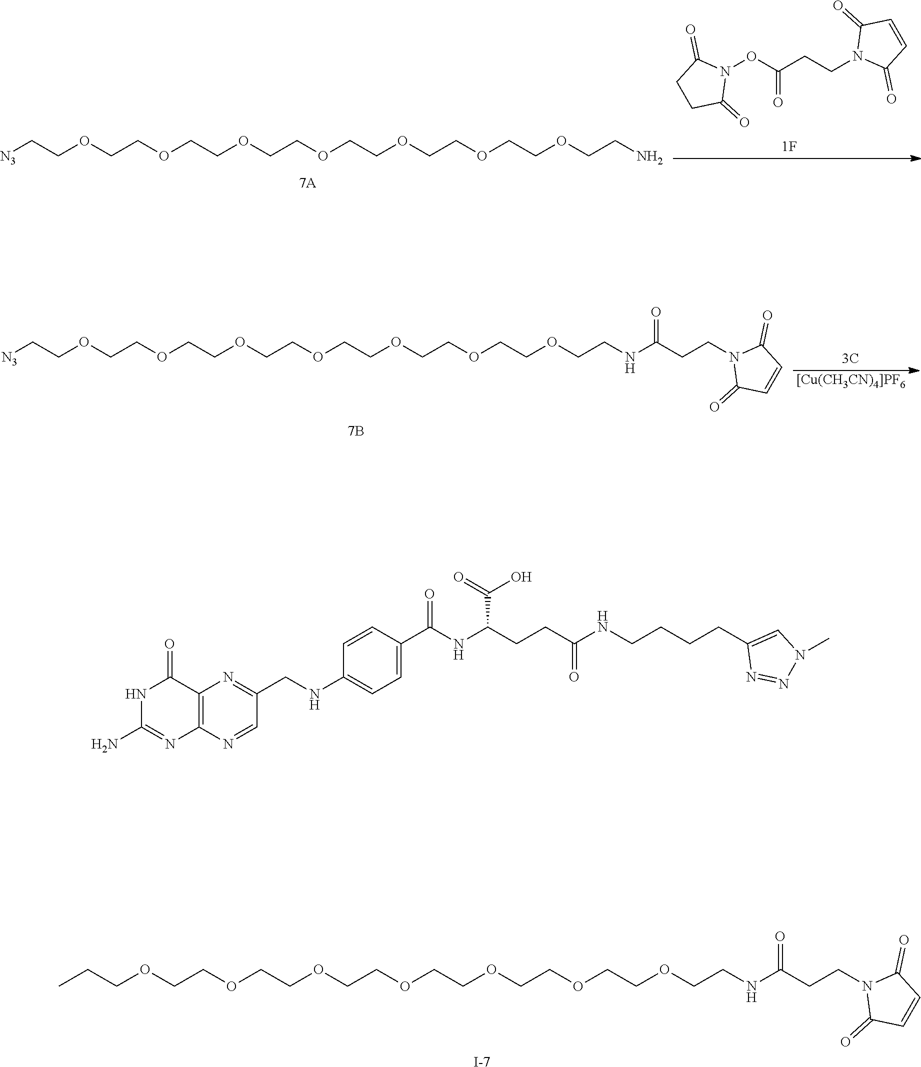

Compound 7B is synthesized by employing the procedure described for Compound 6B using Compound 7A in lieu of Compound 6A.

Compound I-7 is synthesized by employing the procedure described for Compound I-6 using Compound 7B in lieu of Compound 6B.

### 6.1.8. Example 8: Compound I-8

### Example 8: Synthesis of N²-(4-(((2-amino-4-oxo-3,4-dihydropteridin-6-yl)methyl)amino)benzoyl)-N⁵-(4-(1-(39-(2,5-dioxo-2,5-dihydro-1H-pyrrol-1-yl)-37-oxo-3,6,9,12,15,18,21,24,27,30,33-undecaoxa-36-azanonatriacontyl)-1H-1,2,3-triazol-4-yl)butyl)-L-glutamine (Compound I-8)

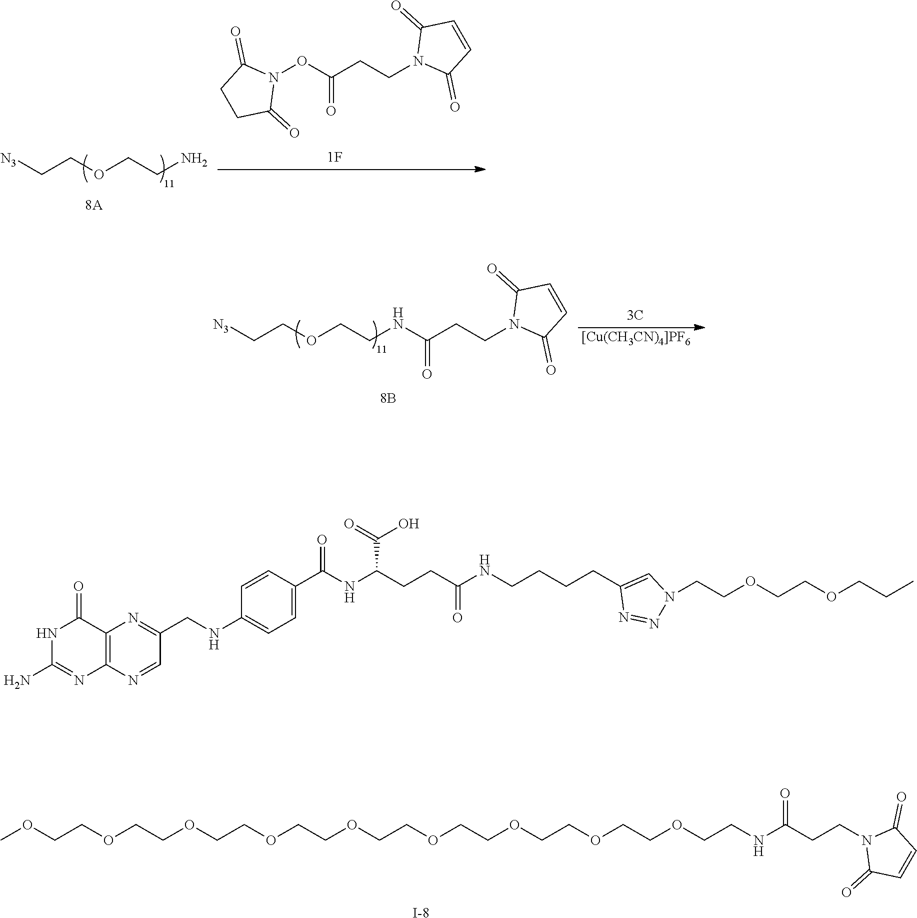

Compound 8B is synthesized by employing the procedure described for Compound 6B using Compound 8A in lieu of Compound 6A.

Compound I-8 is synthesized by employing the procedure described for Compound I-6 using Compound 8B in lieu of Compound 6B.

### 6.1.9. Example 9: Compound I-9

### Example 9: Synthesis of (S)-4-(4-(((2-amino-4-oxo-3,4-dihydropteridin-6-yl)methyl)amino)benzamido)-5-oxo-5-((4-(1-(2-(3-oxo-3-(perfluorophenoxy)propoxy)ethyl)-1H-1,2,3-triazol-4-yl)butyl)amino)pentanoic acid (Compound I-9)

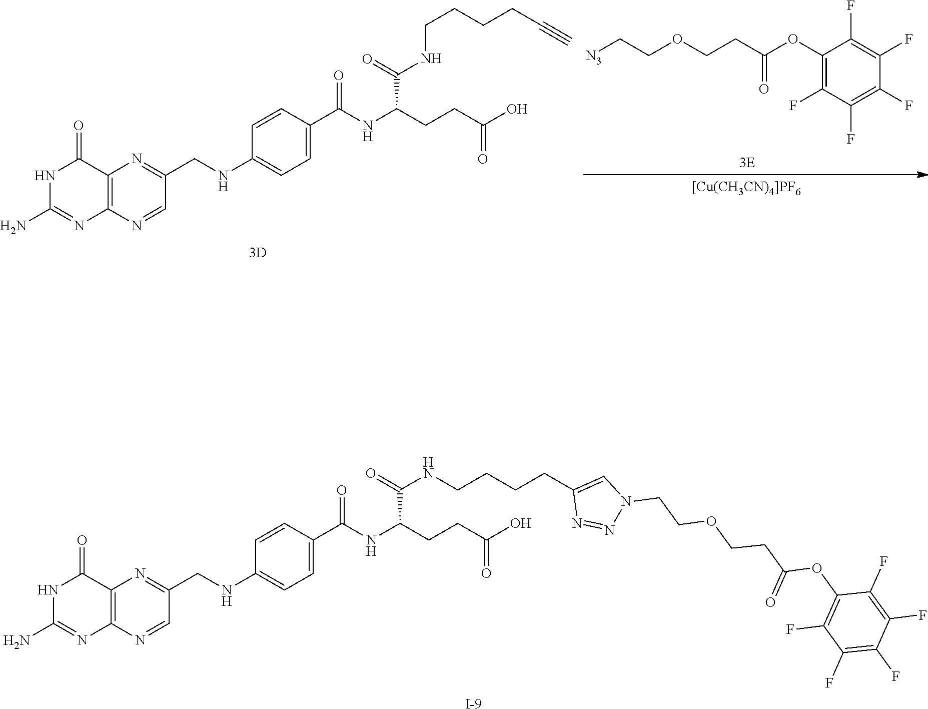

To a solution of Compound 3D (1.0 eq., 0.020 g, 0.038 mmol) in dimethylsulfoxide (0.5 mL), Compound 3E (1.0 eq., 0.012 g, 0.038 mmol) was added and stirred for 5 minutes. Then, tetrakis(acetonitrile)copper(I) hexafluorophosphate (2.8 eq., 0.040 g, 0.108 mmol) was added and reaction mixture was stirred at room temperature for 30 minutes. After completion, reaction mixture was diluted with acetonitrile and purified by prep HPLC (38-47% acetonitrile in water with 0.1% TFA). Fractions containing the desired product were combined and lyophilized to dryness to afford Compound I-9 as a yellow solid. Yield: 0.0052 g, 15.6%; LC-MS m/z 846.1 [M+1]⁺; ¹H NMR (400 MHz, DMSO-d₆) δ 8.65 (s, 1H), 7.92 (d, J=8.0 Hz, 1H), 7.83 (t, J=5.2 Hz, 1H), 7.71 (s, 1H), 7.64 (d, J=8.8 Hz, 2H), 7.20-6.94 (m, 2H), 6.62 (d, J=8.8 Hz, 2H), 4.49 (s, 2H), 4.45 (t, J=5.2 Hz, 2H), 4.34-4.29 (m, 1H), 3.81 (t, J=5.2 Hz, 2H), 3.74 (t, J=6.0 Hz, 2H), 3.05-2.99 (m, 4H), 2.58-2.55 (m, 2H), 2.23 (t, J=10.4 Hz, 2H), 1.96-1.93 (m, 1H), 1.86-1.80 (m, 1H), 1.55-1.49 (m, 2H), 1.43-1.38 (m, 2H).

### 6.1.10. Example 10: Compound I-10

### Example 10: Synthesis of (S)-4-(4-(((2-amino-4-oxo-3,4-dihydropteridin-6-yl)methyl)amino)benzamido)-5-oxo-5-((4-(1-(15-oxo-15-(perfluorophenoxy)-3,6,9,12-tetraoxapentadecyl)-1H-1,2,3-triazol-4-yl)butyl)amino)pentanoic acid (Compound I-10)

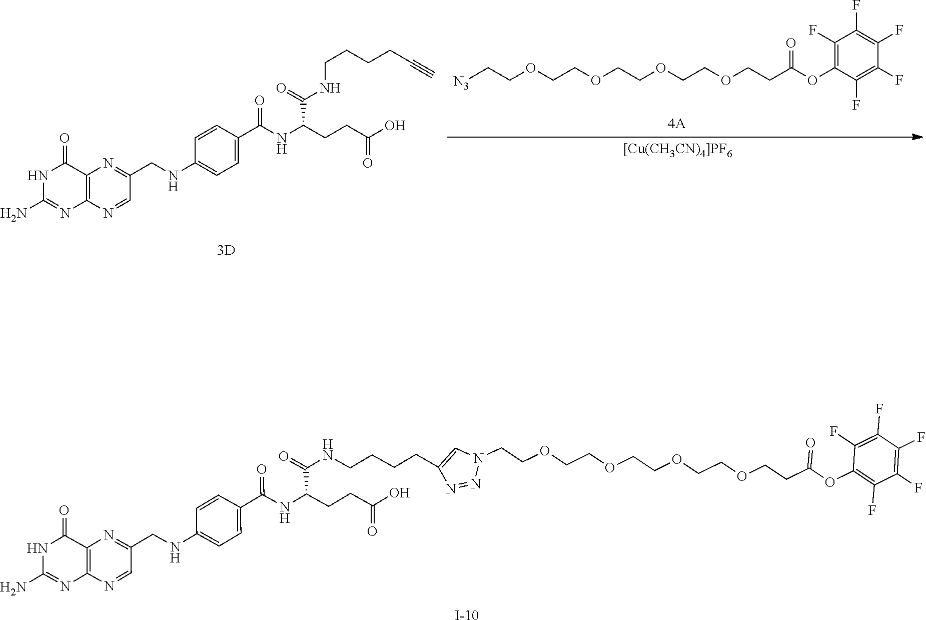

To a solution of Compound 3D (1.0 eq., 0.020 g, 0.038 mmol) in dimethylsulfoxide (0.5 mL), Compound 4A (1.0 eq., 0.017 g, 0.038 mmol) was added and stirred for 5 minutes. Then, tetrakis(acetonitrile)copper(I) hexafluorophosphate (2.8 eq., 0.040 g, 0.108 mmol) was added and reaction mixture was stirred at room temperature for 30 minutes. After completion, reaction mixture was diluted with acetonitrile and purified by prep HPLC (43-55% acetonitrile in water with 0.1% acetic acid). Fractions containing the desired product were combined and lyophilized to dryness to afford Compound I-10 as a light yellow solid. Yield: 0.0033 g, 7.3%; LC-MS m/z 978.3 [M+1]⁺; ¹H NMR (400 MHz, DMSO-d₆) δ 12.05 (bs, 1H), 11.41 (s, 1H), 8.64 (s, 1H), 7.92 (d, J=7.6 Hz, 1H), 7.83 (t, J=5.6 Hz, 1H), 7.76 (s, 1H), 7.64 (d, J=8.8 Hz, 2H), 6.94-6.83 (m, 2H), 6.62 (d, J=8.8 Hz, 2H), 4.48-4.47 (m, 2H), 4.43 (t, J=5.2 Hz, 2H), 4.34-4.29 (m, 1H), 3.77-3.73 (m, 4H), 3.55-3.44 (m, 13H), 3.06-2.99 (m, 4H), 2.58-2.56 (m, 2H), 2.23-2.22 (m, 2H), 1.96-1.93 (m, 1H), 1.86-1.80 (m, 1H), 1.57-1.53 (m, 2H), 1.43-1.39 (m, 2H).

### 6.1.11. Example 11: Compound I-11

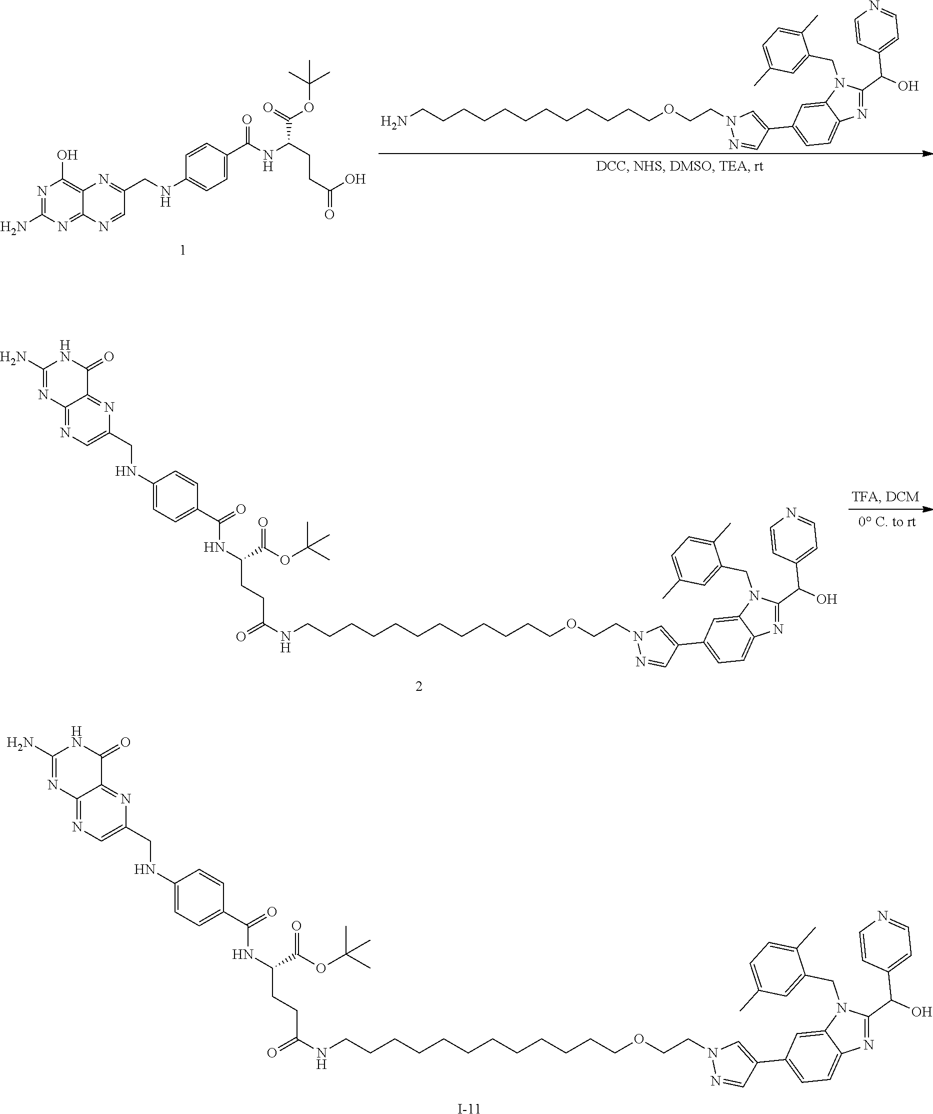

### Synthesis of intermediate: (6-(1-(2-((12-aminododecyl)oxy)ethyl)-1H-pyrazol-4-yl)-1-(2,5-dimethylbenzyl)-1H-benzo[d]imidazol-2-yl)(pyridin-4-yl)methanol

To a solution of 4-bromo-2-fluoro-1-nitrobenzene (1.0 eq, 10.0 g, 45.5 mmol) and (2,5-dimethylphenyl)methanamine (1.1 eq, 6.76 g, 50.0 mmol) in acetonitrile (200 mL), N,N-diisopropylethylamine (3.0 eq, 24.5 mL, 136.0 mmol) was added and reaction was heated at 70° C. for 3 h. After completion, reaction mixture was diluted with water and extracted with ethyl acetate. The organic layer was dried over anhydrous sodium sulfate, filtered and concentrated to get 5-bromo-N-(2,5-dimethylbenzyl)-2-nitroaniline as a yellow solid. Yield: 15.0 g, 98.4%; LCMS m/z 334.9 [M+1]+.

To a solution of 5-bromo-N-(2,5-dimethylbenzyl)-2-nitroaniline (1.0 eq, 15.0 g, 44.7 mmol) in ethanol (360 mL) and water (40 mL), zinc powder (4.0 eq, 11.7 g, 179.0 mmol) and ammonium chloride (7.0 eq, 16.8 g, 313.0 mmol) were added and reaction mixture was heated at 50° C. for 3 h. After completion, reaction mixture was cooled, filtered through celite bed, celite bed was washed with ethyl acetate and filtrate was concentrated. Then, water was added and the mixture extracted with ethyl acetate. The organic layer was dried over anhydrous sodium sulfate, filtered and concentrated to get crude which was purified by column chromatography using silica gel (100-200 mesh) and 0-40% ethyl acetate in hexane to afford 5-bromo-N¹-(2,5-dimethylbenzyl)benzene-1,2-diamine as a brown solid. Yield: 12.0 g, 87.8%; LCMS m/z 305.1 [M+1]+.

5-bromo-N¹-(2,5-dimethylbenzyl)benzene-1,2-diamine (1.0 eq, 12.0 g, 39.3 mmol) and formic acid (120 mL) was stirred at room temperature for 16 h. After completion, reaction mixture was concentrated, ethyl acetate was added, washed with saturated aqueous sodium bicarbonate solution and water. The organic layer was dried over anhydrous sodium sulfate, filtered and concentrated to get crude which was purified by column chromatography using silica gel (100-200 mesh) and 0-50% ethyl acetate in hexane to afford 6-bromo-1-(2,5-dimethylbenzyl)-1H-benzo[d]imidazole as a pink solid. Yield: 8.7 g, 68.0%; LCMS m/z 315.0 [M+1]⁺.

A solution of 6-bromo-1-(2,5-dimethylbenzyl)-1H-benzo[d]imidazole (1.0 eq, 1.1 g, 3.49 mmol) in tetrahydrofuran (30 mL) was cooled at −78° C., lithium diisopropylamide (2.0 M in tetrahydrofuran, 2.5 eq, 4.36 mL, 8.72 mmol) was added and reaction mixture was stirred for 2 h at −78° C., then isonicotinaldehyde (4a′, 1.4 eq, 0.523 g, 4.89 mmol) was added and the reaction mixture was stirred at −78° C. for 30 minutes. After that, the reaction mixture was quenched with saturated aqueous ammonium chloride solution and allowed to warm to room temperature. Then, the reaction mixture was extracted with ethyl acetate. The organic layer was dried over anhydrous sodium sulfate, filtered and concentrated to get crude product which was purified by column chromatography using silica gel (100-200 mesh) and 0-5% methanol in dichloromethane to afford (6-bromo-1-(2,5-dimethylbenzyl)-1H-benzo[d]imidazol-2-yl)(pyridin-4-yl)methanol as a brown solid. Yield: 1.0 g, 67.8%; LCMS m/z 422.0 [M+1]⁺.

A solution of 12-aminododecan-1-ol (1.0 eq, 6.10 g, 30.3 mmol) and di-tert-butyl dicarbonate (1.1 eq, 8.08 mL, 33.3 mmol) in tetrahydrofuran (60 mL) was heated at 50° C. for 3 h. After completion, the reaction mixture was concentrated to get crude material which was purified by column chromatography using silica gel (100-200 mesh) and 0-25% ethyl acetate in hexane to afford tert-butyl (12-hydroxydodecyl)carbamate as a white solid. Yield: 8.0 g, 87.6%; LCMS m/z 302.0 [M+1]⁺.

To a suspension of tert-butyl (12-hydroxydodecyl)carbamate (1.0 eq, 5.0 g, 16.6 mmol) and 2-(2-bromoethoxy)tetrahydro-2H-pyran (2.0 eq, 6.94 g, 33.2 mmol) in 50% aqueous sodium hydroxide solution (50 mL), tetrabutylammonium sulfate (0.1 eq, 1.93 g, 1.66 mmol) was added and reaction mixture was heated at 70° C. for 16 h. After completion, reaction mixture was cooled, water was added and extracted with ethyl acetate. The organic layer was dried over anhydrous sodium sulfate, filtered and concentrated to get crude material which was purified by column chromatography using silica gel (100-200 mesh) and 0-10% ethyl acetate in hexane to afford tert-butyl (12-(2-((tetrahydro-2H-pyran-2-yl)oxy)ethoxy)dodecyl)carbamate as a colorless viscous liquid. Yield: 6.0 g, 82.8%; LCMS m/z 430.2 [M+1]⁺.

To a solution of tert-butyl (12-(2-((tetrahydro-2H-pyran-2-yl)oxy)ethoxy)dodecyl)carbamate (1.0 eq, 6.0 g, 14.0 mmol) in methanol (60 mL), p-toluenesulfonic acid monohydrate (0.1 eq, 0.266 g, 1.40 mmol) was added and reaction mixture was stirred at room temperature for 4 h. After completion, reaction mixture was concentrated, diluted with ethyl acetate, washed with saturated aqueous sodium bicarbonate solution and water. The organic layer was dried over anhydrous sodium sulfate, filtered and concentrated to afford tert-butyl (12-(2-hydroxyethoxy)dodecyl)carbamate as a colorless viscous liquid. Yield: 4.4 g, 91.1%; LCMS m/z 346.0 [M+1]⁺.

A solution of tert-butyl (12-(2-((tetrahydro-2H-pyran-2-yl)oxy)ethoxy)dodecyl)carbamate (1.0 eq, 4.2 g, 12.2 mmol) in dichloromethane (40 mL) was cooled at 0° C., triphenylphosphine (1.5 eq, 4.78 g, 18.2 mmol), imidazole (1.5 eq, 1.24 g, 18.2 mmol) and iodine (1.5 eq, 4.63 g, 18.2 mmol) were added and reaction mixture was stirred at room temperature for 2 h. After that, reaction mixture was diluted with water and extracted with dichloromethane. The organic layer was dried over sodium sulfate, filtered and concentrated to get crude which was purified by column chromatography using silica gel (100-200 mesh) and 0-7% ethyl acetate in hexane to afford tert-butyl (12-(2-iodoethoxy)dodecyl)carbamate as an off white solid. Yield: 4.0 g, 72.2%; LCMS m/z 456.0 [M+1]⁺.

A suspension of 4-(4,4,5,5-tetramethyl-1,3,2-dioxaborolan-2-yl)-1H-pyrazole (1.0 eq, 1.0 g, 5.15 mmol), tert-butyl (12-(2-iodoethoxy)dodecyl)carbamate (0.8 eq, 1.88 g, 4.12 mmol) and cesium carbonate (2.0 eq, 3.36 g, 10.3 mmol) in acetonitrile (20 mL) was heated at 70° C. for 16 h. After completion, reaction mixture was cooled and concentrated to get crude which was purified by column chromatography using silica gel (100-200 mesh) and 0-30% ethyl acetate in hexane to afford tert-butyl (12-(2-(4-(4,4,5,5-tetramethyl-1,3,2-dioxaborolan-2-yl)-1H-pyrazol-1-yl)ethoxy)dodecyl)carbamate as a colorless viscous liquid. Yield: 1.9 g, 70.3%; LCMS m/z 522.2 [M+1]⁺.

A solution of (6-bromo-1-(2,5-dimethylbenzyl)-1H-benzo[d]imidazol-2-yl)(pyridin-4-yl)methanol (1.0 eq, 1.0 g, 2.37 mmol), tert-butyl (12-(2-(4-(4,4,5,5-tetramethyl-1,3,2-dioxaborolan-2-yl)-1H-pyrazol-1-yl)ethoxy)dodecyl)carbamate (1.5 eq, 1.85 g, 3.55 mmol) and potassium carbonate (2.0 eq, 0.654 g, 4.74 mmol) in 1,4-dioxane (8 mL) and water (2 mL) was degassed under nitrogen for 5 minutes. Then, [1,1′-bis(diphenylphosphino)ferrocene]dichloropalladium(II) complex with dichloromethane (0.05 eq, 0.096 g, 0.118 mmol) was added and reaction mixture was heated at 100° C. for 16 h. After completion, reaction mixture was cooled, water was added and extracted with ethyl acetate. The organic layer was dried over anhydrous sodium sulfate, filtered and concentrated to get crude which was purified by column chromatography using silica gel (100-200 mesh) and 0-8% methanol in dichloromethane to afford tert-butyl (12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)carbamate as a brown solid. Yield: 1.25 g, 71.6%; LCMS m/z 737.3 [M+1]⁺.

A solution of tert-butyl (12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)carbamate (1.0 eq, 1.25 g, 1.70 mmol) in dichloromethane (6 mL) was cooled at 0° C., trifluoroacetic acid (6 mL) was added dropwise and reaction mixture was stirred at room temperature for 3 h. After completion, reaction mixture was concentrated, water was added, neutralized with solid sodium bicarbonate and extracted with 10% methanol in dichloromethane. The organic layer was dried over anhydrous sodium sulfate, filtered and concentrated to get crude which was purified by column chromatography using silica gel (100-200 mesh) and 0-20% methanol in dichloromethane to afford (6-(1-(2-((12-aminododecyl)oxy)ethyl)-1H-pyrazol-4-yl)-1-(2,5-dimethylbenzyl)-1H-benzo[d]imidazol-2-yl)(pyridin-4-yl)methanol as a light brown solid. Yield: 0.800 g, 73.1%; LCMS m/z 637.3 [M+1]⁺; ¹H NMR (400 MHz, DMSO-d₆) δ 8.38 (d, J=6.0 Hz, 2H), 8.03 (s, 1H), 7.78 (s, 1H), 7.62 (d, J=8.4 Hz, 1H), 7.44-7.41 (m, 2H), 7.29-7.26 (m, 2H), 7.05 (d, J=7.6 Hz, 1H), 6.86 (d, J=7.6 Hz, 1H), 6.01 (s, 1H), 5.81 (s, 1H), 5.59 (d, J=17.6 Hz, 1H), 5.45 (d, J=17.2 Hz, 1H), 4.20 (t, J=5.2 Hz, 2H), 3.69 (t, J=5.2 Hz, 2H), 2.58-2.54 (m, 2H), 2.32 (s, 3H), 1.90 (s, 3H), 1.38-1.33 (m, 4H), 1.23-1.13 (m, 20H).

### N2-(4-(((2-amino-4-oxo-3,4-dihydropteridin-6-yl)methyl)amino)benzoyl)-N5-(12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)-L-glutamine (Compound I-11)

To a solution of (S)-4-(4-(((2-amino-4-hydroxypteridin-6-yl)methyl)amino)benzamido)-5-(tert-butoxy)-5-oxopentanoic acid (1, 1.0 eq, 0.200 g, 0.402 mmol) in dimethylsulfoxide (20 mL), 1-hydroxypyrrolidine-2,5-dione (2.0 eq, 0.0925 g, 0.804 mmol) and N,N′-dicyclohexylmethanediimine (2.0 eq, 0.166 g, 0.804 mmol) were added under nitrogen atmosphere in dark (covered with aluminum foil). The reaction mixture was stirred at room temperature for 16 h. After the completion of reaction, the reaction mixture was filtered through sintered glass filter. To the filtrate, a solution of (6-(1-(2-((12-aminododecyl)oxy)ethyl)-1H-pyrazol-4-yl)-1-(2,5-dimethylbenzyl)-1H-benzo[d]imidazol-2-yl)(pyridin-4-yl)methanol (1.0 eq, 0.25 g, 0.402 mmol) in dimethylsulfoxide (2 mL) followed by triethylamine (2.0 eq, 0.11 mL, 0.081 mmol) were added. The reaction mixture was stirred under dark condition (covered with Aluminum foil) at 35° C. for 16 h. After the completion of reaction, reaction mixture was purified by reverse phase column chromatography (using 40 g C-18 spherical column 30-40% acetonitrile in water). The desired fractions were concentrated under reduced pressure to afford tert-butyl N2-(4-(((2-amino-4-oxo-3,4-dihydropteridin-6-yl)methyl)amino)benzoyl)-N5-(12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)-L-glutaminate (2) as a yellow solid. Yield 0.08 g, 17%; LCMS m/z 1116.7 [M+1]⁺.

To a stirred solution of tert-butyl N2-(4-(((2-amino-4-oxo-3,4-dihydropteridin-6-yl)methyl)amino)benzoyl)-N5-(12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)-L-glutaminate (2, 1.0 eq, 0.08 g, 0.071 mmol) in dichloromethane (2.5 mL) at 0° C., trifluoroacetic acid (0.5 mL) was added and reaction mixture was stirred at room temperature for 3 h. After completion, reaction mixture was concentrated to get crude. The crude was purified by prep-HPLC using (30-40% acetonitrile in water with 0.1% trifluoroacetic acid). Fractions containing the desired product were combined and lyophilized to dryness to afford N2-(4-(((2-amino-4-oxo-3,4-dihydropteridin-6-yl)methyl)amino)benzoyl)-N5-(12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)-L-glutamine (Cpd. No. I-11) as a yellow solid. Yield 0.003 g, 4%; LCMS m/z 531.2 [M+2]⁺+; ¹H NMR (400 MHz, DMSO-d6) δ 8.64 (s, 1H), 8.55 (d, J=5.2 Hz, 2H), 8.19 (d, J=14.8 Hz, 1H), 8.07 (s, 1H), 7.82-7.79 (m, 2H), 7.66-7.63 (m, 3H), 7.59-7.57 (m, 3H), 7.49 (d, J=8.4 Hz, 1H), 7.07-7.04 (m, 3H), 6.86 (d, J=7.2 Hz, 1H), 6.63 (d, J=8.4 Hz, 2H), 6.23 (s, 1H), 5.76 (s, 1H), 5.64 (d, J=16.8 Hz, 1H), 5.52 (d, J=17.2 Hz, 2H), 4.48 (s, 2H), 4.27-4.19 (m, 3H), 3.70-3.69 (m, 3H), 3.31 (t, J=6.0 Hz, 3H), 3.01-2.95 (m, 2H), 2.33 (s, 3H), 2.17-2.15 (m, 2H), 2.04-2.02 (m, 1H), 1.89 (s, 4H), 1.38-1.32 (m, 4H), 1.15-1.11 (m, 17H).

### 6.1.12. Example 12: Compound I-12

### N5-(12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)-N2-(5-(methyl((2-methyl-4-oxo-1,4-dihydroquinazolin-6-yl)methyl)amino)thiophene-2-carbonyl)-L-glutamine (Compound I-12)

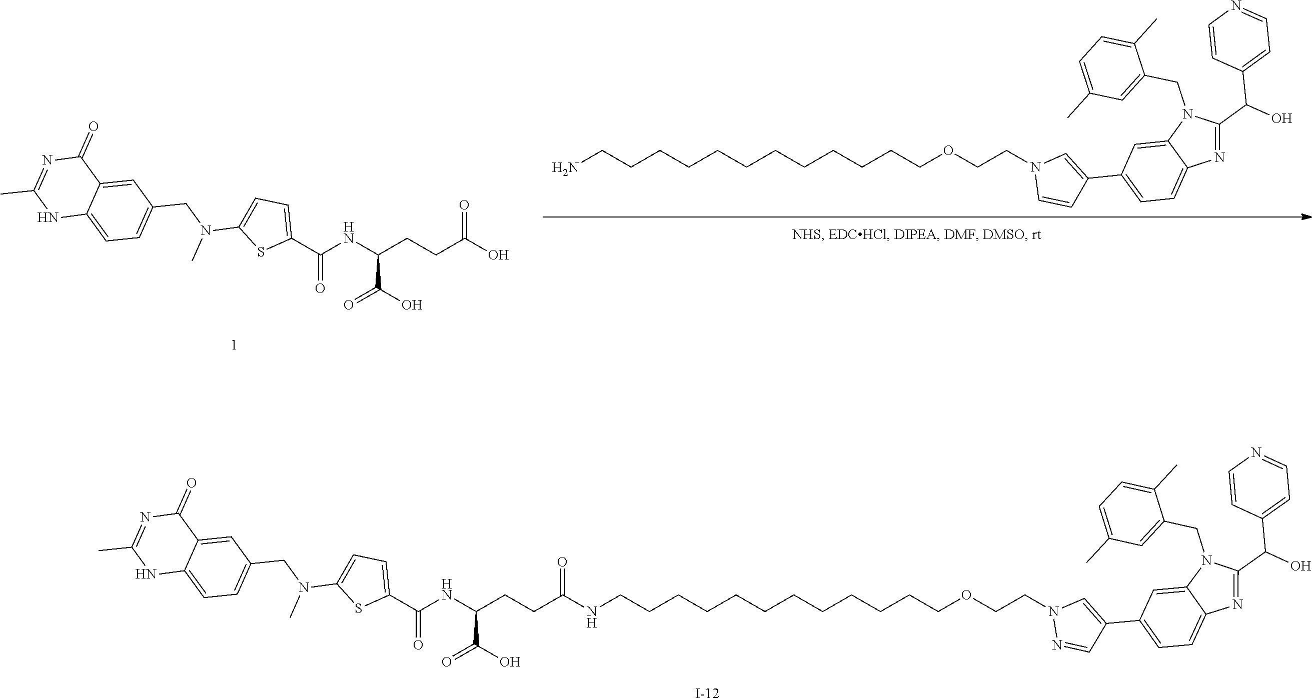

To a solution of (5-(methyl((2-methyl-4-oxo-1,4-dihydroquinazolin-6-yl)methyl)amino)thiophene-2-carbonyl)-L-glutamic acid (1, 1.0 eq, 0.070 g, 0.153 mmol) in N,N-dimethylformamide (1 mL) and dimethyl sulfoxide (1 mL), N-hydroxysuccinimide (1.1 eq, 0.019 g, 0.168 mmol), 1-(3-Dimethylaminopropyl)-3-ethylcarbodiimide hydrochloride (EDC·HCl) (1.1 eq, 0.032 g, 0.168 mmol) and N,N-diisopropylethylamine (3.0 eq, 0.084 mL, 0.458 mmol) were added and reaction mixture was stirred at room temperature for 30 minutes. Then, (6-(1-(2-((12-aminododecyl)oxy)ethyl)-1H-pyrazol-4-yl)-1-(2,5-dimethylbenzyl)-1H-benzo[d]imidazol-2-yl)(pyridin-4-yl)methanol (1.0 eq, 0.097 g, 0.153 mmol) was added and reaction mixture was stirred at room temperature for 16 h. After completion, reaction mixture was directly purified by prep HPLC (30-45% acetonitrile in water with 0.1% TFA). Fractions containing the desired product were combined and lyophilized to dryness to afford N5-(12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)-N2-(5-(methyl((2-methyl-4-oxo-1,4-dihydroquinazolin-6-yl)methyl)amino)thiophene-2-carbonyl)-L-glutamine (Cpd. No. I-12) as a yellow solid. Yield: 0.028 g, 17.0%; LC-MS m/z 539.5 [M+2]⁺+; ¹H NMR (400 MHz, DMSO-d6 with D₂O) δ 8.24 (d, J=5.6 Hz, 2H), 7.90 (s, 1H), 7.85 (s, 1H), 7.72 (s, 1H), 7.61 (d, J=8.4 Hz, 2H), 7.49 (d, J=8.4 Hz, 1H), 7.41-7.39 (m, 2H), 7.28 (s, 1H), 7.22 (d, J=5.6 Hz, 2H), 6.96 (d, J=7.6 Hz, 1H), 6.77 (d, J=7.6 Hz, 1H), 6.04 (s, 1H), 5.87-5.84 (m, 1H), 5.59 (s, 1H), 5.51 (d, J=16.8 Hz, 1H), 5.34 (d, J=16.8 Hz, 1H), 4.53 (s, 2H), 3.63-3.62 (m, 2H), 3.20 (t, J=6.0 Hz, 2H), 2.96 (s, 3H), 2.91 (t, J=6.8 Hz, 2H), 2.29 (s, 3H), 2.21 (s, 3H), 2.12-2.10 (m, 2H), 1.97-1.95 (m, 1H), 1.89 (d, J=4.0 Hz, 1H), 1.86-1.73 (m, 1H), 1.77 (d, J=8.0 Hz, 2H), 1.27-1.21 (m, 5H), 1.04-0.83 (m, 15H).

### 6.1.13. Example 13: Compound I-13

N2-(4-(((2-amino-4-hydroxypteridin-6-yl)methyl)amino)benzoyl)-N5-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-L-glutamine (Compound I-13)

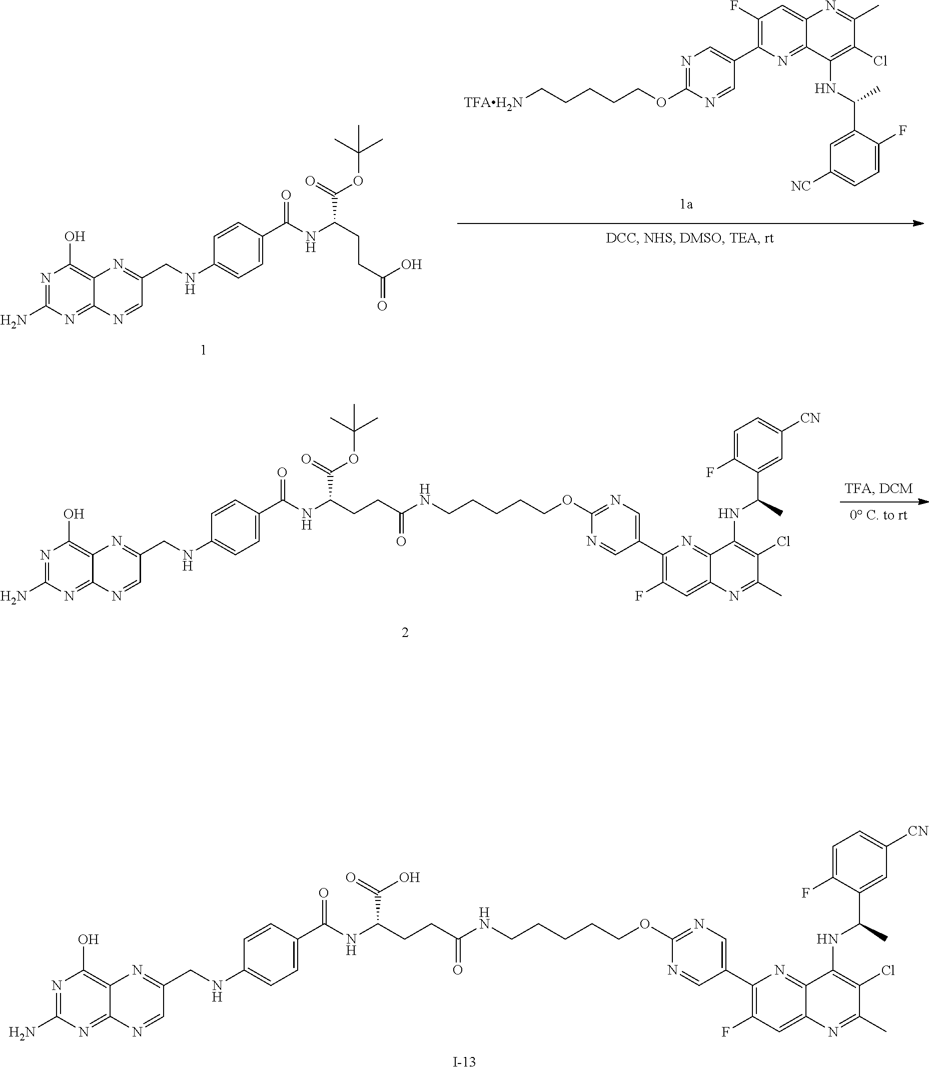

To a solution of (S)-4-(4-(((2-amino-4-hydroxypteridin-6-yl)methyl)amino)benzamido)-5-(tert-butoxy)-5-oxopentanoic acid (1, 1.0 eq, 0.200 g, 0.402 mmol) in dimethylsulfoxide (20 mL), 1-hydroxypyrrolidine-2,5-dione (2.0 eq, 0.0925 g, 0.804 mmol), and N,N′-dicyclohexylmethanediimine (2.0 eq, 0.166 g, 0.804 mmol) were added under nitrogen atmosphere in dark (covered with Aluminum foil). The reaction mixture was stirred at room temperature for 16 h. After completion (monitored by LCMS), the reaction mixture was filtered through sintered. To the filtrate, a solution of (R)-3-(1-((3-chloro-7-fluoro-2-methyl-6-(2-((5-((2,2,2-trifluoroacetyl)-14-azaneyl)pentyl)oxy)pyrimidin-5-yl)-1,5-naphthyridin-4-yl)amino)ethyl)-4-fluorobenzonitrile (TFA salt) (1a, 1.0 eq, 0.25 g, 0.402 mmol) in dimethylsulfoxide (2 mL) followed by triethylamine (4.0 eq, 0.22 mL, 1.61 mmol) were added. The reaction mixture was stirred under dark condition (covered with Aluminium foil) at 35° C. for 16 h. After completion, the reaction mixture was purified by reverse phase column chromatography (using 40 g C-18 spherical column 30-60% acetonitrile in water). The desired fractions were concentrated under reduced pressure to afford tert-butyl N2-(4-(((2-amino-4-hydroxypteridin-6-yl)methyl)amino)benzoyl)-N5-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-L-glutaminate (2) as a yellow solid. Yield: 0.10 g, 24%; LCMS: m/z 1018.5 [M+1]⁺.

To a stirred solution of tert-butyl N2-(4-(((2-amino-4-hydroxypteridin-6-yl)methyl)amino)benzoyl)-N5-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-L-glutaminate (2, 1.0 eq, 0.10 g, 0.098 mmol) in dichloromethane (5 mL) at 0° C., trifluoroacetic acid (1.0 mL) was added and reaction mixture was stirred at room temperature for 3 h. After completion, the reaction mixture was concentrated to get crude product. The crude material was purified by prep-HPLC using (eluting from a C18 column, with 20-60% acetonitrile in water with 0.1% trifluoroacetic acid). Fractions containing desired compound were lyophilized to dryness to afford N2-(4-(((2-amino-4-hydroxypteridin-6-yl)methyl)amino)benzoyl)-N5-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-L-glutamine (Cpd. No. I-13) as a yellow solid. Yield: 0.025 g, 26.4%; LCMS: m/z 481.4 [M+2]⁺+; ¹H NMR (400 MHz, DMSO-d6 with D₂O) δ 8.89 (s, 2H), 8.61 (s, 1H), 8.06 (d, J=10.8 Hz, 1H), 7.90 (d, J=6.0 Hz, 1H), 7.75-7.70 (m, 1H), 7.59 (d, J=8.8 Hz, 2H), 7.24 (t, J=10.0 Hz, 1H), 6.60 (d, J=8.4 Hz, 2H), 6.49-6.46 (m, 1H), 4.44 (s, 2H), 4.34 (t, J=6.4 Hz, 2H), 4.25-4.22 (m, 1H), 3.03-3.01 (m, 2H), 2.67 (s, 3H), 2.19-2.16 (m, 2H), 2.05-2.01 (m, 1H), 1.92-1.90 (m, 1H), 1.73-1.69 (m, 2H), 1.66 (d, J=6.8 Hz, 3H), 1.41-1.36 (m, 4H).

### 6.1.14. Example 14: Compound I-14

N2-(4-(((2-amino-4-hydroxypteridin-6-yl)methyl)amino)benzoyl)-N5-((1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methyl)-L-glutamine (Compound I-14)

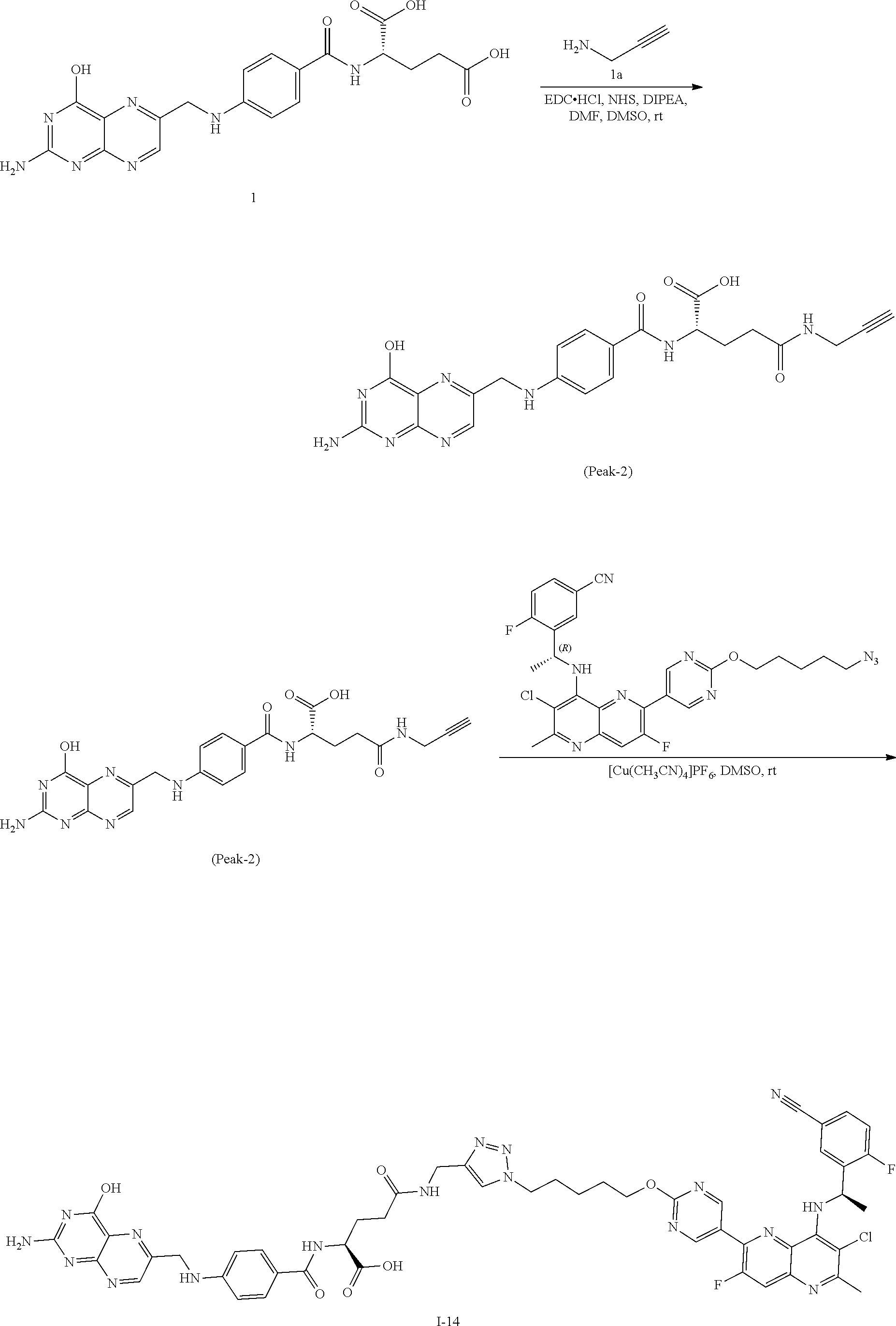

To a suspension of Folic acid (1, 1.0 eq, 2.0 g, 4.53 mmol) in N,N-dimethylformamide (40 mL) and dimethyl sulfoxide (40 mL), N-hydroxysuccinimide (1.5 eq, 0.782 g, 6.80 mmol), 1-(3-Dimethylaminopropyl)-3-ethylcarbodiimide hydrochloride (EDC·HCl) (1.5 eq, 1.3 g, 6.80 mmol) and N,N-diisopropylethylamine (3.0 eq, 2.51 mL, 13.6 mmol) were added and reaction mixture was stirred at room temperature for 30 minutes. Then, prop-2-yn-1-amine (1a, 1.5 eq, 0.374 g, 6.80 mmol) was added and reaction mixture was stirred at room temperature for 16 h. After completion, reaction mixture was directly purified by prep HPLC (10-25% acetonitrile in water with 0.1% acetic acid). Fractions containing the desired product were combined and lyophilized to dryness to afford N2-(4-(((2-amino-4-hydroxypteridin-6-yl)methyl)amino)benzoyl)-N5-(prop-2-yn-1-yl)-L-glutamine (Peak-2) as a yellow solid. Yield: 0.250 g, 11.5%; LCMS m/z 479.3 [M+1]⁺.

To a solution of N2-(4-(((2-amino-4-hydroxypteridin-6-yl)methyl)amino)benzoyl)-N5-(prop-2-yn-1-yl)-L-glutamine (Peak-2, 1.0 eq, 0.030 g, 0.062 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.01 eq, 0.035 g, 0.063 mmol) in dimethylsulfoxide (1.5 mL), tetrakis(acetonitrile)copper(I) hexafluorophosphate (2.8 eq, 0.065 g, 0.176 mmol) was added and reaction mixture was stirred at room temperature for 1 h. After completion, reaction mixture was directly purified by prep HPLC (20-48% acetonitrile in water with 0.1% formic acid). Fractions containing the desired product were combined and lyophilized to dryness to afford N2-(4-(((2-amino-4-hydroxypteridin-6-yl)methyl)amino)benzoyl)-N5-((1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methyl)-L-glutamine (Cpd. No. I-14) as a yellow solid. Yield: 0.015 g, 22.9%; LCMS m/z 1042.3 [M+1]⁺; ¹H-NMR (400 MHz, DMSO-d6 with D₂O) δ 9.00 (s, 2H), 8.61 (s, 1H), 8.11 (d, J=11.6 Hz, 1H), 7.96 (dd, J=1.6 Hz, 6.8 Hz, 1H), 7.86 (s, 1H), 7.76-7.73 (m, 1H), 7.62 (d, J=8.8 Hz, 2H), 7.27 (d, J=10.0 Hz, 1H), 6.62 (d, J=8.8 Hz, 2H), 6.35 (d, J=7.6 Hz, 1H), 4.45 (s, 2H), 4.38 (t, J=6.4 Hz, 2H), 4.33-4.20 (m, 5H), 2.63 (s, 3H), 2.50-2.49 (m, 1H), 2.24-2.20 (m, 2H), 2.08-2.06 (m, 1H), 1.92-1.75 (m, 5H), 1.64 (d, J=6.8 Hz, 3H), 1.39-1.35 (m, 2H).

### 6.1.15. Example 15: Compound I-15

N2-(4-(((2-amino-4-hydroxypteridin-6-yl)methyl)amino)benzoyl)-N5-(2-((1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol 4-yl)methoxy)ethyl)-L-glutamine (Compound I-15)

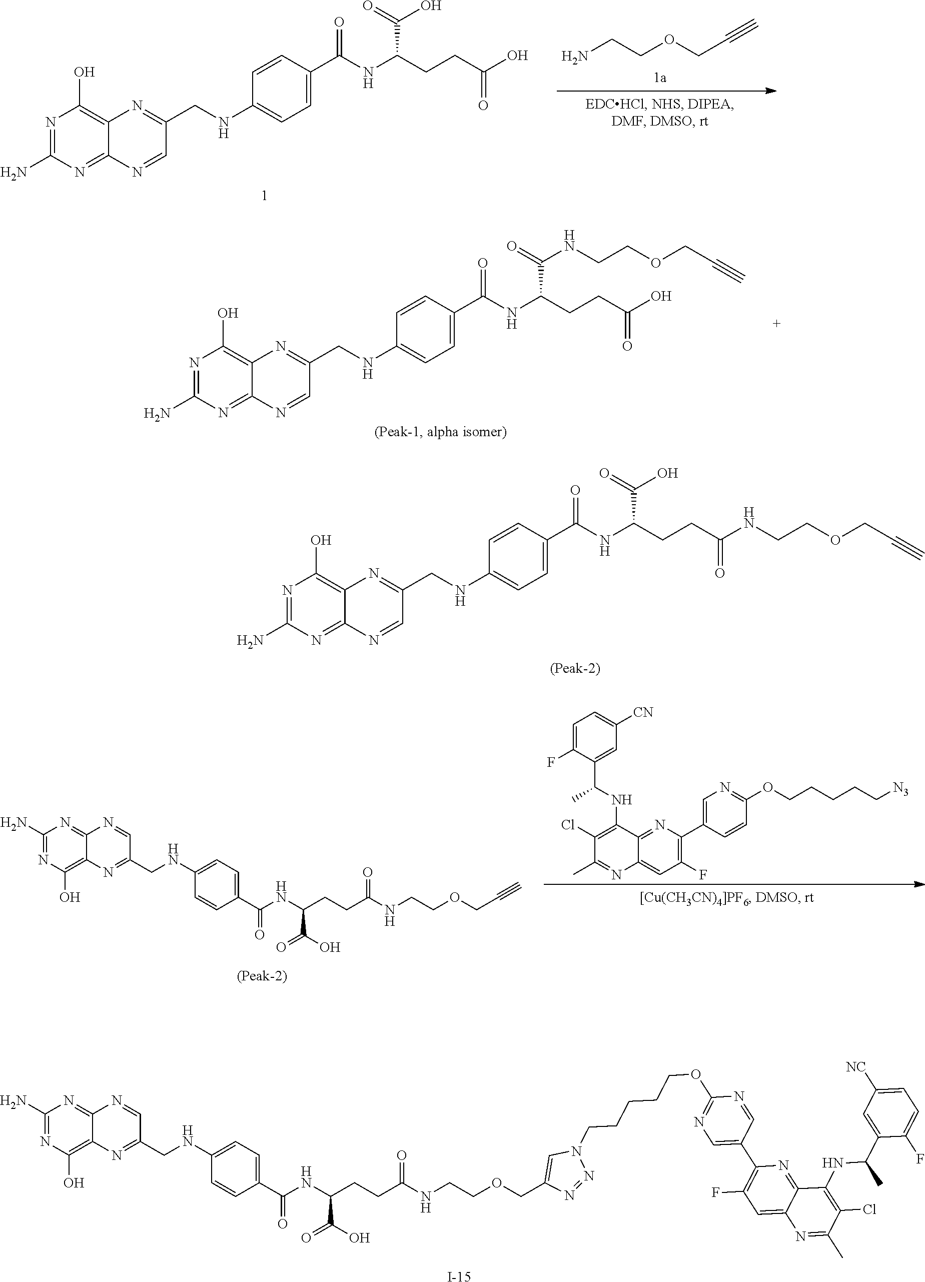

To a suspension of (4-(((2-amino-4-hydroxypteridin-6-yl)methyl)amino)benzoyl)-L-glutamic acid (Folic acid) (1, 1.0 eq, 1.0 g, 2.27 mmol) in N,N-dimethylformamide (20 mL) and dimethyl sulfoxide (20 mL), N-hydroxysuccinimide (1.1 eq, 0.287 g, 2.49 mmol), 1-(3-Dimethylaminopropyl)-3-ethylcarbodiimide hydrochloride (EDC·HCl) (1.1 eq, 0.478 g, 2.49 mmol) and N,N-diisopropylethylamine (3.0 eq, 1.25 mL, 6.80 mmol) were added and reaction mixture was stirred at room temperature for 30 minutes. Then, 2-(prop-2-yn-1-yloxy)ethan-1-amine (1a, 1.0 eq, 0.225 g, 2.27 mmol) was added and reaction mixture was stirred at room temperature for 16 h. After completion, reaction mixture was directly purified by prep HPLC (10-24% acetonitrile in water with 0.1% TFA). Fractions containing the desired product were combined and lyophilized to dryness to afford (S)-4-(4-(((2-amino-4-hydroxypteridin-6-yl)methyl)amino)benzamido)-5-oxo-5-((2-(prop-2-yn-1-yloxy)ethyl)amino)pentanoic acid (Peak-1, alpha isomer) as a yellow solid. Yield: 0.040 g, 3.3%; LCMS m/z 523.32 [M+1]⁺ and N2-(4-(((2-amino-4-hydroxypteridin-6-yl)methyl)amino)benzoyl)-N5-(2-(prop-2-yn-1-yloxy)ethyl)-L-glutamine (Peak-2) as a yellow solid. Yield: 0.065 g, 5.4%; LCMS m/z 523.3 [M+1]⁺.

To a solution of N2-(4-(((2-amino-4-hydroxypteridin-6-yl)methyl)amino)benzoyl)-N5-(2-(prop-2-yn-1-yloxy)ethyl)-L-glutamine (Peak-2, 1.0 eq, 0.040 g, 0.076 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.043 g, 0.076 mmol) in dimethylsulfoxide (1.0 mL), tetrakis(acetonitrile)copper(I) hexafluorophosphate (2.8 eq, 0.079 g, 0.214 mmol) was added and reaction mixture was stirred at room temperature for 1 h. After completion, reaction mixture was directly purified by prep HPLC (22-33% acetonitrile in water with 0.1% TFA). Fractions containing the desired product were combined and lyophilized to dryness to afford N2-(4-(((2-amino-4-hydroxypteridin-6-yl)methyl)amino)benzoyl)-N5-(2-((1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol 4-yl)methoxy)ethyl)-L-glutamine (Cpd. No. I-15) as a yellow solid. Yield: 0.024 g, 28.4%; LCMS m/z 1086.3 [M+1]⁺; ¹H NMR (400 MHz, DMSO-d6 with D₂O) δ 8.88 (s, 2H), 8.61 (s, 1H), 8.05 (d, J=10.4 Hz, 1H), 8.00 (s, 1H), 7.89-7.88 (s, 1H), 7.72 (bs, 1H), 7.58 (d, J=8.4 Hz, 2H), 7.22 (t, J=9.6 Hz, 1H), 6.59 (d, J=6.0 Hz, 2H), 6.49-6.48 (m, 1H), 4.44 (s, 4H), 4.37-4.33 (m, 4H), 4.24-4.20 (m, 1H), 3.37 (t, J=6.0 Hz, 2H), 3.17-3.16 (m, 2H), 2.67 (s, 3H), 2.16-2.14 (m, 2H), 2.01-1.98 (m, 1H), 1.88-1.83 (m, 3H), 1.75-1.73 (m, 2H), 1.66 (d, J=6.4 Hz, 3H), 1.35-1.33 (m, 2H).

### 6.1.16. Example 16: Compound I-16

N2-(4-(((2-amino-4-hydroxypteridin-6-yl)methyl)amino)benzoyl)-N5-(2-(2-((1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methoxy)ethoxy)ethyl)-L-glutamine (Compound I-16)

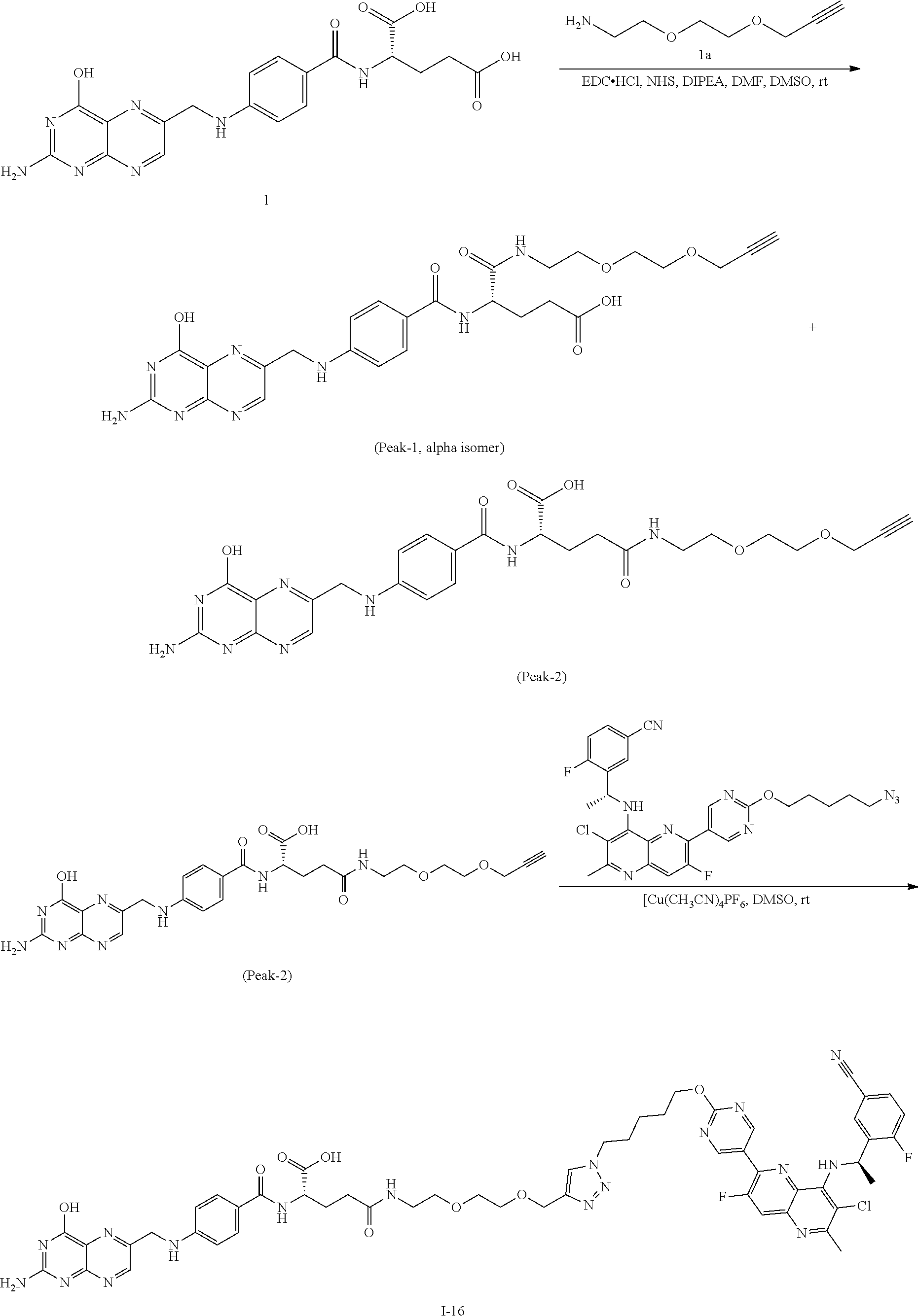

To a suspension of (4-(((2-amino-4-hydroxypteridin-6-yl)methyl)amino)benzoyl)-L-glutamic acid (Folic acid) (1, 1.0 eq, 1.0 g, 2.27 mmol) in N,N-dimethylformamide (20 mL) and dimethyl sulfoxide (20 mL), N-hydroxysuccinimide (1.1 eq, 0.287 g, 2.49 mmol), 1-(3-Dimethylaminopropyl)-3-ethylcarbodiimide hydrochloride (EDC·HCl) (1.1 eq, 0.478 g, 2.49 mmol) and N,N-diisopropylethylamine (3.0 eq, 1.25 mL, 6.80 mmol) were added and reaction mixture was stirred at room temperature for 30 minutes. Then, 2-(2-(prop-2-yn-1-yloxy)ethoxy)ethan-1-amine (1a, 1.0 eq, 0.324 g, 2.27 mmol) was added and reaction mixture was stirred at room temperature for 16 h. After completion, reaction mixture was directly purified by prep HPLC (10-27% acetonitrile in water with 0.1% acetic acid). Fractions containing the desired product were combined and lyophilized to dryness to afford (S)-4-(4-(((2-amino-4-hydroxypteridin-6-yl)methyl)amino)benzamido)-5-oxo-5-((2-(2-(prop-2-yn-1-yloxy)ethoxy)ethyl)amino)pentanoic acid (Peak-1, alpha isomer) as a yellow solid. Yield: 0.165 g, 12.8%; LCMS m/z 567.40 [M+1]⁺. and N2-(4-(((2-amino-4-hydroxypteridin-6-yl)methyl)amino)benzoyl)-N5-(2-(2-(prop-2-yn-1-yloxy)ethoxy)ethyl)-L-glutamine (Peak-2) as a yellow solid. Yield: 0.233 g, 18.1%; LCMS m/z 567.44 [M+1]⁺.

To a solution of N2-(4-(((2-amino-4-hydroxypteridin-6-yl)methyl)amino)benzoyl)-N5-(2-(2-(prop-2-yn-1-yloxy)ethoxy)ethyl)-L-glutamine (Peak-2, 1.0 eq, 0.040 g, 0.070 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.039 g, 0.070 mmol) in dimethyl sulfoxide (1.0 mL), tetrakis(acetonitrile)copper(i) hexafluorophosphate (2.8 eq, 0.073 g, 0.198 mmol) was added and reaction mixture was stirred at room temperature for 1 h. After completion, reaction mixture was directly purified by prep HPLC (25-35% acetonitrile in water with 0.1% TFA). Fractions containing the desired product were combined and lyophilized to dryness to afford N2-(4-(((2-amino-4-hydroxypteridin 6-yl)methyl)amino)benzoyl)-N5-(2-(2-((1-(5-((5-(7-chloro-8-(((R)-1-(5-cyano-2-fluorophenyl)ethyl)amino)-3-fluoro-6-methyl-1,5-naphthyridin-2-yl)pyrimidin 2-yl)oxy)pentyl)-1H-1,2,3-triazol-4-yl)methoxy)ethoxy)ethyl)-L-glutamine (Cpd. No. I-16) as a yellow solid. Yield: 0.018 g, 22.0%; LCMS m/z 1130.99 [M+1]⁺; ¹H NMR (400 MHz, DMSO-d6 with D₂O) δ 8.89 (s, 2H), 8.62 (s, 1H), 8.08-8.03 (m, 2H), 7.90 (d, J=6.8 Hz, 1H), 7.74-7.72 (m, 1H), 7.58 (d, J=8.8 Hz, 2H), 7.23 (t, J=9.6 Hz, 1H), 6.60 (d, J=8.4 Hz, 2H), 6.50 (d, J=7.2 Hz, 1H), 4.46 (s, 4H), 4.35 (d, J=6.4 Hz, 4H), 4.23-4.20 (m, 1H), 3.46 (d, J=9.6 Hz, 4H), 3.32 (t, J=5.2 Hz, 2H), 3.13 (t, J=6.8 Hz, 2H), 2.69 (s, 3H), 2.16-2.15 (m, 2H), 2.06-1.99 (m, 1H), 1.88-1.84 (m, 3H), 1.78-1.76 (m, 2H), 1.66 (d, J=6.8 Hz, 3H), 1.36-1.34 (m, 2H).

### 6.1.17. Example 17: Compound I-17

### (S)-18-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-2,5,8,11-tetraoxa-14-azanonadecan-19-oic acid (Compound I-17)

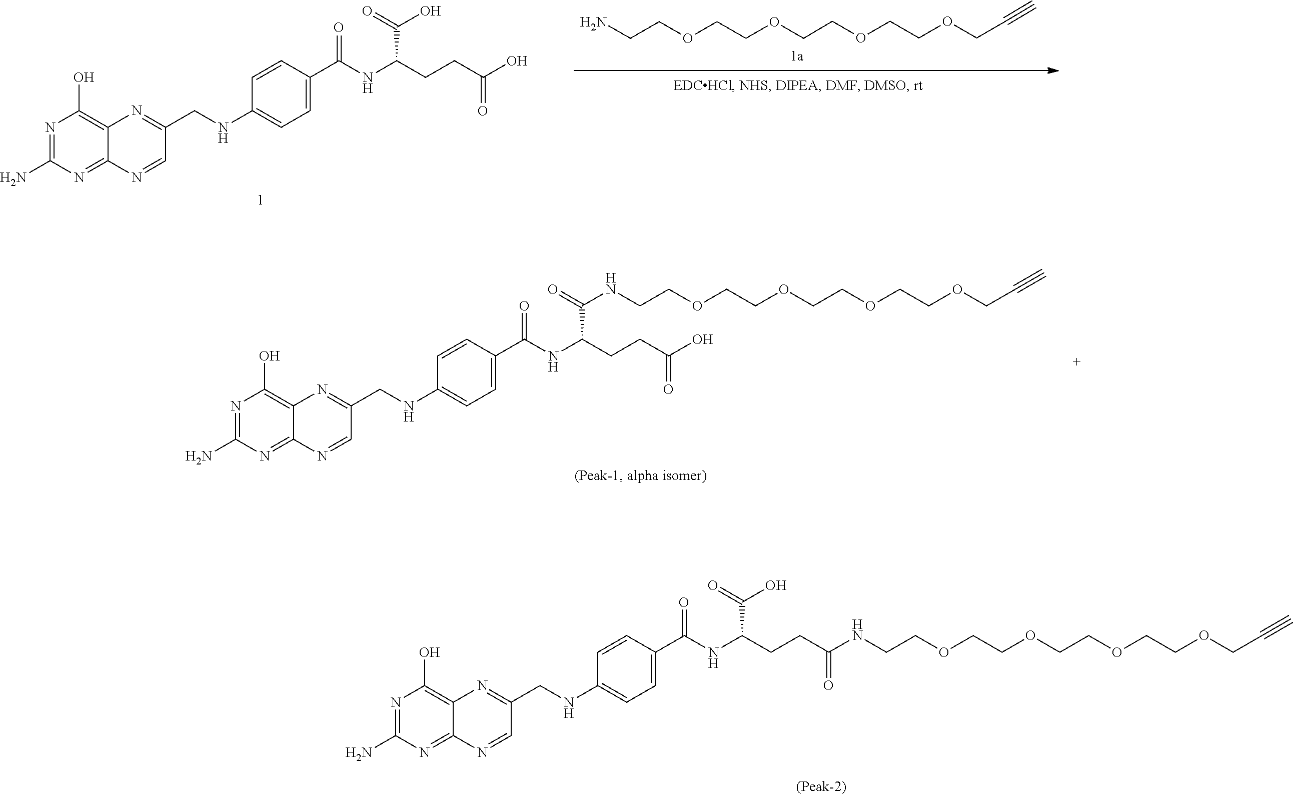 [SMILES: Nc1nc(O)c2nc(CNc3ccc(C(=O)N[C@@H](CCC(=O)O)C(=O)O)cc3)cnc2n1 ; C#CCOCCOCCOCCOCCN ; C#CCOCCOCCOCCOCCNC(=O)[C@H](CCC(=O)O)NC(=O)c1ccc(NCc2cnc3nc(N)nc(O)c3n2)cc1 ; C#CCOCCOCCOCCOCCNC(=O)CC[C@H](NC(=O)c1ccc(NCc2cnc3nc(N)nc(O)c3n2)cc1)C(=O)O]

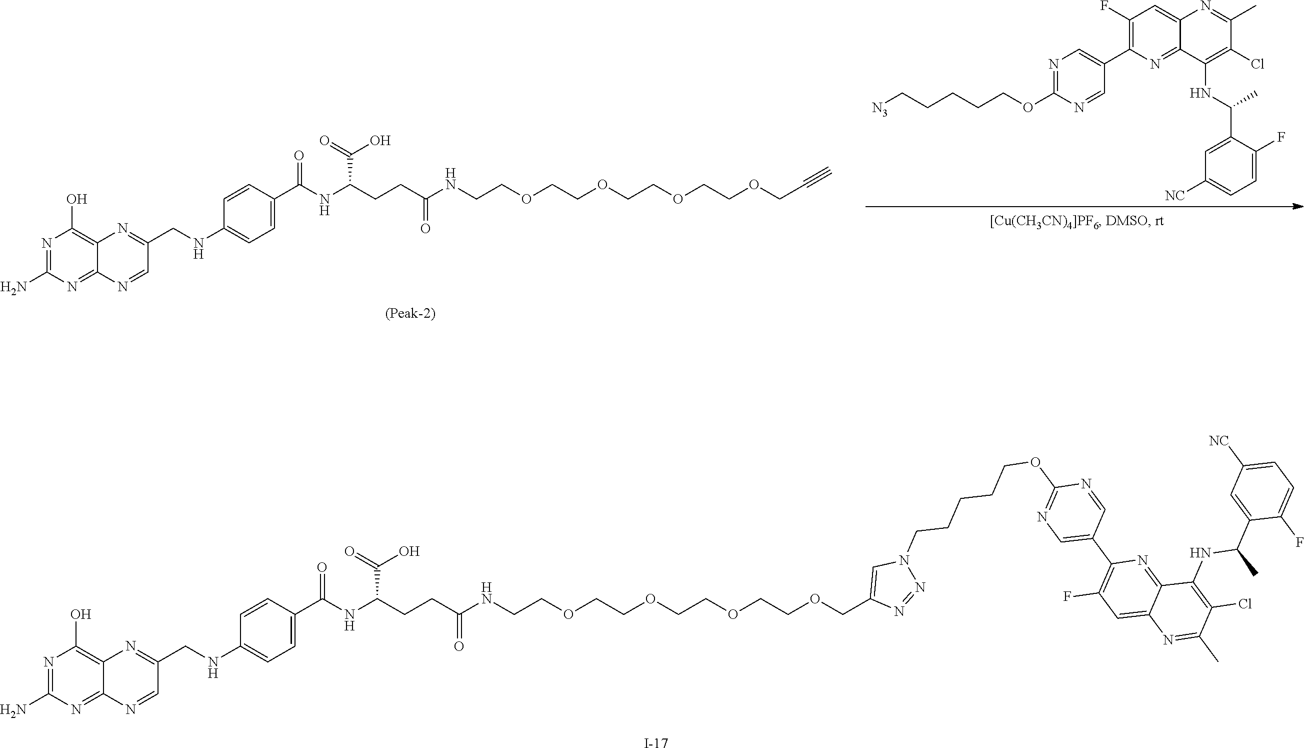

To a suspension of (4-(((2-amino-4-hydroxypteridin-6-yl)methyl)amino)benzoyl)-L-glutamic acid (Folic acid) (1, 1.0 eq, 0.850 g, 1.93 mmol) in N,N-dimethylformamide (20 mL) and dimethyl sulfoxide (20 mL), N-hydroxysuccinimide (1.5 eq, 0.332 g, 2.89 mmol), 1-(3-Dimethylaminopropyl)-3-ethylcarbodiimide hydrochloride (EDC·HCl) (1.5 eq, 0.554 g, 2.89 mmol) and N,N-diisopropylethylamine (3.0 eq, 1.07 mL, 5.78 mmol) were added and reaction mixture was stirred at room temperature for 1 h. Then, 3,6,9,12-tetraoxapentadec-14-yn-1-amine (1a, 1.5 eq, 0.668 g, 2.89 mmol) was added and reaction mixture was stirred at room temperature for 16 h. After completion, reaction mixture was directly purified by prep HPLC (10-27% acetonitrile in water with 0.1% acetic acid). Fractions containing the desired product were combined and lyophilized to dryness to afford (S)-18-(4-(((2-amino-4-hydroxypteridin-6-yl)methyl)amino)benzamido)-17-oxo-4,7,10,13-tetraoxa-16-azahenicos-1-yn-21-oic acid (Peak-1, alpha isomer) as a yellow solid. Yield: 0.270 g, 21.4%; LCMS m/z 655.6 [M+1]⁺ and (S)-20-(4-(((2-amino-4-hydroxypteridin-6-yl)methyl)amino)benzamido)-17-oxo-4,7,10,13-tetraoxa-16-azahenicos-1-yn-21-oic acid (Peak-2) as a yellow solid. Yield: 0.260 g, 20.6%; LCMS m/z 655.3 [M+1]⁺.

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (Peak-2, 1.0 eq, 0.040 g, 0.061 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.034 g, 0.061 mmol) in dimethylsulfoxide (1.0 mL), tetrakis(acetonitrile)copper(I) hexafluorophosphate (2.8 eq, 0.063 g, 0.171 mmol) was added and reaction mixture was stirred at room temperature for 1 h. After completion, reaction mixture was directly purified by prep HPLC (20-35% acetonitrile in water with 0.1% TFA). Fractions containing the desired product were combined and lyophilized to dryness to afford (S)-18-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-2,5,8,11-tetraoxa-14-azanonadecan-19-oic acid (Cpd. No. I-17) as a yellow solid. Yield: 0.044 g, 59.4%; LCMS m/z 1218.6 [M+1]⁺; ¹H NMR (400 MHz, DMSO-d6 with D₂O) δ 8.85 (s, 2H), 8.62 (s, 1H), 8.06-8.02 (m, 2H), 7.87 (d, J=4.8 Hz, 1H), 7.73-7.70 (m, 1H), 7.57 (d, J=8.8 Hz, 2H), 7.22 (t, J=9.6 Hz, 1H), 6.59 (d, J=8.8 Hz, 2H), 6.52 (d, J=6.8 Hz, 1H), 4.46 (s, 4H), 4.35-4.32 (m, 4H), 4.24-4.20 (m, 1H), 3.49-3.44 (m, 4H), 3.41 (d, J=5.6 Hz, 8H), 3.31 (t, J=6.0 Hz, 2H), 3.13-3.12 (m, 2H), 2.67 (s, 3H), 2.16 (t, J=6.8 Hz, 2H), 1.99-1.98 (m, 1H), 1.86 (t, J=6.8 Hz, 3H), 1.77 (t, J=6.4 Hz, 2H), 1.66 (d, J=6.4 Hz, 3H), 1.37-1.35 (m, 2H).

### 6.1.18. Example 18: Compound I-18

(S)-24-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-21-oxo-2,5,8,11,14,17-hexaoxa-20-azapentacosan-25-oic acid (Compound I-18)

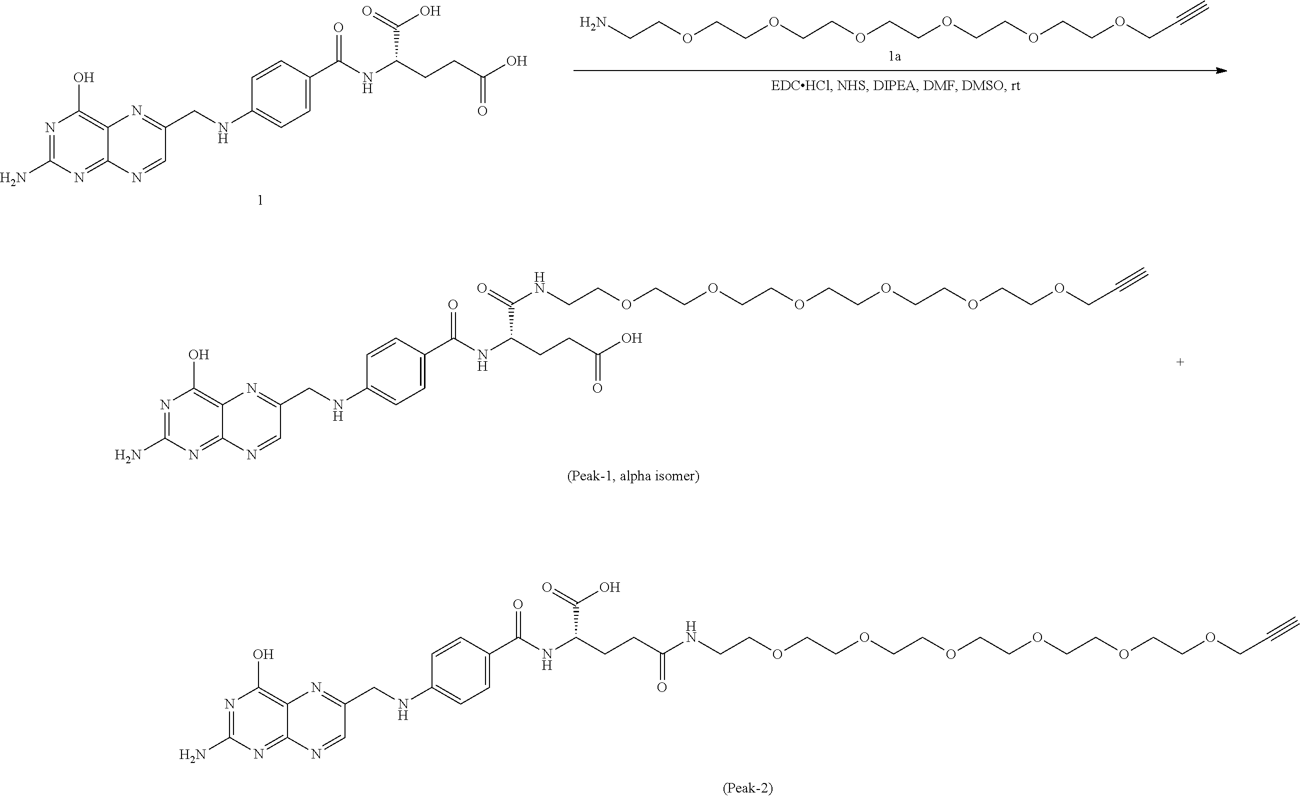 [SMILES: Nc1nc(O)c2nc(CNc3ccc(C(=O)N[C@@H](CCC(=O)O)C(=O)O)cc3)cnc2n1 ; C#CCOCCOCCOCCOCCOCCOCCN ; C#CCOCCOCCOCCOCCOCCOCCNC(=O)[C@H](CCC(=O)O)NC(=O)c1ccc(NCc2cnc3nc(N)nc(O)c3n2)cc1 ; C#CCOCCOCCOCCOCCOCCOCCNC(=O)CC[C@H](NC(=O)c1ccc(NCc2cnc3nc(N)nc(O)c3n2)cc1)C(=O)O]

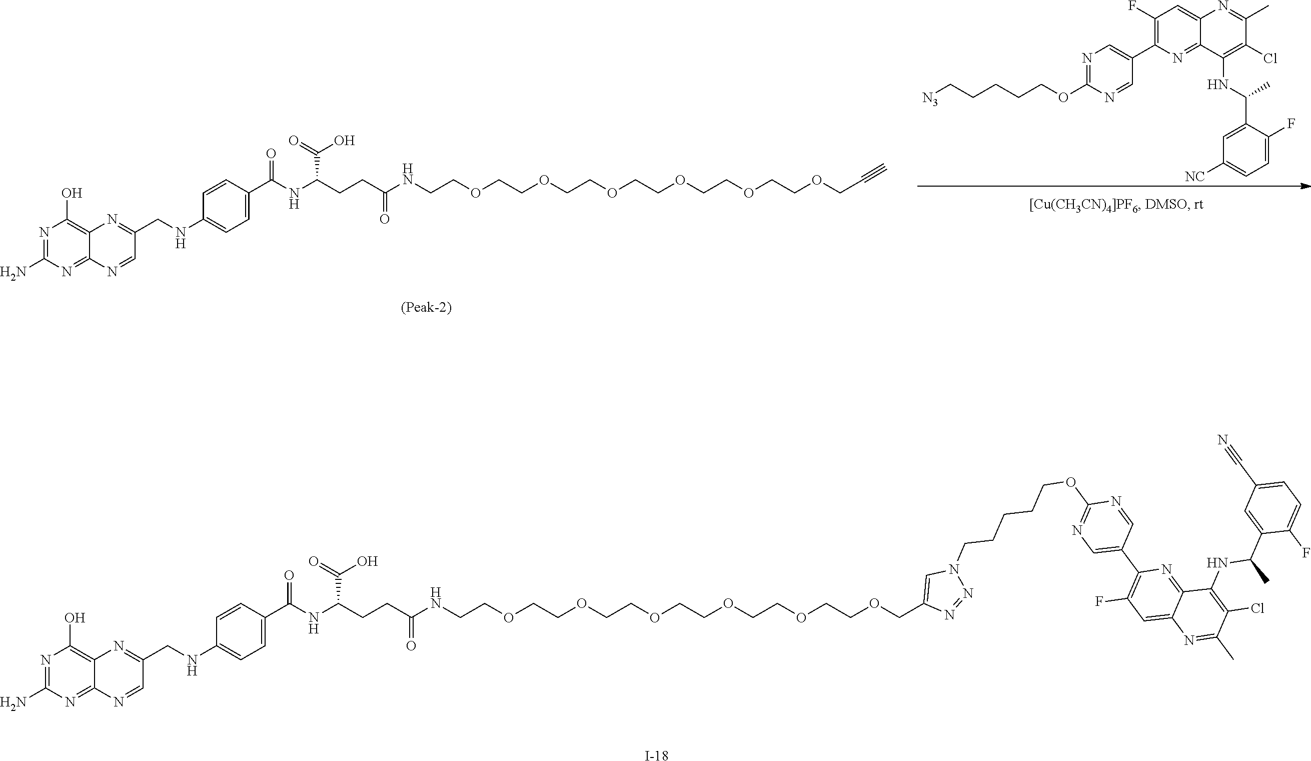

To a suspension of (4-(((2-amino-4-hydroxypteridin-6-yl)methyl)amino)benzoyl)-L-glutamic acid (Folic acid) (1, 1.0 eq, 0.500 g, 1.13 mmol) in N,N-dimethylformamide (10 mL) and dimethyl sulfoxide (10 mL), N-hydroxysuccinimide (1.1 eq, 0.143 g, 1.25 mmol), 1-(3-Dimethylaminopropyl)-3-ethylcarbodiimide hydrochloride (EDC·HCl) (1.1 eq, 0.239 g, 1.25 mmol) and N,N-diisopropylethylamine (3.0 eq, 0.62 mL, 3.40 mmol) were added and reaction mixture was stirred at room temperature for 30 minutes. Then, 3,6,9,12,15,18-hexaoxahenicos-20-yn-1-amine (1a, 1.0 eq, 0.362 g, 1.13 mmol) was added and reaction mixture was stirred at room temperature for 16 h. After completion, reaction mixture was directly purified by prep HPLC (10-30% acetonitrile in water with 0.1% acetic acid). Fractions containing the desired product were combined and lyophilized to dryness to afford (S)-24-(4-(((2-amino-4-hydroxypteridin-6-yl)methyl)amino)benzamido)-23-oxo-4,7,10,13,16,19-hexaoxa-22-azaheptacos-1-yn-27-oic acid (Peak-1, alpha isomer) as a yellow solid. Yield: 0.150 g, 17.83%; LCMS m/z 743.38 [M+1]⁺ and (S)-26-(4-(((2-amino-4-hydroxypteridin-6-yl)methyl)amino)benzamido)-23-oxo-4,7,10,13,16,19-hexaoxa-22-azaheptacos-1-yn-27-oic acid (Peak-2) as a yellow solid. Yield: 0.170 g, 20.2%; LCMS m/z 743.3 [M+1]⁺.

To a solution of (S)-26-(4-(((2-amino-4-hydroxypteridin-6-yl)methyl)amino)benzamido)-23-oxo-4,7,10,13,16,19-hexaoxa-22-azaheptacos-1-yn-27-oic aciPeak-2, 1.0 eq, 0.040 g, 0.053 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.030 g, 0.053 mmol) in dimethylsulfoxide (1.0 mL), tetrakis(acetonitrile)copper(I) hexafluorophosphate (2.8 eq, 0.056 g, 0.151 mmol) was added and reaction mixture was stirred at room temperature for 1 h. After completion, reaction mixture was directly purified by prep HPLC (22-33% acetonitrile in water with 0.1% TFA). Fractions containing the desired product were combined and lyophilized to dryness to afford (S)-24-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-21-oxo-2,5,8,11,14,17-hexaoxa-20-azapentacosan-25-oic acid (Cpd. No. I-18) as a yellow solid. Yield: 0.025 g, 35.7%; LCMS m/z 654.0 [M+2]⁺+; ¹H NMR (400 MHz, DMSO-d6 with D₂O) δ 8.79 (s, 2H), 8.61 (s, 1H), 8.02 (d, J=12.0 Hz, 2H), 7.83 (d, J=12.0 Hz, 1H), 7.69 (bs, 1H), 7.55 (d, J=8.0 Hz, 2H), 7.19 (t, J=9.6 Hz, 1H), 6.58 (d, J=8.0 Hz, 3H), 4.45 (s, 4H), 4.34 (bs, 4H), 4.22-4.20 (m, 1H), 3.47-3.45 (m, 20H), 3.31 (bs, 2H), 3.12 (bs, 2H), 2.68 (s, 3H), 2.17-2.15 (m, 2H), 1.99 (bs, 1H), 1.86-1.85 (m, 3H), 1.74-1.72 (m, 2H), 1.66 (d, J=6.4 Hz, 3H), 1.32 (bs, 2H).

### 6.1.19. Example 19: Compound I-19

(S)-30-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-27-oxo-2,5,8,11,14,17,20,23-octaoxa-26-azahentriacontan-31-oic acid (Compound I-19)

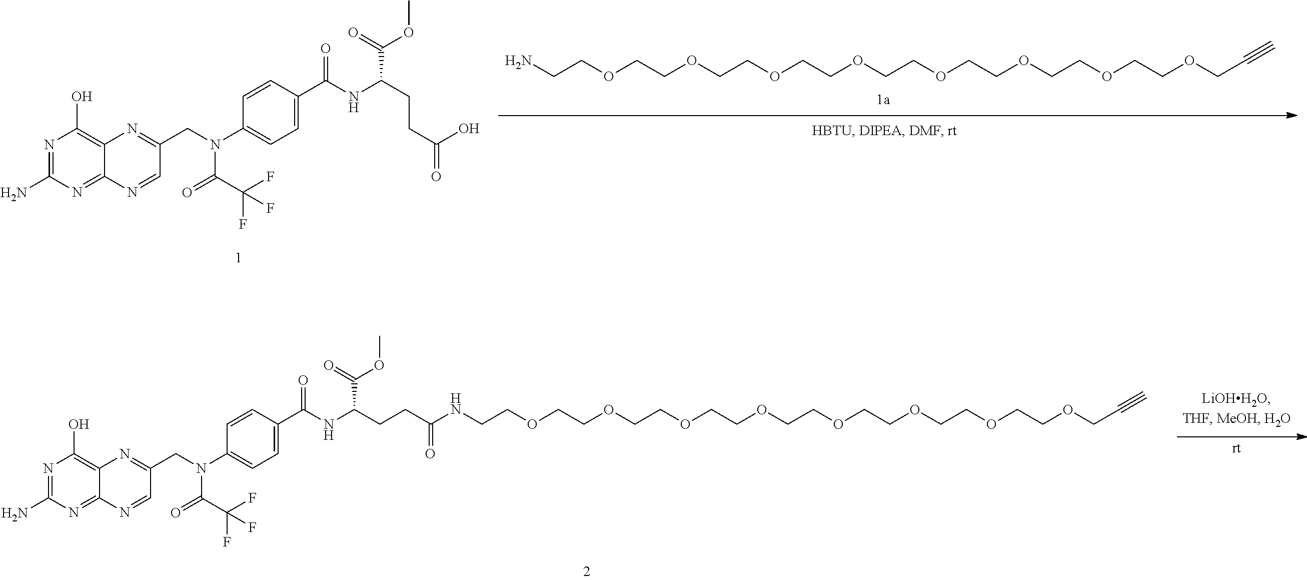 [SMILES: COC(=O)[C@H](CCC(=O)O)NC(=O)c1ccc(N(Cc2cnc3nc(N)nc(O)c3n2)C(=O)C(F)(F)F)cc1 ; C#CCOCCOCCOCCOCCOCCOCCOCCOCCN ; C#CCOCCOCCOCCOCCOCCOCCOCCOCCNC(=O)CC[C@H](NC(=O)c1ccc(N(Cc2cnc3nc(N)nc(O)c3n2)C(=O)C(F)(F)F)cc1)C(=O)OC]

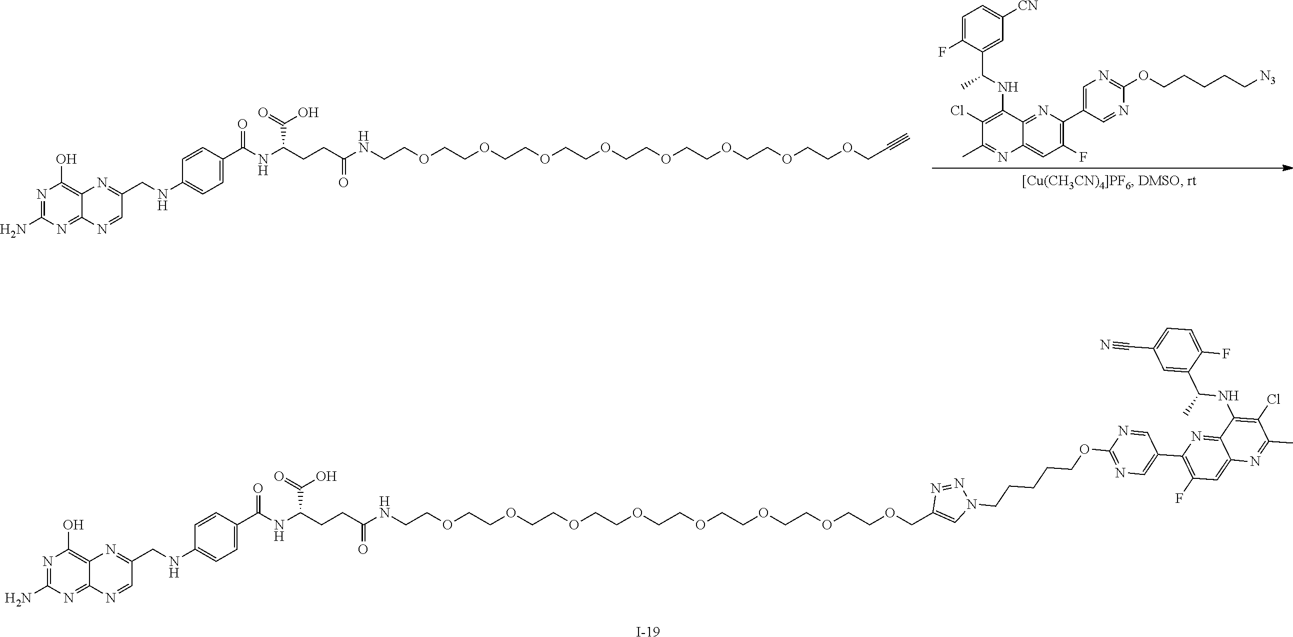

To a solution of (S)-4-(4-(N-((2-amino-4-hydroxypteridin-6-yl)methyl)-2,2,2-trifluoroacetamido)benzamido)-5-methoxy-5-oxopentanoic acid (1, 1.0 eq, 0.31 g, 0.562 mmol) in N,N-dimethylformamide (6 mL), 3,6,9,12,15,18,21,24-octaoxaheptacos-26-yn-1-amine (1a, 1.0 eq, 0.229 g, 0.562 mmol), 2-(1H-benzotriazol-1-yl)-1,1,3,3-tetramethyluronium hexafluorophosphate (HBTU) (1.2 eq, 0.266 g, 0.674 mmol) and N,N-diisopropylethyl amine (1.5 eq, 0.14 mL, 0.843 mmol) were added. The reaction mixture was stirred at room temperature under nitrogen atmosphere in dark (covered with aluminum foil) for 16 h. After the completion of reaction, reaction mixture was purified with reverse phase column chromatography (using 40 g C-18 spherical column eluting 30-60% acetonitrile in water). The desired fractions were concentrated under reduced pressure to afford methyl (S)-32-(4-(N-((2-amino-4-hydroxypteridin-6-yl)methyl)-2,2,2-trifluoroacetamido)benzamido)-29-oxo-4,7,10,13,16,19,22,25-octaoxa-28-azatritriacont-1-yn-33-oate (2) as a yellow solid. Yield 0.320 g, 60%; LCMS m/z 941.28 [M+1]⁺.

To a stirred solution of methyl (S)-32-(4-(N-((2-amino-4-hydroxypteridin-6-yl)methyl)-2,2,2-trifluoroacetamido)benzamido)-29-oxo-4,7,10,13,16,19,22,25-octaoxa-28-azatritriacont-1-yn-33-oate (2, 1.0. eq, 0.250 g, 0.265 mmol) in tetrahydrofuran:methanol:water (1:1:1:) (6 mL), lithium hydroxide monohydrate (10.0 eq, 0.11 g, 2.65 mmol) was added and reaction mixture was stirred for at room temperature 16 h. After completion, reaction mixture was concentrated to get crude. The crude was purified by prep-HPLC using 20-60% acetonitrile in water with 0.1% trifluoroacetic acid). The desired fractions were lyophilized to afford (S)-32-(4-(((2-amino-4-hydroxypteridin-6-yl)methyl)amino)benzamido)-29-oxo-4,7,10,13,16,19,22,25-octaoxa-28-azatritriacont-1-yn-33-oic acid as a yellow solid. Yield 0.092 g, 41%; LCMS m/z 831.19 [M+1]⁺.

To a solution of (S)-32-(4-(((2-amino-4-hydroxypteridin-6-yl)methyl)amino)benzamido)-29-oxo-4,7,10,13,16,19,22,25-octaoxa-28-azatritriacont-1-yn-33-oic acid (1.0 eq, 0.044 g, 0.053 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.05 eq, 0.031 g, 0.055 mmol) in dimethylsulfoxide (1.5 mL), tetrakis(acetonitrile)copper(I) hexafluorophosphate (2.8 eq, 0.055 g, 0.148 mmol) was added and reaction mixture was stirred at room temperature for 0.5 h. After completion, reaction mixture was quenched with acetic acid (0.15 mL) and directly purified by prep HPLC (eluting from C18 column with 30-70% acetonitrile in water with 0.1% trifluoroacetic acid). Fractions containing the desired product were combined and lyophilized to dryness to afford (S)-30-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-27-oxo-2,5,8,11,14,17,20,23-octaoxa-26-azahentriacontan-31-oic acid (Cpd. No. I-19) as a yellow solid. Yield: 0.035 g, 47%; LCMS m/z 698.2 [M+2]⁺+; ¹H-NMR (400 MHz, DMSO-d6 with D₂O) δ 8.98 (s, 2H), 8.66 (s, 1H), 8.15 (d, J=10.8 Hz, 1H), 8.08 (s, 1H), 7.99-7.97 (m, 1H), 7.79-7.76 (m, 1H), 7.63 (d, J=8.4 Hz, 2H), 7.32-7.27 (m, 1H), 6.63 (d, J=8.8 Hz, 2H), 6.49-6.45 (m, 1H), 4.49 (s, 4H), 4.41-4.36 (m, 4H), 4.27-4.23 (m, 1H), 3.52-3.49 (m, 4H), 3.47-3.45 (m, 24H), 3.36-3.33 (m, 2H), 3.16-3.13 (m, 2H), 2.67 (s, 3H), 2.20-2.16 (m, 2H), 2.10-1.92 (m, 1H), 1.93-1.86 (m, 3H), 1.82-1.78 (m, 2H), 1.68 (d, J=6.8 Hz, 3H), 1.41-1.37 (m, 2H).

### 6.1.20. Example 20: Compound I-20

(S)-36-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-33-oxo-2,5,8,11,14,17,20,23,26,29-decaoxa-32-azaheptatriacontan-37-oic acid (Compound I-20)

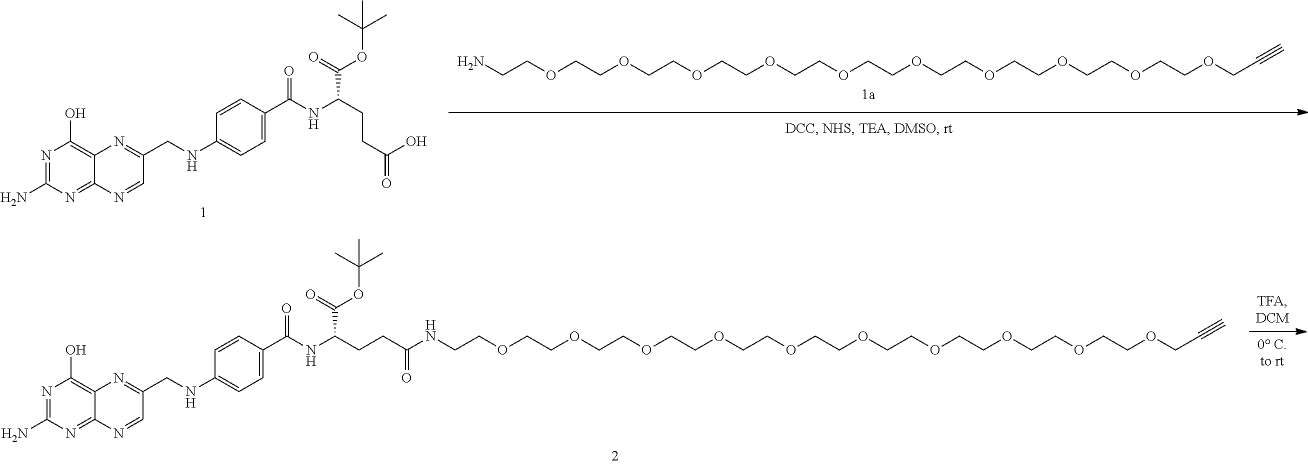 [SMILES: CC(C)(C)OC(=O)[C@H](CCC(=O)O)NC(=O)c1ccc(NCc2cnc3nc(N)nc(O)c3n2)cc1 ; C#CCOCCOCCOCCOCCOCCOCCOCCOCCOCCOCCN ; C#CCOCCOCCOCCOCCOCCOCCOCCOCCOCCOCCNC(=O)CC[C@H](NC(=O)c1ccc(NCc2cnc3nc(N)nc(O)c3n2)cc1)C(=O)OC(C)(C)C]

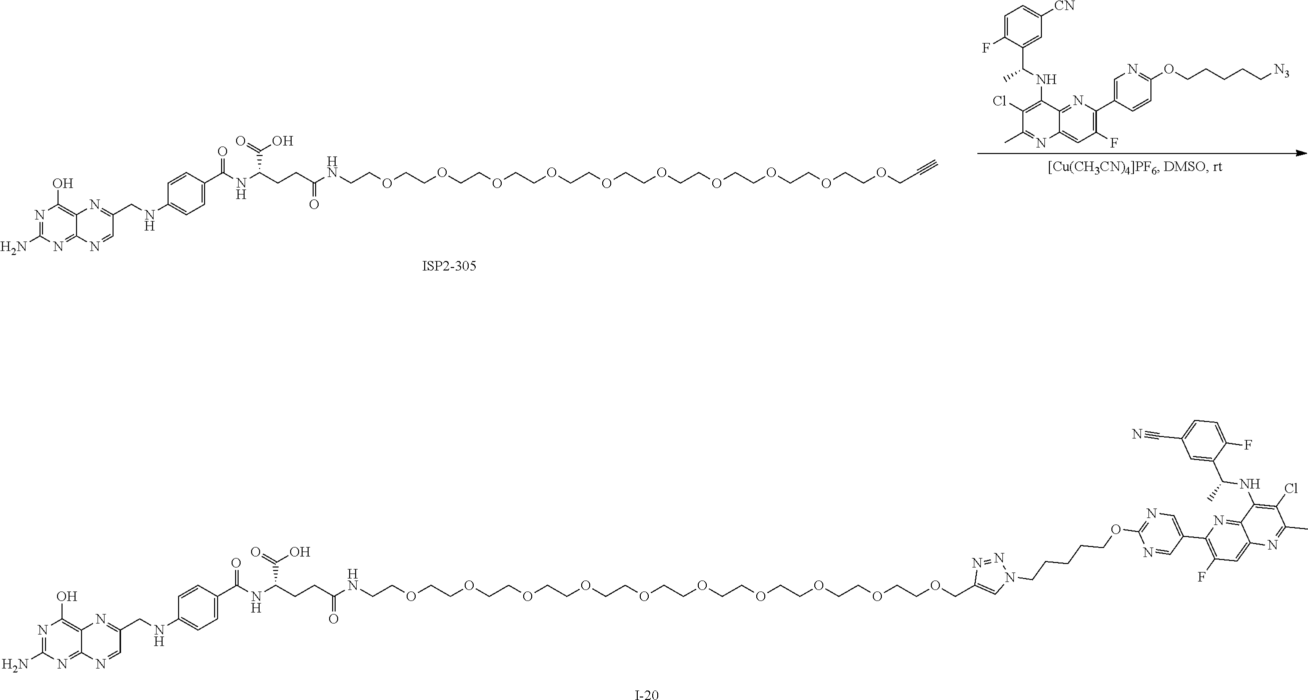

To a solution of (S)-4-(4-(((2-amino-4-hydroxypteridin-6-yl)methyl)amino)benzamido)-5-(tert-butoxy)-5-oxopentanoic acid (1, 1.0 eq, 0.200 g, 0.402 mmol) in dimethylsulfoxide (20 mL), 1-hydroxypyrrolidine-2,5-dione (2.0 eq, 0.0925 g, 0.804 mmol), N,N′-dicyclohexylmethanediimine (2.0 eq, 0.166 g, 0.804 mmol) were added under nitrogen atmosphere in dark (covered with Aluminium foil). The reaction mixture was stirred at room temperature for 16 h. After the completion of reaction, reaction mixture was filtered through sintered glass funnel. To the filtrate solution, 3,6,9,12,15,18,21,24,27,30-decaoxatritriacont-32-yn-1-amine (1a, 1.0 eq, 0.20 g, 0.402 mmol) in dimethylsulfoxide (2 mL) followed by triethyl amine (2.0 eq, 0.11 mL, 0.804 mmol) were added. The reaction mixture was stirred at 35° C. for 16 h. After the completion of reaction, reaction mixture was purified with reverse phase column chromatography (using 40 g C-18 spherical column eluting 30-60% acetonitrile in water). The desired fractions were concentrated under reduced pressure to afford tert-butyl (S)-38-(4-(((2-amino-4-hydroxypteridin-6-yl)methyl)amino)benzamido)-35-oxo-4,7,10,13,16,19,22,25,28,31-decaoxa-34-azanonatriacont-1-yn-39-oate (2) as a yellow solid. Yield 0.200 g, 51%; LCMS m/z 975.62 [M+1]⁺.

To a stirred solution of tert-butyl (S)-38-(4-(((2-amino-4-hydroxypteridin-6-yl)methyl)amino)benzamido)-35-oxo-4,7,10,13,16,19,22,25,28,31-decaoxa-34-azanonatriacont-1-yn-39-oate (2, 1.0 eq, 0.20 g, 0.205 mmol) in dichloromethane (10 mL) at 0° C., trifluroacetic acid (2.0 mL) was added and reaction mixture was stirred at room temperature for 3 h. After completion, reaction mixture was concentrated. The crude was purified by prep-HPLC using 20-60% acetonitrile in water with 0.1% triethylamine. The desired fractions were lyophilized to afford (S)-38-(4-(((2-amino-4-hydroxypteridin-6-yl)methyl)amino)benzamido)-35-oxo-4,7,10,13,16,19,22,25,28,31-decaoxa-34-azanonatriacont-1-yn-39-oic acid as a yellow solid. Yield 0.025 g, 13.6%; LCMS m/z 919.70 [M+1]⁺.

To a solution of (S)-38-(4-(((2-amino-4-hydroxypteridin-6-yl)methyl)amino)benzamido)-35-oxo-4,7,10,13,16,19,22,25,28,31-decaoxa-34-azanonatriacont-1-yn-39-oic acid (1.0 eq, 0.023 g, 0.025 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.01 eq, 0.014 g, 0.025 mmol) in dimethylsulfoxide (1.5 mL), tetrakis(acetonitrile)copper(I) hexafluorophosphate (2.8 eq, 0.026 g, 0.070 mmol) was added and reaction mixture was stirred at room temperature for 0.5 h. After completion, reaction mixture was quenched with acetic acid (0.15 mL) and directly purified by prep HPLC (30-70% acetonitrile in water with 0.1% trifluoroacetic acid). Fractions containing the desired product were combined and lyophilized to dryness to afford (S)-36-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-33-oxo-2,5,8,11,14,17,20,23,26,29-decaoxa-32-azaheptatriacontan-37-oic acid (Cpd. No. I-20) as a yellow solid. Yield: 0.013 g, 35%; LCMS m/z 742.25 [M+2]⁺+; ¹H NMR (400 MHz, DMSO-d6 with D₂O) δ 8.81 (s, 2H), 8.58 (s, 1H), 7.79 (d, J=14.8 Hz, 2H), 7.80 (d, J=5.6 Hz, 1H), 7.65 (t, J=4.4 Hz, 1H), 7.54 (d, J=8.4 Hz, 2H), 7.16 (t, J=10.0 Hz, 1H), 6.58 (d, J=8.8 Hz, 2H), 6.43 (t, J=5.6 Hz, 1H), 4.44 (d, J=2.0 Hz, 4H), 4.33 (d, J=6.4 Hz, 4H), 3.47-3.40 (m, 35H), 3.31 (t, J=9.2 Hz, 2H), 3.13 (d, J=5.2 Hz, 2H), 2.64 (d, J=16.4 Hz, 3H), 2.17 (t, J=7.2 Hz, 2H), 2.01-1.99 (m, 2H), 1.93-1.85 (m, 4H), 1.75-1.72 (m, 2H), 1.63-1.62 (m, 3H), 1.39-1.31 (m, 2H).

### 6.1.21. Example 21: Compound I-21

### (S)-16-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-2,5,8,11-tetraoxa-14-azanonadecan-19-oic acid (Compound I-21)

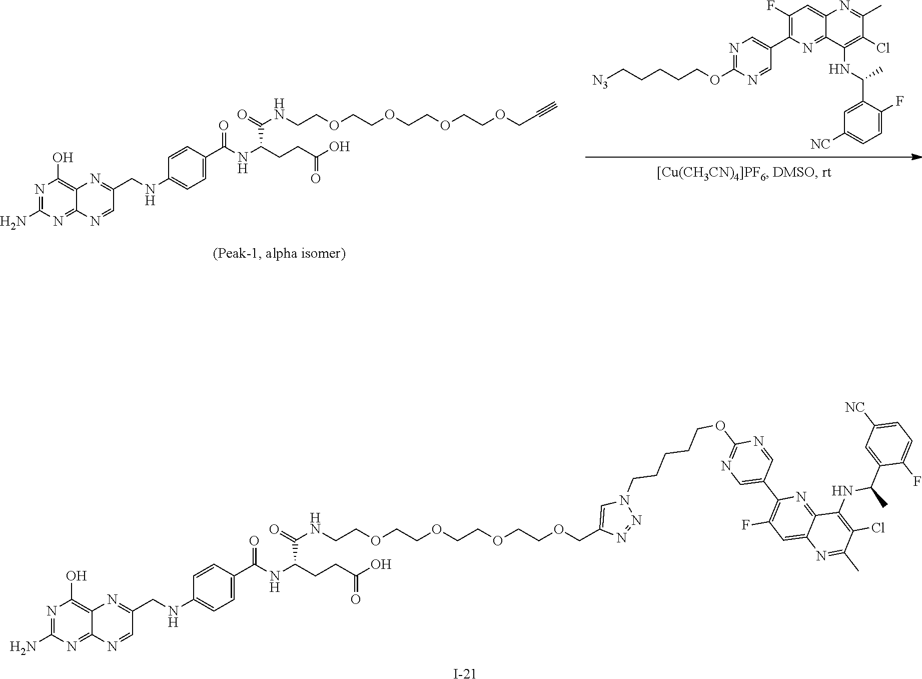

To a solution of (S)-18-(4-(((2-amino-4-hydroxypteridin-6-yl)methyl)amino)benzamido)-17-oxo-4,7,10,13-tetraoxa-16-azahenicos-1-yn-21-oic acid (Peak-1, alpha isomer, 1.0 eq, 0.040 g, 0.061 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.034 g, 0.061 mmol) in dimethylsulfoxide (1.0 mL), tetrakis(acetonitrile)copper(I) hexafluorophosphate (2.8 eq, 0.063 g, 0.171 mmol) was added and reaction mixture was stirred at room temperature for 1 h. After completion, reaction mixture was directly purified by prep HPLC (20-35% acetonitrile in water with 0.1% TFA). Fractions containing the desired product were combined and lyophilized to dryness to afford (S)-16-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-2,5,8,11-tetraoxa-14-azanonadecan-19-oic acid (Cpd. No. I-21) as a yellow solid. Yield: 0.020 g, 27.0%; LCMS m/z 610.0 [M+1]⁺; ¹H NMR (400 MHz, DMSO-d6 with D₂O) δ 8.82 (s, 2H), 8.60 (s, 1H), 8.02-8.00 (m, 2H), 7.84 (d, J=7.6 Hz, 1H), 7.69 (bs, 1H), 7.55 (d, J=8.0 Hz, 2H), 7.19 (t, J=9.6 Hz, 1H), 6.57 (d, J=8.8 Hz, 2H), 6.53-6.48 (m, 1H), 4.44 (t, J=10.0 Hz, 3H), 4.35-4.29 (m, 5H), 3.47-3.34 (m, 12H), 3.21-3.09 (m, 3H), 2.66 (s, 3H), 2.23-2.21 (m, 2H), 1.91-1.85 (m, 6H), 1.74 (bs, 2H), 1.65 (d, J=6.4 Hz, 3H), 1.31 (bs, 2H).

### 6.1.22. Example 22: Compound I-22

### Synthesis of rac-(R)-18-(4-(((2-amino-4-hydroxypteridin-6-yl)methyl)amino)benzamido)-1-(1-(2-(2-(4-(5-(3-(2-(difluoromethoxy)benzyl)-2-methylimidazo[1,2-a]pyridin-6-yl)pyridin-2-yl)piperazin-1-yl)ethoxy)ethyl)-1H-1,2,3-triazol-4-yl)-15-oxo-2,5,8,11-tetraoxa-14-azanonadecan-19-oic acid (Compound I-22)

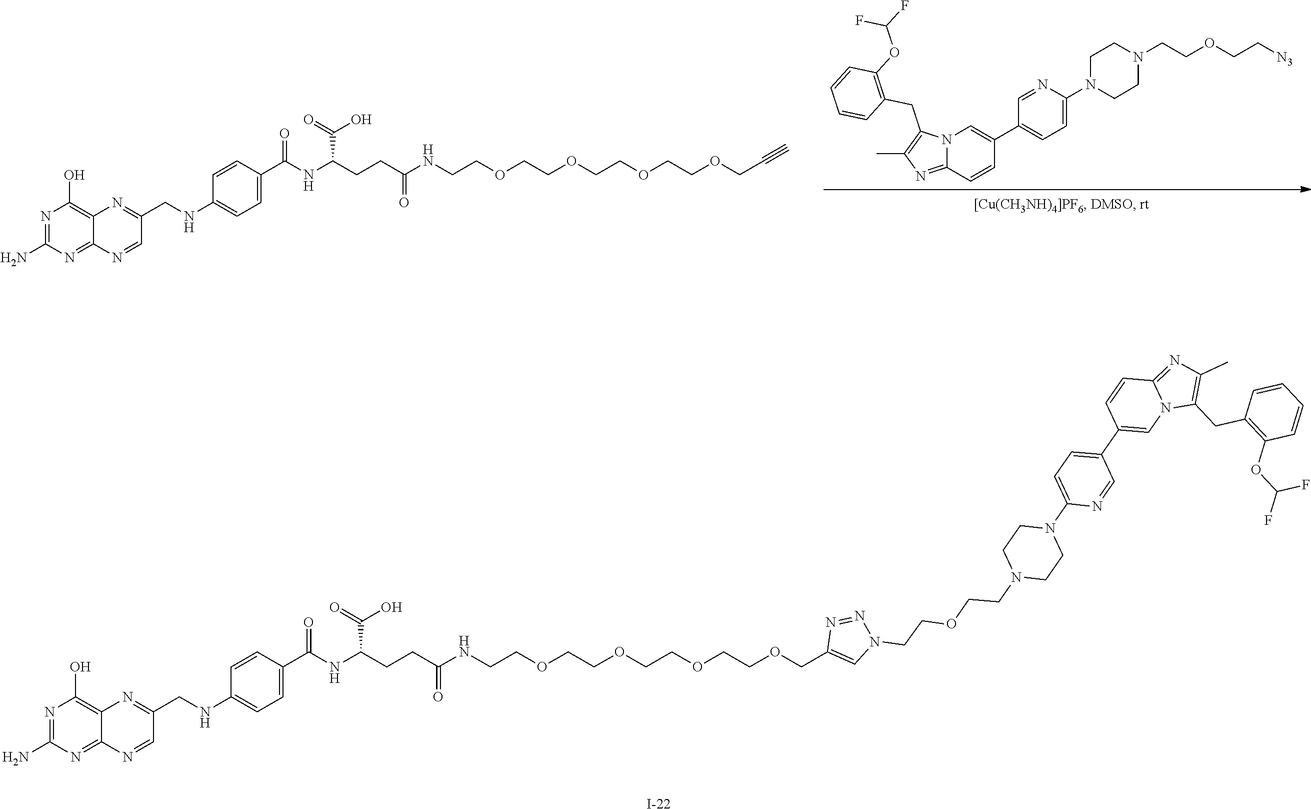

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq, 0.030 g, 0.0458 mmol) and 6-(6-(4-(2-(2-azidoethoxy)ethyl)piperazin-1-yl)pyridin-3-yl)-3-(2-(difluoromethoxy)benzyl)-2-methylimidazo[1,2-a]pyridine (1.0 eq, 0.0258 g, 0.0458 mmol) in anhydrous dimethylsulfoxide (1.5 mL), tetrakis(acetonitrile)copper(I) hexafluorophosphate (2.8 eq, 0.0478 g, 0.128 mmol) was added and reaction mixture was stirred at room temperature for 0.5 h. After completion, the reaction mixture was quenched with acetic acid (0.2 mL) and directly purified by prep HPLC (15-35% acetonitrile in water with 0.1% trifluoroacetic acid). All the fractions containing desired compound were combined and lyophilized to afford rac-(R)-18-(4-(((2-amino-4-hydroxypteridin-6-yl)methyl)amino)benzamido)-1-(1-(2-(2-(4-(5-(3-(2-(difluoromethoxy)benzyl)-2-methylimidazo[1,2-a]pyridin-6-yl)pyridin-2-yl)piperazin-1-yl)ethoxy)ethyl)-1H-1,2,3-triazol-4-yl)-15-oxo-2,5,8,11-tetraoxa-14-azanonadecan-19-oic acid (Cpd. No. I-22) as a yellow solid. Yield: 0.017 g, 30.48%; LCMS m/z 1217.97 [M+1]⁺; ¹H NMR (400 MHz, DMSO-d6 with D₂O) δ 8.73 (s, 1H), 8.61 (s, 1H), 8.51 (d, J=2.4 Hz, 1H), 8.16 (d, J=9.2 Hz, 1H), 8.08 (s, 1H), 7.97 (dd, J=7.6 and 2.4 Hz, 1H), 7.91 (d, J=9.2 Hz, 1H), 7.60 (d, J=8.4 Hz, 2H), 7.36-6.96 (m, 6H), 6.61 (d, J=8.8 Hz, 2H), 4.56-4.55 (m, 2H), 4.49-4.46 (m, 6H), 4.47-4.32 (m, 1H), 4.26-4.22 (m, 1H), 3.86 (t, J=4.8 Hz, 2H), 3.53-3.52 (m, 2H), 3.47-3.46 (m, 2H), 3.40-3.39 (m, 9H), 3.32-3.29 (m, 4H), 3.25-3.18 (m, 2H), 3.13-3.05 (m, 3H), 3.09-2.95 (m, 3H), 2.39 (s, 3H), 2.18-2.15 (m, 2H), 2.02-2.00 (m, 1H), 1.90-1.86 (m, 1H).

### 6.1.23. Example 23: Compound I-23

(S)-18-(4-(((2-amino-4-hydroxypteridin-6-yl)methyl)amino)benzamido)-1-(1-(4-(5-(3-chloro-4-((2,5-dimethylphenyl)amino)-2-methylquinolin-6-yl)picolinamido)butyl)-1H-1,2,3-triazol-4-yl)-15-oxo-2,5,8,11-tetraoxa-14-azanonadecan-19-oic acid. (Compound I-23)

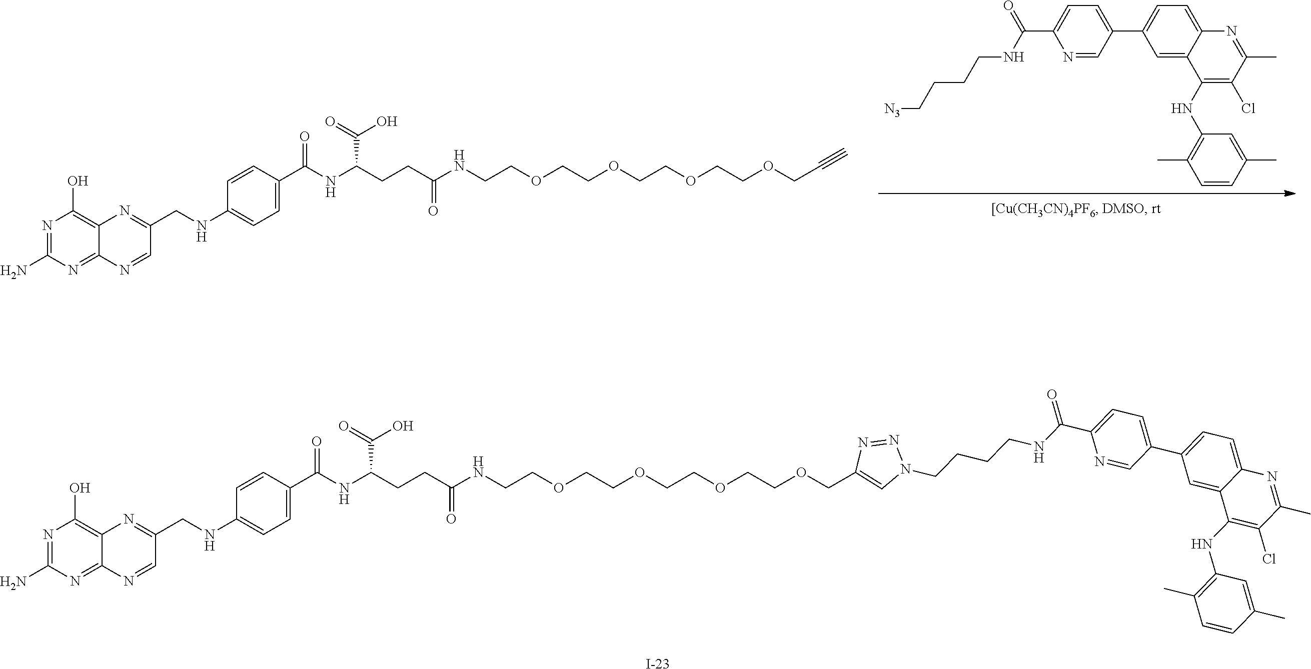

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq, 0.03 g, 0.0458 mmol) and N-(4-azidobutyl)-5-(3-chloro-4-((2,5-dimethylphenyl)amino)-2-methylquinolin-6-yl)picolinamide (1.0 eq, 0.0236 g, 0.0458 mmol) in dimethylsulfoxide (1.5 mL), tetrakis(acetonitrile)copper(I) hexafluorophosphate (2.8 eq, 0.0478 g, 0.128 mmol) was added. The reaction mixture was stirred at room temperature for 1 h and reaction mixture was directly purified by prep HPLC (15-37% acetonitrile in water with 0.1% trifluoroacetic acid). Fractions containing the desired product were combined and lyophilized to dryness to afford (S)-18-(4-(((2-amino-4-hydroxypteridin-6-yl)methyl)amino)benzamido)-1-(1-(4-(5-(3-chloro-4-((2,5-dimethylphenyl)amino)-2-methylquinolin-6-yl)picolinamido)butyl)-1H-1,2,3-triazol-4-yl)-15-oxo-2,5,8,11-tetraoxa-14-azanonadecan-19-oic acid (Cpd. No. I-23) as a yellow solid. Yield: 0.019 g; 35.48%. LCMS: m/z 585.21 [M+2]⁺+; ¹H NMR (400 MHz, DMSO-d6 with D₂O) δ 8.68 (s, 1H), 8.62 (s, 1H), 8.30 (d, J=9.2 Hz, 1H), 8.09 (s, 1H), 8.06-7.99 (m, 3H), 7.94 (d, J=7.2 Hz, 1H), 7.60 (d, J=7.2 Hz, 2H), 7.30 (d, J=8 Hz, 1H), 7.21 (d, J=7.6 Hz, 1H), 7.09 (s, 1H), 6.60 (d, J=8.4 Hz, 2H), 4.46 (s, 4H), 4.35 (t, J=6.8 Hz, 2H), 4.25-4.21 (m, 1H), 3.48 (d, J=7.6 Hz, 4H), 3.42-3.41 (m, 8H), 3.32-3.30 (m, 4H), 3.13-3.11 (m, 2H), 2.75 (s, 3H), 2.24 (s, 3H), 2.17-2.14 (m, 5H), 2.03-2.00 (m, 2H), 1.90-1.81 (m, 4H), 1.49-1.47 (m, 2H).

### 6.1.24. Example 24: Compound I-24

### (S)-18-(4-(((2-amino-4-hydroxypteridin-6-yl)methyl)amino)benzamido)-1-(1-(2-(2-((5-(1-(2-(difluoromethoxy)benzyl)-2-methyl-1H-benzo[d]imidazol-6-yl)pyrimidin-2-yl)oxy)ethoxy)ethyl)-1H-1,2,3-triazol-4-yl)-15-oxo-2,5,8,11-tetraoxa-14-azanonadecan-19-oic acid (Compound I-24)

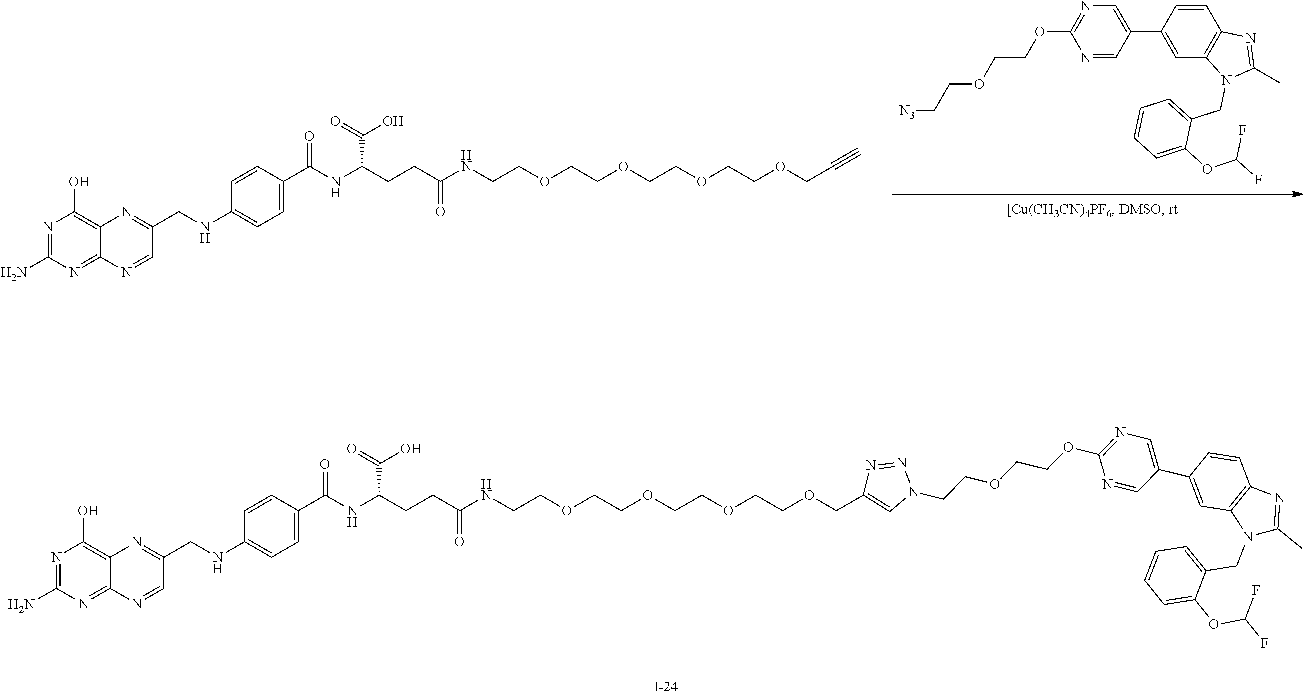

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq, 0.03 g, 0.0458 mmol) and 6-(2-(2-(2-azidoethoxy)ethoxy)pyrimidin-5-yl)-1-(2-(difluoromethoxy)benzyl)-2-methyl-1H-benzo[d]imidazole (1.0 eq, 0.0227 g, 0.0458 mmol) in dimethylsulfoxide (1.5 mL), tetrakis(acetonitrile)copper(I) hexafluorophosphate (2.8 eq, 0.0478 g, 0.128 mmol) was added. The reaction mixture was stirred at room temperature for 1 h and reaction mixture was directly purified by prep HPLC (20-42% acetonitrile in water with 0.1% trifluoroacetic acid). Fractions containing the desired product were combined and lyophilized to dryness to afford (S)-18-(4-(((2-amino-4-hydroxypteridin-6-yl)methyl)amino)benzamido)-1-(1-(2-(2-((5-(1-(2-(difluoromethoxy)benzyl)-2-methyl-1H-benzo[d]imidazol-6-yl)pyrimidin-2-yl)oxy)ethoxy)ethyl)-1H-1,2,3-triazol-4-yl)-15-oxo-2,5,8,11-tetraoxa-14-azanonadecan-19-oic acid (Cpd. No. I-24) as a yellow solid. Yield: 0.023 g; 43.66%; LC-MS; m/z 575.98 [M+2]⁺+; ¹H NMR (400 MHz, DMSO-d6 with D₂O) δ 8.90 (s, 2H), 8.64 (s, 1H), 8.13 (s, 1H), 8.01 (s, 1H), 7.87 (s, 2H), 7.61 (d, J=8.4 Hz, 2H), 7.45-7.05 (m, 5H), 6.62 (d, J=8.8 Hz, 2H), 5.73 (s, 2H), 4.51-4.44 (m, 8H), 4.24-4.23 (m, 1H), 3.86 (bs, 2H), 3.76 (bs, 2H), 3.47-3.41 (m, 13H), 3.32-3.30 (m, 2H), 3.13-3.11 (m, 2H), 2.78 (s, 3H), 2.18-2.16 (m, 2H), 2.10-2.00 (m, 1H), 1.90-1.88 (m, 1H).

### 6.1.25. Example 25: Compound I-25

(S)-18-(4-(((2-amino-4-hydroxypteridin-6-yl)methyl)amino)benzamido)-1-(1-(5-((5-(1-(2-(difluoromethoxy)benzyl)-2-((3-(2-oxopyrrolidin-1-yl)phenoxy)methyl)-1H-benzo[d]imidazol-6-yl)pyridin-2-yl)oxy)pentyl)-1H-1,2,3-triazol-4-yl)-15-oxo-2,5,8,11-tetraoxa-14-azanonadecan-19-oic acid (Compound I-25)

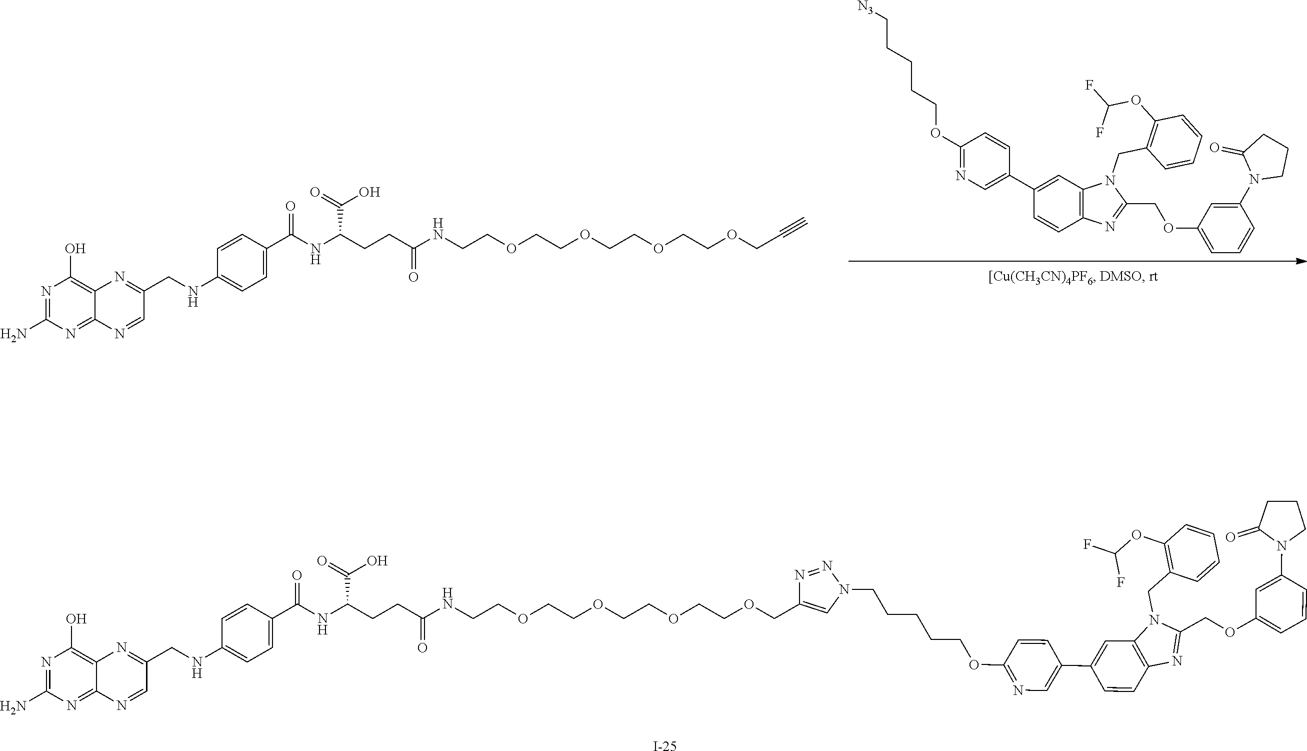

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq, 0.03 g, 0.0458 mmol) and 1-(3-((6-(6-((5-azidopentyl)oxy)pyridin-3-yl)-1-(2-(difluoromethoxy)benzyl)-1H-benzo[d]imidazol-2-yl)methoxy)phenyl)pyrrolidin-2-one (1.0 eq, 0.0306 g, 0.0458 mmol) in dimethylsulfoxide (1.5 mL), tetrakis(acetonitrile)copper(I) hexafluorophosphate (2.8 eq, 0.0478 g, 0.128 mmol) was added. The reaction mixture was stirred at room temperature for 1 h and reaction mixture was directly purified by prep HPLC (25-38% acetonitrile in water with 0.1% trifluoroacetic acid). Fractions containing the desired product were combined and lyophilized to dryness to afford (S)-18-(4-(((2-amino-4-hydroxypteridin-6-yl)methyl)amino)benzamido)-1-(1-(5-((5-(1-(2-(difluoromethoxy)benzyl)-2-((3-(2-oxopyrrolidin-1-yl)phenoxy)methyl)-1H-benzo[d]imidazol-6-yl)pyridin-2-yl)oxy)pentyl)-1H-1,2,3-triazol-4-yl)-15-oxo-2,5,8,11-tetraoxa-14-azanonadecan-19-oic acid (Cpd. No. I-25) as a yellow solid. Yield: 0.032 g; 52%; LCMS; m/z 662.28 [M+2]⁺+; ¹H NMR (400 MHz, DMSO-d6 with D₂O) δ 8.67 (s, 1H), 8.40 (s, 1H), 8.04 (s, 1H), 7.97 (dd, J=8.8 and 2.4 Hz, 1H), 7.82 (d, J=8.4 Hz, 2H), 7.66-7.61 (m, 3H), 7.38-7.01 (m, 7H), 6.95 (d, J=7.6 Hz, 1H), 6.86 (d, J=8.4 Hz, 1H), 6.74 (d, J=8.4 Hz, 1H), 6.62 (d, J=8.8 Hz, 2H), 5.71 (s, 2H), 5.48 (s, 2H), 4.49 (d, J=14.0 Hz, 4H), 4.33 (t, J=6.8 Hz, 2H), 4.26-4.21 (m, 3H), 3.76-3.74 (m, 2H), 3.49-3.47 (m, 4H), 3.43-3.42 (m, 8H), 3.32 (t, J=5.6 Hz, 2H), 3.13 (t, J=5.6 Hz, 2H), 2.17 (t, J=6.8 Hz, 2H), 2.03 (t, J=7.6 Hz, 3H), 1.86-1.82 (m, 3H), 1.73-1.69 (m, 2H), 1.34-1.32 (m, 2H).

### 6.1.26. Example 26: Compound I-26

(S)-18-(4-(((2-amino-4-hydroxypteridin-6-yl)methyl)amino)benzamido)-1-(1-(6-(4-(3-(2,5-dichlorobenzyl)-2-methylimidazo[1,2-a]pyridin-6-yl)-1H-pyrazol-1-yl)hexyl)-1H-1,2,3-triazol-4-yl)-15-oxo-2,5,8,11-tetraoxa-14-azanonadecan-19-oic acid (Compound I-26)

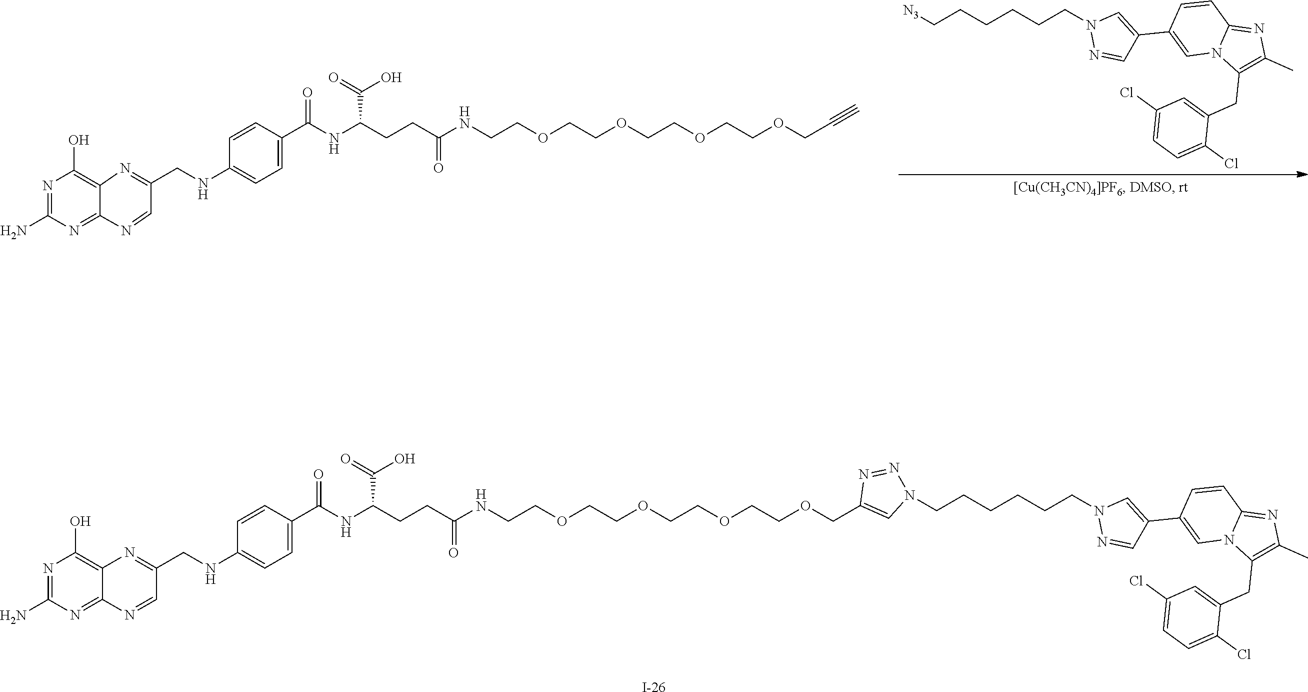

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq, 0.03 g, 0.0458 mmol) and 6-(1-(6-azidohexyl)-1H-pyrazol-4-yl)-3-(2,5-dichlorobenzyl)-2-methylimidazo[1,2-a]pyridine (1.0 eq, 0.022 g, 0.0458 mmol) in dimethylsulfoxide (1.5 mL), tetrakis(acetonitrile)copper(I) hexafluorophosphate (2.8 eq, 0.0478 g, 0.128 mmol) was added. The reaction mixture was stirred at room temperature for 1 h and reaction mixture was directly purified by prep HPLC (15-35% acetonitrile in water with 0.1% TFA). Fractions containing the desired product were combined and lyophilized to dryness to afford (S)-18-(4-(((2-amino-4-hydroxypteridin-6-yl)methyl)amino)benzamido)-1-(1-(6-(4-(3-(2,5-dichlorobenzyl)-2-methylimidazo[1,2-a]pyridin-6-yl)-1H-pyrazol-1-yl)hexyl)-1H-1,2,3-triazol-4-yl)-15-oxo-2,5,8,11-tetraoxa-14-azanonadecan-19-oic acid (Cpd. No. I-26) as a yellow solid. Yield: 0.033 g; 63%: LCMS; m/z 569.18 [M+2]⁺+; ¹H NMR (400 MHz, DMSO-d6 with D₂O) δ 8.79 (s, 1H), 8.65 (s, 1H), 8.36 (s, 1H), 8.19 (d, J=7.4 Hz, 1H), 8.15 (d, J=9.2 Hz, 1H), 8.05 (d, J=8.4 Hz, 2H), 7.96 (d, J=9.2 Hz, 1H), 7.89 (t, J=5.6 Hz, 1H), 7.64 (d, J=8.8 Hz, 2H), 7.57 (d, J=8.4 Hz, 1H), 7.41 (dd, J=8.4 & 2.4 Hz, 1H), 7.34 (d, J=2.4 H, 2H), 7.09 (brs, 1H), 6.62 (d, J=8.4 H, 1H), 4.55 (s, 2H), 4.48 (s, 4H), 4.32-4.26 (m, 5H), 4.11-4.09 (m, 3H), 3.52-3.50 (m, 4H), 3.36-3.33 (m, 2H), 3.17-3.13 (m, 2H), 2.33 (s, 3H), 2.20-2.16 (m, 2H), 2.05-2.01 (m, 1H), 1.91-1.86 (m, 1H), 1.79-1.75 (m, 4H), 1.24-1.23 (m, 4H).

### 6.1.27. Example 27: Compound I-27

(S)-2-(4-(((2-amino-4-hydroxypteridin-6-yl)methyl)amino)benzamido)-4-(5-(1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-2,5,8,11-tetraoxatridecan-13-yl)-1,3,4-oxadiazol-2-yl)butanoic acid (Compound I-27)

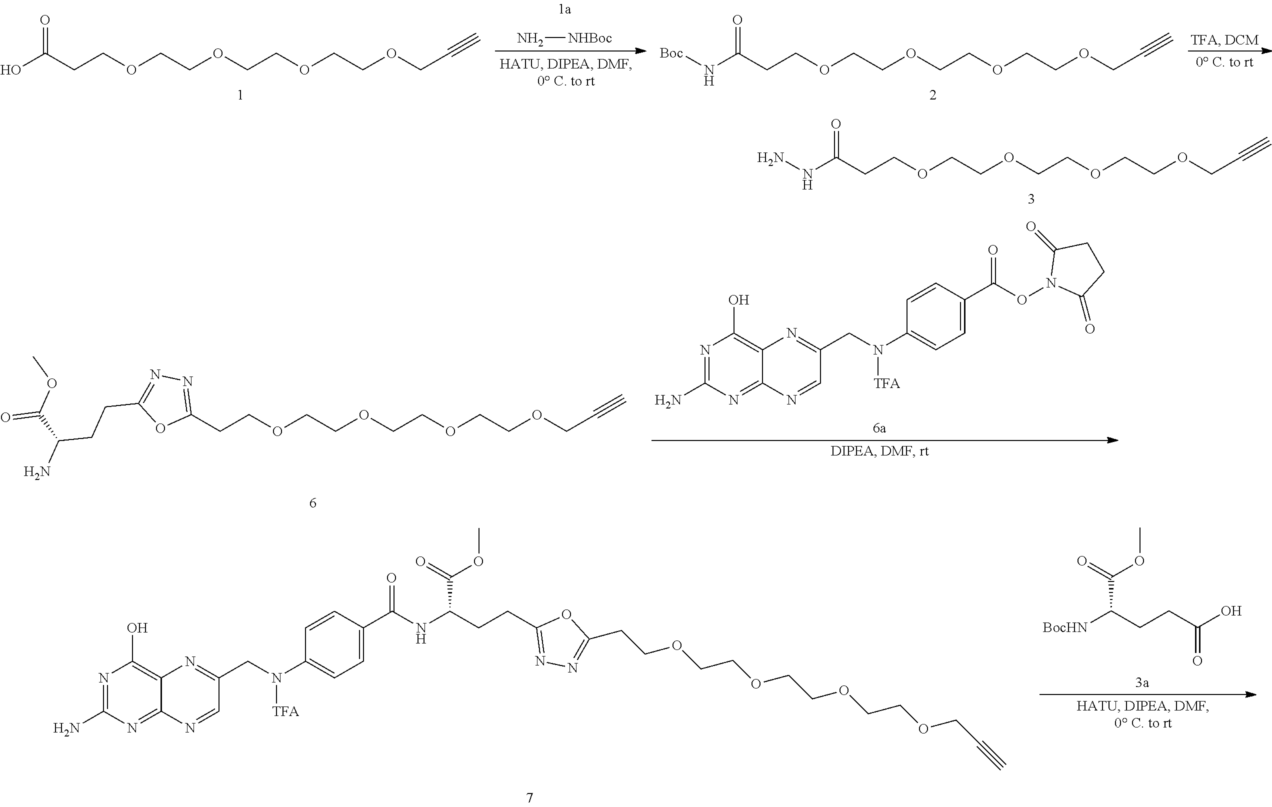

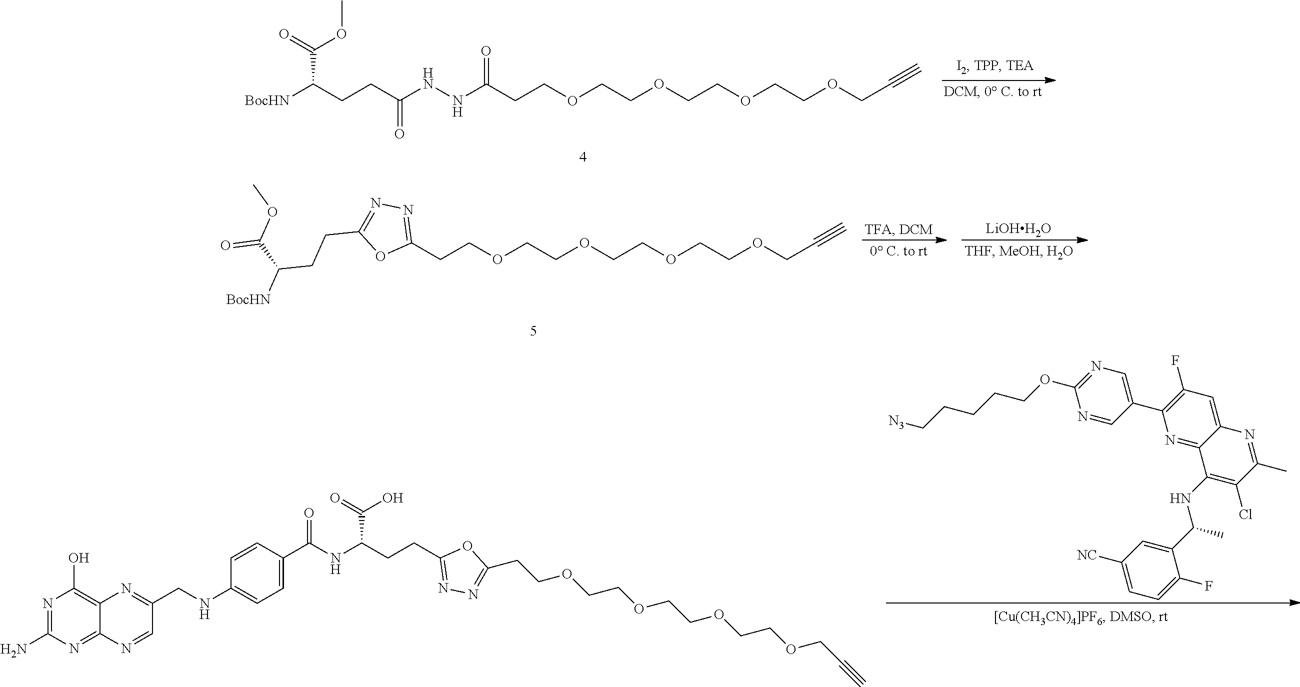 [SMILES: C#CCOCCOCCOCCOCCC(=O)NNC(=O)CC[C@H](NC(=O)OC(C)(C)C)C(=O)OC ; C#CCOCCOCCOCCOCCc1nnc(CC[C@H](NC(=O)OC(C)(C)C)C(=O)OC)o1 ; "LiOH·H2O" ; C#CCOCCOCCOCCOCCc1nnc(CC[C@H](NC(=O)c2ccc(NCc3cnc4nc(N)nc(O)c4n3)cc2)C(=O)O)o1 ; Cc1nc2cc(F)c(-c3cnc(OCCCCCN=[N+]=[N-])nc3)nc2c(N[C@H](C)c2cc(C#N)ccc2F)c1Cl]

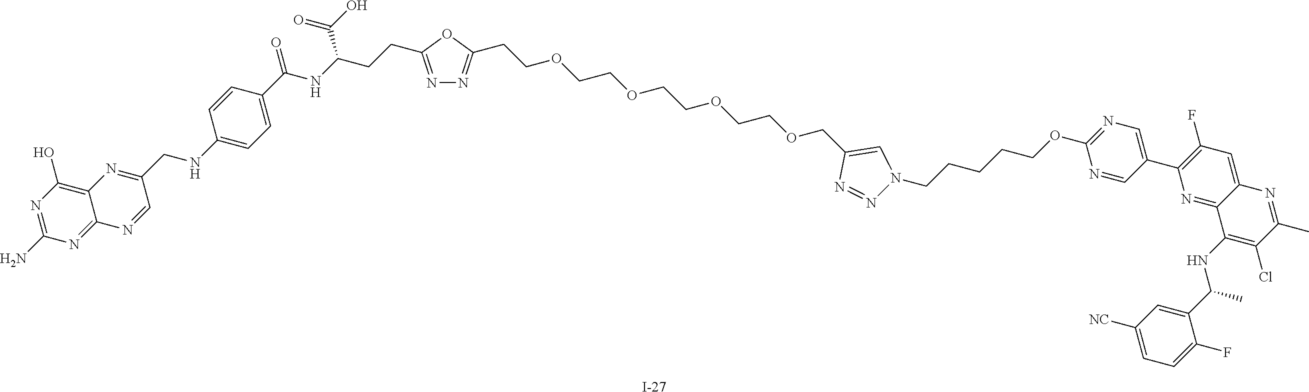

A solution of 4,7,10,13-tetraoxahexadec-15-ynoic acid (1, 1.0 eq, 1.0 g, 3.84 mmol) and tert-butyl hydrazinecarboxylate (1a, 1.0 eq, 0.762 g, 5.76 mmol) in N,N-dimethylformamide (20 mL) was cooled at 0° C., N,N-diisopropylethylamine (3.0 eq, 2.13 mL, 11.5 mmol) and 1-[bis(dimethylamino)methylene]-1H-1,2,3-triazolo[4,5-b]pyridinium 3-oxid hexafluorophosphate (HATU) (1.5 eq, 2.19 g, 5.76 mmol) were added and the reaction mixture was stirred at room temperature for 16 h. After completion, water was added to the reaction mixture and product extracted with ethyl acetate. The organic layer was washed with water, dried over anhydrous sodium sulfate, filtered and concentrated to get crude material which was purified by column chromatography using silica gel (100-200 mesh) and 0-6% methanol in dichloromethane to afford tert-butyl 4-oxo-7,10,13,16-tetraoxa-2,3-diazanonadec-18-ynoate (2) as a light brown viscous liquid. Yield: 0.780 g, 54.2%; LCMS m/z 375.2[M+1]⁺.

A solution of tert-butyl 4-oxo-7,10,13,16-tetraoxa-2,3-diazanonadec-18-ynoate (2, 1.0 eq, 0.780 g, 2.08 mmol) in dichloromethane (4 mL) was cooled at 0° C. then trifluoroacetic acid (4 mL) was added and the reaction mixture was stirred at room temperature for 3 h. After that, the reaction mixture was concentrated, azeotroped with dichloromethane (2-3 times) and dried to afford 4,7,10,13-tetraoxahexadec-15-ynehydrazide (3) as a light brown viscous liquid. Yield: 0.950 g (crude); LCMS m/z 275.0 [M+1]⁺.

A solution of (S)-4-((tert-butoxycarbonyl)amino)-5-methoxy-5-oxopentanoic acid (3a, 1.0 eq, 0.150 g, 0.574 mmol) in N,N-dimethylformamide (2 mL) was cooled at 0° C., 4,7,10,13-tetraoxahexadec-15-ynehydrazide (3, 1.1 eq, 0.173 g, 0.632 mmol), N,N-diisopropylethylamine (3.0 eq, 0.31 mL, 1.72 mmol) and 1-[bis(dimethylamino)methylene]-1H-1,2,3-triazolo[4,5-b]pyridinium 3-oxid hexafluorophosphate (HATU) (1.5 eq, 0.327 g, 0.861 mmol) were added and the reaction mixture was stirred at room temperature for 16 h. After completion, water was added to the reaction mixture and products extracted with ethyl acetate. The organic layer was washed with water, dried over anhydrous sodium sulfate, filtered and concentrated to get crude material which was purified by column chromatography using silica gel (100-200 mesh) and 04% methanol in dichloromethane to afford methyl (S)-22-((tert-butoxycarbonyl)amino)-16,19-dioxo-4,7,10,13-tetraoxa-17,18-diazatricos-1-yn-23-oate (4) as a colorless viscous liquid. Yield: 0.085 g, 26.5%; LCMS m/z 518.2[M+1]⁺.

A solution of triphenylphosphine (2.0 eq, 0.203 g, 0.773 mmol) and iodine (2.0 eq, 0.196 g, 0.773 mmol) in dichloromethane (2 mL) was stirred at room temperature for 10 minutes. Then, the solution was cooled to 0° C. before adding triethylamine (4.0 eq, 0.22 mL, 1.55 mmol) and the reaction was again warmed and stirred at room temperature for 10 minutes. A solution of methyl (S)-22-((tert-butoxycarbonyl)amino)-16,19-dioxo-4,7,10,13-tetraoxa-17,18-diazatricos-1-yn-23-oate (4, 1.0 eq, 0.200 g, 0.386 mmol) in dichloromethane (2 mL) was added at 0° C. and the reaction mixture was warmed and stirred at room temperature for 1 h. After completion, water was added to the reaction mixture and products extracted with dichloromethane. The organic layer was dried over anhydrous sodium sulfate, filtered and concentrated to get crude which was purified by column chromatography using silica gel (100-200 mesh) and 0-5% methanol in dichloromethane to afford methyl (S)-4-(5-(3,6,9,12-tetraoxapentadec-14-yn-1-yl)-1,3,4-oxadiazol-2-yl)-2-((tert-butoxycarbonyl)amino)butanoate (5) as a light yellow viscous liquid. Yield: 0.200 g, 94.5%; LCMS m/z 500.2 [M+1]⁺.

A solution of methyl (S)-4-(5-(3,6,9,12-tetraoxapentadec-14-yn-1-yl)-1,3,4-oxadiazol-2-yl)-2-((tert-butoxycarbonyl)amino)butanoate (5, 1.0 eq, 0.190 g, 0.380 mmol) in dichloromethane (1.5 mL) was cooled at 0° C. then trifluoroacetic acid (1.5 mL) was added and the reaction mixture was stirred at room temperature for 3 h. The reaction was then concentrated, azeotroped with dichloromethane (2-3 times), washed with diethyl ether (2-3 times) and dried to afford methyl (S)-4-(5-(3,6,9,12-tetraoxapentadec-14-yn-1-yl)-1,3,4-oxadiazol-2-yl)-2-aminobutanoate (6) as a colorless viscous liquid. Yield: 0.210 g (crude); LCMS m/z 400.2 [M+1]⁺.

To a suspension of 2,5-dioxopyrrolidin-1-yl 4-(N-((2-amino-4-hydroxypteridin-6-yl)methyl)-2,2,2-trifluoroacetamido)benzoate (6a, 1.0 eq) and methyl (S)-4-(5-(3,6,9,12-tetraoxapentadec-14-yn-1-yl)-1,3,4-oxadiazol-2-yl)-2-aminobutanoate (6, 1.2 eq) in N,N-dimethylformamide (10 vol.), N,N-diisopropylethylamine (5.0 eq) is added and the reaction mixture is stirred at room temperature for 16 h. After completion, the reaction mixture is concentrated to get crude material which is purified to afford methyl (S)-4-(5-(3,6,9,12-tetraoxapentadec-14-yn-1-yl)-1,3,4-oxadiazol-2-yl)-2-(4-(N-((2-amino-4-hydroxypteridin-6-yl)methyl)-2,2,2-trifluoroacetamido)benzamido)butanoate (7). LCMS m/z 790.2 [M+1]⁺.

To a suspension of methyl (S)-4-(5-(3,6,9,12-tetraoxapentadec-14-yn-1-yl)-1,3,4-oxadiazol-2-yl)-2-(4-(N-((2-amino-4-hydroxypteridin-6-yl)methyl)-2,2,2-trifluoroacetamido)benzamido)butanoate (7, 1.0 eq) in tetrahydrofuran:methanol:water (3:2:1) (10 vol), lithium hydroxide monohydrate (4.0 eq) is added and the reaction mixture is stirred at room temperature for 4 h. After completion, the reaction mixture is concentrated to give crude material which is purified to afford methyl (S)-4-(5-(3,6,9,12-tetraoxapentadec-14-yn-1-yl)-1,3,4-oxadiazol-2-yl)-2-(4-(N-((2-amino-4-hydroxypteridin-6-yl)methyl)-2,2,2-trifluoroacetamido)benzamido)butanoate. LCMS m/z 680.2 [M+1]⁺.

To a solution of methyl (S)-4-(5-(3,6,9,12-tetraoxapentadec-14-yn-1-yl)-1,3,4-oxadiazol-2-yl)-2-(4-(N-((2-amino-4-hydroxypteridin-6-yl)methyl)-2,2,2-trifluoroacetamido)benzamido)butanoate (1.0 eq) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq) in dimethylsulfoxide (20 vol), tetrakis(acetonitrile)copper(I) hexafluorophosphate (2.8 eq) is added and the reaction mixture is stirred at room temperature for 1 h. After completion, was added the reaction mixture is quenched with acetic acid and purified to afford (S)-2-(4-(((2-amino-4-hydroxypteridin-6-yl)methyl)amino)benzamido)-4-(5-(1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-2,5,8,11-tetraoxatridecan-13-yl)-1,3,4-oxadiazol-2-yl)butanoic acid (Cpd. No. I-27). LCMS m/z 622.2 [M+2]⁺⁺.

### 6.1.28. Example 28: Compound I-28

### 4-(((2-amino-4-hydroxypteridin-6-yl)methyl)amino)-N-((S)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-18-(2H-tetrazol-5-yl)-2,5,8,11-tetraoxa-14-azaoctadecan-18-yl)benzamide (I-28)

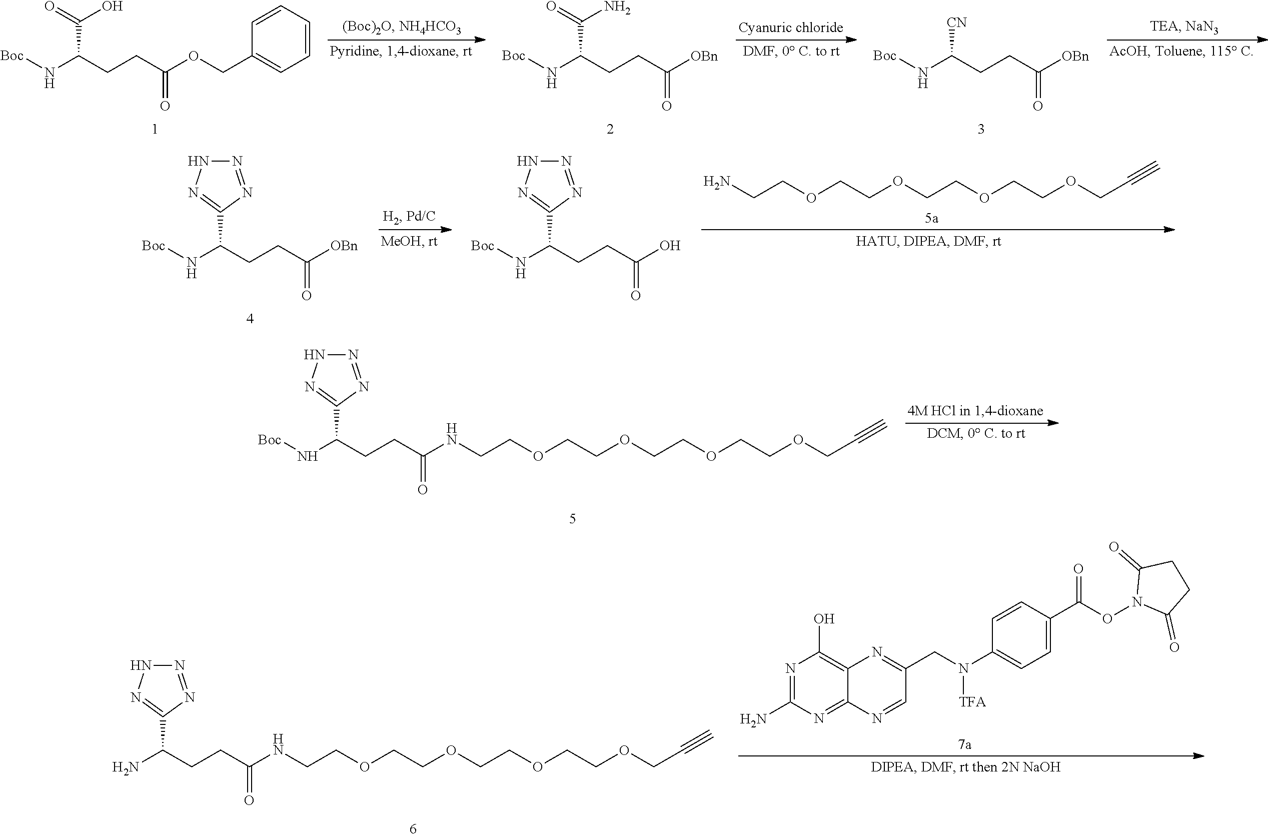

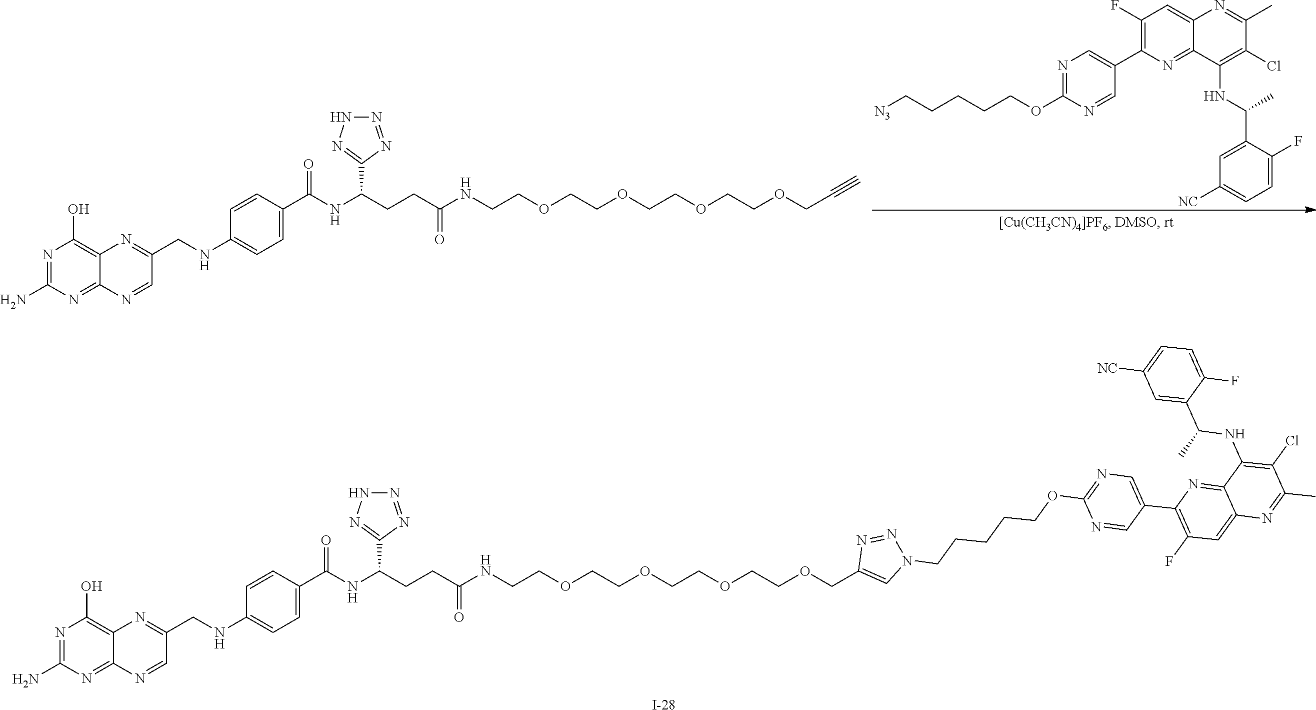

To a solution of (S)-5-(benzyloxy)-2-((tert-butoxycarbonyl)amino)-5-oxopentanoic acid (1, 1.0 eq, 1.0 g, 2.96 mmol) in 1,4-dioxane (20 mL), Boc anhydride (1.3 eq., 0.885 mL, 3.85 mmol), ammonium bicarbonate (1.3 eq., 0.305 g, 3.85 mmol) and pyridine (1.3 eq., 0.31 mL, 3.85 mmol) were added and the reaction mixture was stirred at room temperature for 16 h. After completion, the reaction mixture was concentrated, water was added and products extracted with ethyl acetate. The organic layer was dried over anhydrous sodium sulfate, filtered and concentrated to give a solid which was washed with hexane (2-3 times) and dried to afford benzyl (S)-5-amino-4-((tert-butoxycarbonyl)amino)-5-oxopentanoate (2) as a white solid. Yield: 1.05 g (crude), LCMS m/z 337.2 [M+1]⁺.

To a solution benzyl (S)-5-amino-4-((tert-butoxycarbonyl)amino)-5-oxopentanoate (2, 1.0 eq, 0.100 g, 0.297 mmol) in N,N-dimethylformamide (1 mL) at 0° C. was added cyanuric chloride (0.65 eq, 0.035 g, 0.193 mmol) and the reaction mixture was stirred at room temperature for 16 h. After completion, water was added to the reaction mixture and products extracted with ethyl acetate. The organic layer was washed with water, dried over anhydrous sodium sulfate, filtered and concentrated to get crude material which was purified by column chromatography using silica gel (100-200 mesh) and 0-8% methanol in dichloromethane to afford benzyl (S)-4-((tert-butoxycarbonyl)amino)-4-cyanobutanoate (3) as a white solid. Yield: 0.075 g, 79.24%; LCMS m/z 319.2 [M+1]⁺.

To a solution of triethylamine (4.0 eq, 1.34 mL, 9.30 mmol) in toluene (10 mL) was added acetic acid (4.0 eq, 0.55 mL, 9.30 mmol) and the reaction mixture was stirred at room temperature for 5 minutes. Next, benzyl (S)-4-((tert-butoxycarbonyl)amino)-4-cyanobutanoate (3, 1.0 eq, 0.740 g, 2.32 mmol) and sodium azide (4.0 eq, 0.604 g, 9.30 mmol) were added and the reaction mixture was heated at 115° C. for 16 h. After completion, the reaction mixture was cooled and filtered and the filtrate was concentrated to give crude material which was purified by column chromatography using silica gel (100-200 mesh) and 0-10% methanol with 0.1% acetic acid in dichloromethane to afford benzyl (S)-4-((tert-butoxycarbonyl)amino)-4-(2H-tetrazol-5-yl)butanoate (4) as a white solid. Yield: 0.690 g, 81.44%; LCMS m/z 362.2 [M+1]⁺.

To a solution of benzyl (S)-4-((tert-butoxycarbonyl)amino)-4-(2H-tetrazol-5-yl)butanoate (4, 1.0 eq, 0.690 g, 1.91 mmol) in methanol (10 mL), 10% palladium on carbon (0.300 g) was added and the reaction mixture was stirred at room temperature under hydrogen gas atmosphere for 3 h. After completion, the reaction mixture was filtered through sintered glass funnel and the filtrate was concentrated, washed with diethyl ether and dried to afford (S)-4-((tert-butoxycarbonyl)amino)-4-(2H-tetrazol-5-yl)butanoic acid as a white solid. Yield: 0.500 g, 96.54%; LCMS m/z 272.0 [M+1]⁺.

To a solution of (S)-4-((tert-butoxycarbonyl)amino)-4-(2H-tetrazol-5-yl)butanoic acid (0.200 g, 0.737 mmol) in N,N-dimethylformamide (2 mL) at 0° C. was added 3,6,9,12-tetraoxapentadec-14-yn-1-amine (5a, 1.0 eq, 0.171 g, 0.737 mmol), N,N-diisopropylethylamine (3.0 eq, 0.392 mL, 2.21 mmol) and 1-[bis(dimethylamino)methylene]-1H-1,2,3-triazolo[4,5-b]pyridinium 3-oxid hexafluorophosphate (HATU) (1.5 eq, 0.420 g, 1.11 mmol) and the reaction mixture was stirred at room temperature for 16 h. Water was added to the reaction mixture and products extracted with 10% methanol in dichloromethane. The organic layer was dried over anhydrous sodium sulfate, filtered and concentrated to give crude material which was purified by column chromatography using silica gel (100-200 mesh) and 0-15% methanol in dichloromethane to afford tert-butyl (S)-(17-oxo-20-(2H-tetrazol-5-yl)-4,7,10,13-tetraoxa-16-azaicos-1-yn-20-yl)carbamate (5) as a light brown viscous liquid. Yield: 0.080 g, 22.39%; LCMS m/z 485.2 [M+1]⁺.

A solution of tert-butyl N-[(1S)-3-[(3,6,9,12-tetraoxapentadec-14-yn-1-yl)carbamoyl]-1-(2H-1,2,3,4-tetrazol-5-yl)propyl]carbamate (5, 1.0 eq, 0.050 g, 0.103 mmol) in dichloromethane (0.5 mL) at 0° C. was treated with 4M hydrochloric acid in 1,4-dioxane (0.5 mL) and the reaction mixture was stirred at room temperature for 3 h. After completion, the reaction mixture was concentrated, azeotroped with dichloromethane (2-3 times), washed with diethyl ether (2-3 times) and dried to afford (S)-4-amino-N-(3,6,9,12-tetraoxapentadec-14-yn-1-yl)-4-(2H-tetrazol-5-yl)butanamide (6) as a light brown viscous liquid. Yield: 0.050 g (crude); LCMS m/z 385.2 [M+1]⁺.

To a solution of (S)-4-amino-N-(3,6,9,12-tetraoxapentadec-14-yn-1-yl)-4-(2H-tetrazol-5-yl)butanamide (6, 1.2 eq) and 2,5-dioxopyrrolidin-1-yl 4-(N-((2-amino-4-hydroxypteridin-6-yl)methyl)-2,2,2-trifluoroacetamido)benzoate (7a, 1.0 eq) in N,N-dimethylformamide (10 vol.), N,N-diisopropylethylamine (5.0 eq) is added and the reaction mixture is stirred at room temperature for 16 h. Next, 2M aqueous sodium hydroxide solution (3 vol.) is added and the reaction mixture is stirred at room temperature for 30 minutes then purified to afford (S)-4-(((2-amino-4-hydroxypteridin-6-yl)methyl)amino)-N-(17-oxo-20-(2H-tetrazol-5-yl)-4,7,10,13-tetraoxa-16-azaicos-1-yn-20-yl)benzamide. LCMS m/z 679.3 [M+1]⁺.

To a solution of (S)-4-(((2-amino-4-hydroxypteridin-6-yl)methyl)amino)-N-(17-oxo-20-(2H-tetrazol-5-yl)-4,7,10,13-tetraoxa-16-azaicos-1-yn-20-yl)benzamide (1.0 eq) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq) in dimethylsulfoxide (20 vol) is added tetrakis(acetonitrile)copper(I) hexafluorophosphate (2.8 eq) and the reaction mixture is stirred at room temperature for 1 h. After completion, the reaction mixture is quenched with acetic acid and purified to afford 4-(((2-amino-4-hydroxypteridin-6-yl)methyl)amino)-N-((S)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-18-(2H-tetrazol-5-yl)-2,5,8,11-tetraoxa-14-azaoctadecan-18-yl)benzamide (Cpd. No. I-28). LCMS m/z 621.7 [M+2]⁺⁺.

### 6.1.29. Example 29: Compound I-29

(S)-18-(4-(((2-amino-4-hydroxypteridin-6-yl)methyl)amino)benzamido)-1-(1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)ethoxy)ethyl)-1H-1,2,3-triazol-4-yl)-15-oxo-2,5,8,11-tetraoxa-14-azanonadecan-19-oic acid (Compound I-29)

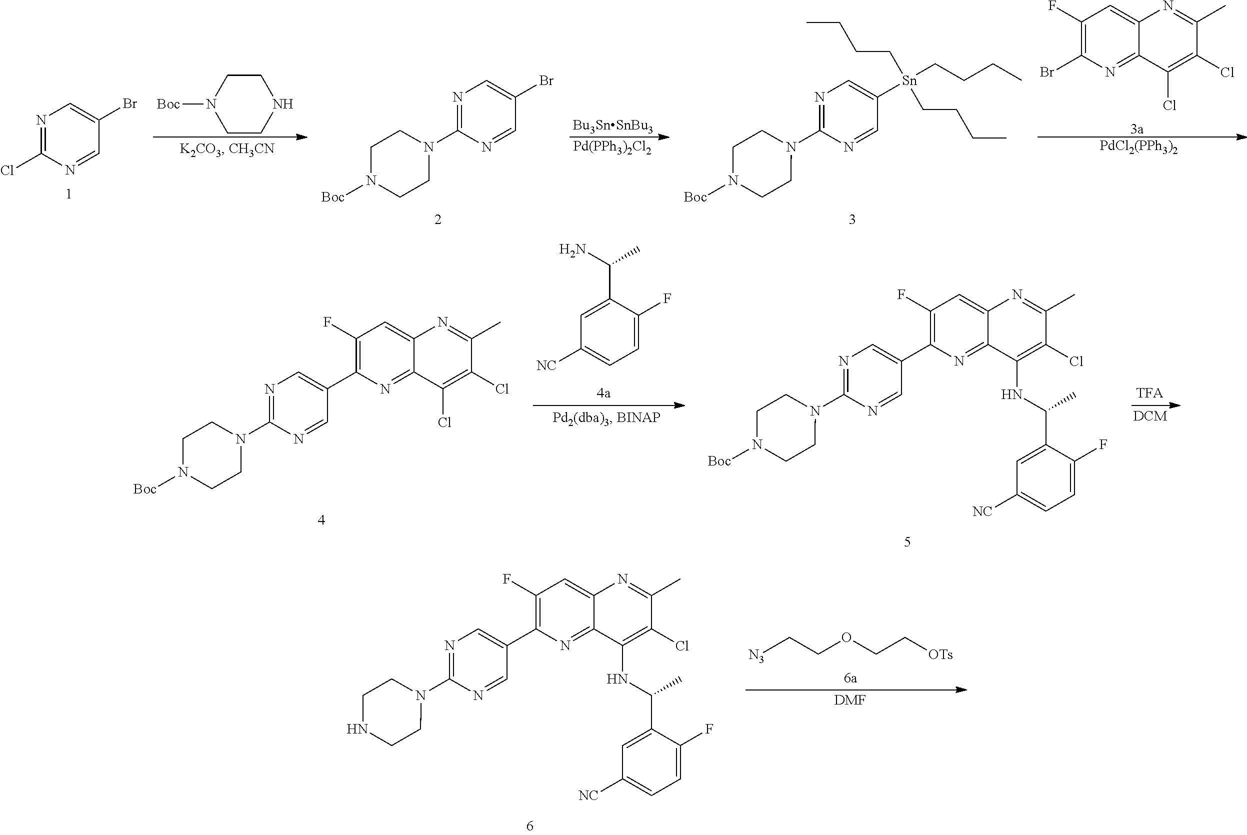

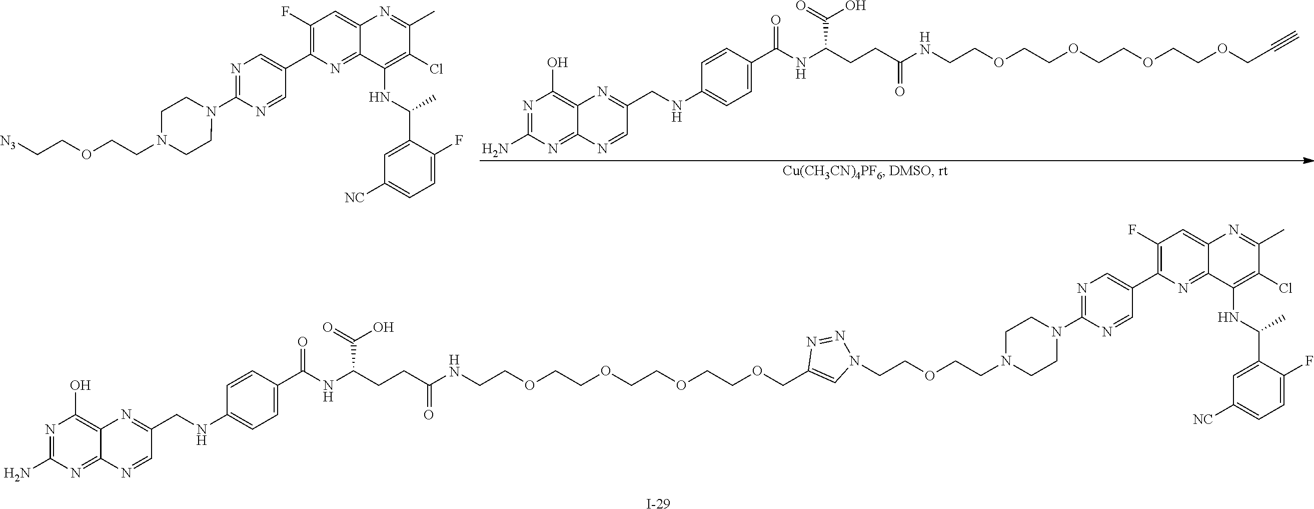

To a solution of 5-bromo-2-chloropyrimidine (1, 1.0 eq, 2.00 g, 10.3 mmol) and tert-butyl piperazine-1-carboxylate (1a, 1.0 eq, 1.93 g, 10.3 mmol) in acetonitrile (16 mL), potassium carbonate (2.0 eq, 2.86 g., 20.7 mmol) was added at room temperature, and the reaction mixture was heated at 110° C. for 16 h. After completion, the reaction mixture was concentrated under reduced pressure to give crude material which was purified by flash column chromatography over silica gel using 5-30% ethyl acetate in hexane as eluent to afford tert-butyl 4-(5-bromopyrimidin-2-yl)piperazine-1-carboxylate (2) as a white solid. Yield 2.60 g, 73.3%; LCMS m/z 243.0 [M-Boc+1]⁺.

A solution of tert-butyl 4-(5-bromopyrimidin-2-yl)piperazine-1-carboxylate (2, 1.0 eq, 3.20 g, 9.32 mmol) and bis(tributylstannane)(1.2 eq, 6.51 g, 11.2 mmol) in 1,4-dioxane (90 mL) was purged with nitrogen for 10 minutes then palladium (II) bis(triphenylphosphane) dichloride (0.05 eq, 0.327 g, 0.466 mmol) was added and reaction mixture was again purged with nitrogen. The reaction mixture was then heated at 100° C. for 16 h. After completion, the reaction mixture was filtered through a pad of celite and the filtrate was concentrated under reduced pressure to give crude material which was purified by flash column chromatography over silica gel using 5-30% ethyl acetate in hexane as eluent to afford tert-butyl 4-[5-(tributylstannyl)pyrimidin-2-yl]piperazine-1-carboxylate (3) as colorless viscous liquid. Yield 1.30 g, 25.1%; LCMS m/z 555.26 [M+1]⁺.

A solution of 6-bromo-3,4-dichloro-7-fluoro-2-methyl-1,5-naphthyridine (3a, 1.0 eq, 1.70 g, 5.48 mmol) and tert-butyl 4-[5-(tributylstannyl)pyrimidin-2-yl]piperazine-1-carboxylate (3, 1.1 eq, 3.34 g, 6.03 mmol) in 1,4-dioxane (20 mL) was purged with nitrogen gas for 10 minutes then, palladium(II) bis(triphenylphosphane) dichloride (0.05 eq 0.192 g, 0.27.4 mmol) was added and again purged with nitrogen for 10 minutes. The reaction mixture was stirred at 100° C. under a nitrogen atmosphere for 4 h. After completion, the reaction mixture was filtered through a pad of celite and the filtrate was concentrated under reduced pressure to give crude material which was purified by flash column chromatography over silica gel using 15-50% ethyl acetate in hexane as eluent to afford tert-butyl 4-(5-(7,8-dichloro-3-fluoro-6-methyl-1,5-naphthyridin-2-yl)pyrimidin-2-yl)piperazine-1-carboxylate (4) as off white solid. Yield 1.0 g, 37.0%; LCMS m/z 491.08 [M−1]⁻.

A solution of (R)-3-(1-aminoethyl)-4-fluorobenzonitrile hydrochloride (4a, 1.0 eq, 0.20 g, 1.22 mmol) and tert-butyl 4-(5-(7,8-dichloro-3-fluoro-6-methyl-1,5-naphthyridin-2-yl)pyrimidin-2-yl)piperazine-1-carboxylate (4, 1.0 eq, 0.60 g, 1.22 mmol), cesium carbonate (1.5 eq, 0.59 g, 1.83 mmol), tris(dibenzylideneacetone)-dipalladium(0) (0.11 g, 0.1 eq, 0.122 mmol), 2,2′-bis(diphenylphosphino)-1,1′-binaphthyl (0.20 eq, 0.15 g, 0.244 mmol) in anhydrous toluene (12 mL) and 1,4-dioxane (12 mL) was purged with nitrogen for 10 minutes. The reaction mixture was then stirred at 100° C. under a nitrogen atmosphere for 4 h. After the completion of reaction, reaction mixture was filtered through celite and washed with ethyl acetate, water was added, and extracted with ethyl acetate. The combined organic layer was dried over sodium sulfate, filtered, and concentrated under reduced pressure to get crude which was purified by flash chromatography over silica gel using 20 to 50% of ethyl acetate in hexanes as eluent to afford tert-butyl (R)-4-(5-(7-chloro-8-((1-(5-cyano-2-fluorophenyl)ethyl)amino)-3-fluoro-6-methyl-1,5-naphthyridin-2-yl)pyrimidin-2-yl)piperazine-1-carboxylate (5) as an off white solid. Yield 0.40 g, 40.2%; LCMS m/z 621.33 [M+1]⁺.

To a stirred solution of tert-butyl (R)-4-(5-(7-chloro-8-((1-(5-cyano-2-fluorophenyl)ethyl)amino)-3-fluoro-6-methyl-1,5-naphthyridin-2-yl)pyrimidin-2-yl)piperazine-1-carboxylate (5, 1.0 eq, 0.40 g, 0.644 mmol) in dichloromethane (8 mL) at 0° C., trifluoroacetic acid (2 mL) was added. The reaction mixture was then stirred at room temperature for 4 h. After completion, the reaction mixture was concentrated under reduced pressure to afford crude (R)-3-(1-((3-chloro-7-fluoro-2-methyl-6-(2-(piperazin-1-yl)pyrimidin-5-yl)-1,5-naphthyridin-4-yl)amino)ethyl)-4-fluorobenzonitrile (6) as a brown viscous liquid. Yield 0.40 g, LCMS m/z 521.28 [M+1]⁺.

A solution of (R)-3-(1-((3-chloro-7-fluoro-2-methyl-6-(2-(piperazin-1-yl)pyrimidin-5-yl)-1,5-naphthyridin-4-yl)amino)ethyl)-4-fluorobenzonitrile (6, 1.0 eq, 0.16 g, 0.307 mmol) and 2-(2-azidoethoxy)ethyl 4-methylbenzenesulfonate (1.1 eq, 0.096 g, 0.338 mmol) in N,N-dimethyl formamide (4.0 mL), was stirred at 60° C. for 16 h. After completion, the reaction mixture was diluted with ice water and extracted with ethyl acetate. The organic layer was dried over anhydrous sodium sulphate, filtered and concentrated under reduced pressure to give crude material which was purified by flash column chromatography over silica gel using 0-5% methanol in dichloromethane as eluent to afford (R)-3-(1-((6-(2-(4-(2-(2-azidoethoxy)ethyl)piperazin-1-yl)pyrimidin-5-yl)-3-chloro-7-fluoro-2-methyl-1,5-naphthyridin-4-yl)amino)ethyl)-4-fluorobenzonitrile. Yield 0.045 g, 23.1%; LCMS m/z 634.44 [M+1]⁺.

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq, 0.030 g, 0.045 mmol) and (R)-3-(1-((6-(2-(4-(2-(2-azidoethoxy)ethyl)piperazin-1-yl)pyrimidin-5-yl)-3-chloro-7-fluoro-2-methyl-1,5-naphthyridin-4-yl)amino)ethyl)-4-fluorobenzonitrile (1.2 eq, 0.045 g, 0.055 mmol) in dimethyl sulfoxide (1.5 mL), tetrakis(acetonitrile)copper(I) hexafluorophosphate (2.8 eq, 0.047 g, 0.128 mmol) was added and the reaction mixture was stirred at room temperature for 0.5 h. After completion, the reaction mixture was quenched with acetic acid (0.10 mL) and directly purified by prep HPLC (26-40% acetonitrile in water with 0.10% TFA). Fractions containing the desired product were combined and lyophilized to dryness to afford (S)-18-(4-(((2-amino-4-hydroxypteridin-6-yl)methyl)amino)benzamido)-1-(1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)ethoxy)ethyl)-1H-1,2,3-triazol-4-yl)-15-oxo-2,5,8,11-tetraoxa-14-azanonadecan-19-oic acid (Cpd. No. I-29) as a yellow solid. Yield: 0.025 g, 42.3%; LCMS m/z 645.05 [M+2]*+; ¹H NMR (400 MHz, DMSO-d₆ with D₂O) δ 8.83 (s, 2H), 8.63 (s, 1H), 8.09 (s, 1H), 8.05 (d, J=10.8 Hz, 1H), 7.95 (d, J=6.0 Hz, 1H), 7.77 (bs, 1H), 7.59 (d, J=8.4 Hz, 2H), 7.31 (t, J=9.6 Hz, 1H), 6.60 (d, J=8.0 Hz, 2H), 6.50 (d, J=6.4 Hz, 1H), 4.75 (bs, 2H), 4.57 (bs, 2H), 4.50 (s, 2H), 4.46 (s, 2H), 4.25-4.20 (m, 1H), 3.87 (s, 2H), 3.54 (s, 2H), 3.48 (s, 3H), 3.40 (d, J=6.8 Hz, 10H), 3.30 (t, J=4.8 Hz, 5H), 3.13-3.08 (m, 2H), 2.67-2.66 (m, 3H), 2.15 (t, J=6.8 Hz, 2H), 2.02-1.99 (m, 1H), 1.90-1.87 (m, 1H), 1.67 (d, J=6.4 Hz, 3H).

### 6.1.30. Example 30: Compound I-30

(S)-18-(4-(((2-amino-4-hydroxypteridin-6-yl)methyl)amino)benzamido)-1-(1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)-4-hydroxypiperidin-1-yl)ethoxy)ethyl)-1H-1,2,3-triazol-4-yl)-15-oxo-2,5,8,11-tetraoxa-14-azanonadecan-19-oic acid (Compound I-30)

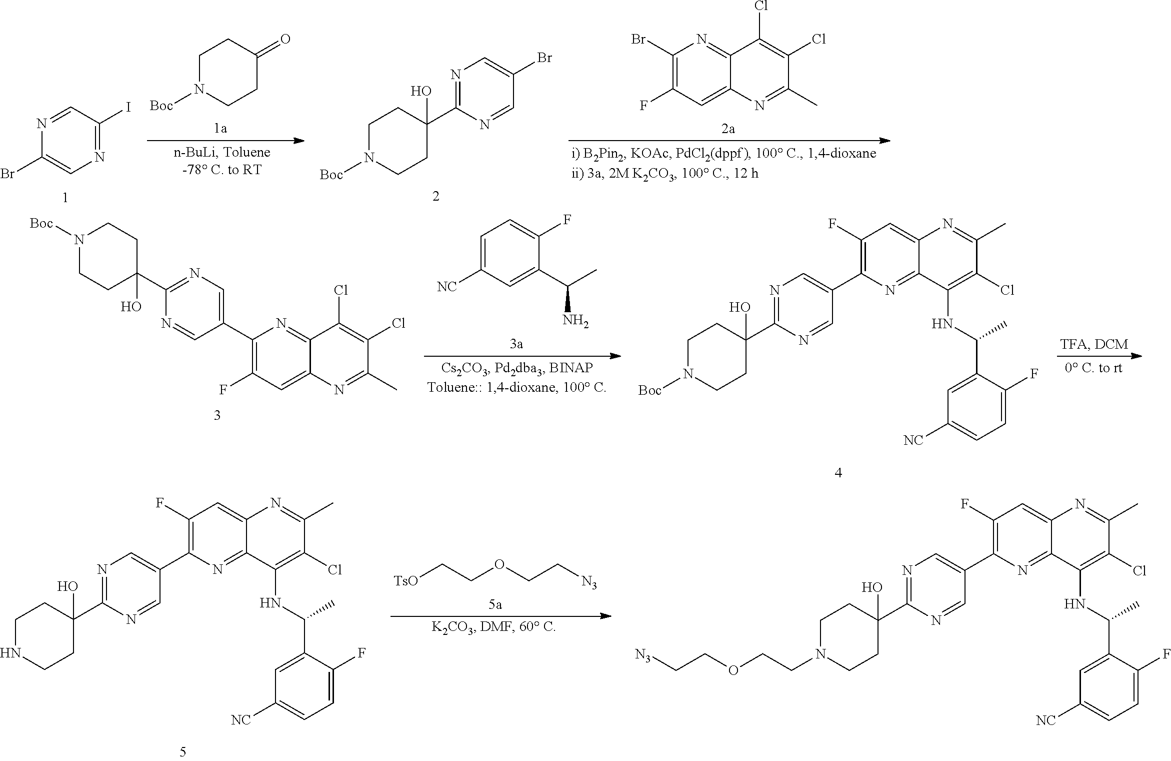

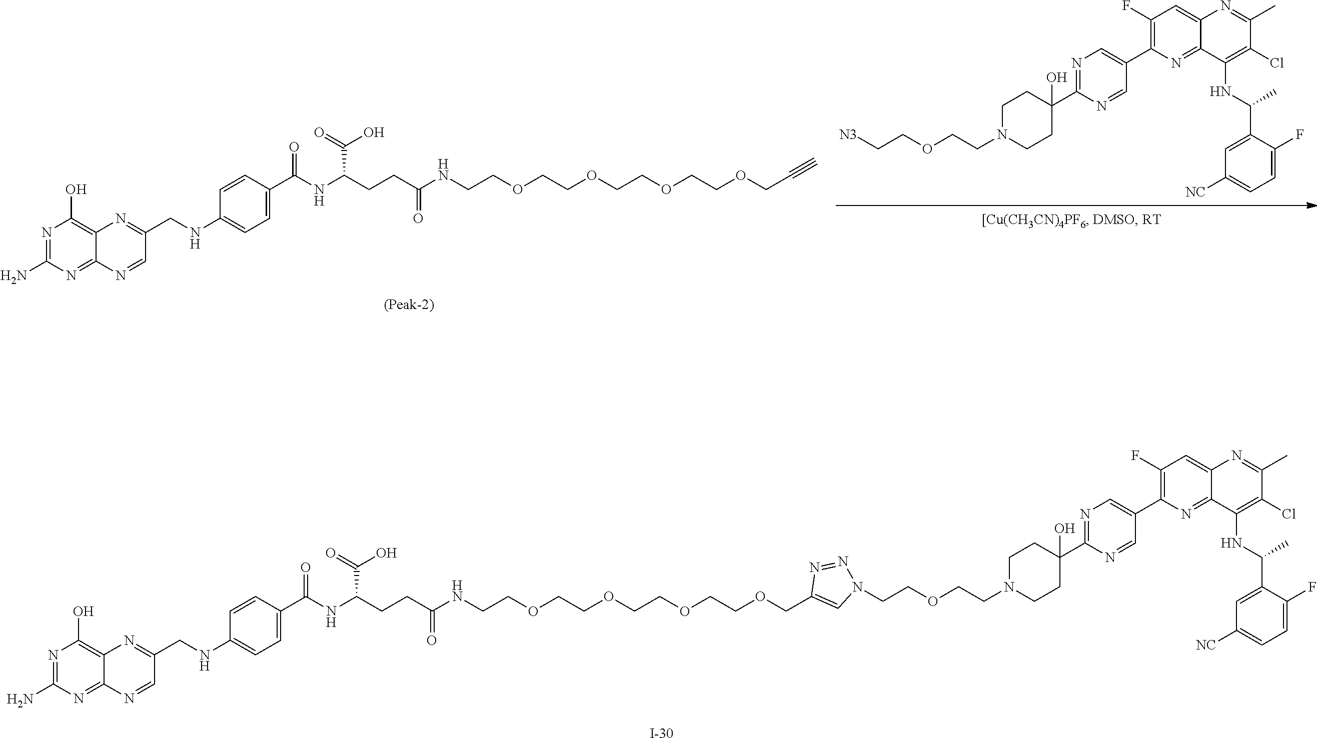

A solution of 5-bromo-2-iodopyrimidine (1, 1.0 eq, 5.0 g, 17.6 mmol) in toluene (100 mL) was cooled at −78° C., n-butyl lithium (2.5 M in Hexane, 1.1 eq, 7.72 mL, 19.3 mmol) was added and the reaction mixture was stirred at −78° C. for 30 minutes. A solution of tert-butyl 4-oxopiperidine-1-carboxylate (1a, 1.1 eq, 3.85 g, 19.3 mmol) in toluene (25 mL) was added and the reaction mixture was stirred at room temperature for 16 h. After completion, the reaction mixture was again cooled at −78° C., quenched with saturated aqueous ammonium chloride and extracted with ethyl acetate. The organic layer was dried over anhydrous sodium sulfate, filtered and concentrated to give crude material which was purified by column chromatography using silica gel (100-200 mesh) and 0-20% ethyl acetate in hexane to afford tert-butyl 4-(5-bromopyrimidin-2-yl)-4-hydroxypiperidine-1-carboxylate (2) as a light yellow viscous liquid. Yield: 1.8 g, 23.5%; LC-MS m/z 358.13 [M+1]⁺.

A mixture of tert-butyl 4-(5-bromopyrimidin-2-yl)-4-hydroxypiperidine-1-carboxylate (2, 1.2 eq, 1.59 g, 4.45 mmol), 4,4,5,5-tetramethyl-2-(4,4,5,5-tetramethyl-1,3,2-dioxaborolan-2-yl)-1,3,2-dioxaborolane (1.5 eq, 1.15 g, 3.71 mmol) and potassium acetate (1.8 eq, 0.655 g, 6.68 mmol) in 1,4-dioxane (20.0 mL) was purged with nitrogen for 5 minutes. [1,1′-Bis(diphenylphosphino)ferrocene]palladium(II) dichloride dichloromethane complex (0.125 eq, 0.378 g, 0.464 mmol) was then added and the reaction mixture was again purged with nitrogen for 5 minutes. The reaction mixture was heated at 80° C. for 3 h. After cooling to room temperature, 6-bromo-3,4-dichloro-7-fluoro-2-methyl-1,5-naphthyridine (2a, 1.0 eq, 1.15 g, 3.71 mmol) and potassium carbonate (2M aqueous solution) (2.5 eq, 4.64 mL, 9.28 mmol) were added to the reaction. The reaction mixture was purged with nitrogen and again heated at 100° C. for 12 h. After completion, the reaction mixture was diluted with ethyl acetate and water. The organic layer was separated, dried over sodium sulfate, filtered and concentrated under reduced pressure to obtain crude material which was purified by flash chromatography (silica mesh: 100-200; elution: 15-20% ethyl acetate in hexane) to afford tert-butyl 4-(5-(7,8-dichloro-3-fluoro-6-methyl-1,5-naphthyridin-2-yl)pyrimidin-2-yl)-4-hydroxypiperidine-1-carboxylate (3) as light pink solid. Yield: 0.420 g, 22.3%; LC-MS m/z 508.12 [M+1]⁺.

A solution of tert-butyl 4-(5-(7,8-dichloro-3-fluoro-6-methyl-1,5-naphthyridin-2-yl)pyrimidin-2-yl)-4-hydroxypiperidine-1-carboxylate (3, 1.0 eq, 0.49 g, 0.964 mmol), 3-[(1R)-1-aminoethyl]-4-fluorobenzonitrile hydrochloride (3a, 1.2 eq, 0.190 g, 1.16 mmol) and cesium carbonate (1.5 eq, 0.471 g, 1.45 mmol) in 1,4-dioxane:toluene (1:1) (5.0 mL: 5.0 mL) was purged with nitrogen for 5 minutes. [2′-(diphenylphosphanyl)-[1,1′-binaphthalen]-2-yl]diphenylphosphane (0.2 eq, 0.12 g, 0.193 mmol) and tris((1E,4E)-1,5-diphenylpenta-1,4-dien-3-one) palladium (0.1 eq, 0.078 g, 0.096 mmol) were added and the reaction mixture was again purged with nitrogen for 5 minutes. The reaction mixture was heated at 100° C. for 12 h. After completion, the reaction mixture was diluted with ethyl acetate and water. The organic layer was separated, dried over sodium sulfate, filtered, and concentrated under reduced pressure to obtain crude. The crude was purified by flash chromatography (silica mesh: 100-200: elution: 15 to 30% ethyl acetate in hexanes) to afford tert-butyl (R)-4-(5-(7-chloro-8-((1-(5-cyano-2-fluorophenyl)ethyl)amino)-3-fluoro-6-methyl-1,5-naphthyridin-2-yl)pyrimidin-2-yl)-4-hydroxypiperidine-1-carboxylate (4) as yellow solid. Yield: 0.35 g, 57%: LC-MS m/z 637.1 [M+1]⁺.

To a solution of tert-butyl (R)-4-(5-(7-chloro-8-((1-(5-cyano-2-fluorophenyl)ethyl)amino)-3-fluoro-6-methyl-1,5-naphthyridin-2-yl)pyrimidin-2-yl)-4-hydroxypiperidine-1-carboxylate (4, 1.0 eq, 0.1 g, 0.157 mmol) in dichloromethane (0.5 mL) was added 2,2,2-trifluoroacetic acid (0.5 mL) at 0° C. The reaction mixture was stirred at room temperature for 4 h. After that, the reaction mixture was concentrated under reduced pressure, azeotroped with dichloromethane (2 or 3 times) and dried to afford (R)-3-(1-((3-chloro-7-fluoro-6-(2-(4-hydroxypiperidin-4-yl)pyrimidin-5-yl)-2-methyl-1,5-naphthyridin-4-yl)amino)ethyl)-4-fluorobenzonitrile (5) as yellow viscous liquid. Yield: 0.15 g (crude); LC-MS m/z 537.1 [M+1]⁺.

To a solution of (R)-3-(1-((3-chloro-7-fluoro-6-(2-(4-hydroxypiperidin-4-yl)pyrimidin-5-yl)-2-methyl-1,5-naphthyridin-4-yl)amino)ethyl)-4-fluorobenzonitrile (5, 1.0 eq, 0.15 g, 0.280 mmol) in N,N dimethylformamide (1.5 mL) was added potassium carbonate (3.0 eq, 0.116 g, 0.840 mmol). A solution of 2-(2-azidoethoxy)ethyl 4-methylbenzenesulfonate (1.1 eq, 0.087 g, 0.308 mmol) in N,N-dimethylformamide (0.5 mL) was added and the reaction mixture was heated at 60° C. for 5 h. After completion, the reaction mixture was diluted with water and extracted with ethyl acetate. The organic layer was dried over sodium sulfate, filtered and concentrated under reduced pressure to obtain crude. The crude was purified by flash chromatography (silica mesh: 100-200; elution: 2-3% methanol in dichloromethane) to afford (R)-3-(1-((6-(2-(1-(2-(2-azidoethoxy)ethyl)-4-hydroxypiperidin-4-yl)pyrimidin-5-yl)-3-chloro-7-fluoro-2-methyl-1,5-naphthyridin-4-yl)amino)ethyl)-4-fluorobenzonitrile as yellow liquid. Yield: 0.06 g, 33%; LCMS: 649.23 [M+1]⁺.

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq) and (R)-3-(1-((6-(2-(1-(2-(2-azidoethoxy)ethyl)-4-hydroxypiperidin-4-yl)pyrimidin-5-yl)-3-chloro-7-fluoro-2-methyl-1,5-naphthyridin-4-yl)amino)ethyl)-4-fluorobenzonitrile (1.0 eq) in dimethylsulfoxide (20 vol), tetrakis(acetonitrile)copper(I) hexafluorophosphate (2.8 eq) is added and the reaction mixture is stirred at room temperature for 1 h. After completion, the reaction mixture is quenched with acetic acid and purified to afford (S)-18-(4-(((2-amino-4-hydroxypteridin-6-yl)methyl)amino)benzamido)-1-(1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)-4-hydroxypiperidin-1-yl)ethoxy)ethyl)-1H-1,2,3-triazol-4-yl)-15-oxo-2,5,8,11-tetraoxa-14-azanonadecan-19-oic acid (Cpd. No. I-30). 6.1.31. Example 31: Compound I-31

(S)-1-(1-(5-((5-(7-chloro-8-(((S)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-18-(5-(methyl((2-methyl-4-oxo-1,4-dihydroquinazolin-6-yl)methyl)amino)thiophene-2-carboxamido)-15-oxo-2,5,8,11-tetraoxa-14-azanonadecan-19-oic acid (Compound I-31)

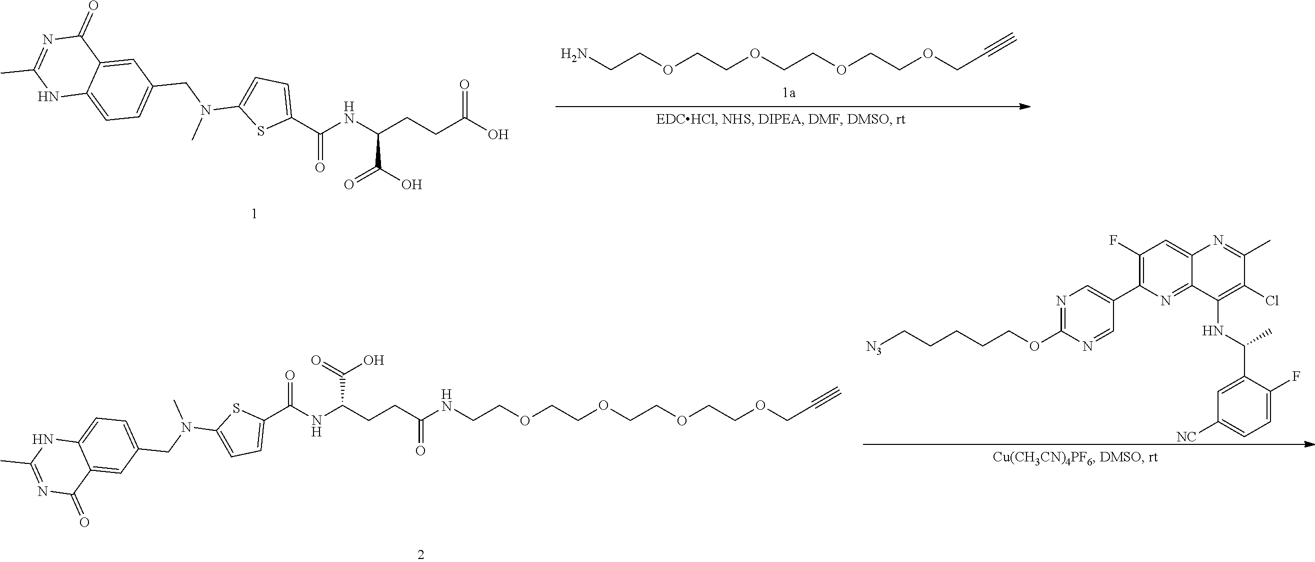 [SMILES: Cc1nc(=O)c2cc(CN(C)c3ccc(C(=O)N[C@@H](CCC(=O)O)C(=O)O)s3)ccc2[nH]1 ; C#CCOCCOCCOCCOCCN ; C#CCOCCOCCOCCOCCNC(=O)CC[C@H](NC(=O)c1ccc(N(C)Cc2ccc3[nH]c(C)nc(=O)c3c2)s1)C(=O)O ; Cc1nc2cc(F)c(-c3cnc(OCCCCCN=[N+]=[N-])nc3)nc2c(N[C@H](C)c2cc(C#N)ccc2F)c1Cl]

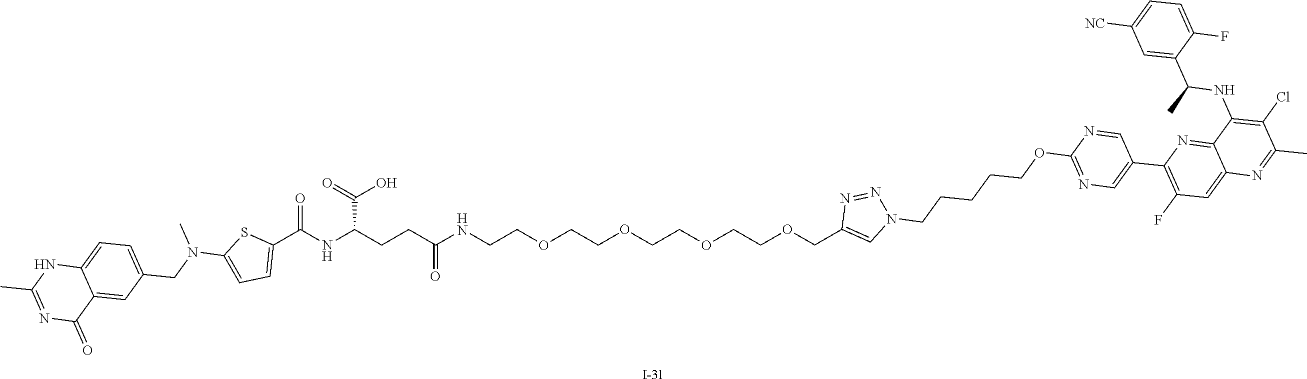

To a solution of (5-(methyl((2-methyl-4-oxo-1,4-dihydroquinazolin-6-yl)methyl)amino)thiophene-2-carbonyl)-L-glutamic acid (Raltitrexed) (1, 1.0 eq, 0.400 g, 0.872 mmol) in N,N-dimethylformamide (8 mL) and dimethyl sulfoxide (8 mL), N-hydroxysuccinimide (1.1 eq, 0.110 g, 0.960 mmol), 1-(3-Dimethylaminopropyl)-3-ethylcarbodiimide hydrochloride (EDC·HCl) (1.1 eq, 0.184 g, 0.960 mmol) and N,N-diisopropylethylamine (3.0 eq, 0.48 mL, 2.62 mmol) were added and the reaction mixture was stirred at room temperature for 1 h. Then, 3,6,9,12-tetraoxapentadec-14-yn-1-amine (1a, 1.0 eq, 0.202 g, 0.872 mmol) was added and the reaction mixture was stirred at room temperature for 16 h. After completion, the reaction mixture was directly purified by prep HPLC (24-47% acetonitrile in water with 0.1% acetic acid). Fractions containing the desired product were combined and lyophilized to dryness to afford (S)-20-(5-(methyl((2-methyl-4-oxo-1,4-dihydroquinazolin-6-yl)methyl)amino)thiophene-2-carboxamido)-17-oxo-4,7,10,13-tetraoxa-16-azahenicos-1-yn-21-oic acid (2) as a light yellow sticky solid. Yield: 0.050 g, 8.53%; LCMS m/z 672.57 [M+1]⁺.

To a solution of (S)-20-(5-(methyl((2-methyl-4-oxo-1,4-dihydroquinazolin-6-yl)methyl)amino)thiophene-2-carboxamido)-17-oxo-4,7,10,13-tetraoxa-16-azahenicos-1-yn-21-oic acid (2, 1.0 eq, 0.048 g, 0.071 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.040 g, 0.071 mmol) in dimethylsulfoxide (1.0 mL), tetrakis(acetonitrile)copper(I) hexafluorophosphate (2.8 eq, 0.074 g, 0.200 mmol) was added and reaction mixture was stirred at room temperature for 1 h. After completion, the reaction mixture was quenched with acetic acid (0.2 mL) and purified by prep HPLC (48-60% acetonitrile in water with 0.10% acetic acid). Fractions containing the desired product were combined and lyophilized to dryness to afford (S)-1-(1-(5-((5-(7-chloro-8-(((S)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-18-(5-(methyl((2-methyl-4-oxo-1,4-dihydroquinazolin-6-yl)methyl)amino)thiophene-2-carboxamido)-15-oxo-2,5,8,11-tetraoxa-14-azanonadecan-19-oic acid (Cpd. No. I-31) as an off white solid. Yield: 0.015 g, 17%; LCMS m/z 618.55 [M+2]*+; ¹H NMR (400 MHz, DMSO-d₆ with D₂O) δ 8.93 (s, 2H), 8.01 (d, J=11.2 Hz, 2H), 7.90-7.85 (m, 2H), 7.72-7.68 (m, 1H), 7.61 (d, J=8.0 Hz, 1H), 7.51-7.47 (m, 2H), 7.22 (t, J=8.8 Hz, 1H), 6.30 (d, J=8.0 Hz, 1H), 5.91 (d, J=4.0 Hz, 1H), 4.57 (s, 2H), 4.46 (s, 2H), 4.35 (bs, 4H), 4.18-4.17 (m, 1H), 4.10-4.08 (m, 1H), 3.48-3.41 (m, 14H), 3.33-3.31 (m, 2H), 3.13 (d, J=6.0 Hz, 2H), 2.99 (s, 3H), 2.59 (s, 3H), 2.29 (s, 3H), 2.14-2.13 (m, 2H), 2.05-1.95 (m, 2H), 1.90-1.79 (m, 4H), 1.80-1.70 (m, 2H), 1.61 (d, J=6.8 Hz, 3H), 1.40-1.24 (m, 2H).

### 6.1.32. Example 32: Compound I-32

(S)-18-(4-(2-(2-amino-4-oxo-4,7-dihydro-1H-pyrrolo[2,3-d]pyrimidin-5-yl)ethyl)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-2,5,8,11-tetraoxa-14-azanonadecan-19-oic acid

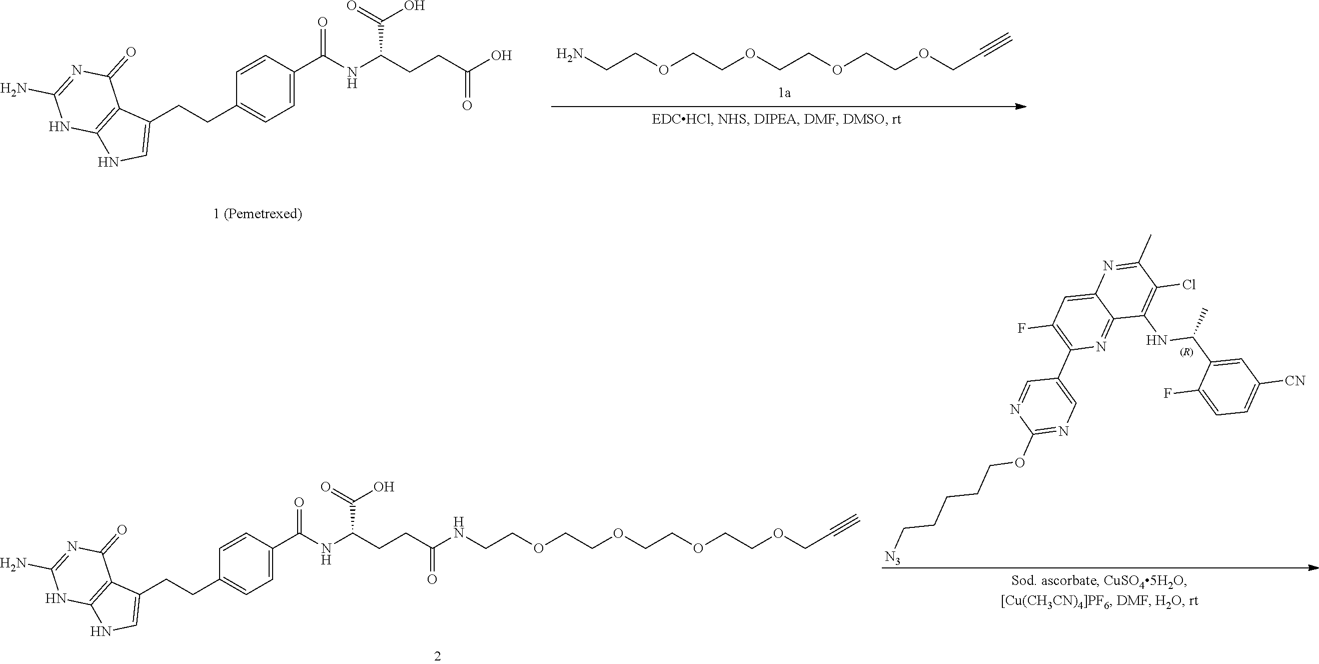

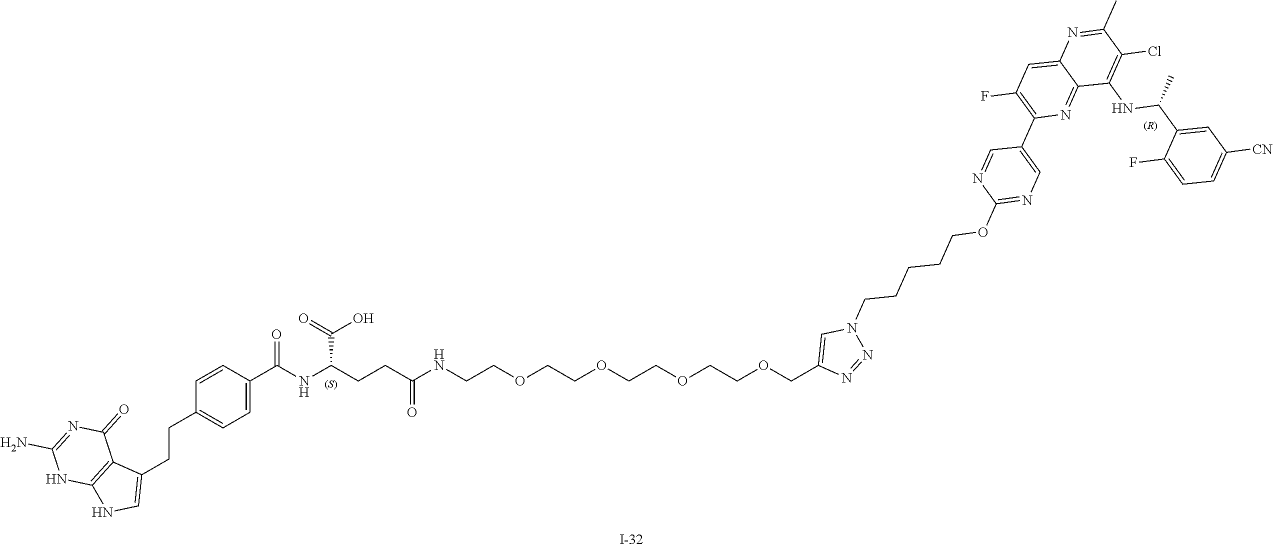

To a solution of (4-(2-(2-amino-4-oxo-4,7-dihydro-1H-pyrrolo[2,3-d]pyrimidin-5-yl)ethyl)benzoyl)-L-glutamic acid (Pemetrexed) (1, 1.0 eq, 0.500 g, 1.17 mmol) in N,N-dimethylformamide (10 mL) and dimethyl sulfoxide (10 mL), N-hydroxysuccinimide (1.1 eq, 0.148 g, 1.29 mmol), 1-(3-Dimethylaminopropyl)-3-ethylcarbodiimide hydrochloride (EDC·HCl) (1.1 eq, 0.247 g, 1.29 mmol) and N,N-diisopropylethylamine (3.0 eq, 0.64 mL, 3.51 mmol) were added and the reaction mixture was stirred at room temperature for 30 minutes. Then, 3,6,9,12-tetraoxapentadec-14-yn-1-amine (1a, 1.1 eq, 0.298 g, 1.29 mmol) was added and the reaction mixture was stirred at room temperature for 16 h. After completion, the reaction mixture was directly purified by prep HPLC (20-55% acetonitrile in water with 0.1% acetic acid). Fractions containing the desired product were combined and lyophilized to dryness to afford (S)-20-(4-(2-(2-amino-4-oxo-4,7-dihydro-1H-pyrrolo[2,3-d]pyrimidin-5-yl)ethyl)benzamido)-17-oxo-4,7,10,13-tetraoxa-16-azahenicos-1-yn-21-oic acid (2) as a yellow sticky solid. Yield: 0.100 g, 12.6%; LCMS m/z 641.2 [M+1]⁺.

To a solution of (S)-20-(4-(2-(2-amino-4-oxo-4,7-dihydro-1H-pyrrolo[2,3-d]pyrimidin-5-yl)ethyl)benzamido)-17-oxo-4,7,10,13-tetraoxa-16-azahenicos-1-yn-21-oic acid (2, 1.0 eq, 0.070 g, 0.109 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.061 g, 0.109 mmol) in N,N-dimethylformamide (1.8 mL) and water (0.2 mL), copper sulfate pentahydrate (0.05 eq, 0.001 g, 0.005 mmol) and sodium ascorbate (0.5 eq, 0.010 g, 0.054 mmol) were added and the reaction mixture was stirred at room temperature for 16 h. Then, tetrakis(acetonitrile)copper(I) hexafluorophosphate (1.0 eq, 0.040 g, 0.109 mmol) was added, and the reaction mixture was stirred at room temperature for another 1 h. After completion, the reaction mixture was quenched with acetic acid (0.2 mL) and directly purified by prep HPLC (33-65% acetonitrile in water with 0.1% formic acid). Fractions containing the desired compound were combined and lyophilized to afford (S)-18-(4-(2-(2-amino-4-oxo-4,7-dihydro-1H-pyrrolo[2,3-d]pyrimidin-5-yl)ethyl)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-2,5,8,11-tetraoxa-14-azanonadecan-19-oic acid (Cpd. No. I-32) as a light green solid. Yield: 0.015 g, 11.17%; LCMS m/z 602.76 [M+2]⁺; ¹H NMR (400 MHz, DMSO-d₆) δ 12.54 (bs, 1H), 10.60 (s, 1H), 10.14 (s, 1H), 9.04 (s, 2H), 8.56 (d, J=7.6 Hz, 1H), 8.17 (d, J=11.6 Hz, 1H), 8.10 (s, 1H), 8.01 (d, J=5.2 Hz, 1H), 7.91 (t, J=5.6 Hz, 1H), 7.77 (d, J=8.0 Hz, 3H), 7.33-7.26 (m, 3H), 6.98-6.96 (m, 1H), 6.35-6.29 (m, 2H), 6.00 (s, 2H), 4.50 (s, 2H), 4.42-4.36 (m, 4H), 4.35-4.29 (m, 1H), 3.52-3.46 (m, 14H), 3.19-3.16 (m, 2H), 2.98-2.94 (m, 2H), 2.85-2.82 (m, 2H), 2.63 (s, 3H), 2.24-2.20 (m, 2H), 2.10-2.06 (m, 1H), 1.94-1.88 (m, 3H), 1.83 (t, J=7.2 Hz, 2H), 1.65 (d, J=6.8 Hz, 3H), 1.40 (t, J=7.2 Hz, 2H).

### 6.1.33. Example 33: Compound I-33

### N5-(2-(2-((1-(5-((5-(7-chloro-8-(((S)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methoxy)ethoxy)ethyl)-N2-(4-(((2,4-diaminopteridin-6-yl)methyl)(methyl)amino)benzoyl)-L-glutamine (Compound I-33)

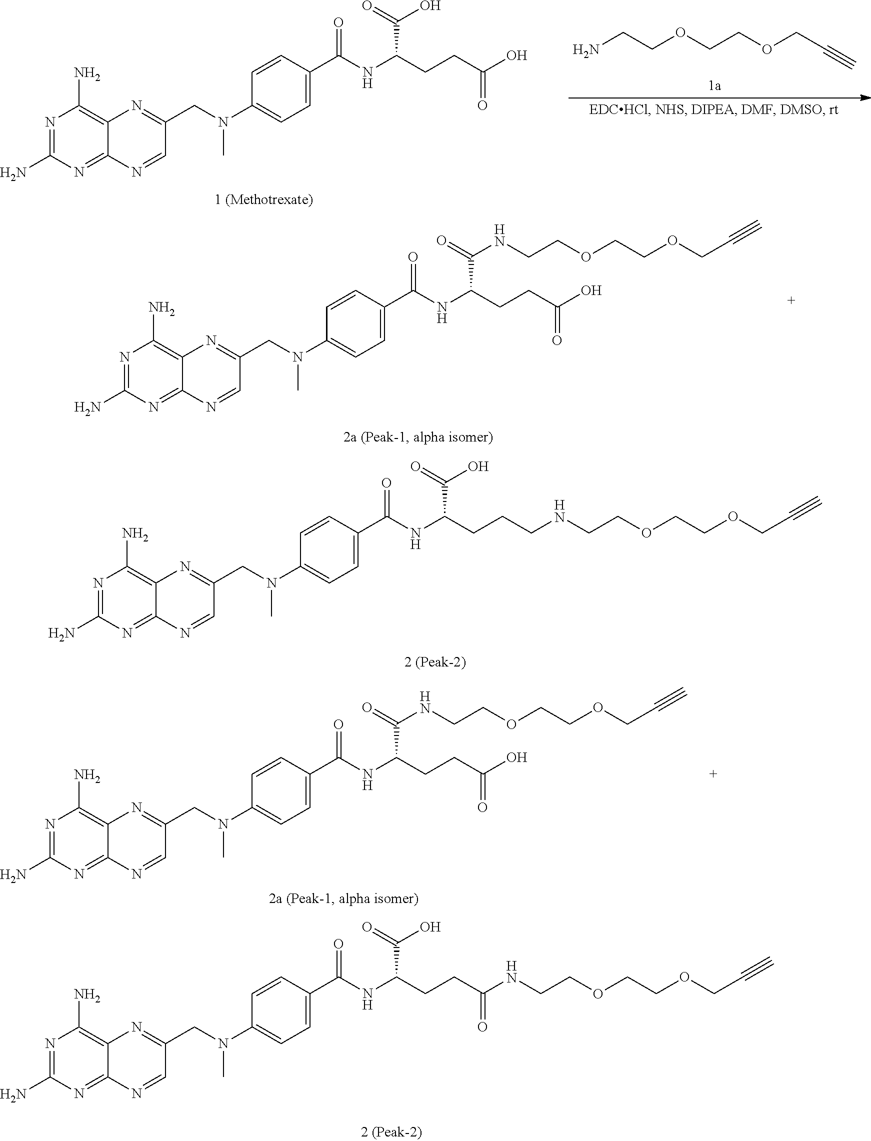

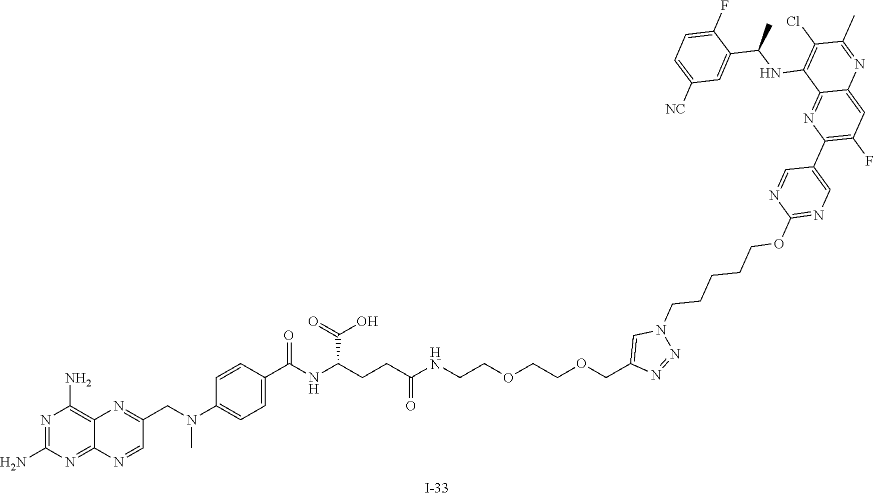

To a solution of (4-(((2,4-diaminopteridin-6-yl)methyl)(methyl)amino)benzoyl)-L-glutamic acid (Methotrexate) (1, 1.0 eq, 0.900 g, 1.98 mmol) in N,N-dimethylformamide (18 mL) and dimethyl sulfoxide (18 mL), N-hydroxysuccinimide (1.1 eq, 0.251 g, 2.18 mmol), 1-(3-dimethylaminopropyl)-3-ethylcarbodiimide hydrochloride (EDC·HCl) (1.1 eq, 0.418 g, 2.18 mmol) and N,N-diisopropylethylamine (3.0 eq, 1.1 mL, 5.94 mmol) were added and the reaction mixture was stirred at room temperature for 1 h. Then, 2-(2-(prop-2-yn-1-yloxy)ethoxy)ethan-1-amine (1a, 1.1 eq, 0.312 g, 2.18 mmol) was added and the reaction mixture was stirred at same temperature for 16 h. After completion, the reaction mixture was directly purified by prep HPLC (10-210% acetonitrile in water with 0.1% acetic acid). The faster eluting fractions were combined and lyophilized to dryness to afford (S)-4-(4-(((2,4-diaminopteridin-6-yl)methyl)(methyl)amino)benzamido)-5-oxo-5-((2-(2-(prop-2-yn-1 yloxy)ethoxy)ethyl)amino)pentanoic acid (2a, Peak-1, alpha isomer) as a yellow solid. Yield: 0.110 g, 9.6%; LCMS m/z 580.55 [M+1]⁺. The slower eluting fractions were combined and lyophilized to dryness to afford N²-(4-(((2,4-diaminopteridin-6-yl)methyl)(methyl)amino)benzoyl)-N5-(2-(2-(prop-2-yn-1-yloxy)ethoxy)ethyl)-L-glutamine (2, Peak-2) as a yellow solid. Yield: 0.180 g, 15.7%; LCMS m/z 580.64 [M+1]⁺.

To a solution of N²-(4-(((2,4-diaminopteridin-6-yl)methyl)(methyl)amino)benzoyl)-N⁵-(2-(2-(prop-2-yn-1-yloxy)ethoxy)ethyl)-L-glutamine (2, 1.0 eq, 0.050 g, 0.086 mmol) and (S)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.048 g, 0.086 mmol) in dimethyl sulfoxide (1.5 mL), tetrakis(acetonitrile)copper(I) hexafluorophosphate (2.8 eq, 0.090 g, 0.242 mmol) was added and the reaction mixture was stirred at room temperature for 1 h. After completion, the reaction mixture was quenched with acetic acid (0.2 mL) and directly purified by prep HPLC (32-50% acetonitrile in water with 0.1% acetic acid). Fractions containing the desired product were combined and lyophilized to dryness to afford N⁵-(2-(2-((1-(5-((5-(7-chloro-8-(((S)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methoxy)ethoxy)ethyl)-N²-(4-(((2,4-diaminopteridin-6-yl)methyl)(methyl)amino)benzoyl)-L-glutamine (Cpd. No. I-33) as a yellow solid. Yield: 0.008 g, 8.1%; LCMS m/z 572.71 [M+2]⁺⁺; ¹H NMR (400 MHz, DMSO-d₆ with D₂O) δ 8.99 (s, 2H), 8.56 (bs, 1H), 8.09-8.06 (m, 2H), 7.94 (d, J=4.8 Hz, 1H), 7.72-7.67 (m, 3H), 7.26 (t, J=8.4 Hz, 1H), 6.78 (d, J=8.8 Hz, 2H), 6.30 (d, J=6.4 Hz, 1H), 4.77 (s, 2H), 4.47 (s, 2H), 4.36 (d, J=6.4 Hz, 4H), 4.25 (bs, 1H), 3.48-3.45 (m, 6H), 3.33-3.30 (m, 2H), 3.17 (s, 3H), 3.16-3.12 (m, 3H), 2.61 (s, 3H), 2.18-2.15 (m, 2H), 2.06-1.99 (m, 2H), 1.88-1.84 (m, 2H), 1.78-1.76 (m, 2H), 1.62 (d, J=6.8 Hz, 3H), 1.36-1.34 (m, 2H).

### 6.1.34. Example 34: Compound I-34

(S)-22-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-21-oxo-2,5,8,11,14,17-hexaoxa-20-azapentacosan-25-oic acid (Compound I-34)

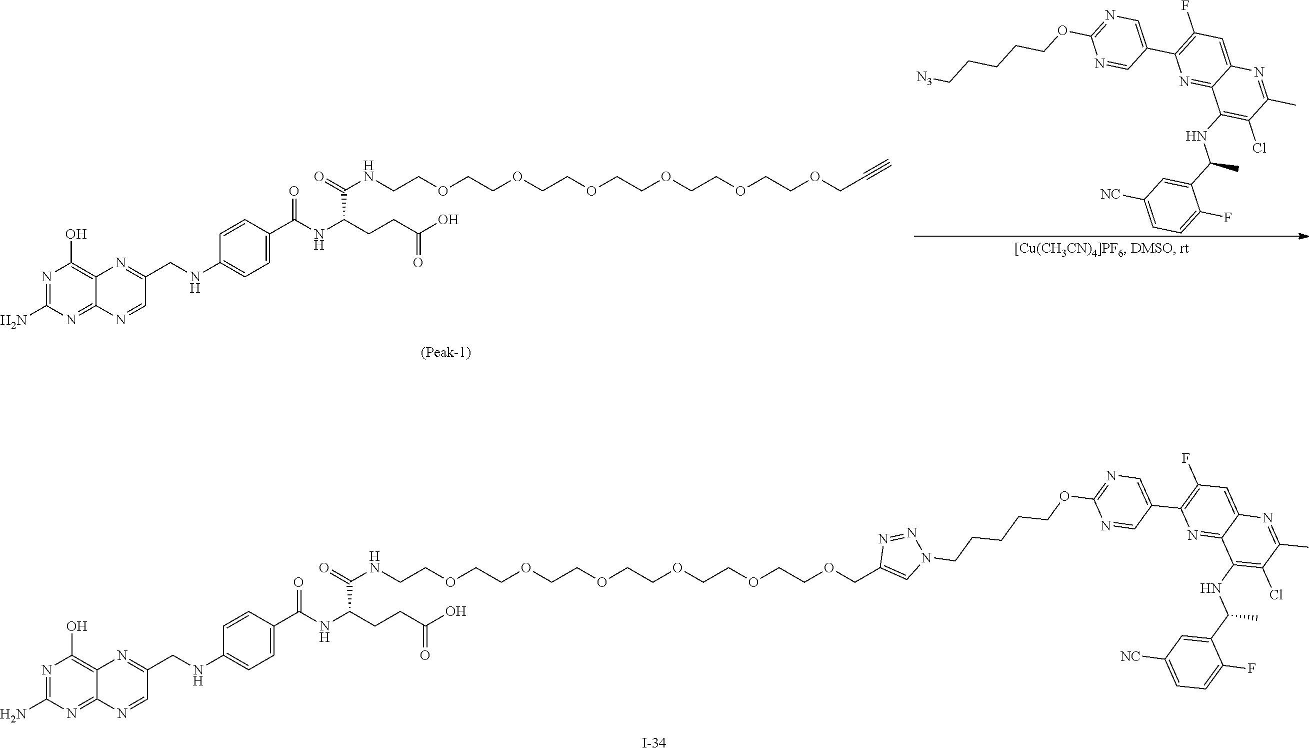

To a solution of (S)-24-(4-(((2-amino-4-hydroxypteridin-6-yl)methyl)amino)benzamido)-23-oxo-4,7,10,13,16,19-hexaoxa-22-azaheptacos-1-yn-27-oic acid (Peak-1, 4.0 eq, 0.132 g, 0.177 mmol) and (S)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.025 g, 0.0443 mmol) in dimethylsulfoxide (2 mL), tetrakis(acetonitrile)copper(I) hexafluorophosphate was added (2.8 eq, 0.0463 g, 0.124 mmol) was added and the reaction mixture was stirred at room temperature for 1 h. After completion, the reaction mixture was quenched with acetic acid (0.2 mL) and directly purified by prep HPLC (35-63% acetonitrile in water with 0.1% acetic acid). Fractions containing the desired compound were combined and lyophilized to afford (S)-22-(4-(((2-amino-4-hydroxypteridin-6-yl)methyl)amino)benzamido)-1-(1-(5-((5-(7-chloro-8-(((S)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-21-oxo-2,5,8,11,14,17-hexaoxa-20-azapentacosan-25-oic acid (Cpd. No. I-34) as a yellow solid. Yield: 0.012 g, 20.7%; LCMS m/z 1306.74 [M+1]⁺; ¹H NMR (400 MHz, DMSO-d₆ with D₂O) δ 8.85 (s, 2H), 8.58 (s, 1H), 8.01-7.90 (m, 2H), 7.79-7.78 (m, 1H), 7.65-7.58 (m, 1H), 7.55-7.53 (m, 2H), 7.21-7.10 (m, 1H), 6.57 (d, J=7.6 Hz, 1H), 6.29 (bs, 1H), 4.45-4.41 (m, 4H), 4.33 (bs, 4H), 3.44-3.36 (m, 26H), 3.16-3.14 (m, 2H), 2.24 (bs, 4H), 1.98-1.92 (m, 2H), 1.88-1.82 (m, 3H), 1.80-1.70 (m, 4H), 1.63-1.57 (m, 3H), 1.35-1.27 (m, 2H).

### 6.1.35. Example 35: Compound I-35

(S)-4-(4-(((2-amino-4-hydroxypteridin-6-yl)methyl)amino)benzamido)-5-((2-(2-((1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methoxy)ethoxy)ethyl)amino)-5-oxopentanoic acid (Compound I-35)

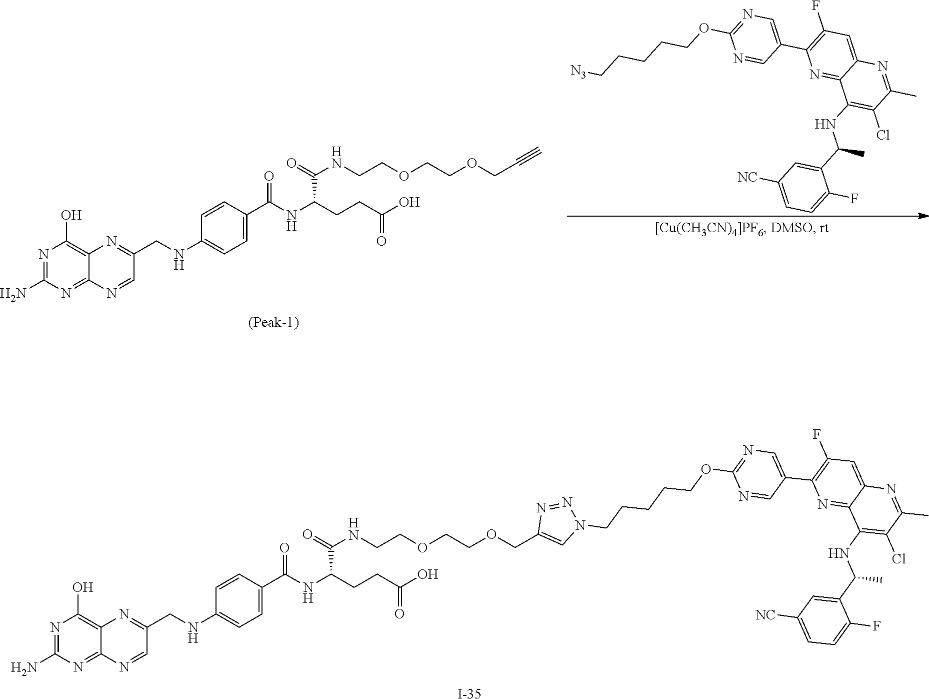

To a solution of (S)-4-(4-(((2-amino-4-hydroxypteridin-6-yl)methyl)amino)benzamido)-5-oxo-5-((2-(2-(prop-2-yn-1-yloxy)ethoxy)ethyl)amino)pentanoic acid (Peak-1, 4.0 eq, 0.092 g, 0.163 mmol) and (S)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.023 g, 0.040 mmol) in dimethylsulfoxide (2 mL), tetrakis(acetonitrile)copper(I) hexafluorophosphate was added (2.8 eq, 0.042 g, 0.114 mmol) and the reaction mixture was stirred at room temperature for 1 h. After completion, the reaction mixture was quenched with acetic acid (0.2 mL) and directly purified by prep HPLC (25-47% acetonitrile in water with 0.1% acetic acid). Fractions containing the desired compound were combined and lyophilized to afford (S)-4-(4-(((2-amino-4-hydroxypteridin-6-yl)methyl)amino)benzamido)-5-((2-(2-((1-(5-((5-(7-chloro-8-(((S)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methoxy)ethoxy)ethyl)amino)-5-oxopentanoic acid (Cpd. No. I-35) as a yellow solid. Yield: 0.003 g, 6.5%; LCMS m/z 566.27 [M+2]⁺⁺; ¹H NMR (400 MHz, DMSO-d₆ with D₂O) δ 8.99 (s, 2H), 8.62 (s, 1H), 8.11 (d, J=11.2 Hz, 1H), 8.07 (s, 1H), 7.96-7.94 (m, 1H), 7.80-7.70 (m, 1H), 7.62 (d, J=8.4 Hz, 1H), 7.28 (t, J=8.4 Hz, 1H), 6.61 (d, J=8.4 Hz, 2H), 6.35-6.32 (m, 1H), 4.47 (s, 5H), 4.38-4.30 (m, 6H), 3.50-3.48 (m, 4H), 3.37-3.35 (m, 2H), 3.25-3.15 (m, 2H), 2.63 (bs, 4H), 2.25-2.23 (m, 4H), 1.95-1.88 (m, 5H), 1.80-1.75 (m, 4H), 1.64 (d, J=6.8 Hz, 3H), 1.40-1.30 (m, 2H).

### 6.1.36. Example 36: Compound I-36

(S)-2-(4-(((2-amino-4-hydroxypteridin-6-yl)methyl)amino)benzamido)-3-(4-(2-((1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methoxy)ethoxy)phenyl)propanoic acid (Compound I-36)

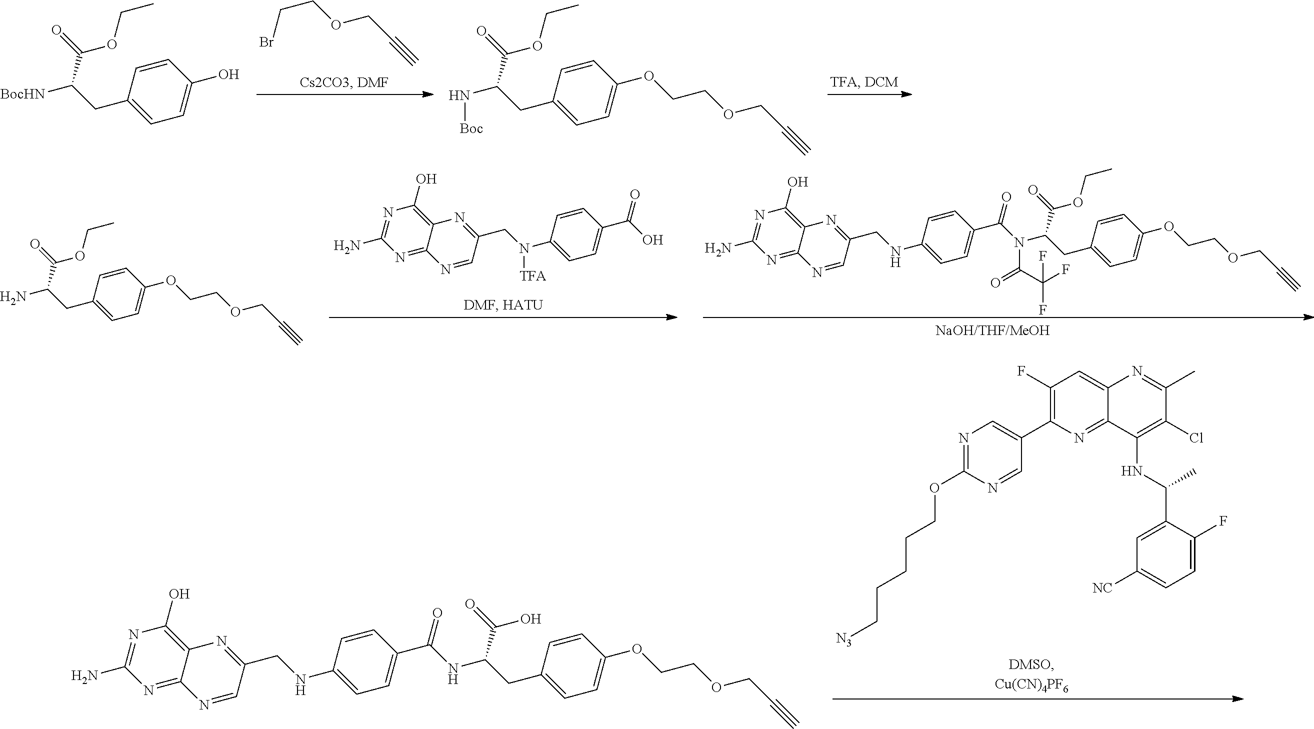

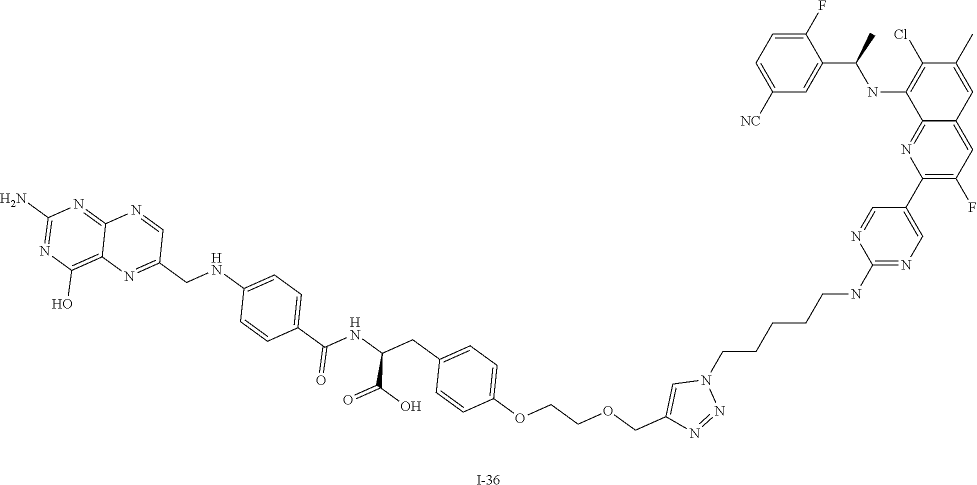

To a room temperature solution of (s)-ethyl 2-((tert-butoxycarbonyl)amino)-3-(4-hydroxyphenyl)propanoate (1.00 eq, 1000 mg, 3.23 mmol) in 8 mL of anhydrous DMF was added Cs₂CO₃ (1.50 eq, 1580 mg, 4.85 mmol) followed by 3-(2-bromoethoxy)prop-1-yne (1.50 eq, 790 mg, 4.85 mmol) and the reaction mixture was stirred at 45° C. for 20 hours. The mixture was diluted with EtOAc (150 mL) washed twice with water, once with brine solution, and the organic fraction dried over sodium sulfate and concentrated to 2 g yellow oil. The crude product was purified by column chromatography, eluting from 80 g silica gel with a gradient of 0-70% EtOAc/hexanes to give 1.2 g of a white solid. 95% yield; LCMS (m/z): 414.2 [M+Na]

Ethyl (2S)-2-(tert-butoxycarbonylamino)-3-[4-(2-prop-2-ynoxyethoxy)phenyl]propanoate (1.00 eq, 1200 mg, 3.07 mmol) in DCM (24 mL) was cooled to 0 C and TFA (12 mL) as added. The resulting solution was stirred for 1 hr at 0° C. and 1 hr at RT. The solution was concentrated to a residue, which was diluted and concentrated from DCE twice. The crude oil was placed under high vacuum overnight to give 1.5 g of semi-solid. Quantitative yield; LCMS (m/z): 292.1 [M+1]

To a solution of 4-[(2-amino-4-hydroxy-pteridin-6-yl)methyl-(2,2,2-trifluoroacetyl)amino]benzoic acid (1.20 eq, 60.4 mg, 0.148 mmol) in anhydrous DMF (200 μL) was added N,N-Diisopropylethylamine (3.00 eq, 0.064 mL, 0.370 mmol) and HATU (1.30 eq, 61.0 mg, 0.160 mmol) followed by a solution of ethyl (2S)-2-amino-3-[4-(2-prop-2-ynoxyethoxy)phenyl]propanoate;2,2,2-trifluoroacetic acid (1.00 eq, 50.0 mg, 0.123 mmol) and 25 μL N,N-Diisopropylethylamine in 123 μL anhydrous DMF. The mixture was stirred at room temperature for 4 hours. The crude reaction mixture was purified by HPLC eluting from a 20 mm×150 C18 column with 15-100% CH3CN/water to give 29 mg of a white solid. 34% yield; LCMS m/z=682.2 [M+1]⁺.

To a slurry of ethyl (2S)-2-[[4-[(2-amino-4-hydroxy-pteridin-6-yl)methylamino]benzoyl]-(2,2,2-trifluoroacetyl)amino]-3-[4-(2-prop-2-ynoxyethoxy)phenyl]propanoate (1.00 eq, 15.0 mg, 0.0220 mmol) in 100 μL MeOH and 100 μL THF was added 55 μL of 1N NaOH and the mixture was stirred at room temperature for 18 hours. And additional 22 μL of 1N NaOH was added, and the mixture was stirred for 20 hours. 75 μL of 1 N HCl was added to precipitate an orange solid. The solid was washed with water and dried under high-vacuum to give 10 mg of desired product. 81.5% yield; LCMS m/z: 558 [M+1]

To a solution of (2S)-2-[[4-[(2-amino-4-hydroxy-pteridin-6-yl)methylamino]benzoyl]amino]-3-[4-(2-prop-2-ynoxyethoxy)phenyl]propanoic acid (1.00 eq, 10.0 mg, 0.0179 mmol) and 3-[(1R)-1-[[6-[2-(5-azidopentoxy)pyrimidin-5-yl]-3-chloro-7-fluoro-2-methyl-1,5-naphthyridin-4-yl]amino]ethyl]-4-fluoro-benzonitrile (1.00 eq, 10.1 mg, 0.0179 mmol) in dimethylsulfoxide 200 uL, tetrakis(acetonitrile)copper(I) hexafluorophosphate (0.500 eq, 3.4 mg, 0.00894 mmol) was added and reaction mixture was stirred at room temperature for 1 h. After completion, reaction mixture was directly purified by prep HPLC (eluting from a C18 column with 35-100% acetonitrile in water with 0.1% FA). Fractions containing the desired product were combined and lyophilized to dryness to afford the desired product (Compound I-36). Yield: 5.8 mg, 27%; LCMS m/z 1221.3 [M+1]⁺.

### 6.1.37. Example 37: Compound I-37

### (S)-2-(4-(((2-amino-4-hydroxypteridin-6-yl)methyl)amino)benzamido)-5,16-dioxo-20-((3aS,4S,6aR)-2-oxohexahydro-1H-thieno[3,4-d]imidazol-4-yl)-9,12-dioxa-6,15-diazaicosanoic acid (Compound I-37)

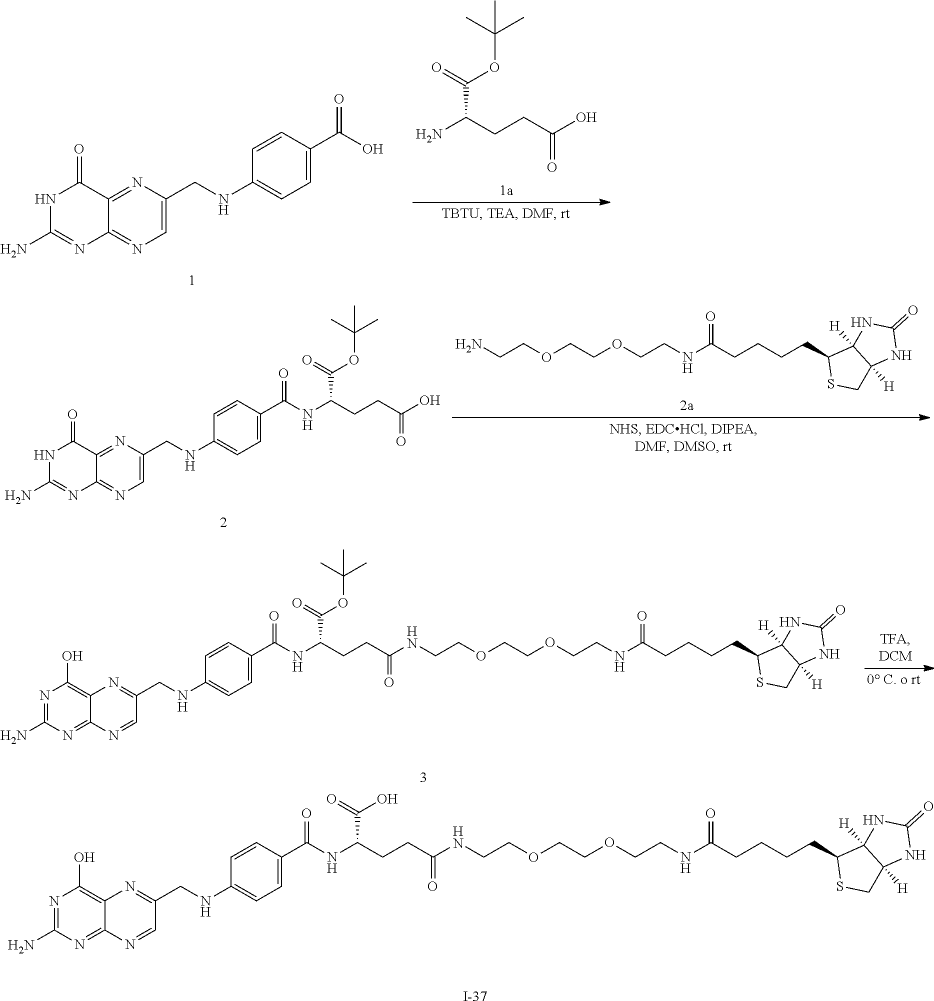

To a suspension of 4-(((2-amino-4-oxo-3,4-dihydropteridin-6-yl)methyl)amino)benzoic acid (pteroic acid) (1, 1.0 eq, 4.0 g, 12.8 mmol) in N,N-dimethylformamide (80 mL), triethylamine (2.0 eq, 3.57 mL, 25.6 mmol) and 2-(1H-Benzotriazole-1-yl)-1,1,3,3-tetramethylaminium tetrafluoroborate (TBTU) (1.0 eq, 4.11 g, 12.8 mmol) were added and reaction mixture was stirred at room temperature for 2.5 h. In another round bottom flask, a suspension of (S)-4-amino-5-(tert-butoxy)-5-oxopentanoic acid (1a, 1.15 eq, 2.99 g, 14.7 mmol) and triethylamine (1.2 eq, 2.13 mL, 15.4 mmol) in N,N-dimethylformamide (60 mL) was prepared. The active ester was added to this suspension and reaction mixture was stirred at room temperature for 16 h. After completion (monitored by LCMS), reaction mixture was concentrated, ethyl acetate was added and stirred for 30 minutes. The resulting brown solid was collected by filtration, washed with chloroform and dried to get crude (4.0 g). This crude was dissolved in aqueous 1N sodium hydroxide solution (16 mL) and water (24 mL) which was purified by prep. HPLC (22-55% acetonitrile in water with 5 mM ammonium bicarbonate). Fractions containing the desired product were combined and lyophilized to dryness to afford (S)-4-(4-(((2-amino-4-oxo-3,4-dihydropteridin-6-yl)methyl)amino)benzamido)-5-(tert-butoxy)-5-oxopentanoic acid (2) as a yellow solid. Yield: 1.3 g, 20.4%; LCMS m/z 498.13 [M+1]⁺.

To a solution of (S)-4-(4-(((2-amino-4-oxo-3,4-dihydropteridin-6-yl)methyl)amino)benzamido)-5-(tert-butoxy)-5-oxopentanoic acid (2, 1.0 eq, 0.100 g, 0.201 mmol) in N,N-dimethylformamide (2 mL) and dimethyl sulfoxide (2 mL), N-hydroxysuccinimide (1.5 eq, 0.034 g, 0.302 mmol), N-(3-Dimethylaminopropyl)-N′-ethylcarbodiimide hydrochloride (EDC·HCl) (1.5 eq, 0.057 g, 0.302 mmol) and N,N-diisopropylethylamine (3.0 eq, 0.1 mL, 0.603 mmol) were added and reaction mixture was stirred at room temperature for 30 minutes. Then, N-(2-(2-(2-aminoethoxy)ethoxy)ethyl)-5-((3aS,4S,6aR)-2-oxohexahydro-1H-thieno[3,4-d]imidazol-4-yl)pentanamide (2a, 1.2 eq, 0.090 g, 0.241 mmol) was added and reaction mixture was stirred at room temperature for 16 h. After that, reaction mixture was directly purified by prep HPLC (20-32% acetonitrile in water with 0.1% acetic acid). Fractions containing the desired product were combined and lyophilized to dryness to afford tert-butyl (S)-2-(4-(((2-amino-4-hydroxypteridin-6-yl)methyl)amino)benzamido)-5,16-dioxo-20-((3aS,4S,6aR)-2-oxohexahydro-1H-thieno[3,4-d]imidazol-4-yl)-9,12-dioxa-6,15-diazaicosanoate (3) as a yellow solid. Yield: 0.007 g, 3.68%; LCMS m/z 854.51 [M+1]⁺.

A solution of tert-butyl (S)-2-(4-(((2-amino-4-hydroxypteridin-6-yl)methyl)amino)benzamido)-5,16-dioxo-20-((3aS,4S,6aR)-2-oxohexahydro-1H-thieno[3,4-d]imidazol-4-yl)-9,12-dioxa-6,15-diazaicosanoate (3, 1.0 eq, 0.006 g, 0.007 mmol) in dichloromethane (0.5 mL) was cooled at 0° C., trifluoroacetic acid (0.5 ml) was added and reaction mixture was stirred at room temperature for 3 h. After completion, reaction mixture was concentrated to get crude which was purified by prep HPLC (10-30% acetonitrile in water with 0.1% TFA). Fractions containing the desired product were combined and lyophilized to dryness to afford (S)-2-(4-(((2-amino-4-hydroxypteridin-6-yl)methyl)amino)benzamido)-5,16-dioxo-20-((3aS,4S,6aR)-2-oxohexahydro-1H-thieno[3,4-d]imidazol-4-yl)-9,12-dioxa-6,15-diazaicosanoic acid (Cpd. No. I-37) as a yellow solid. Yield: 0.0015 g, 27.27%; LCMS m/z 798.56 [M+1]⁺; ¹H NMR (400 MHz, DMSO-d6 with D₂O) δ 8.62 (s, 1H), 7.59 (d, J=8.8 Hz, 2H), 6.62 (d, J=8.8 Hz, 2H), 4.47 (s, 2H), 4.33-4.29 (m, 1H), 4.19-4.17 (m, 1H), 4.14-4.11 (m, 1H), 3.42 (s, 4H), 3.36-3.32 (m, 4H), 3.16-3.13 (m, 4H), 3.07-3.05 (m, 1H), 2.80-2.75 (m, 1H), 2.57-2.54 (m, 1H), 2.16-2.12 (m, 2H), 2.05-2.02 (m, 3H), 1.92-1.81 (m, 1H), 1.65-1.50 (m, 1H), 1.46-1.43 (m, 3H), 1.25-1.23 (m, 2H).

### 6.1.38. Example 38: Compound I-38

### N2-(4-(((2-amino-4-hydroxypteridin-6-yl)methyl)amino)benzoyl)-N5-(2-(2-((1-(2-(2-(2-(5-((3aS,4S,6aR)-2-oxohexahydro-1H-thieno[3,4-d]imidazol-4-yl)pentanamido)ethoxy)ethoxy)ethyl)-1H-1,2,3-triazol-4-yl)methoxy)ethoxy)ethyl)-L-glutamine (Compound I-38)

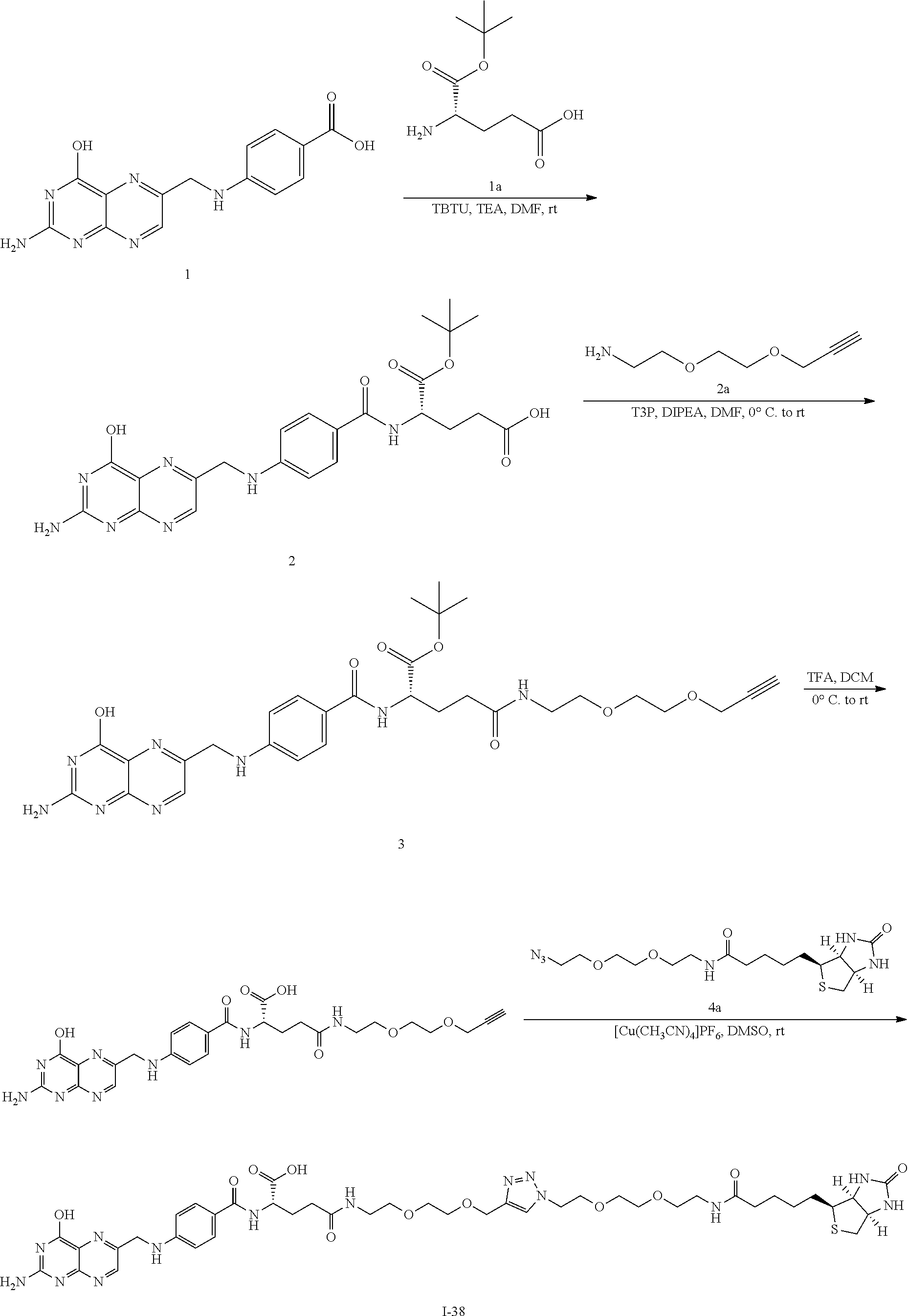

To a suspension of 4-(((2-amino-4-hydroxypteridin-6-yl)methyl)amino)benzoic acid (pteroic acid) (1, 1.0 eq, 4.0 g, 12.8 mmol) in N,N-dimethylformamide (80.0 mL), triethylamine (2.0 eq, 3.57 mL, 25.6 mmol) and 2-(1H-Benzotriazole-1-yl)-1,1,3,3-tetramethylaminium tetrafluoroborate (TBTU) (1.0 eq, 4.11 g, 12.8 mmol) were added and reaction mixture was stirred at room temperature for 2.5 h. In another round bottom flask, a suspension of (S)-4-amino-5-(tert-butoxy)-5-oxopentanoic acid (1a, 1.15 eq, 2.99 g, 14.7 mmol) and triethylamine (1.2 eq, 2.13 mL, 15.4 mmol) in N,N-dimethylformamide (60 mL) was prepared. The active ester was added to this suspension and reaction mixture was stirred at room temperature for 16 h. After completion, N,N-dimethylformamide was concentrated, ethyl acetate was added and stirred for 30 minutes. The solid was collected by filtration, washed with chloroform to get crude (4.0 g). This crude was dissolved in aqueous 1N sodium hydroxide solution (16 mL) and water (24 mL) which was purified by prep. HPLC (22-55% acetonitrile in water with 5 mM ammonium bicarbonate). Fractions containing the desired product were combined and lyophilized to dryness to afford (S)-4-(4-(((2-amino-4-hydroxypteridin-6-yl)methyl)amino)benzamido)-5-(tert-butoxy)-5-oxopentanoic acid (2) as a yellow solid. Yield: 1.3 g, 20.4%; LCMS m/z 498.13 [M+1]⁺.

A solution of (S)-4-(4-(((2-amino-4-hydroxypteridin-6-yl)methyl)amino)benzamido)-5-(tert-butoxy)-5-oxopentanoic acid (2, 1.0 eq, 0.100 g, 0.201 mmol) in N,N-dimethylformamide (2.0 mL) was cooled at 0° C., 2-(2-(prop-2-yn-1-yloxy)ethoxy)ethan-1-amine (2a, 1.5 eq, 0.043 g, 0.302 mmol), N,N-diisopropylethylamine (3.0 eq, 0.1 mL, 0.603 mmol) and 1-propanephosphonic anhydride (T3P) (1.5 eq, 0.18 mL, 0.302 mmol) were added and reaction mixture was stirred at room temperature for 72 h. After that, reaction mixture was directly purified by prep HPLC (20-32% acetonitrile in water with 0.1% acetic acid). Fractions containing the desired product were combined and lyophilized to dryness to afford tert-butyl N2-(4-(((2-amino-4-hydroxypteridin-6-yl)methyl)amino)benzoyl)-N5-(2-(2-(prop-2-yn-1-yloxy)ethoxy)ethyl)-L-glutaminate (3) as a yellow solid. Yield: 0.014 g, 11.19%; LCMS m/z 623.19 [M+1]⁺.

A solution of tert-butyl N2-(4-(((2-amino-4-hydroxypteridin-6-yl)methyl)amino)benzoyl)-N5-(2-(2-(prop-2-yn-1-yloxy)ethoxy)ethyl)-L-glutaminate (1.0 eq, 0.014 g, 0.022 mmol) in dichloromethane (0.5 mL) was cooled at 0° C., trifluoroacetic acid (0.5 mL) was added and reaction mixture was stirred at room temperature for 2 h. After completion, reaction mixture was concentrated, azeotroped with dichloromethane (2-3 times) and dried to afford N2-(4-(((2-amino-4-hydroxypteridin-6-yl)methyl)amino)benzoyl)-N5-(2-(2-(prop-2-yn-1-yloxy)ethoxy)ethyl)-L-glutamine as a yellow solid. Yield: 0.012 g (Crude); LCMS m/z 567.14 [M+1]⁺.

To a solution of N2-(4-(((2-amino-4-hydroxypteridin-6-yl)methyl)amino)benzoyl)-N5-(2-(2-(prop-2-yn-1-yloxy)ethoxy)ethyl)-L-glutamine (1.0 eq, 0.012 g, 0.021 mmol) and N-(2-(2-(2-azidoethoxy)ethoxy)ethyl)-5-((3aS,4S,6aR)-2-oxohexahydro-1H-thieno[3,4-d]imidazol-4-yl)pentanamide (4a, 1.0 eq, 0.008 g, 0.021 mmol) in dimethylsulfoxide (0.5 mL), tetrakis(acetonitrile)copper(I) hexafluorophosphate (2.8 eq., 0.022 g, 0.059 mmol) was added and reaction mixture was stirred at room temperature for 1 h. After completion, reaction mixture was directly purified by prep HPLC (13-25% acetonitrile in water with 0.1% TFA). Fractions containing the desired product were combined and lyophilized to dryness to afford N2-(4-(((2-amino-4-hydroxypteridin-6-yl)methyl)amino)benzoyl)-N5-(2-(2-((1-(2-(2-(2-(5-((3aS,4S,6aR)-2-oxohexahydro-1H-thieno[3,4-d]imidazol-4-yl)pentanamido)ethoxy)ethoxy)ethyl)-1H-1,2,3-triazol-4-yl)methoxy)ethoxy)ethyl)-L-glutamine (Cpd. No. I-38) as a yellow solid. Yield: 0.003 g, 14.15%; LCMS m/z 967.67 [M+1]⁺; ¹H NMR (400 MHz, DMSO-d6) δ 12.46 (s, 1H), 11.40 (s, 1H), 8.64 (s, 1H), 8.19 (d, J=7.6 Hz, 1H), 8.04 (s, 1H), 7.91 (t, J=6.0 Hz, 1H), 7.80 (t, J=4.8 Hz, 1H), 7.64 (d, J=8.8 Hz, 2H), 6.93 (t, J=7.6 Hz, 1H), 6.63 (d, J=6.0 Hz, 1H), 6.42 (s, 1H), 6.36 (s, 1H), 4.50-4.47 (m, 6H), 4.32-4.25 (m, 2H), 4.13-4.10 (m, 1H), 3.79 (t, J=5.2 Hz, 2H), 3.59-3.45 (m, 10H), 3.39-3.35 (m, 4H), 3.20-3.3.15 (m, 5H), 3.10-3.07 (m, 1H), 2.82-2.78 (m, 1H), 2.24-2.18 (m, 3H), 2.05 (t, J=7.2 Hz, 2H), 1.95-1.85 (m, 1H), 1.61-1.57 (m, 1H), 1.52-1.42 (m, 3H), 1.28-1.23 (m, 2H).

### 6.1.39. Example 39: Compound I-39

### Synthesis of (S)-30-(4-(((2-amino-4-hydroxypteridin-6-yl)methyl)amino)benzamido)-27-oxo-1-(1-(2-(2-(2-(5-((3aS,4S,6aR)-2-oxohexahydro-1H-thieno[3,4-d]imidazol-4-yl)pentanamido)ethoxy)ethoxy)ethyl)-1H-1,2,3-triazol-4-yl)-2,5,8,11,14,17,20,23-octaoxa-26-azahentriacontan-31-oic acid (Compound I-39)

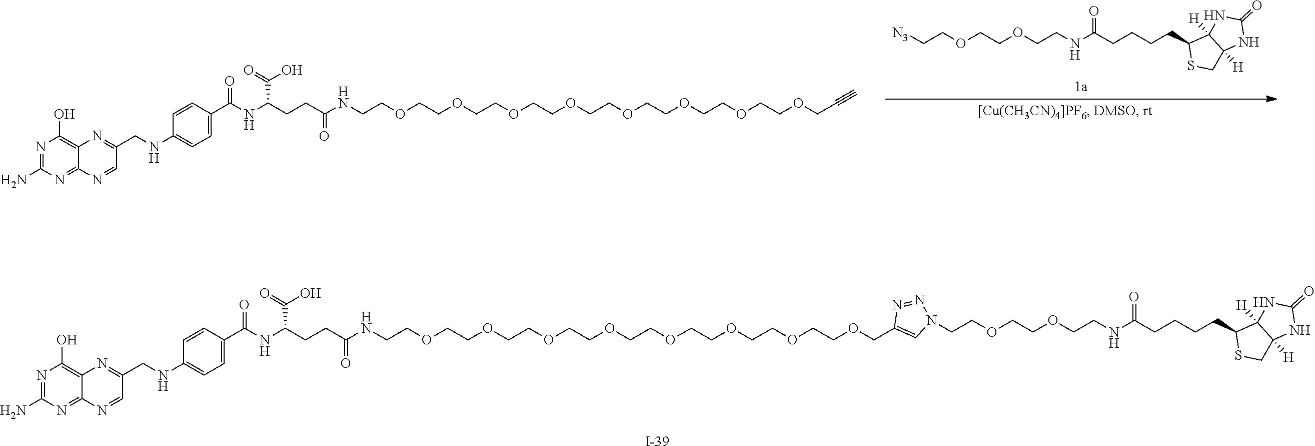

To a solution of(S)-32-(4-(((2-amino-4-hydroxypteridin-6-yl)methyl)amino)benzamido)-29-oxo-4,7,10,13,16,19,22,25-octaoxa-28-azatritriacont-1-yn-33-oic acid (1.0 eq, 0.040 g, 0.081 mmol) and N-(2-(2-(2-azidoethoxy)ethoxy)ethyl)-5-((3aS,4S,6aR)-2-oxohexahydro-1H-thieno[3,4-d]imidazol-4-yl)pentanamide (1a, 1.05 eq, 0.0202 g, 0.0505 mmol) in anhydrous dimethylsulfoxide (1.5 mL), tetrakis(acetonitrile)copper(I) hexafluorophosphate was added (2.8 eq, 0.0405 g, 0.226 mmol) and reaction mixture was stirred at room temperature for 0.5 h. After completion, the reaction mixture was quenched with acetic acid (0.2 mL) and directly purified by prep HPLC (20-42% acetonitrile in water with 0.1% trifluoroacetic acid). All the fractions containing desired compound were combined and lyophilized to afford (S)-30-(4-(((2-amino-4-hydroxypteridin-6-yl)methyl)amino)benzamido)-27-oxo-1-(1-(2-(2-(2-(5-((3aS,4S,6aR)-2-oxohexahydro-H-thieno[3,4-d]imidazol-4-yl)pentanamido)ethoxy)ethoxy)ethyl)-1H-1,2,3-triazol-4-yl)-2,5,8,11,14,17,20,23-octaoxa-26-azahentriacontan-31-oic acid (Cpd. No. I-39) as a yellow solid. Yield: 0.020 g, 33.73%; LCMS m/z 616.73 [M+2]⁺⁺; ¹H NMR (400 MHz, DMSO-d6 with D₂O) δ 8.66 (s, 1H), 8.01 (s, 1H), 7.62 (d, J=8.4 Hz, 2H), 6.63 (d, J=8.8 Hz, 2H), 4.48-4.46 (m, 6H), 4.31-4.28 (m, 1H), 4.25-4.23 (m, 1H), 4.13-4.10 (m, 1H), 3.78 (t, J=4.8 Hz, 2H), 3.53-3.49 (m, 6H), 3.47-3.45 (m, 26H), 3.37-3.32 (m, 4H), 3.17-3.13 (m, 4H), 3.08-3.06 (m, 1H), 2.80 (dd, J=12.6 and 4.8 Hz, 1H), 2.58 (s, 1H), 2.24-2.16 (m, 2H), 2.06-2.00 (m, 3H), 1.92-1.86 (m, 1H), 1.59-1.56 (m, 1H), 1.47-1.39 (m, 3H), 1.31-1.24 (m, 2H).

### 6.1.40. Example 40: Compound I-40

### N2-(4-(((2-amino-4-hydroxypteridin-6-yl)methyl)amino)benzoyl)-N5-(2-(2-((1-(2-((2-(5-((3aS,4S,6aR)-2-oxohexahydro-1H-thieno[3,4-d]imidazol-4-yl)pentanamido)ethyl)disulfaneyl)ethyl)-1H-1,2,3-triazol-4-yl)methoxy)ethoxy)ethyl)-L-glutamine (Compound I-40)

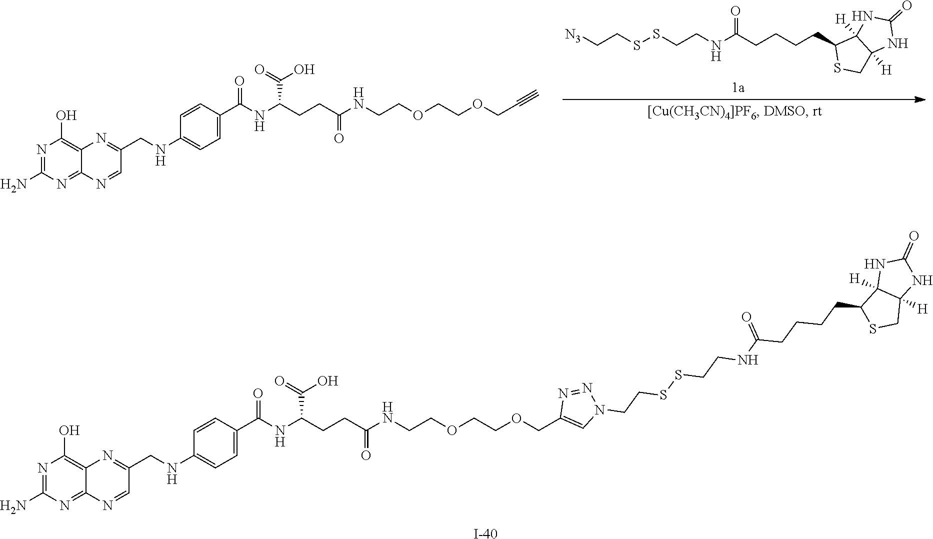

To a solution of N2-(4-(((2-amino-4-hydroxypteridin-6-yl)methyl)amino)benzoyl)-N5-(2-(2-(prop-2-yn-1-yloxy)ethoxy)ethyl)-L-glutamine (1.0 eq, 0.040 g, 0.007 mmol) and N-(2-((2-azidoethyl)disulfaneyl)ethyl)-5-((3aS,4S,6aR)-2-oxohexahydro-1H-thieno[3,4-d]imidazol-4-yl)pentanamide (1a, 1.0 eq, 0.028 g, 0.007 mmol) in anhydrous dimethylsulfoxide (1.5 mL), tetrakis(acetonitrile)copper(I) hexafluorophosphate (2.8 eq, 0.073 g, 0.198 mmol) was added and reaction mixture was stirred at room temperature for 30 minutes. After completion, the reaction mixture was quenched with acetic acid (0.2 mL) and directly purified by prep HPLC (14-25% acetonitrile in water with 0.1% trifluoroacetic acid). All the fractions containing desired compound were combined and lyophilized to afford N2-(4-(((2-amino-4-hydroxypteridin-6-yl)methyl)amino)benzoyl)-N5-(2-(2-((1-(2-((2-(5-((3aS,4S,6aR)-2-oxohexahydro-1H-thieno[3,4-d]imidazol-4-yl)pentanamido)ethyl)disulfaneyl)ethyl)-1H-1,2,3-triazol-4-yl)methoxy)ethoxy)ethyl)-L-glutamine as a yellow solid. Yield: 0.024 g, 35%; LCMS m/z 486.59 [M+2]⁺⁺; ¹H-NMR (400 MHz, DMSO-d6 with D₂O) δ 8.66 (s, 1H), 8.06 (s, 1H), 7.62 (d, J=8.8 Hz, 2H), 6.63 (d, J=8.8 Hz, 2H), 4.61 (t, J=6.8 Hz, 2H), 4.49 (d, J=9.2 Hz, 4H), 4.30-4.28 (m, 1H), 4.29-4.26 (m, 1H), 4.13-4.12 (m, 1H), 3.55-3.45 (m, 4H), 3.34 (t, J=5.6 Hz, 2H), 3.28 (t, J=6.4 Hz, 2H), 3.19-3.13 (m, 4H), 3.10-3.05 (m, 1H), 2.79-2.74 (m, 3H), 2.57 (s, 1H), 2.18-2.16 (m, 2H), 2.07-2.03 (m, 3H), 1.95-1.85 (m, 1H), 1.65-1.55 (m, 1H), 1.53-1.37 (m, 3H), 1.32-1.22 (m, 2H).

### 6.2. Conjugation Examples

### 6.2.1. Example 41: Conjugation of Isothiocyanate-Based Ligand-Linker Compounds with Antibodies

This example provides a general protocol for the conjugation of the isothiocyanate-based ligand-linker compounds described herein with the primary amines on lysine residues of the antibodies described herein.

The antibody is buffer exchanged into 100 mM sodium bicarbonate buffer pH 9.0 at 5 mg/mL concentration, after which about 10-30 equivalents of the isothiocyanate-based ligand-linker compound (freshly prepared as 20 mM stock solution in DMSO) is added and incubated overnight at ambient temperature in a tube revolver at 10 rpm.

The conjugates containing on average eight ligand-linker moieties per antibody are purified using a PD-10 desalting column (GE Healthcare) and followed with formulating the final conjugate into PBS pH 7.4 with Amicon Ultra 15 mL Centrifugal Filters with 30 kDa molecular weight cutoff.

### 6.2.2. Example 42: Conjugation of Perfluorophenoxy-Based Ligand-Linker Compounds with Antibodies

This example provides a general protocol for the conjugation of the perfluorophenoxy-based ligand-linker compounds described herein with the primary amines on lysine residues of the antibodies described herein.

The antibody is buffer exchanged into 50 mM sodium phosphate buffer pH 8.0 at 5 mg/mL concentration, after which about 22 equivalents of perfluorophenoxy-based ligand-linker compound (freshly prepared as 20 mM stock solution in DMSO) is added and incubated for 3 hours at ambient temperature in a tube revolver at 10 rpm.

The conjugates containing on average eight ligand-linker moieties per antibody are purified using a PD-10 desalting column (GE Healthcare) and followed with formulating the final conjugate into PBS pH 7.4 with Amicon Ultra 15 mL Centrifugal Filters with 30 kDa molecular weight cutoff.

### 6.2.3. Example 43: Determination of Drug to Antibody Ratio (DAR) by Mass Spectrometry

This example provides the method for determining DAR values for the conjugates prepared as described in Examples 41 and 42. To determine the DAR value, 10 μg of the antibody (unconjugated or conjugated) is treated 2 μL of non-reducing denaturing buffer (10×, New England Biolabs) for 10 minutes at 75° C. The denatured antibody solution is then deglycosylated by adding 1.5 μL of Rapid-PNGase F (New England Biolabs) and incubated for 10 minutes at 50° C. Deglycosylated samples are diluted 50-fold in water and analyzed on a Waters ACQUITY UPLC interfaced to Xevo G2-S QToF mass spectrometer. Deconvoluted masses are obtained using Waters MassLynx 4.2 Software. DAR values are calculated using a weighted average of the peak intensities corresponding to each loading species using the formula below:

DAR=Σ(drug load distribution (%) of each Ab with drug load n)(n)/100

### 6.2.4. Example 44: Determination of Purity of Conjugates by SEC Method

Purity of the conjugates prepared as described in Examples 41 and 42 are determined through size exclusion high performance liquid chromatography (SEC-HPLC) using a 20 minute isocratic method with a mobile phase of 0.2 M sodium phosphate, 0.2 M potassium chloride, 15 w/v isopropanol, pH 6.8. An injection volume of 10 μL is loaded to a TSKgel SuperSW3000 column, at a constant flow rate of 0.35 mL/min. Chromatographs are integrated based on elution time to calculate the purity of monomeric conjugate species.

### 6.2.5. Example 45: Antibody Disulfide Reduction and Ligand-Linker Conjugation to Antibody

This example provides an exemplary protocol for reduction of the disulfides of the antibodies described herein, and conjugation of the reduced antibodies to the ligand-linker compounds described herein.

Protocol:

Antibody Disulfide Reduction
A) Dilute antibody to 15 mg/mL (0.1 mM IgG) in PBS, pH 7.4.
B) Prepare a fresh 20 mM (5.7 mg/mL) stock solution of tris(2 carboxyethyl)phosphine (TCEP) in H₂O.
C) Add 25 μL of TCEP stock solution from step B) above to 1 mL of antibody from step A) above (0.5 mM final concentration TCEP).
D) Incubate at 37° C. for 2 hours (check for free thiols using 5,5′-dithiobis-(2-nitrobenzoic acid) (DTNB) test).
E) Aliquot the reduced antibody into 4 tubes (250 μL each).

Ligand-linker conjugation to antibody
A) Prepare 10 mM stock solution of ligand-linker compound in DMSO (DMA, DMF or CH₃CN are also acceptable).
B) Add 5 equivalents of 12.5 μL stock solution from step A) above to each tube of reduced antibody (0.5 mM final concentration ligand-linker compound stock solution).
C) Incubate overnight at 4° C. for 4 hours at room temperature; check for free thiols using DTNB test.
D) Run analytical hydrophobic interaction chromatography (HIC) to determine DAR and homogeneity.

### Biological Examples

### 6.2.6. Example 46: Folate Receptor α and Folate Receptor β KO Generation

This example provides the protocol for generation of folate receptor knockout (KO) cells. Cells are washed with PBS and detached using TrypLE. Media is added to the flask to deactivate trypsin. Cells are collected and counted. A total of 1×10⁶ cells are then centrifuged at 300×g for 5 minutes. The cell pellet is washed once with PBS and centrifuged at 300×g for 5 minutes. The cell pellet is resuspended in Lonza SE buffer supplemented with supplement 1 and electroporation enhancer (5 μM final). CRISPR RNP reaction begins by combining equal volumes of 100 μM crRNA and tracrRNA in a PCR tube. Using a thermocycler, this mixture is heated to 95° C. for 5 minutes and allowed slowly to cool to room temperature. Using sgRNA specific for either folate receptor α or folate receptor R, the annealed sgRNA product is combined with TrueCut Cas9 and allowed to incubate at RT for 15 minutes. Resuspended cells in SE buffer are mixed with the RNP reaction and allowed to incubate for 5 minutes. The entire reaction contents is then placed in a single well of a 16-well electroporation cuvette. Using a Lonza Amaxa cells are pulsed with code CA-163. After pulsing, cells are plated into a 10 cm dish. Six days post-RNP, a portion of cells are collected and lysates are prepared to test for knock-out by western.

### 6.2.7. Example 47: Alexa Fluor 647 Conjugation

Antibodies are conjugated to Alexa Fluor 647 using Alexa Fluor™ 647 Protein Labeling Kit (Invitrogen) per the manufacturer's protocol. In brief, antibodies to be labeled are diluted to 2 mg/mL in PBS to a total volume of 500 μL. A 15 DOL (degree of labeling) is used for the conjugation with the fluorophore. Free dye is removed by pre-wetting an Amicon 30 kDa filter with PBS. After incubation, the conjugation reaction is then added to the filter and spun at high speed for 10 minutes. Retained solution is then resuspended in PBS to a final volume of 1 mL and stored at 4° C. indefinitely.

### 6.2.8. Example 48: Live-Cell Surface Staining by Flow Cytometry

This example provides a protocol for the determination of the effect of the conjugates described herein on EGFR levels measured by surface staining using flow cytometry.

Hela parental or folate receptor knockout cells are plated in 6 well plates and treated with vehicle (PBS), unconjugated antibody (e.g., anti-EGFR), or the conjugates prepared as described in Examples 41 and 42 for the indicated period of time.

After incubation, media is aspirated and cells are washed three times with PBS, lifted using Accutase and pelleted by centrifugation at 300×g for 5 minutes. Cells are resuspended in cold FACS buffer and kept cold for the remainder of the staining procedure. A portion of cells are excluded from staining procedure as an unstained control. Cells are stained with either human IgG Isotype-AF647 or cetuximab-AF647 conjugates for 1 h on ice in the dark. Cells are then spun at 300×g for 5 min at 4° C. and washed with cold FACS buffer for a total of three washes. After the final wash, cells are resuspended in 100 μL of FACS buffer with DAPI added at a final concentration of 5 g/mL. Cells are analyzed using a BioRad ZE5 flow cytometer and data is analyzed using Flowlo software. Cells are first gated to remove debris, doublets and dead cells (DAPI negative). EGFR cell surface levels are determined based on AF647 mean fluorescence intensity (MFI).

### 6.2.9. Example 49: Measurement of Total EGFR Levels by Traditional Western Blotting

This example provides the protocol for the measurement of the time course activity of the conjugates prepared as described in Examples 41 and 42 on total EGFR levels in Hela parental and folate receptor KO cells measured by traditional Western blotting.

Once all time-points are collected, all cell pellets are resuspended in 50 μL of radioimmunoprecipitation assay (RIPA) buffer (+protease/phosphatase inhibitor +nuclease).

Lysates are incubated on ice for 1 h.

Lysates are then spun at high-speed for 10 min at 4° C.

40 μL of cleared lysate is transferred to a 96 well plate.

All lysate concentrations are calculated using BCA assay (1:3 dilution).

All lysates are equalized to 2 mg/mL using RIPA as diluent.

Equal volumes (15 μL) of lysate are then mixed with LDS sample buffer (3×LDS+2.5× reducing agent).

Samples are incubated at 98° C. for mins and allowed to cool.

Samples are vortexed and spun down.

15 μL of sample is loaded onto a 26-well bis-tris 4-12% midi-gel.

Gel is allowed to run at 180V for 20 mins.

Gels are transferred to nitrocellulose membrane using iBlot 2 (20V constant, 7 mins).

Membranes are washed 1× in PBS and then placed in Odyssey blocking buffer for 1 h RT with shaking.

Primary antibodies mouse anti-f-actin (SCB) and rabbit anti-EGFR (CST) are diluted 1:1000 in blocking buffer and allowed to incubate overnight at 4° C. with shaking.

Membranes are washed thrice with PBS-T (Tween20 0.1%), at least 5 mins each wash.

Secondary antibodies anti-mouse 680rd and anti-rabbit 800cw are diluted 1:5000 in blocking buffer and allowed to incubate for 1 h at RT with shaking.

Membranes are washed thrice with PBS-T (Tween20 0.1%), at least 5 mins each wash.

Membranes are imaged using licor odyssey scanner.

### 6.2.10. Example 50: Measurement of Cellular EGFR Protein Levels Evaluated by Immunocytochemistry

This example provides an exemplary protocol for the determination of the effect of the conjugates prepared as described in Examples 41 and 42 on cellular EGFR protein levels evaluated by immunocytochemistry.

Hela parental or folate receptor knockout cells are plated in 6 well plates and treated with vehicle (PBS), unconjugated antibody, or the conjugates prepared as described in Examples 41 and 42 at 37° C. for 24 hours. After incubation, media is aspirated and cells are washed thrice with PBS. Cells are fixed with 4% PFA for 10 minutes at room temperature, washed three times with PBS and then blocked with 5% BSA in PBS for 1 hour at RT. Cells are permeabilized with 0.2% Triton-X100 in PBS for 15 minutes. After washing, cells are stained with goat anti-EGFR (AF321; R&D Systems) in blocking buffer overnight at 4 C. After washing, cells are stained with anti-goat 800CW secondary or CellTag700, and imaged on Licor scanner.

### 6.3. Biology Methods

### 6.3.1. Example 51: SPR Binding Kinetics of Subject Compounds to Folate Receptor

A Biacore 8K+ was used to measure the association and dissociation rates of example compounds to FOLR2 (Acro Biosystems, Cat: FO2-H5223-100 μg). Briefly, FOLR2 was reconstituted according to the manufacture's instructions to 200 μg/mL. Both the active and reference surfaces of a Cytiva CM5 Series S Sensor Chip were activated with a 1:1 mixture of 400 mM EDC and 100 mM NHS for 420 s at 10 μL/min. 25 μg/mL FOLR2 diluted in Sodium Acetate pH 5.5 was injected on the active channel (Flow Cell 2) for 420 s at 10p/min. Both the reference and active surfaces were then capped with 1M ethanolamine, pH 8.5 for 420 s, achieving an immobilization level of ˜4200 RU. Example compounds were serially diluted 1:1 in running buffer (10 mM HEPES, 150 mM NaCl, 0.05% T20, pH 7.5, 2% DMSO) from 100 nM to 3.125 nM and flowed over the active and reference surfaces for 120 s, and then allowed to dissociate in running buffer for 600 s, at 30 uL/min. The FOLR2 immobilized active surface and the reference surface were regenerated with a 30 s pulse of Sodium Acetate pH 5.5 at 30 μL/min. Sensorgrams were solvent corrected, double-referenced, and the data fitted and analyzed in Biacore Insight Evaluation Software Version 3.0.11.15423.

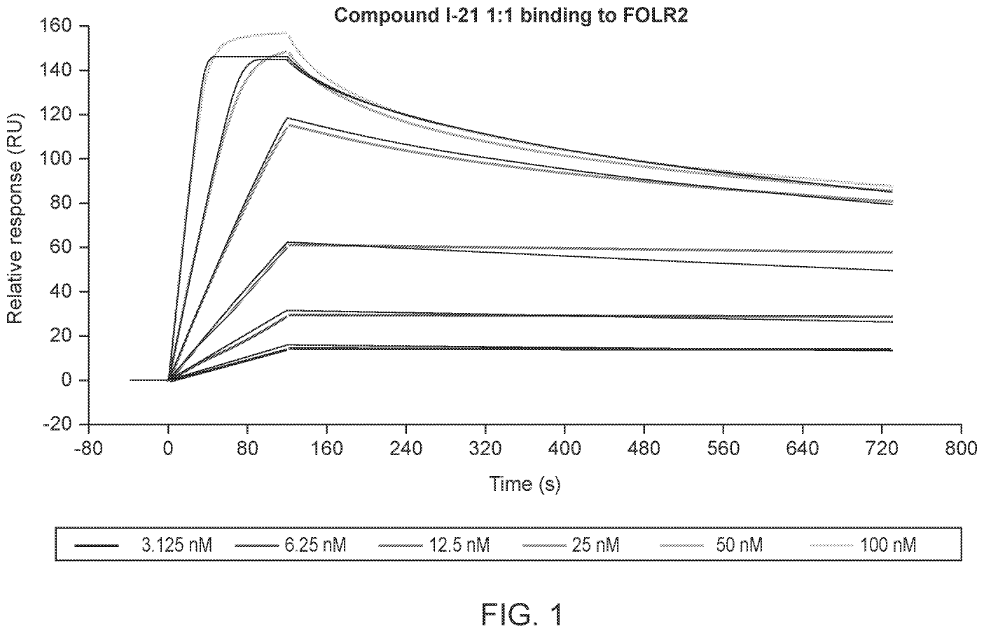

FIG. 1 illustrates the SPR sensorgrams for various concentrations of compound I-21 (3.125 nM to 100 nM) to folate receptor 2 (FOLR2), illustrating 1:1 binding.

TABLE 10
SPR results of compound I-21 biding to FOLR2

| | | Ligand | Amount | | |
|---|---|---|---|---|---|
| | Immobilized | MW | Immobilized | Analyte 1 | MW |
| Channel | ligand | (Da) | (RU) | Solution | (Da) |
| 1 | FOLR2 | 32500 | 4213.2 | Example # | 1218.7 |

| Kinetics | | | | | | Expected | |
|---|---|---|---|---|---|---|---|
| Chi² | ka | kd | KD | Rmax | | Rmax | |
| (RU²) | (1/Ms) | (1/s) | (M) | (RU) | tc | (RU) | Stoichiometry |
| 7.10E+00 | 5.57E+06 | 4.64E−03 | 8.33E−10 | 147.5 | 1.47E+07 | 158.0 | 0.9 |

### 6.3.2. Example 52: SPR Binding Kinetics of Subject Compounds to TNFα Trimer

Streptavidin 100 μg/mL Streptavidin (Invitrogen Cat #:434302) diluted in 10 mM sodium acetate, pH 4.5) was immobilized to both the reference and active surfaces as described in Example 51. The surfaces were conditioned with three injections of running Buffer (10 mM HEPES, 150 mM NaCl, 0.05% T20, pH 7.5, 2% DMSO) at a flow rate of 10 μL/min, Contact time: 60 s. Flow rate: 10 μL/min. The active surface was treated with biotinylated linked TNFα timer 20 μg/mL diluted in running buffer at a flow rate of 5 μL/min contact time: 600 s, yielding a final response of ˜3000 RU. Example compounds were then serially diluted 1:1 in running buffer (10 mM HEPES, 150 mM NaCl, 0.05% T20, pH 7.5, 2% DMSO) from 30 uM to 937.5 nM and flowed over the active and reference surfaces from lowest concentration to greatest concentration in the single-cycle kinetics format, and then allowed to dissociate in running buffer for 7200 s, at 30 μL/min. Sensorgrams were solvent corrected, double-referenced, and the data fitted and analyzed in Biacore Insight Evaluation Software Version 3.0.11.15423.

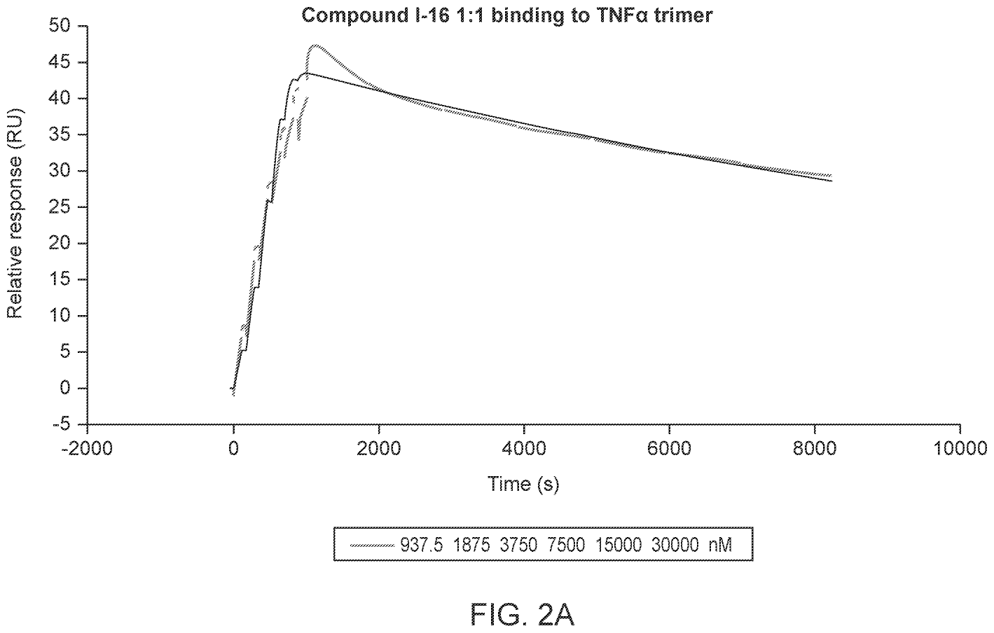

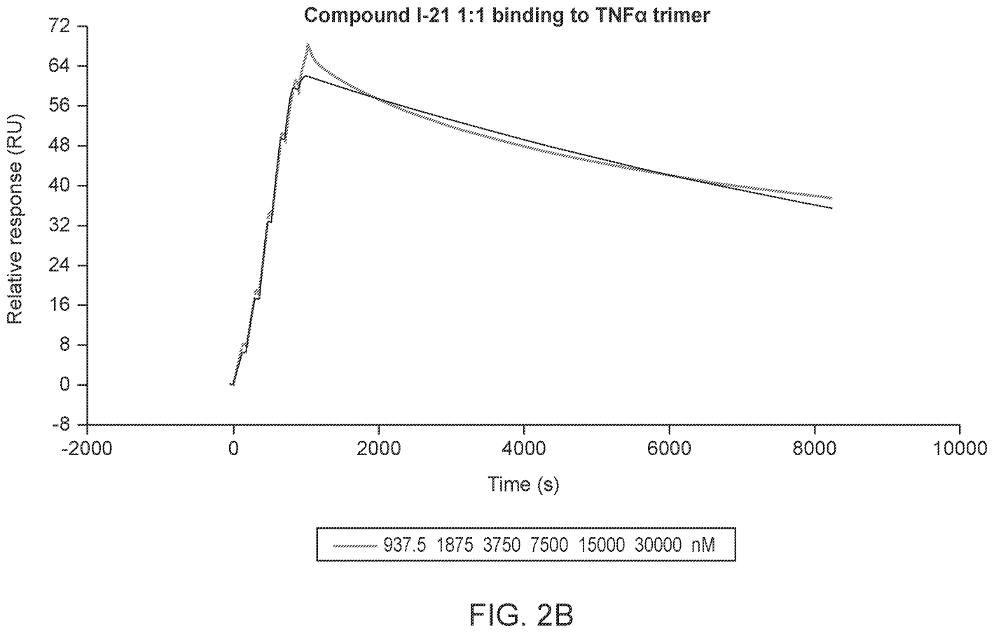

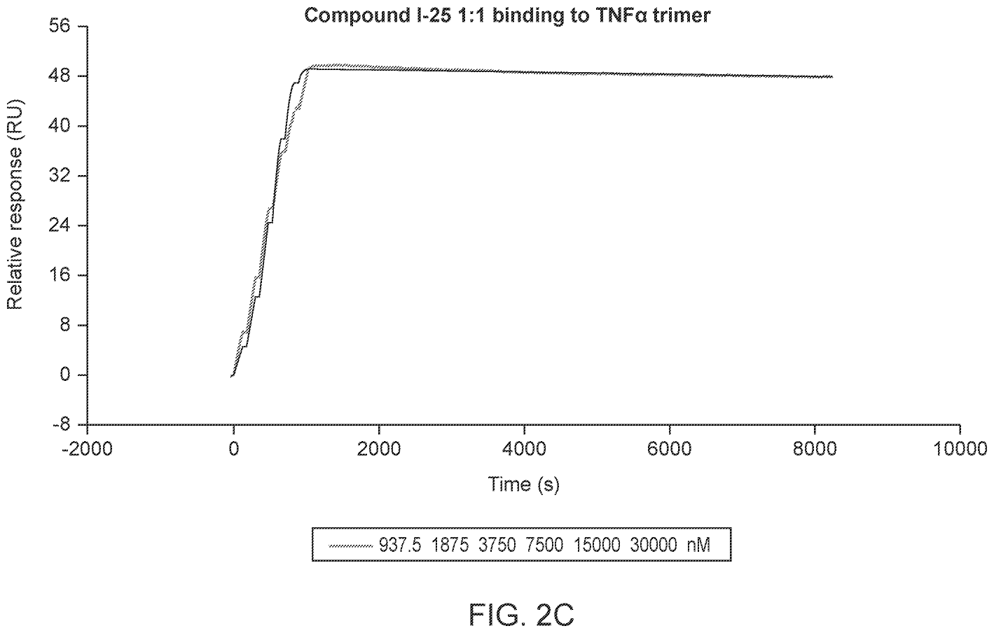

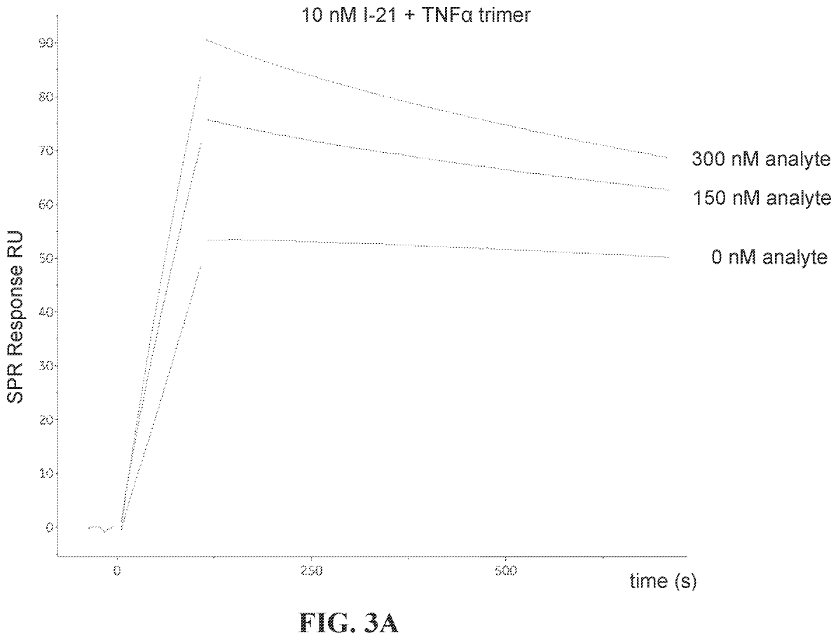

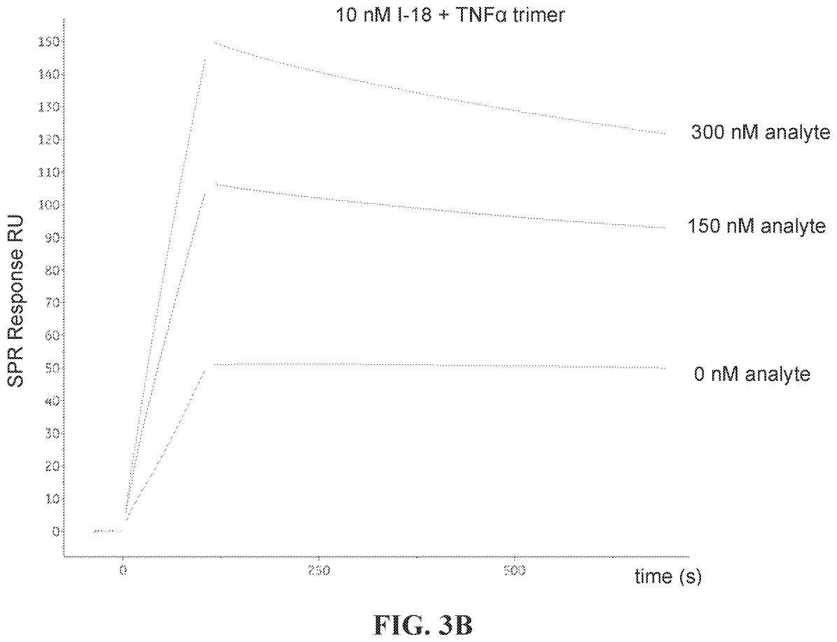

FIG. 2A illustrates the SPR sensorgrams for compound I-16 binding to TNF-alpha trimer, illustrating 1:1 binding.

TABLE 11
SPR results of compound I-16 biding to TNF-alpha trimer

| | | Ligand | Amount | Single cycle | Analyte |
|---|---|---|---|---|---|
| | Immobilized | MW | Immobilized | kinetics 1 | MW |
| Channel | ligand | (Da) | (RU) | Solution | (Da) |
| 6 | (TNFalpha trimer) | 53729.8 | 2847.5 | Example # | 1130.5 |

| Kinetics | | | | | | Expected | |
|---|---|---|---|---|---|---|---|
| Chi² | ka | kd | KD | Rmax | | Rmax | |
| (RU²) | (1/Ms) | (1/s) | (M) | (RU) | tc | (RU) | Stoichiometry |
| 2.50E+00 | 1.16E+03 | 5.81E−05 | 5.02E−08 | 43.7 | 9.74E+09 | 59.9 | 0.7 |

FIG. 2B illustrates the SPR sensorgrams for compound I-21 binding to TNF-alpha trimer, illustrating 1:1 binding.

TABLE 12
SPR results of compound I-21 biding to TNF-alpha trimer

| | | Ligand | | Single cycle | Analyte |
|---|---|---|---|---|---|
| | Immobilized | MW | Amount | kinetics 1 | MW |
| Channel | ligand | (Da) | Immobilized | Solution | (Da) |
| 3 | (TNFalpha trimer) | 53729.8 | 2960.9 | | 1218.7 |

| Kinetics | | | | | | Expected | |
|---|---|---|---|---|---|---|---|
| Chi² | ka | kd | KD | Rmax | | Rmax | |
| (RU²) | (1/Ms) | (1/s) | (M) | (RU) | tc | (RU) | Stoichiometry |
| 1.76E+00 | 9.63E+02 | 7.75E−05 | 8.05E−08 | 62.3 | 2.62E+09 | 67.2 | 0.9 |

FIG. 2C illustrates the SPR sensorgrams for compound I-25 binding to TNF-alpha trimer, illustrating 1:1 binding.

TABLE 13
SPR results of compound I-25 biding to TNF-alpha trimer

| | | Ligand | | Single cycle | Analyte |
|---|---|---|---|---|---|
| | Immobilized | MW | Amount | kinetics 1 | MW |
| Channel | ligand | (Da) | Immobilized | Solution | (Da) |
| 4 | (TNFa trimer) | 53729.8 | 3023.6 | Example # | 1322.4 |

| Kinetics | | | | | | Expected | |
|---|---|---|---|---|---|---|---|
| Chi² | ka | kd | KD | Rmax | | Rmax | |
| (RU²) | (1/Ms) | (1/s) | (M) | (RU) | tc | (RU) | Stoichiometry |
| 9.56E−01 | 8.74E+02 | 3.65E−06 | 4.18E−09 | 49.3 | 1.10E+10 | 74.4 | 0.7 |

### 6.3.3. Example 53: SPR Co-Engagement of Subject Compound-TNFα Trimer Complex to Folate Receptor

The active (Flow cell 2) surfaces of a Cytiva CM5 Series S Sensor Chip functionalized with FOLR2 as described in Example 47. Example compounds were diluted to 10 nM with 50 and 100 nM TNFα single chain trimer or 150 nM and 300 nM TNFα native monomer in running buffer (10 mM HEPES, 150 mM NaCl, 0.05% T20, pH 7.5, 2% DMSO), allowed to incubate for 5 hours at 25° C. and then flowed over the active and reference surfaces for 110 s, and then allowed to dissociate in running buffer for 600 s, at a flow rate of 30 μL/min. The FOLR2 immobilized active surface and the reference surface were regenerated with a 30 s pulse of 4M MgCl₂ at 30 μL/min then allowed to equilibrate 240 s. Sensorgrams were solvent corrected, double-referenced, and the data fitted and analyzed in Biacore Insight Evaluation Software Version 3.0.11.15423.

### 6.3.4. Example 54: Subject Compound Mediated Uptake by TNFα in THP-1 Cells

THP-1 cells, a monocytic leukemia cell line that endogenously express folate receptor beta, were used as a model system to measure example compound stimulated uptake of in-house produced TNFα conjugated to the pH sensitive fluorescent dye pHrodo green (ThermoFisher Scientific #P35369).

THP-1 cells were maintained in RPMI (Gibco #61870143) with 10% v/v FBS (VWR #89510-188) with 2 mM L-alanyl-L-glutamine dipeptide, 100 units/ml penicillin and 100 ug/ml streptomycin (Gibco #15140148). Cells were pelleted, resuspended in folate-free RPMI (Gibco #27016021) and seeded at 50,000 cells per well of a 96 well plate. Example compound was added to each well as a log 2 dilution series ranging from 20 μM to 125 nM final concentration. Cells were incubated for 24 hrs and washed/resuspended in phosphate buffered saline (pH 7.4) with 1% w/v BSA. Uptake was measured using a Novocyte Advanteon Flow Cytometer (Agilent Technologies) with the 488 nM laser and FITC detection configuration.

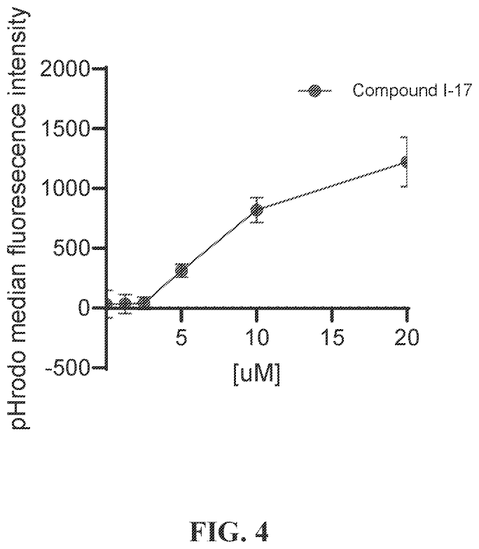

As shown in FIG. 4, treatment with an example compound stimulated uptake of TNFα-pHrodo in a dose-dependent manner as measured by median fluorescence intensity of pHrodo dye.

### 6.3.5. Example 55: Subject Compound Mediated Degradation of TNFα in THP-1 Cells

To measure degradation of internalized TNFα, a pulse-chase experiment was performed whereby cells were treated with +/− example compounds and/or +/− protease inhibitors and incubated for 1 hr in media containing TNFα-biotin to allow uptake and then washed and incubated 90 minutes in fresh media without TNFα to allow degradation. Samples were collected after the pulse phase (60 min) and chase phase (90 min) to measure uptake and degradation, respectively.

THP-1 cells were seeded at 100,000 cells per well of 12 well plate with 1 mL RPMI+10% w/w FBS+1× penicillin/streptomycin and differentiated using 20 ng/mL phorbol myristate acetate (Sigma-Aldrich #P8139) for 72 hrs. After 72 hrs media was replaced with 1 ml RPMI+10% v/v FBS+1× penicillin/streptomycin and TNFα-biotin (AcroBiosystems #50-201-9879) was added to 12.5 nM final concentration. Where noted example compound was added at 10 μM final concentration and protease inhibitors were added at 100 ug/ml final concentration for leupeptin (Sigma-Aldrich #L2884) or 1 μg/ml final concentration for pepstatin A (Sigma-Aldrich #BP26715). While cysteine protease inhibitors such as leupeptin alone have been used to inhibit lysosomal proteases, degradation early in the endocytic pathway in macrophages is initiated by the endosome resident aspartyl protease Cathepsin D that is inhibited by pepstatin A (Diment, J B C, 1985; Diment, J B C, 1988). The plate was incubated for 1 hr at 37° C. in with 5% CO₂. After 1 hr the plate was transferred to ice and washed 5× with cold phosphate buffered saline (pH 7.4). Cells for +/− example compound conditions were lysed in well with 50 ul RIPA buffer (Pierce #89900)+1 mM MgCl₂+25 U/ml benzonase (Sigma-Aldrich #E1014) for 10 min on ice. Lysates were frozen at −80° C. as the uptake samples. 1 ml pre-warmed RPMI+10% FBS+1× penicillin/streptomycin was added to each well and plates were incubated for 90 min at 37° C. in 5% CO₂. The plate was transferred to ice and washed 3× with cold phosphate buffered saline (pH 7.4). Cells were lysed by adding 50 ul RIPA+1 mM MgCl₂+25 U/ml benzonase and incubating on ice for 10 min. Lysates were frozen at −80° C. as the degradation samples. 3.66 ug of each sample was loaded onto to a 4-20% TGX pre-cast gradient gel (BioRad #4561093DC) and separated at 200V for 1 hr using a Mini-PROTEAN gel electrophoresis system (BioRad). Proteins were transferred to PVDF membrane using Transblot Turbo (BioRad) at 1.3 amps for 10 min. The membrane was blocked in tris-buffered saline (pH 7.4) with 5% w/v BSA for 2 hr and incubated overnight at 4° C. with Neutravidin-HRP (ThermoFisher #31001) at 0.4 μg/ml in tris-buffered saline (pH 7.4) with 5% w/v BSA. The blot was developed with ECL Plus Western Detection Substrate (Pierce #32132) and imaged using the ChemiDoc MP Imaging System (BioRad). Relative quantification of bands was performed using the ImageLab software (BioRad) with endogenously biotinylated proteins serving as endogenous controls.

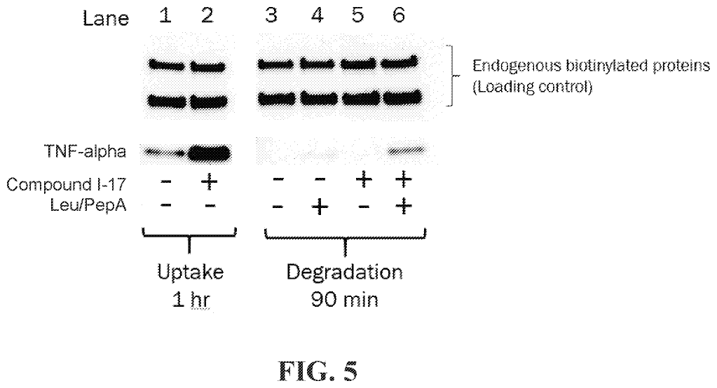

As shown in FIG. 5, treatment with example compounds stimulated the uptake of TNFα during the pulse phase (lane 1 compared to lane 2). After the chase phase, the pool of internalized TNFα was undetectable in the absence of protease inhibitors (lanes 3 and 5), consistent with degradation of internalized TNFα occurring within 90 min. Partial rescue of TNFα level when uptake is in the presence of leupeptin and pepstatin A was consistent with degradation occurring within the endolysosomal pathway (lane 2 compared to lane 6).

### 6.3.6. Example 56: Compound Mediated Depletion of TNFα from the Medium of THP-1 Cells

An in-house recombinantly expressed and purified N-terminal fusion of HiBiT with TNFα was used to measure example compound-stimulated depletion from the media. HiBiT and largeBiT are split nanoluciferase polypeptides that result in complementation of luciferase activity in the presence of both polypeptides. The level of HiBit-TNFα in the media over time can be measure by sampling the media and mixing with the Nano-Glo HiBiT Extracellular Detection Reagent (Promega Corporation) containing the largeBiT polypeptide and nanoluciferase substrate.

THP-1 cells were seeded at 100,000 cells per well of 12 well plate with 1 mL RPMI+10% w/w FBS+1× penicillin/streptomycin and differentiated using 20 ng/mL phorbol myristate acetate (Sigma-Aldrich #P8139) for 72 hrs. After 72 hrs the growth medium was replaced with 1 ml RPMI+10% v/v FBS+1× penicillin/streptomycin containing 12.5 nM HiBit-TNFα. Where noted example compound was added at 10 μM final concentration. The medium was sampled (50 μl) at 0, 24, 48 and 96 hr and luciferase activity was measured using the Nano-Glo HiBiT Extracellular Detection Reagent.

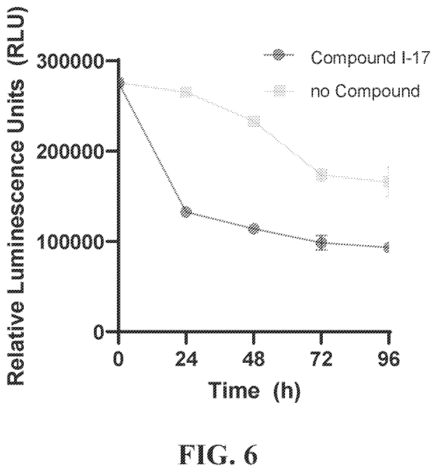

As shown in FIG. 6, treatment with an example compound (I-17) resulted in more rapid depletion of HiBiT-TNFα over the first 24 hours and greater total depletion of HiBiT-TNFα from the culture medium.

### 6.3.7. Example 57: Compound Mediated Uptake of Target Protein IgE in THP-1 Cells

Assessment of a folate receptor-mediated pathway for internalization of a target protein via an exemplary antibody/folate receptor-binding conjugate was performed. An exemplary folate receptor ligand-omalizumab conjugate was generated by non-specific conjugation to lysine residues of in-house produced omalizumab anti-IgE antibody with pentafluorophenyl ester compound (I-4B) N-2-(4-(((2-amino-4-oxo-3,4-dihydropteridin-6-yl)methyl)amino)benzoyl)-N5-(4-(1-(27-oxo-27-(perfluorophenoxy)-3,6,9,12,15,18,21,24-octaoxaheptacosyl)-1H-1,2,3-triazol-4-yl)butyl)-L-glutamine. The conjugate produced yielded an average ligand/antibody ratio (e.g., “DAR”) of 3.71.

IgE (BioRad #HCA171G) was labelled with Alexa-647 (Invitrogen #A20173) according to manufacturer's instructions to produce a fluorescently labelled target protein (IgE) with degree of labeling of 8.25. Uptake of the labelled IgE-Alexa647 was evaluated in a cell-based competition cell uptake assay using wild type (WT) THP-1 cells and folate receptor 2 (FOLR2)-overexpressing THP-1 cells. The THP-1 cells overexpressing FOLR2 were generated by stable transduction with lentivirus encoding FOLR2-IRES-GFP. THP-1 WT and FOLR2 overexpressing cells were maintained in RPMI (Gibco #61870143) with 10% v/v FBS (VWR #89510-188) with 2 mM L-alanyl-L-glutamine dipeptide, 100 units/ml penicillin and 100 ug/ml streptomycin (Gibco #27016021). Stable FOLR2 over-expression cells were mixed at a 50:50 ratio with WT THP-1 cells and seeded in a 96 well plate at 50,000 cells in 100 μl folate-free RPMI (Gibco #61870143) with 10% v/v FBS, 2 mM L-alanyl-L-glutamine dipeptide, 100 units/ml penicillin and 100 ug/ml streptomycin. In a separate 96 well plate, IgE-Alexa647 was mixed with unmodified omalizumab or the exemplary omalizumab-folate receptor ligand conjugate at a concentration of 600 nM each in 100 μl folate-free RPMI+10% FBS+2 μM non-targeting human IgG antibody (to block background binding due to Fc receptor expression in THP-1 cells) and incubated at room temperature for 30 mins. The immune complex was serially diluted 2-fold to produce a dose response range from 300 nM-9.375 nM. The IgE/FR ligand-omalizumab conjugate immune complex mixture was transferred to the plate containing a 50:50 mixture of THP-1 WT and FOLR2 over expressing cells and incubated for 2 h at 37° C. with 5% CO₂.

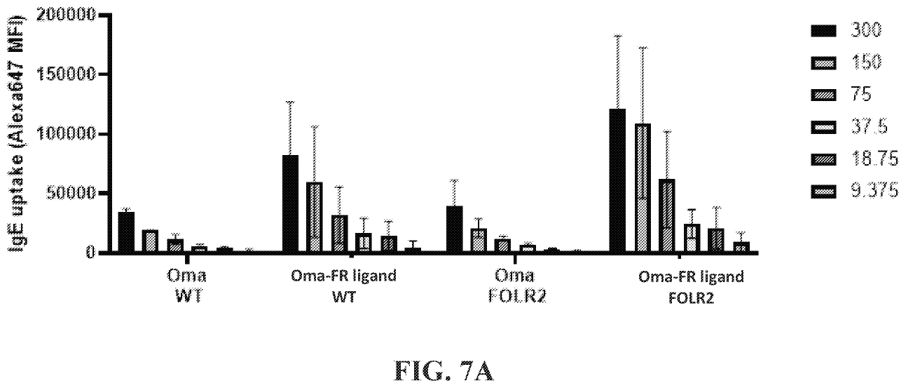

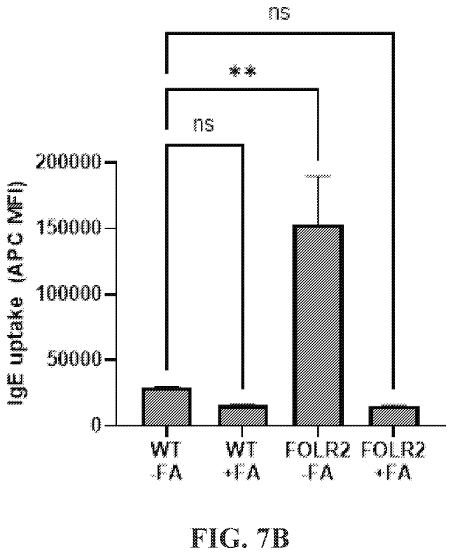

Uptake in WT and FOLR2 overexpressing cells was evaluated using flow cytometry by comparing the median fluorescence intensity of IgE-Alexa647 in the GFP negative (WT) and GFP positive (FOLR2) populations. As shown in FIG. 7A, uptake of IgE-Alexa647 was enhanced across the dose range in both the WT and FOLR2 over expressing cells with the exemplary omalizumab-folate receptor ligand conjugate as compared to unmodified omalizumab control.

To demonstrate that the enhanced IgE-Alexa647 uptake observed with the exemplary conjugate is dependent on folate receptor, the competition experiment, as described above, was performed with 100 nM omalizumab-folate receptor ligand conjugate in the presence or absence of excess folic acid (2 μM). Results were analyzed using one-way ANOVA with multiple comparisons with THP-1 WT and no folic acid condition as the control. As shown in FIG. 7B, increased uptake (p=0.0055) was observed with the exemplary conjugate in FOLR2 overexpressing THP-1 cells compared to WT cells, and addition of folic acid decreased uptake to the WT level. Taken together, these results demonstrated folate receptor dependent uptake of IgE using the omalizumab-folate receptor ligand conjugate.

### 6.3.8. Example 58: Degradation of Target Protein IgE Using an Exemplary Antibody-Folate Receptor Ligand Conjugate in PMA Differentiated THP-1 Cells

To determine that conjugate-mediated cell uptake leads to degradation of target protein the fate of a self-quenched fluorescently labeled target protein was monitored. DQ-BSA-FL (Invitrogen #D12050) is bovine serum albumin (BSA) conjugated with BODIPY dye with a high degree of labeling to produce a self-quenched fluorescent BSA reagent. Upon proteolysis within the endolysosomal pathway of the cell, dequenching of the BODIPY fluorophores results in the appearance of bright fluorescence which can be monitored by live cell imaging. Since DQ-BSA is generally used as a reagent to monitor endocytosis, it was expected that complexing DQ-BSA with anti-BSA antibody conjugated to folate receptor ligand of this disclosure would enhance cell uptake of DQ-BSA, and degradation would be inferred by increased signal that is diminished when treatment was performed in the presence of endolysomal protease inhibitors leupeptin (Sigma-Aldrich #L2884) and pepstatin A (Sigma-Aldrich #BP26715).

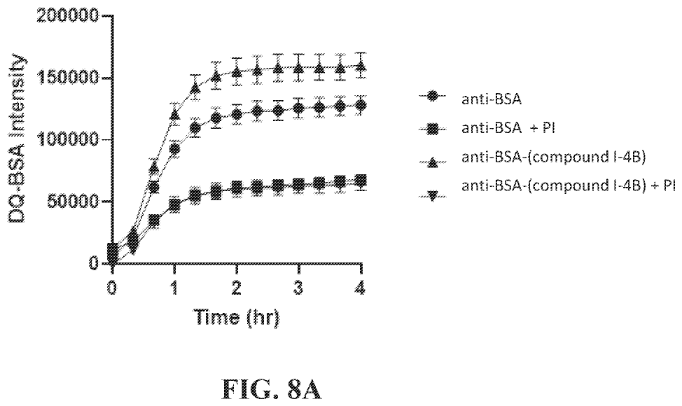

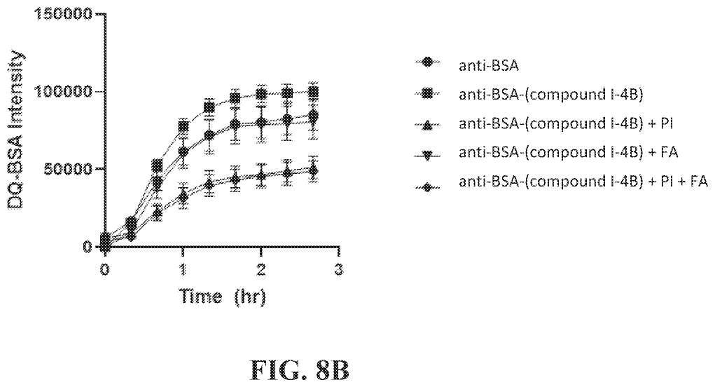

Folate receptor ligand-conjugated anti-BSA was generated by non-specific conjugation to lysine residues of anti-BSA antibody (Invitrogen #A11133) with Compound I-4B. To monitor uptake and degradation, 250,000 THP-1 cells were seeded into a 12 well tissue culture treated plate and differentiated to macrophage-like cells using 20 ng/ml phorbol myristate acetate (Sigma-Aldrich #P8139) for 72 hrs. PMA increases FOLR2 expression (Samaniego, 2020) and facilitates THP-1 live cell imaging by adopting an adherent phenotype upon differentiation. Cells were rinsed 3 times with PBS and 1 ml folate-free RPMI with no serum was added to each well. 100 nM of anti-BSA antibody with or without folate conjugation, 100 nM DQ-BSA and 1 μM of non-targeting antibody was added to each well in the presence or absence of pepstatin A (1 μg/ml) and leupeptin (10 μM) The plate was immediately transferred to the Incucyte live cell imaging system and imaged every 20 m for 4 h. As shown in FIG. 8A, in the presence of protease inhibitors (PI), the intracellular fluorescent signal was diminished. These results demonstrate that the exemplary conjugate enhanced uptake of DQ-BSA, and resulted in proteolysis and dequenching of BODIPY dye in the endolysosomal pathway.

To confirm folate receptor dependence, the experiment above was repeated in the presence or absence of folic acid (2 μM) and/or pepstatin A (1 μg/ml) and leupeptin (10 μM). Images were collected using the Incucyte live cell imaging system every 20 m for 2 h 40 m. As shown in FIG. 8B, in the presence of folic acid (FA), the fluorescent signal of anti-BSA control antibody without folate (anti-BSA) was the same as anti-BSA conjugate with the folate receptor ligand (anti-BSA/Compound I-4B). These results indicate that the stimulation of uptake and degradation of target protein DQ-BSA with the exemplary conjugate is folate receptor-mediated.

### 6.3.9. Example 59: Inhibition of IL-6 Induced by TNFα

TNF signaling leads to the induction of serum IL-6, which can be blocked with biologics such as etanercept (Enbrel). Eight-week-old female C57BL/6 are purchased from Charles River Labs and allowed to acclimate for one week prior to start of study. After the acclimation period, mice are ear tagged, weighed, and orally dosed with vehicle or 3, 10, or 30 mg/kg of example compound or dosed by intraperitoneal (IP) injection with 10 mg/kg positive control (mouse Enbrel). One hour later, mouse TNF-α is administered by intravenous (IV) injection. Two hours after mouse TNF-α treatment, blood is collected via cardiac puncture to measure for serum mouse IL-6 by ELISA from R&D systems.

### 6.3.10. Example 60: Efficacy in Mouse Model of Rheumatoid Arthritis Induced by Collagen Antibodies

Collagen antibodies can be used to induce rheumatoid arthritis in mice. Eight-week-old female BALB/c are purchased from Charles River Labs and allowed to acclimate for one week prior to start of study. After the acclimation period, mice are ear tagged, weighed, and dosed by intraperitoneal (IP) injection with a mixture of four monoclonal anti-mouse type II collagen antibodies at 1 mg per antibody. Three days after antibody administration, 10 μg of lipopolysaccharide (E. coli O111:B4) from Sigma are dosed IP and six hours after lipopolysaccharide injection, vehicle or 3, 10, or 30 mg/kg of example compound is orally dosed twice a day. As a positive control, 10 mg/kg of mouse Enbrel is dosed SC twice a week until the end of the study. Mice are weighed and monitored daily for the development and severity of paw inflammation. The development and severity of paw inflammation is measured by two methods. The first method to measure paw inflammation is to visually evaluate the paws and score based on the severity of inflammation/swelling of the digits and paws. The clinical score is based on the following numbering system: (1) one or more swollen digits per paw; (2) mild paw swelling; (3) moderate paw swelling; (4) fusion of joints/ankylosis with a maximum score of 16 per mouse. The second method to measure paw inflammation is to use the plethysmometer from World Precision Instruments to measure each paw.

### 6.3.11. Example 61: Efficacy in Human Transgenic TNFα Mouse Model of Rheumatoid Arthritis

Human transgenic TNF-α mice express the human TNFα transgene, which develop severe chronic arthritis of the forepaws and hindpaw by approximately 20 weeks of age. Twelve week old female human transgenic TNFα mice are purchased from Taconic and allowed to acclimate for one week prior to start of study. After the acclimation period, mice are ear tagged, weighed, and orally dosed with vehicle or 3, 10, or 30 mg/kg of example compound is orally dosed twice a day or 10 mg/kg of a positive control (mouse Enbrel) is dosed SC twice a week until the end of the study. Mice are weighed and monitored daily for the development and severity of paw inflammation. The development and severity of paw inflammation is measured by two methods. The first method to measure the development and severity of paw inflammation is to visually evaluate the paws and score based on the severity of inflammation/swelling of the digits and paws. The clinical score is based on the following numbering system: (1) one or more swollen digits per paw; (2) mild paw swelling; (3) moderate paw swelling; (4) fusion of joints/ankylosis with a maximum score of 16 per mouse. The second method to measure the development and severity of paw inflammation is to use the plethysmometer from World Precision Instruments to measure each paw.

### 6.3.12. Example 62: Efficacy in Mouse Model of Colitis Induced by Dextran Sodium Sulfate

Dextran Sodium Sulfate is used to induce colitis in mice. Eight-week-old female C57BL/6 are purchased from Charles River Labs and allowed to acclimate for one week prior to start of study. After the acclimation period, mice are ear tagged, weighed, and allowed to drink regular drinking water or 3% DSS in drinking water for the duration of the study. One week after switching the drinking water, vehicle or 3, 10, or 30 mg/kg of example compound is orally dosed twice a day or 10 mg/kg of a positive control (mouse Enbrel) is dosed SC twice a week until the end of the study. Mice are weighed and monitored daily for the development and severity of colitis. The development and severity of colitis is measured by a clinical score composed of body weight, rectal bleeding, and diarrhea and histological score of the colon.

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A cell surface folate receptor binding compound of formula (I): 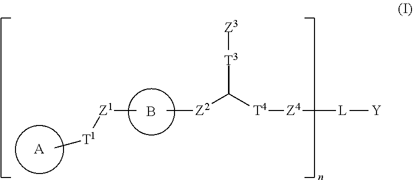
or a salt thereof,
wherein:
T¹ is an optionally substituted (C₁-C₃)alkylene;
Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;
B is a ring system selected from optionally substituted aryl, optionally substituted heteroaryl, optionally substituted heterocycle, optionally substituted cycloalkyl, and optionally substituted bridged bicycle;
Z² is absent, or a linking moiety selected from optionally substituted amide, optionally substituted sulfonamide, optionally substituted urea, optionally substituted thiourea, —NR²¹—, —O—, —S—, and optionally substituted (C₁-C₆)alkylene;
Z³ is carboxyl or carboxyl bioisostere, or a prodrug thereof;
T³ is absent, or is selected from optionally substituted (C₁-C₆)alkylene;
T⁴ is optionally substituted (C₁-C₆)alkylene, or is absent;
Z⁴ is a linking moiety;
each R²¹ is independently selected from H, and optionally substituted (C₁-C₆)alkyl;
n is 1 to 100;
L is a linker;
Y is a moiety of interest; and
A is a ring system of formula (II): 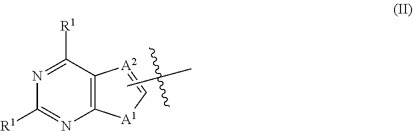
or a tautomer thereof, wherein:
R¹ and R² are independently selected from H, OH, NR²¹, and optionally substituted (C₁-C₆)alkyl;
A¹ is selected from —N═CR³—, —CR³═N—, —CR³═CR³—, NR²¹, S, O, and C(R⁴)₂;
A² is selected from N, and CR³;
each R³ is independently selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each R⁴ is independently selected from H, halogen, and optionally substituted (C₁-C₆)alkyl;
with the proviso that at least one of following applies:
1) T³ is optionally substituted (C₁-C₆)alkylene;
2) L is a non-cleavable linker and Y is an extracellular target-binding moiety;
3) when A is of formula (II-A) or (II-A′), or a tautomer thereof: 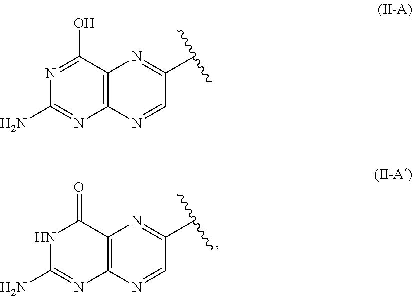 [SMILES: *c1cnc2nc(N)nc(O)c2n1 ; *c1cnc2nc(N)[nH]c(=O)c2n1]
then Z¹ is not NR²¹, and/or B is not 1,4-linked phenyl;
4) when A is of formula (II-B), or a tautomer thereof: 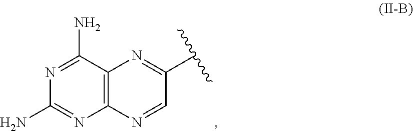 [SMILES: *c1cnc2nc(N)nc(N)c2n1]
then Z¹ is not NR²¹, and/or B is not 1,4-linked phenyl; and/or
5) when A is of formula (II-C) or (II-C′), or a tautomer thereof: 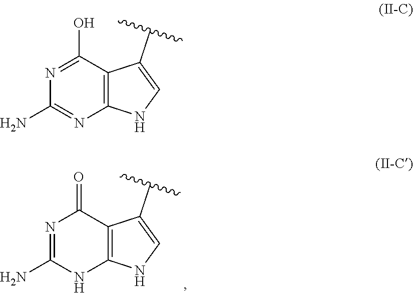 [SMILES: *c1c[nH]c2nc(N)nc(O)c12 ; *c1c[nH]c2[nH]c(N)nc(=O)c12]
then T¹-Z¹ is not —CH₂CH₂—, and/or B is not phenyl.

2-5. (canceled)

6. The compound of claim 1, wherein the compound is of formula (IIIA): 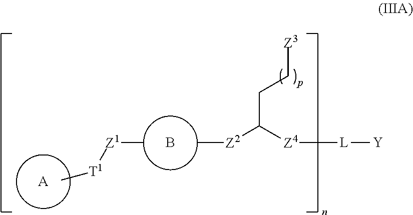
wherein p is 0 or 1.

7-10. (canceled)

11. The compound of claim 1, wherein the compound is of formula (IIIB): 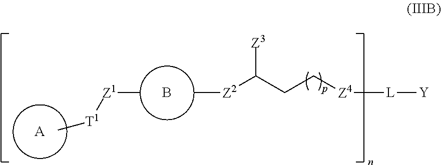
wherein p is 0 or 1.

12. (canceled)

13. The compound of claim 1, wherein Z³ is selected from: 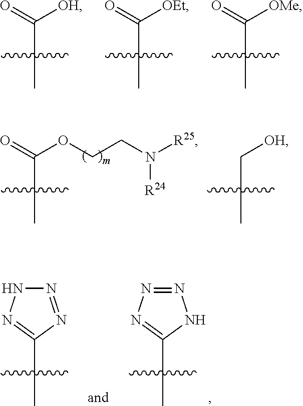
wherein:
R²⁴ and R²⁵ are independently selected from H and optionally substituted (C₁-C₆)alkyl, or R²⁴ and R²⁵ are cyclically linked to provide an optionally substituted 5 or 6-membered heterocycle; and
m is 1 to 5.

14-17. (canceled)

18. The compound of claim 1, wherein Z² is —CONR²¹—, —NR²¹C₀—, —SO₂NR²¹—, —NR²¹C(═O)NR²¹—, or —NR²¹C(═S)NR²¹, wherein each R²¹ is independently selected from H, and optionally substituted (C₁-C₆)alkyl.

19. (canceled)

20. The compound of claim 1, wherein Z⁴ is a linking group selected from:

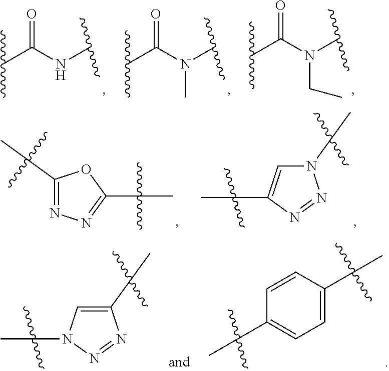

21. The compound of claim 1, wherein —Z²CH(-T³-Z³)T⁴Z⁴— of formula (I) is selected from the following structures: 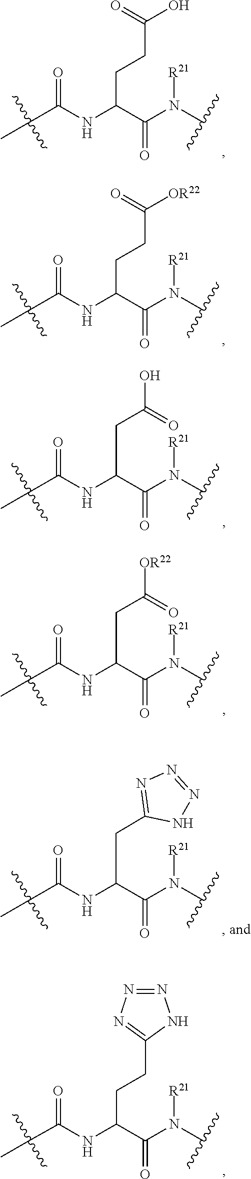
or a tautomer thereof, or a salt thereof.

22. The compound of claim 1, wherein —Z²CH(-T³-Z³)T⁴Z⁴— of formula (I) is selected from the following structures: 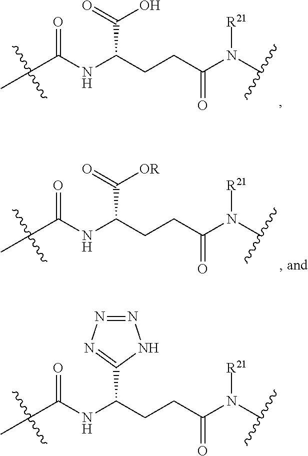
or a tautomer thereof, or a salt thereof.

23. (canceled)

24. The compound of claim 1, wherein A is of formula (IIA): 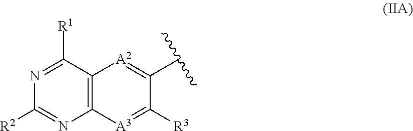
or a tautomer thereof, or a salt thereof, wherein:
A² is selected from N, and CR³;
A³ is independently selected from N, and CR²¹.

25-31. (canceled)

32. The compound of claim 1, wherein A is selected from: 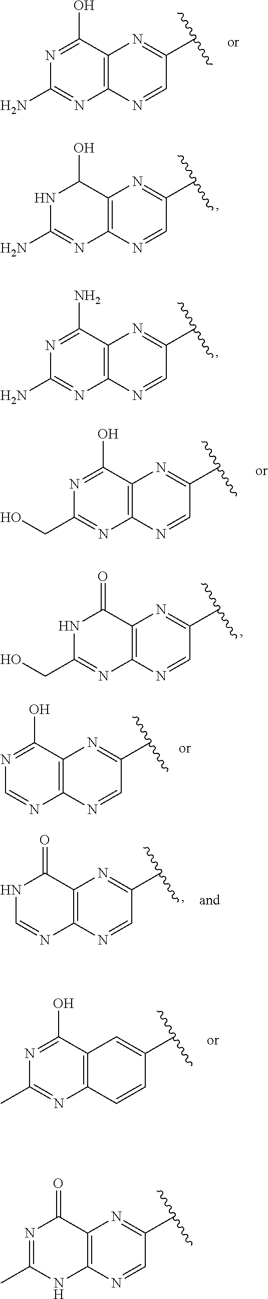
or a tautomer thereof.

33. (canceled)

34. The compound of claim 1, wherein A is of formula (IIB) or (IIC): 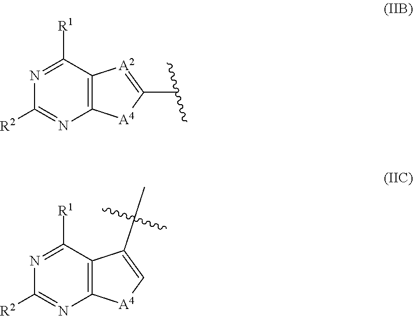
or a tautomer thereof, or a salt thereof, wherein A⁴ is selected from NR²¹, S, and O.

35-39. (canceled)

40. The compound of claim 33, wherein A is: 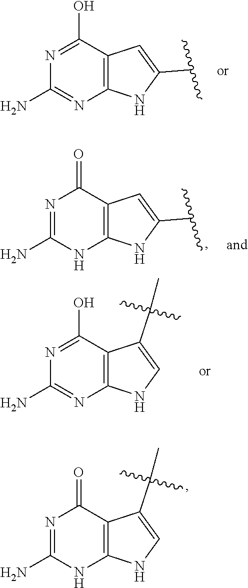 [SMILES: *c1cc2c(O)nc(N)nc2[nH]1 ; *c1cc2c(=O)nc(N)[nH]c2[nH]1 ; *c1c[nH]c2nc(N)nc(O)c12 ; *c1c[nH]c2[nH]c(N)nc(=O)c12]
or a tautomer thereof.

41. The compound of claim 1, wherein:
T¹ is CH₂; and
Z¹ is NR²¹, O or S, wherein R²¹ is H, methyl, ethyl, propyl, or propargyl; or
T¹-Z¹ is optionally substituted (C₁-C₆)alkylene.

42-50. (canceled)

51. The compound of claim 1, wherein —B—Z²— is selected from: 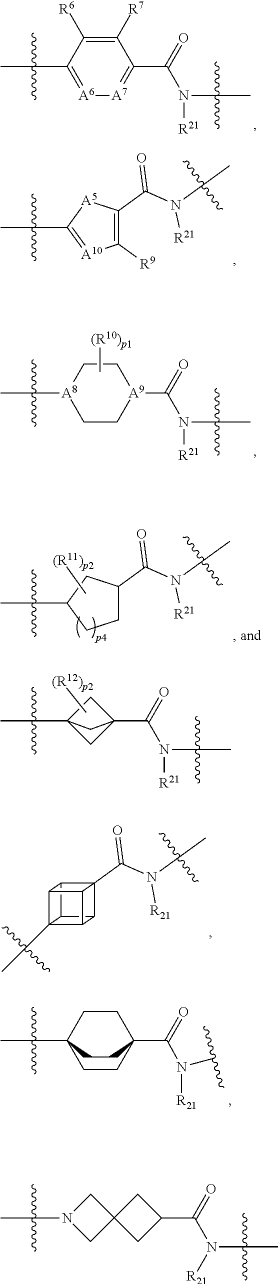
wherein:
A⁵ is selected from NR²¹, S, O, C(R⁵)₂;
A⁶-A⁹ are independently selected from N, and CR⁵; R²¹ is selected from H, and optionally substituted (C₁-C₆)alkyl;
A¹⁰ is selected from N, and CR⁸;
each R⁵ to R¹² is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
p1 is 0 to 10;
p2 is 0 to 14;
p3 is 0 to 4; and
p4 0 to 4.

52. (canceled)

53. The compound of claim 1, wherein A-T¹-Z¹—B— is selected from one of the following: 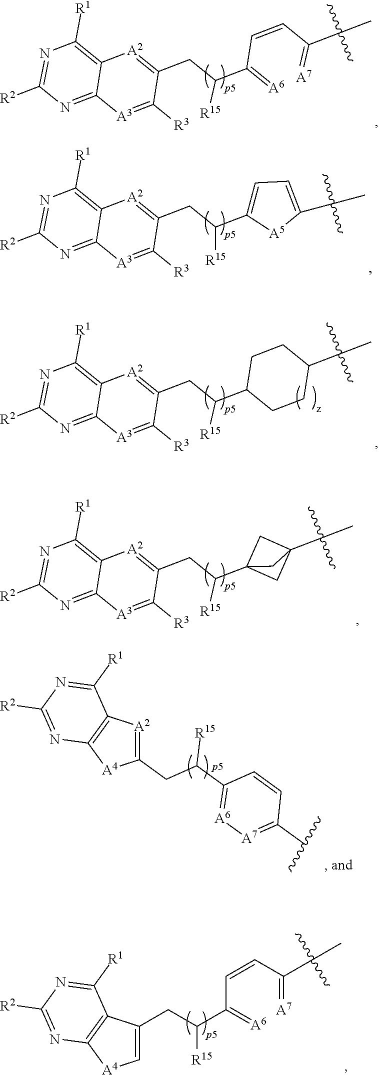
wherein:
A⁵ is selected from NR²¹, S, O, C(R⁵)₂;
A⁶ and A⁷ are independently selected from N, and, CR⁵;
z is 0 to 3
each R⁵ and R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
each p5 is independently 1 to 3.

54. The compound of claim 1, wherein the compound comprises a cell surface folate receptor ligand selected from: 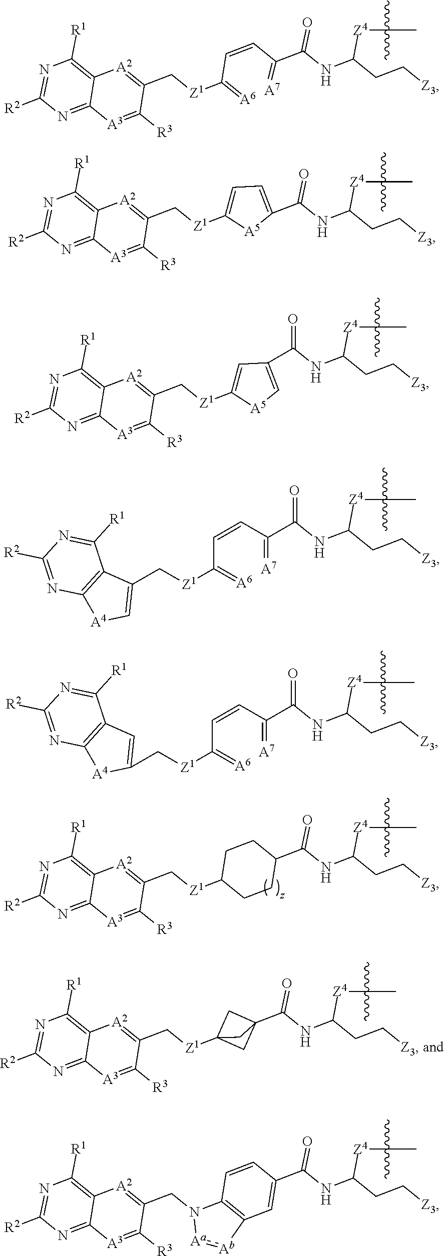
wherein:
A⁵ is selected from NR²¹, S, O, C(R⁵)₂;
A⁶ and A⁷ are independently selected from N, and, CR⁵;
z is 0 to 3; [symbol]
is a single bond or a double bond;
wherein when [symbol] is a single bond A^{a} is selected from C(R⁵)₂, and C═O, and A^{b} is selected from C(R⁵)₂, and NR²¹; and
when [symbol] is a double bond A^{a} is CR⁵, and A^{b} is selected from CR⁵ and N; and
wherein each R⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵.

55. The compound of claim 1, wherein the compound comprises a cell surface folate receptor ligand selected from: 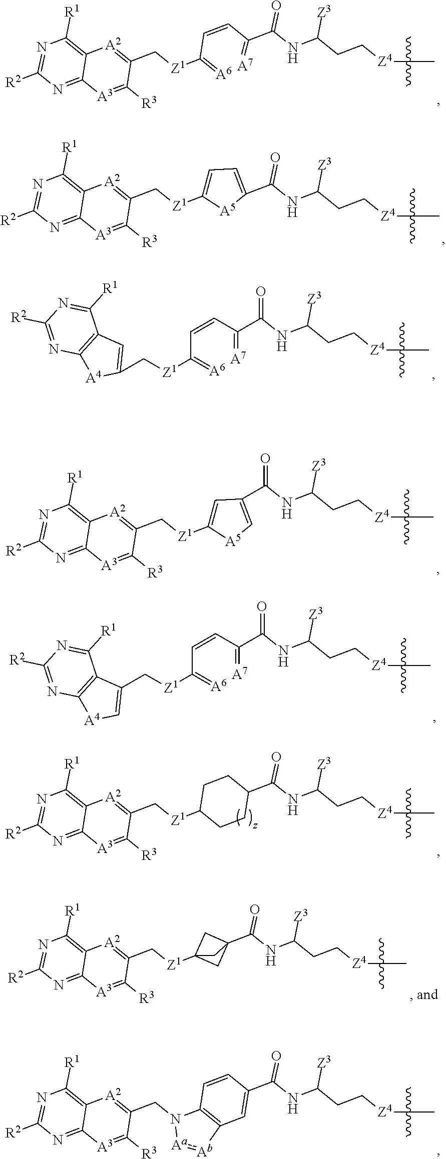
wherein:
A⁵ is selected from NR²¹, S, O, C(R⁵)₂;
A⁶ and A⁷ are independently selected from N, and, CR⁵;
z is 0 to 3; [symbol]
is a single bond or a double bond;
wherein when [symbol] is a single bond A^{a} is selected from C(R⁵)₂, and C═O, and A^{b} is selected from C(R⁵)₂, and NR²¹; and
when [symbol] is a double bond A^{a} is CR⁵, and A^{b} is selected from CR⁵ and N; and
wherein each R⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵.

56. The compound of claim 1, wherein n is 1.

57. The compound of claim 1, wherein n is 2 to 20.

58. The compound of claim 1, wherein L comprises a backbone of at least 10 consecutive atoms.

59-61. (canceled)

62. The compound of claim 1, wherein L is of formula (IV): 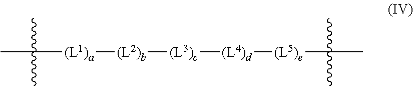
wherein
each L¹ to L⁵ is independently a linking moiety which together provide a linear or branched linker between Z⁴ and Y;
a is 1 or 2; and
b, c, d, and e are each independently 0, 1, or 2.

63-72. (canceled)

73. The compound of claim 1, wherein the linker L is selected from any one of the structures of Table 3.

74. The compound of claim 1, wherein the compound comprises a cell surface folate receptor ligand of one of the structures of Tables 1 or 2.

75. The compound of claim 1, wherein Y is selected from small molecule, dye, fluorophore, monosaccharide, polysaccharide, lipid, enzyme, enzyme substrate and chemoselective ligation group or precursor thereof.

76. The compound of claim 1, wherein Y is a moiety that specifically binds an extracellular target protein.

77-78. (canceled)

79. The compound of claim 1, wherein Y is a small molecule inhibitor or ligand of the target protein.

80. (canceled)

81. The compound of claim 1, wherein Y is a target-binding biomolecule selected from peptide, protein, glycoprotein, polynucleotide, aptamer, and antibody or antibody fragment.

82-83. (canceled)

84. The compound of claim 82, wherein Y is antibody or antibody fragment that specifically binds the target protein and the compound is of formula (VIIIa): 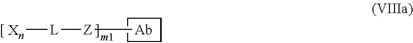
or a pharmaceutically acceptable salt thereof, wherein:
n is 1 to 20;
m1 is an average loading of 1 to 80;
each X is a moiety that binds to a cell surface folate receptor;
each L is a linker;
each Z is a residual moiety resulting from the covalent linkage of a chemoselective ligation group to a compatible group of Ab; and
Ab is the antibody or antibody fragment that specifically binds the target protein.

85. The compound of claim 84, wherein X is not folic acid, methotrexate, or pemetrexed.

86-87. (canceled)

88. The compound of claim 85, wherein n is 1 to 6.

89-93. (canceled)

94. The compound of claim 85, wherein m1 is 1 to 20.

95-98. (canceled)

99. The compound of claim 85, wherein:
Z is a residual moiety resulting from the covalent linkage of a thiol-reactive chemoselective ligation group to one or more cysteine residue(s) of Ab; or
Z is a residual moiety resulting from the covalent linkage of an amine-reactive chemoselective ligation group to one or more lysine residue(s) of Ab.

100-104. (canceled)

105. A method of internalizing a target protein in a cell comprising a cell surface folate receptor, the method comprising:
contacting a cellular sample comprising the cell and the target protein with an effective amount of a compound according to claim 1, wherein the compound specifically binds the target protein and specifically binds the cell surface folate receptor to facilitate cellular uptake of the target protein.

106-114. (canceled)

115. A method of treating a disease or disorder associated with a target protein, the method comprising:
administering to a subject in need thereof an effective amount of a compound according to claim 1, wherein the compound specifically binds the target protein.

116-118. (canceled)


SEQUENCE LISTING (6 sequences)

SEQ ID NO: 1 - protein, 449 aa, Artificial Sequence
  Cetuximab heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Cetuximab light chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 223 aa, Artificial Sequence
  Matuzumab heavy chain Fab
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 4 - protein, 212 aa, Artificial Sequence
  Matuzumab light chain
DIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 5 - protein, 448 aa, Artificial Sequence
  Atezolizumab heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  Atezolizumab light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC